SEQUENCE LISTING (15 sequences)

SEQ ID NO: 1 - DNA, 17 bp, synthetic construct
  source 1..17: mol_type = other DNA
taatacgactcactata

SEQ ID NO: 2 - DNA, 1920 bp, synthetic construct
  source 1..1920: mol_type = other DNA
gggaaataagagagaaaagaagagtaagaagaaatataagagccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgataagctgccttctgcggggcttgccttctggccatgcccttcttctctcccttgcacctgtacctcttggtctttgaataaagcctgagtaggaaggcggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - DNA, 1921 bp, synthetic construct
  source 1..1921: mol_type = other DNA
agggaaataagagagaaaagaagagtaagaagaaatataagagccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgataagctgccttctgcggggcttgccttctggccatgcccttcttctctcccttgcacctgtacctcttggtctttgaataaagcctgagtaggaaggcggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - DNA, 17 bp, synthetic construct
  source 1..17: mol_type = other DNA
taatacgactcactatt

SEQ ID NO: 5 - DNA, 1921 bp, synthetic construct; identical to SEQ ID NO: 3
  source 1..1921: mol_type = other DNA

SEQ ID NO: 6 - DNA, 23 bp, synthetic construct
  source 1..23: mol_type = other DNA
nnnnnnnatagtgagtcgtatta

SEQ ID NO: 7 - DNA, 23 bp, synthetic construct
  source 1..23: mol_type = other DNA
taatacgactcactatagggaga

SEQ ID NO: 8 - DNA, 23 bp, synthetic construct
  source 1..23: mol_type = other DNA
atttaggtgacactatagaagaa

SEQ ID NO: 9 - DNA, 23 bp, synthetic construct
  source 1..23: mol_type = other DNA
aattaaccctcactaaagggaga

SEQ ID NO: 10 - DNA, 1921 bp, synthetic construct
  source 1..1921: mol_type = other DNA
cgggaaataagagagaaaagaagagtaagaagaaatataagagccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgataagctgccttctgcggggcttgccttctggccatgcccttcttctctcccttgcacctgtacctcttggtctttgaataaagcctgagtaggaaggcggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 11 - DNA, 1921 bp, synthetic construct
  source 1..1921: mol_type = other DNA
tgggaaataagagagaaaagaagagtaagaagaaatataagagccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgataagctgccttctgcggggcttgccttctggccatgcccttcttctctcccttgcacctgtacctcttggtctttgaataaagcctgagtaggaaggcggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 12 - DNA, 1921 bp, synthetic construct
  source 1..1921: mol_type = other DNA
aaggaaataagagagaaaagaagagtaagaagaaatataagagccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgataagctgccttctgcggggcttgccttctggccatgcccttcttctctcccttgcacctgtacctcttggtctttgaataaagcctgagtaggaaggcggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - DNA, 1921 bp, synthetic construct
  source 1..1921: mol_type = other DNA
acggaaataagagagaaaagaagagtaagaagaaatataagagccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgataagctgccttctgcggggcttgccttctggccatgcccttcttctctcccttgcacctgtacctcttggtctttgaataaagcctgagtaggaaggcggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - DNA, 1921 bp, synthetic construct
  source 1..1921: mol_type = other DNA
atggaaataagagagaaaagaagagtaagaagaaatataagagccaccatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtgataagctgccttctgcggggcttgccttctggccatgcccttcttctctcccttgcacctgtacctcttggtctttgaataaagcctgagtaggaaggcggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - DNA, 23 bp, synthetic construct
  source 1..23: mol_type = other DNA
taatacgactcactataagggaa

(19) United States
(12) Patent Application Publication
(10) Pub. No.: US 2026/0185136 A1
(43) Pub. Date: Jul. 2, 2026

(54) 5'-CAP MODIFICATIONS OF RNA

(71) Applicant: Eisai R&D Management Co., Ltd., Tokyo (JP)

(72) Inventors: Kosuke NAKAMOTO, Tsukuba (JP); Koki OTSUKA, Katori-Shi (JP)

(21) Appl. No.: 19/432,726

(22) Filed: Dec. 24, 2025

Related U.S. Application Data
(60) Provisional application No. 63/739,479, filed on Dec. 27, 2024.
(60) Provisional application No. 63/890,089, filed on Sep. 29, 2025.

(51) Int. Cl. C12P 19/34; A61K 31/7105; A61K 31/7115; C12N 9/12; C12N 15/11

(52) U.S. Cl. CPC C12P 19/34; A61K 31/7105; A61K 31/7115; C12N 9/1247; C12N 15/11; C12Y 207/07006

(57) ABSTRACT

The present disclosure provides capping primers and systems thereof for use in methods of synthesizing 5′-capped RNAs. The disclosure further provides 5′-capped RNAs generated by the methods and systems described herein.

### CROSS REFERENCE TO RELATED APPLICATION

This application claims priority to U.S. Provisional Patent Application No. 63/739,479, filed Dec. 27, 2024, and U.S. Provisional Patent Application No. 63/890,089, filed Sep. 29, 2025, all of which are incorporated herein by reference in their entirety.

### REFERENCE TO AN ELECTRONIC SEQUENCE LISTING

The contents of the electronic sequence listing (EISA_001_01US_SeqList_ST26.xml; Size: 29,498 bytes; and Date of Creation: Dec. 10, 2025) are herein incorporated by reference in their entirety.

### BACKGROUND

Engineered RNAs used in therapeutic and industrial applications commonly include a 5′-cap structure. For example, protein-encoding messenger RNAs (mRNAs), such as mRNA-based vaccines and therapeutics (e.g., therapeutics designed for enzyme replacement, cancer immunotherapy, gene editing, and other applications), include a 5′-cap that is an important feature for stability and function. The 2′—OH groups on the first two transcribed nucleosides at the 5′-end are generally methylated in natural 5′-capped mRNA. 5′-capped mRNA having zero, one, or two 2′-O-methyl (OMe) groups at the first two transcribed nucleosides are known in the art as mRNA having Cap-0, Cap-1, or Cap-2 structures, respectively. Self-amplifying RNA is another example of a protein-coding 5′-capped RNA that is used to produce large amounts of a protein of interest and is useful, e.g., in transient protein expression systems.

In vitro transcription (IVT) is typically used for synthesizing RNA (e.g., mRNA), particularly RNA having lengths greater than about 200 nucleotides. The 5′-cap is either introduced concurrently during IVT (in a one-step process termed “co-transcriptional capping”), or enzymatically after IVT (in a two-step process termed “enzymatic capping”). Co-transcriptional capping is generally better than enzymatic capping in terms of cost-efficiency and quality control.

5′-capped mRNA comprising a Cap-2 structure offers benefits compared to 5′-capped mRNA comprising a Cap-0 or Cap-1 structure (e.g., reduced immunogenicity and/or increased translational efficiency). However, conventional methods for co-transcriptional capping to generate RNA comprising a Cap-2 structure suffer from poor efficiency, which in turn limits their application in large-scale synthesis of RNA therapeutics due to increased cost and labor expenditures.

Accordingly, there remains a need for capping primers and systems for use in methods of synthesizing 5′-capped RNA with improved capping efficiency. The capping primers, systems, kits, and methods of the present disclosure address this need.

### SUMMARY

In some aspects, the present disclosure provides a capping primer comprising a structure according to Formula II:

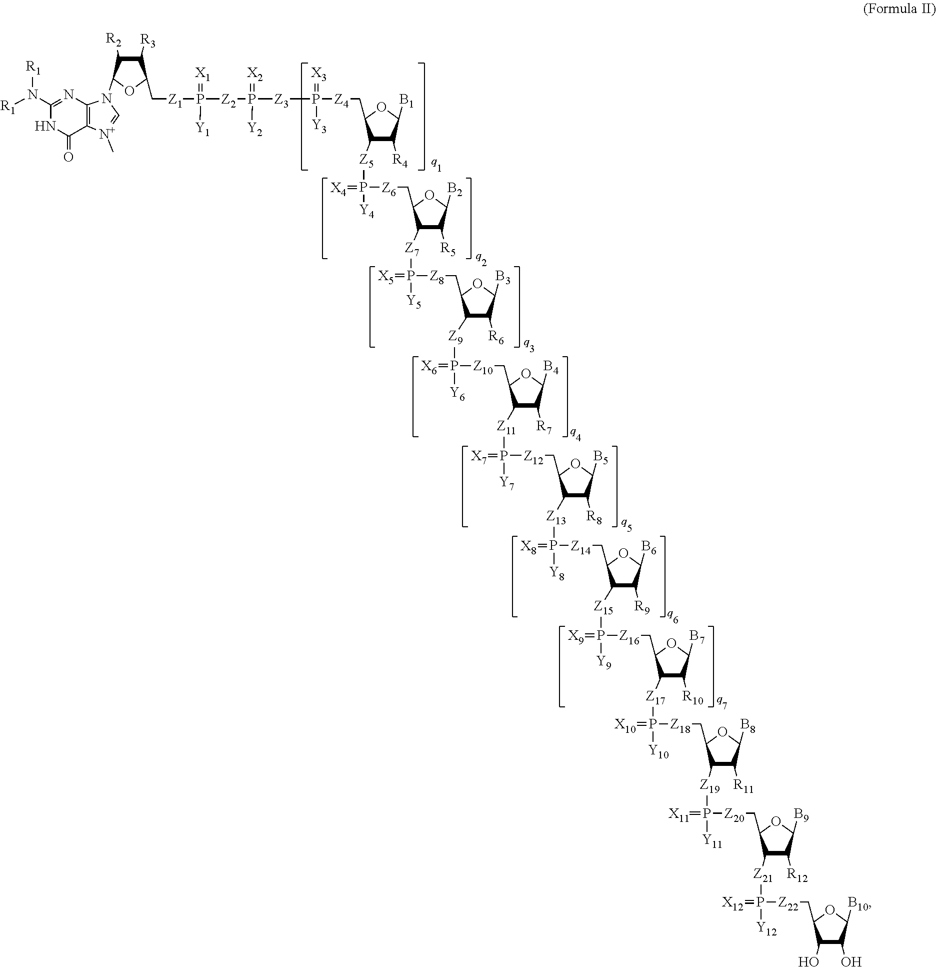

wherein, q1 to q7, are each independently 0 or 1; B1 through B7, if present, are each a nucleobase independently selected from the group consisting of adenine, guanine, thymine, uracil, cytosine and any derivative or analog of the foregoing; B8 is a nucleobase selected from the group consisting of adenine, guanine, thymine, uracil, cytosine and any derivative or analog of the foregoing; B9 is a nucleobase selected from the group consisting of adenine, uracil, cytosine and any derivative or analog of the foregoing; B10 is a nucleobase selected from the group consisting of adenine, guanine, uracil, cytosine and any derivative or analog of the foregoing; R1 is each independently H, alkyl, acyl, benzyl, a cleavable unit; R2 and R3 are each independently H, OH, alkyl, O-alkyl, O-acyl, O-benzyl, halogen, LNA, a linker, a cleavable unit or a detectable marker; R4 to R12, if present, are each independently H, OH, alkyl, O-alkyl, O-acyl, O-benzyl, halogen, LNA, a linker, a cleavable unit or a detectable marker; X1 to X12, if present, are each independently O or S; Y1 to Y12, if present, are each independently O, S, BH₃, aryl, alkyl, O-alkyl or O-aryl; and Z1 to Z22, if present, are each independently O, S, NH, CH₂, C(halogen)₂ or CH(halogen); wherein, the capping primer hybridizes to a polynucleotide template, wherein said polynucleotide template comprises a nucleobase at position −1 immediately adjacent to and downstream (3′) of a transcriptional start site and a nucleobase at each of position +1, position +2 and position +3 immediately adjacent to and upstream (5′) of the transcriptional start site; and wherein, B8 is complementary or mismatched to a nucleobase at position −1 of the polynucleotide template; B9 is complementary to a nucleobase at position +1 of the polynucleotide template; and B10 is complementary to a nucleobase at position +2 of the polynucleotide template. In some embodiments, B8, B9, and B10 are not fully complementary to nucleobases at positions +1 to +3 of the polynucleotide template.

In some aspects, the disclosure provides a capping primer comprising a structure according to Formula II, or a pharmaceutically acceptable salt thereof, wherein, q1 to q7, are each independently 0 or 1; B1 through B7, if present, are each a nucleobase independently selected from the group consisting of adenine, guanine, thymine, uracil, cytosine, hypoxanthine, and any derivative or analog of the foregoing; B8 is a nucleobase selected from the group consisting of adenine, guanine, thymine, uracil, cytosine, hypoxanthine, and any derivative or analog of the foregoing; B9 is a nucleobase selected from the group consisting of adenine, uracil, cytosine, and any derivative or analog of the foregoing; B10 is a nucleobase selected from the group consisting of adenine, guanine, uracil, cytosine, and any derivative or analog of the foregoing; R1 are each independently H, alkyl, acyl, benzyl, or a cleavable unit; R2 is H, OH, alkyl, O-alkyl, O-acyl, O-benzyl, O-methoxyethyl (MOE), halogen, LNA, a linker, a cleavable unit, or a detectable marker; R3 is H, OH, alkyl, O-alkyl, O-acyl, O-benzyl, O-methoxyethyl (MOE), halogen, a linker, a cleavable unit, or a detectable marker; R4 to R12, if present, are each independently H, OH, alkyl, O-alkyl, O-methoxyethyl (MOE), halogen, LNA, a linker, or a detectable marker; X1 to X12, if present, are each independently O or S; Y1 to Y12, if present, are each independently O⁻, S⁻, BH₃⁻, aryl, alkyl, O-alkyl, or O-aryl; and Z1 to Z22, if present, are each independently O, S, NH, CH₂, C(halogen)₂, or CH(halogen); wherein, the capping primer hybridizes to a polynucleotide template] said polynucleotide template comprises a nucleobase at position −1 immediately adjacent to and downstream (3′) of a transcriptional start site, and a nucleobase at each of position +1, position +2 and position +3 immediately adjacent to and upstream (5′) of the transcriptional start site; and wherein, B8 is complementary or mismatched to a nucleobase at position −1 of the polynucleotide template; B9 is complementary to a nucleobase at position +1 of the polynucleotide template; and B10 is complementary to a nucleobase at position +2 of the polynucleotide template; and B8, B9, and B10 are not fully complementary to nucleobases at positions +1 to +3 of the polynucleotide template. In some aspects, the disclosure provides a system comprising a capping primer and a polynucleotide template, wherein the capping primer comprises a structure according to Formula II, wherein q1 to q7, are each independently 0 or 1; B1 through B7, if present, are each a nucleobase independently selected from the group consisting of adenine, guanine, thymine, uracil, cytosine and any derivative or analog of the foregoing; B8 is a nucleobase selected from the group consisting of adenine, guanine, thymine, uracil, cytosine and any derivative or analog of the foregoing; B9 is a nucleobase selected from the group consisting of adenine, uracil, cytosine and any derivative or analog of the foregoing; B10 is a nucleobase selected from the group consisting of adenine, guanine, uracil, cytosine and any derivative or analog of the foregoing; R1 is each independently H, alkyl, acyl, benzyl, a cleavable unit; R2 and R3 are each independently H, OH, alkyl, O-alkyl, O-acyl, O-benzyl, halogen, LNA, a linker, a cleavable unit or a detectable marker; R4 to R12, if present, are each independently H, OH, alkyl, O-alkyl, O-acyl, O-benzyl, halogen, LNA, a linker, a cleavable unit or a detectable marker; X1 to X12, if present, are each independently O or S; Y1 to Y12, if present, are each independently O, S, BH₃, aryl, alkyl, O-alkyl or O-aryl; and Z1 to Z22, if present, are each independently O, S, NH, CH₂, C(halogen)₂ or CH(halogen); wherein the polynucleotide template comprises a nucleobase at position −1 immediately adjacent to and downstream (3′) of a transcriptional start site and a nucleobase at each of position +1, position +2 and position +3 immediately adjacent to and upstream (5′) of the transcriptional start site, and wherein B8 is complementary or mismatched to a nucleobase at positions −1 of the polynucleotide template; B9 is complementary to a nucleobase at position +1 of the polynucleotide template; and B10 is complementary to a nucleobase at position +2 of the polynucleotide template. In some embodiments, B8, B9, and B10 are not fully complementary to nucleobases at positions +1 to +3 of the polynucleotide template.

In some aspects, the disclosure provides a system comprising a capping primer and a polynucleotide template, wherein the capping primer comprises a structure according to Formula II, or a pharmaceutically acceptable salt thereof, wherein q1 to q7, are each independently 0 or 1; B1 through B7, if present, are each a nucleobase independently selected from the group consisting of adenine, guanine, thymine, uracil, cytosine, hypoxanthine, and any derivative or analog of the foregoing; B8 is a nucleobase selected from the group consisting of adenine, guanine, thymine, uracil, cytosine, hypoxanthine, and any derivative or analog of the foregoing; B9 is a nucleobase selected from the group consisting of adenine, uracil, cytosine, and any derivative or analog of the foregoing; B10 is a nucleobase selected from the group consisting of adenine, guanine, uracil, cytosine, and any derivative or analog of the foregoing; R1 are each independently H, alkyl, acyl, benzyl, or a cleavable unit; R2 is H, OH, alkyl, O-alkyl, O-acyl, O-benzyl, O-MOE, halogen, LNA, a linker, a cleavable unit, or a detectable marker; R3 is H, OH, alkyl, O-alkyl, O-acyl, O-benzyl, O-MOE, halogen, a linker, a cleavable unit, or a detectable marker; R4 to R12, if present, are each independently H, OH, alkyl, O-alkyl, O-MOE, halogen, LNA, a linker, or a detectable marker; X1 to X12, if present, are each independently O or S; Y1 to Y12, if present, are each independently O⁻, S⁻, BH₃⁻, aryl, alkyl, O-alkyl, or O-aryl; and Z1 to Z22, if present, are each independently O, S, NH, CH₂, C(halogen)₂, or CH(halogen); wherein the polynucleotide template comprises a nucleobase at position −1 immediately adjacent to and downstream (3′) of a transcriptional start site, and a nucleobase at each of position +1, position +2 and position +3 immediately adjacent to and upstream (5′) of the transcriptional start site, and wherein B8 is complementary or mismatched to a nucleobase at positions −1 of the polynucleotide template; B9 is complementary to a nucleobase at position +1 of the polynucleotide template; B10 is complementary to a nucleobase at position +2 of the polynucleotide template; and B8, B9, and B10 are not fully complementary to nucleobases at positions +1 to +3 of the polynucleotide template.

In some embodiments of the foregoing or related aspects, the capping primer consists of the structure according to Formula II.

In some embodiments of the foregoing or related aspects, q1 to q7 are 0; R1 is each independently H, alkyl, acyl, benzyl or a cleavable unit; R2 and R3 are each independently H, OH, alkyl, O-alkyl, O-acyl, O-benzyl, halogen, LNA, a linker, a cleavable unit or a detectable marker; and R11 and R12 are O-alkyl.

In some embodiments of the foregoing or related aspects, at least one of q1 to q7 is 1. In some embodiments, at least two of q1 to q7 is 1. In some embodiments, at least three of q1 to q7 is 1. In some embodiments, at least four of q1 to q7 is 1. In some embodiments, at least five of q1 to q7 is 1. In some embodiments, at least six of q1 to q7 is 1. In some embodiments, at least seven of q1 to q7 is 1.

In some embodiments of the foregoing or related aspects, q1 is 1 and q2 to q7 are each 0. In some embodiments, B1 is mismatched to a nucleobase at position −2 of the polynucleotide template. In some embodiments, B1 is complimentary to a nucleobase at position −2 of the polynucleotide template. In some embodiments, B1 is selected from the group consisting of adenine, uracil, thymine, cytosine, 5-methylcytosine, guanine, and hypoxanthine. In some embodiments, B1 is adenine, or a derivative or analog thereof. In some embodiments, B1 is uracil, or a derivative or analog thereof. In some embodiments, B1 is guanine, or a derivative or analog thereof. In some embodiments, B1 is cytosine, or a derivative or analog thereof.

In some embodiments of the foregoing or related aspects, B8 is adenine or N6-methyladenine. In some embodiments, B8 is not N6-modified adenine. In some embodiments, B8 is a nucleobase selected from the group consisting of adenine, uracil, thymine, cytosine, 5-methylcytosine, guanine, and hypoxanthine. In some embodiments, B8 is uracil.

In some embodiments of the foregoing or related aspects, B9 is selected from the group consisting of adenine, cytosine, 5-methylcytosine, thymine and uracil. In some embodiments, B9 is selected from the group consisting of adenine, cytosine, and uracil. In some embodiments, B9 is adenine or uracil. In some embodiments, B9 is adenine.

In some embodiments of the foregoing or related aspects, B10 is selected from the group consisting of adenine, guanine, cytosine, 5-methylcytosine, thymine and uracil. In some embodiments, B10 is selected from the group consisting of adenine, guanine, cytosine, and uracil. In some embodiments, B10 is guanine.

In some embodiments of the foregoing or related aspects, B9 is adenine, or a derivative or analog thereof, and B10 is guanine, or a derivative or analog thereof. In some embodiments, (i) the nucleobase at position +1 of the polynucleotide template is thymine, and (ii) the nucleobase at position +2 of the polynucleotide template is cytosine. In some embodiments, B9 is cytosine, or a derivative or analog thereof; and B10 is guanine, or a derivative or analog thereof. In some embodiments, (i) the nucleobase at position +1 of the polynucleotide template is guanine, and (ii) the nucleobase at position +2 of the polynucleotide template is cytosine. In some embodiments, B9 is uracil, or a derivative or analog thereof; and B10 is guanine, or a derivative or analog thereof. In some embodiments, (i) the nucleobase at position +1 of the polynucleotide template is adenine, and (ii) the nucleobase at position +2 of the polynucleotide template is cytosine. In some embodiments, B9 is adenine, or a derivative or analog thereof, and B10 is adenine, or a derivative or analog thereof. In some embodiments, (i) the nucleobase at position +1 of the polynucleotide template is thymine, and (ii) the nucleobase at position +2 of the polynucleotide template is thymine. In some embodiments, B9 is adenine, or a derivative or analog thereof; and B10 is cytosine, or a derivative or analog thereof. In some embodiments, (i) the nucleobase at position +1 of the polynucleotide template is thymine, and (ii) the nucleobase at position +2 of the polynucleotide template is guanine. In some embodiments, B9 is adenine, or a derivative or analog thereof; and B10 is uracil, or a derivative or analog thereof. In some embodiments, (i) the nucleobase at position +1 of the polynucleotide template is thymine, and (ii) the nucleobase at position +2 of the polynucleotide template is adenine. In some embodiments, the nucleobase at position −1 of the polynucleotide template is thymine or adenine.

In some embodiments of the foregoing or related aspects, R2 is H, OH, O—(C1-C6 alkyl), O-MOE, fluoro, or LNA. In some embodiments, R2 is OH.

In some embodiments of the foregoing or related aspects, R3 is H, OH, O—(C1-C6 alkyl), O-MOE, or fluoro. In some embodiments, R3 is OH or OMe. In some embodiments, R3 is OMe.

In some embodiments of the foregoing or related aspects, R4 to R10, if present, and R11 and R12 are each independently H, OH, O—(C1-C6 alkyl), O-MOE, fluoro, or LNA. In some embodiments, R4 to R10, if present, are each independently H, OH, OMe, O-MOE, fluoro, or LNA, R11 is H, OH, OMe, O-MOE, fluoro, or LNA, and R12 is OH or OMe. In some embodiments, R4 to R10, if present, and R11 and R12 are OMe.

In some embodiments of the foregoing or related aspects, R1 are each independently H or C1-C6 alkyl. In some embodiments, R1 are each H.

In some embodiments of the foregoing or related aspects, X1 to X12, if present, are each independently O; Y1 to Y12, if present, are each independently O⁻ or S⁻; and Z1 to Z22, if present, are each independently O.

In some aspects, the disclosure provides a stereoisomer, tautomer or salt form of a capping primer described herein.

In some embodiments of the foregoing or related aspects, B8 is complementary to a nucleobase at position −1 of the polynucleotide template. In some embodiments, B8 is mismatched to a nucleobase at position −1 of the polynucleotide template.

In some aspects, the disclosure provides a method of producing 5′-capped RNA molecules in an IVT reaction comprising the steps of: mixing a capping primer described herein with a polynucleotide template, nucleoside 5′-triphosphates and a RNA polymerase; and incubating the mixture under transcription conditions, thereby resulting in synthesis of 5′-capped RNA molecules.

In some aspects, the disclosure provides a method of producing 5′-capped RNA molecules in an IVT reaction comprising the steps of: mixing a system described herein with nucleoside 5′-triphosphates and a RNA polymerase; and incubating the mixture under transcription conditions, thereby resulting in synthesis of 5′-capped RNA molecules.

In some embodiments of the foregoing or related aspects, the RNA polymerase is a T7 RNA polymerase or a variant thereof, a T3 RNA polymerase or a variant thereof, or a SP6 RNA polymerase or a variant thereof. In some embodiments, the RNA polymerase is a T7 RNA polymerase or a variant thereof.

In some embodiments of the foregoing or related aspects, the capping primer and the nucleoside 5′-triphosphates (NTPs) are present in the IVT reaction at approximately equimolar concentrations. In some embodiments, the NTPs comprise adenosine triphosphate (ATP) or a derivative or analog thereof, guanosine triphosphate (GTP) or a derivative or analog thereof, cytosine triphosphate (CTP) or a derivative or analog thereof, and uridine triphosphate (UTP) or a derivative or analog thereof, each at an approximately equimolar concentration, optionally wherein the derivative or analog of UTP is pseudouridine triphosphate (pseudo-UTP) or N1-methylpseudouridine triphosphate (N1-pseudo-UTP). In some embodiments, the NTPs comprise ATP, GTP, CTP, and UTP at an approximately equimolar concentration, optionally wherein UTP is pseudo-UTP or N1-pseudo-UTP, and wherein (i) each NTP is present in the IVT reaction at a molar concentration that is about 1. 1-fold, 1. 2-fold, 1. 3-fold, 1. 4-fold, 1. 5-fold, 2-fold, 2. 5-fold, 3-fold, 4-fold, 5-fold, or 10-fold higher than a molar concentration of the capping primer, or (ii) the capping primer is present in the IVT reaction at a molar concentration that is about 1. 1-fold, 1. 2-fold, 1. 3-fold, 1. 4-fold, 1. 5-fold, 1. 6-fold, 1. 7-fold, 1. 8-fold, 1. 9-fold, 2-fold, 2. 5-fold, 3-fold, 4-fold, or 5-fold higher than a molar concentration of each NTP.

In some embodiments of the foregoing or related aspects, at least about 70%, about 75%, about 80%, about 85%, about 90%, about 91%, about 92%, about 93%, about 94%, about 95%, about 96%, about 97%, about 98%, or about 99% of the RNA molecules produced by the IVT reaction are 5′-capped. In some embodiments, at least about 65%, about 70%, about 75%, about 80%, about 85%, about 90%, about 91%, about 92%, about 93%, about 94%, about 95%, about 96%, about 97%, about 98%, or about 99% of the RNA molecules produced by the IVT reaction are 5′-capped. In some embodiments, the synthesis results in a yield of RNA molecules that is at least about 60%, about 70%, about 75%, about 80%, about 85%, about 90%, about 91%, about 92%, about 93%, about 94%, about 95%, about 96%, about 97%, about 98%, or about 99% relative to the RNA yield obtained in the reaction without using a primer. In some embodiments, the synthesis results in a yield of RNA molecules that is at least about 1. 1-fold, 1. 5-fold, 2-fold, 3-fold, 4-fold, 5-fold, 6-fold, 7-fold, 8-fold, 9-fold, or 10-fold higher relative to the RNA yield obtained in the reaction without using a primer. In some embodiments, the synthesis results in (a) a yield of RNA molecules that is at least about 30% relative to the RNA yield obtained in the reaction without using a primer; and (b) at least about 30% of the RNA molecules produced are 5′-capped. In some embodiments, the synthesis results in (a) a yield of RNA molecules that is at least about 50% relative to the RNA yield obtained in the reaction without using a primer; and (b) at least about 50% of the RNA molecules produced are 5′-capped. In some embodiments, the synthesis results in (a) a yield of RNA molecules that is at least about 70% relative to the RNA yield obtained in the reaction without using a primer; and (b) at least about 70% of the RNA molecules produced are 5′-capped.

In some embodiments of the foregoing or related aspects, the method further comprises a step of purifying the 5′-capped RNA molecules.

In some embodiments of the foregoing or related aspects, the polynucleotide template is a DNA template. In some embodiments, the DNA template is a partially or fully double-stranded DNA template. In some embodiments, the polynucleotide template is linear DNA. In some embodiments, the DNA template comprises a sequence encoding a 5′ untranslated region (UTR), an open-reading frame (ORF), a 3′UTR, and a poly-A sequence. In some embodiments, the polynucleotide template is an RNA template. In some embodiments, the polynucleotide template is a template comprising RNA and DNA.

In some aspects, the disclosure provides 5′-capped RNA molecules produced by a method described herein. In some embodiments, the 5′-capped RNA molecules are 5′-capped mRNAs.

In some aspects, the disclosure provides a cell comprising 5′-capped RNA molecules described herein.

In some aspects, the disclosure provides a pharmaceutical composition comprising 5′-capped RNA molecules described herein. In some embodiments, the pharmaceutical composition comprises the 5′-capped RNA molecules, wherein the pharmaceutical composition is formulated as a lipid nanoparticle (LNP).

In some aspects, the disclosure provides an LNP comprising a 5′-capped RNA molecule described herein.

In some aspects, the disclosure provides a kit comprising a capping primer described herein, or a system described herein, and instructions for use in an IVT reaction to synthesize 5′-capped RNA molecules.

In some aspects, the disclosure provides a kit for producing a 5′-capped RNA molecule, comprising: (a) a capping primer comprising a structure according to Formula IV:

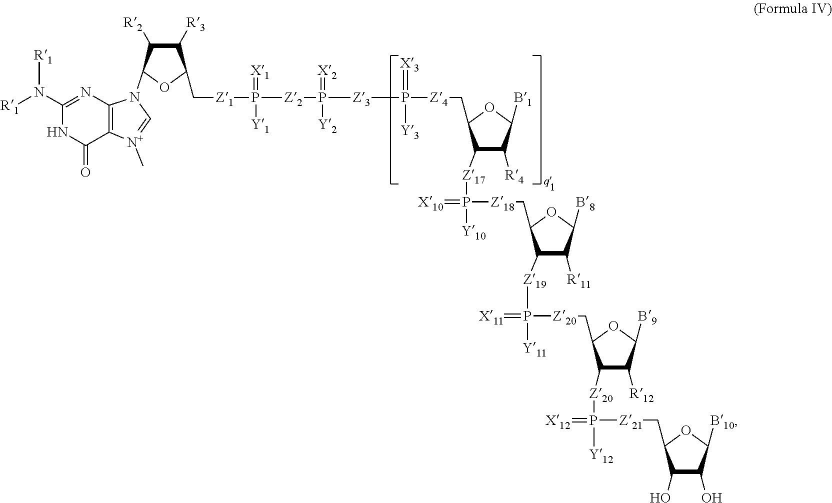

or a pharmaceutically acceptable salt thereof, wherein, q′1 is 0 or 1; B′1, if present, is a nucleobase independently selected from the group consisting of adenine, guanine, thymine, uracil, cytosine, and hypoxanthine, and any derivative or analog of the foregoing; B′8 is a nucleobase selected from the group consisting of adenine, guanine, thymine, uracil, cytosine, and hypoxanthine and any derivative or analog of the foregoing; B′9 is a nucleobase selected from the group consisting of adenine, uracil, cytosine, and any derivative or analog of the foregoing; B′10 is a nucleobase selected from the group consisting of adenine, guanine, uracil, cytosine, and any derivative or analog of the foregoing; R′1 are each independently H, alkyl, acyl, benzyl, or a cleavable unit; R′2 is H, OH, alkyl, O-alkyl, O-acyl, O-benzyl, O-MOE, halogen, LNA, a linker, a cleavable unit, or a detectable marker; R′3 is H, OH, alkyl, O-alkyl, O-acyl, O-benzyl, O-MOE, halogen, a linker, a cleavable unit, or a detectable marker; R′4, if present, R′11 and R′12 are each independently H, OH, alkyl, O-alkyl, O-MOE, halogen, LNA, a linker, or a detectable marker; X′1 to X′2, X′3, if present, and X′10 to X′12 are each independently O or S; Y′1 to Y′2, Y′3, if present, and Y′10 to Y′12 are each independently O⁻, S⁻, BH₃⁻, aryl, alkyl, O-alkyl, or O-aryl; and Z′1 to Z′3, Z′4 if present, Z′17, if present, and Z′18 to Z′21 are each independently O or S, NH, CH₂, C(halogen)₂ or CH(halogen); and (b) instructions for use of the capping primer in an IVT reaction with a polynucleotide template to synthesize 5′-capped RNA molecules, wherein: the polynucleotide template comprises a nucleobase at position −2 and position −1 immediately adjacent to and downstream (3′) of a transcriptional start site, and a nucleobase at each of position +1, position +2 and position +3 immediately adjacent to and upstream (5′) of the transcriptional start site; B′1, if present, is complementary or mismatched to a nucleobase at positions −1 of the polynucleotide template; B′8 is complementary or mismatched to a nucleobase at position −1 of the polynucleotide template; B′9 is complementary to a nucleobase at position +1 of the polynucleotide template; B′10 is complementary to a nucleobase at position +2 of the polynucleotide template; and B′8, B′9, and B′10 are not fully complementary to nucleobases at positions +1 to +3 of the polynucleotide template. In some embodiments, the capping primer consists of the structure according to Formula III. In some embodiments, q′1 is 0.

In some embodiments of the foregoing or related aspects, q′1 is 1. In some embodiments, B′1 is mismatched to a nucleobase at position −2 of the polynucleotide template. In some embodiments, B′1 is complimentary to a nucleobase at position −2 of the polynucleotide template. In some embodiments, B′1 is selected from the group consisting of adenine, uracil, thymine, cytosine, 5-methylcytosine, guanine, and hypoxanthine. In some embodiments, B′1 is adenine, or a derivative or analog thereof. In some embodiments, B′1 is uracil, or a derivative or analog thereof. In some embodiments, B′1 is guanine, or a derivative or analog thereof. In some embodiments, B′1 is cytosine, or a derivative or analog thereof.

In some embodiments of the foregoing or related aspects, B′8 is not N6-modified adenine. In some embodiments, B′8 is a nucleobase selected from the group consisting of adenine, uracil, thymine, cytosine, 5-methylcytosine, guanine, and hypoxanthine.

In some embodiments of the foregoing or related aspects, B′9 is selected from the group consisting of adenine, cytosine, 5-methylcytosine, thymine and uracil. In some embodiments, B′9 is selected from the group consisting of adenine, cytosine and uracil. In some embodiments, B′9 is adenine or uracil.

In some embodiments of the foregoing or related aspects, B′10 is selected from the group consisting of adenine, guanine, cytosine, 5-methylcytosine, thymine and uracil. In some embodiments, B′10 is selected from the group consisting of adenine, guanine, cytosine and uracil.

In some embodiments of the foregoing or related aspects, B′9 is adenine, or a derivative or analog thereof, and B10 is guanine, or a derivative or analog thereof. In some embodiments, (i) the nucleobase at position +1 of the polynucleotide template is thymine, and (ii) the nucleobase at position +2 of the polynucleotide template is cytosine. In some embodiments, B′9 is cytosine, or a derivative or analog thereof; and B′10 is guanine, or a derivative or analog thereof. In some embodiments, (i) the nucleobase at position +1 of the polynucleotide template is guanine, and (ii) the nucleobase at position +2 of the polynucleotide template is cytosine. In some embodiments, B′9 is uracil, or a derivative or analog thereof; and B′10 is guanine, or a derivative or analog thereof. In some embodiments, (i) the nucleobase at position +1 of the polynucleotide template is adenine, and (ii) the nucleobase at position +2 of the polynucleotide template is cytosine. In some embodiments, B′9 is adenine, or a derivative or analog thereof, and B′10 is adenine, or a derivative or analog thereof. In some embodiments, (i) the nucleobase at position +1 of the polynucleotide template is thymine, and (ii) the nucleobase at position +2 of the polynucleotide template is thymine. In some embodiments, B′9 is adenine, or a derivative or analog thereof; and B′10 is cytosine, or a derivative or analog thereof. In some embodiments, (i) the nucleobase at position +1 of the polynucleotide template is thymine, and (ii) the nucleobase at position +2 of the polynucleotide template is guanine. In some embodiments, B′9 is adenine, or a derivative or analog thereof, and B′10 is uracil, or a derivative or analog thereof. In some embodiments, (i) the nucleobase at position +1 of the polynucleotide template is thymine, and (ii) the nucleobase at position +2 of the polynucleotide template is adenine. In some embodiments, the nucleobase at position −1 of the polynucleotide template is thymine or adenine.

In some embodiments of the foregoing or related aspects, R′1 are each independently H or C1-C6 alkyl. In some embodiments, R′ are each H. In some embodiments, R′2 is H, OH, O—(C1-C6 alkyl), O-MOE, fluoro, or LNA. In some embodiments, R′2 is OH. In some embodiments, R′3 is H, OH, O—(C1-C6 alkyl), O-MOE, or fluoro. In some embodiments, R′3 is OH or OMe. In some embodiments, R′4, if present, and R′11 and R′12 are each independently H, OH, O—(C1-C6 alkyl), O-MOE, fluoro, or LNA. In some embodiments, R′4, if present, is H, OH, OMe, O-MOE, or fluoro; R′11 is H, OH, OMe, O-MOE, or fluoro; and R′12 is OH or OMe. In some embodiments, R′4, if present, is OMe; R′11 is OMe; and R′12 is OMe.

In some embodiments of the foregoing or related aspects, B′8 is complementary to a nucleobase at position −1 of the polynucleotide template. In some embodiments, B′8 is mismatched to a nucleobase at position −1 of the polynucleotide template.

In some embodiments of the foregoing or related aspects, the polynucleotide template is a DNA template. In some embodiments, the polynucleotide template is linear DNA. In some embodiments, the polynucleotide template comprises a sequence encoding a 5′-untranslated region (UTR), an open-reading frame (ORF), a 3′-UTR, and a polyA sequence.

### BRIEF DESCRIPTION OF THE FIGURES

FIGS. 1A-1X provide exemplary tetranucleotide or pentanucleotide capping primers of the disclosure, including ^{m7}GpppA_{2′OMe}pA_{2′OMe}pG “GAAG” (FIG. 1A), ^{m7}G_{3′OMe}pppA_{2′OMe}pA_{2′OMe}pG “G_{3′OMe}AAG” (FIG. 1), ^{m7}GpppU_{2′OMe}pA_{2′OMe}pG “GUAG” (FIG. 1C), ^{m7}GpppA_{2′OMe}pG_{2′OMe}pG “GAGG” (FIG. 1D), ^{m7}GpppC_{2′OMe}pA_{2′OMe}pG “GCAG” (FIG. 1E), ^{m7}GpppG_{2′OMe}pA_{2′OMe}pG “GGAG” (FIG. 1F), ^{m7}GpppA_{2′OMe}pC_{2′OMe}pG “GACG” (FIG. 1G), ^{m7}GpppA_{2′OMe}pU_{2′OMe}pG “GAUG” (FIG. 1H), ^{m7}GpppA_{2′OMe}pA_{2′OMe}pA “GAAA” (FIG. 1I), ^{m7}GpppA_{2′OMe}pA_{2′OMe}pC “GAAC” (FIG. 1J), ^{m7}GpppA_{2′OMe}pA_{2′OMe}pU “GAAU” (FIG. 1K), ^{m7}GpppdTpA_{2′OMe}pG “GTAG” (FIG. 1L), ^{m7}GpppI_{2′OMe}pA_{2′OMe}pG “GIAG” (FIG. 1M), ^{m7}GpppA_{2′F}pA_{2′OMe}pG “GA_{F}AG” (FIG. 1N), ^{m7}GpppA_{2′MOE}A_{2′OMe}pG “GA_{MOE}AG” (FIG. 1O), ^{m7}GppprApA_{2′OMe}pG “GrAAG” (FIG. 1P), ^{m7}Gppp^{m5}C_{2′OMe}pA_{2′OMe}pG “G^{m5}CAG” (FIG. 1Q), ^{m7}Gppp^{m5}U_{2′OMe}pA_{2′OMe}pG “G^{m5}UAG” (FIG. 1R), ^{m7}G_{3′OMe}ppp^{m6}A_{2′OMe}pA_{2′OMe}pG “G_{3′OMe}^{m6}AAG” (FIG. 1S), ^{m7}G_{3′OMe}pppA_{2′OMe}pA_{2′OMe}pA_{2′OMe}pG “G_{3′OMe}AAAG” (FIG. 1T), ^{m7}GpppG_{Ina}pA_{2′OMe}pG “GG_{Ina}AG” (FIG. 1U), ^{m7}GpppG_{2′OMe}psA_{2′OMe}psG “GGpsApsG” (FIG. 1V), ^{m7}GpppG_{2′OMe}pA_{2′OMe}pA_{2′OMe}pG “GGAAG” (FIG. 1W) and ^{m7}GpppU_{2′OMe}pA_{2′OMe}pA_{2′OMe}pG “GUAAG” (FIG. 1X).

FIGS. 2A-211 provide capping efficiency analysis for mRNA prepared by IVT using the indicated capping primers and a DNA polynucleotide template having 2′-deoxycytidine at position +1 and position +2 (“CC Template”; FIG. 2A), a DNA template having 2′-deoxythymidine at position +1 and 2′-deoxycytidine at position +2 (and 2′-deoxythymidine at position −1 “TC template” or 2′-deoxyadenosine at position −1 “TCΦ2.5 Template”; FIGS. 2B, 2C, 2E, 2F, 2G, and 2H), a DNA template having 2′-deoxyguanosine at position +1 and 2′-deoxycytidine at position +2 (“GC DNA template”), 2′-deoxyadenosine at position +1 and 2′-deoxycytidine at position +2 (“AC DNA template”), 2′-deoxythymidine at position +1 and position +2 (“TT DNA template”), 2′-deoxythymidine at position +1 and 2′-deoxyguanosine at position +2 (“TG DNA template”), or 2′-deoxythymidine at position +1 and 2′-deoxyadenosine at position +2 (“TA DNA template”) (FIG. 2D). The IVT reaction was performed using equimolar ratio of capping agent to NTPs (capping primer:NTP of 1:1), “Low GTP” (capping primer:GTP:NTP molar ratio of 3:1:4) or “High Capping Primer” (capping primer:NTP of 2:1). Wild-type T7 RNA polymerase was used for the IVT reaction unless otherwise specified. A PrimeCap T7 RNA Polymerase (Takara Bio) was used for the IVT reaction (under the condition where the molar ratio of capping primer:NTP of 2:5) in FIG. 2F.

FIG. 3 provides a schematic showing an exemplary transcription system of the disclosure comprising a capping primer and a polynucleotide template. The capping primer comprises a 3′-end comprising nucleotides “B8,” “B9,” and “B10,” wherein B8 is complementary or mismatched to position −1 of the polynucleotide template, B9 is complementary to position +1 of the polynucleotide template, and B10 is complementary to position +2 of the polynucleotide template. The template sequence in FIG. 3 (3′-ATTATGCTGAGTGATAX⁻¹X¹X²X³X⁴X⁵X⁶-5′; SEQ ID NO: 6) is an exemplary antisense strand sequence of the polynucleotide template which corresponds to the consensus sequence for T7 RNA polymerase.

### DETAILED DESCRIPTION

The present disclosure provides capping primers, and transcription systems comprising a capping primer described herein, for performing in vitro transcription (IVT) for synthesis of 5′-capped RNAs (e.g., for synthesis of 5′-capped mRNAs). In some embodiments, the transcription system comprises a capping primer and a polynucleotide template described herein. In some embodiments, synthesis of the 5′-capped RNA occurs upon contacting the transcription system with an RNA polymerase (RNAP) described herein under transcription conditions (e.g., conditions comprising nucleoside 5′-triphosphates and reaction buffer).

Most eukaryotic mRNA, and other non-coding RNAs, are capped at the 5′-terminus. The mRNA 5′-cap includes a guanine nucleoside joined via its 5′-carbon by a triphosphate to the 5′-end of the mRNA molecule. In most eukaryotes, the nitrogen at position 7 of guanine in the cap nucleotide is methylated (i.e., 7-methylguanosine, which is interchangeably referred to as “^{m7}G” or “^{m7}G” or “7mG” or “m7G”). This 5′-cap moiety is generally represented by ^{7m}G5′(ppp)5′N₁. The ^{7m}Gppp moiety contributes to mRNA maturation, cellular transport, assembly of the translation initiation complex, and protection from degradation by exonucleases (see, e.g., Kang, et al (2023) Adv. Drug Deliv. 199:114961).

Generally, eukaryotic transcription occurs in three stages: (i) initiation phase (i.e., wherein the RNAP interacts with the promoter and transitions into formation of a stable elongation complex), (ii) elongation, and (iii) termination. Capping typically occurs after initiation through an enzymatic process. Transcribed mRNA is processed by enzymes to affix the ^{7m}Gppp moiety to the first nucleoside. The cap is further processed by 2′-O-methylation at the first transcribed nucleoside (i.e., transcript position 1) to produce a Cap-1 structure, or at the first and second transcribed nucleosides (i.e., transcript positions 1 and 2 respectively) to form the Cap-2 structure. The Cap-1 structure is generally represented by ^{7m}G5′(ppp)5′N₁m and the Cap-2 structure by ^{7m}G5′(ppp)5′N₁mN₂m. Cap 2′-O-methylation distinguishes self RNA from foreign RNA (see, e.g., Daffis et al (2010) Nature 468:452). Additional cap structures include Cap-3 structure, having 2′-O-methylation at each of the first, second, and third transcribed nucleotides, and Cap-4 structure, having 2′-O-methylation at each of the first, second, third, and fourth transcribed nucleotides. Cap-3 and Cap-4 structures are generally represented as ^{7m}G5′(ppp)5′N₁mN₂ mN₃m and ^{7m}G5′(ppp)5′N₁mN₂mN₃mN₄m respectively.

The IVT reaction is a procedure that provides polynucleotide template-directed synthesis of RNA molecules. The IVT reaction is typically performing using a single subunit phage RNAP derived from T7, T3, SP6, K1-5, K1E, K1F, or K11 bacteriophages, and the polynucleotide template includes a phage promoter sequence upstream the sequence of interest. The T7 RNAP is commonly used for its high activity and fidelity for RNA synthesis. T7 RNAP is a monomeric bacteriophage-encoded DNA directed RNA polymerase that catalyzes formation of RNA in the 5′ to 3′ direction. In the process of transcription initiation, the T7 RNAP recognizes a specific region comprising a consensus sequence. The consensus sequence for most T7 RNAP, as present in the sense strand, encompasses about 17 bp of sequence upstream (5′) to about 6 bp downstream (3′) of the transcription start site (TSS). The T7 promoter sequence, as present in the sense strand, is the portion of the consensus sequence that is upstream (5′) of the TSS. During transcription initiation, T7 RNAP binds to the promoter DNA from nucleotide position minus 17 to position minus 5 (i.e., position −17 to position −5) with high specificity while the DNA double strand is melted from position minus 4 to position plus 3 (i.e., position −4 to position +3) to prime RNA synthesis from the nucleotide at position plus 1 (i.e., position +1) (see, e.g., Cheetham, et al (1999) Nature 399: 80; Yin, et al (2002) Science 286:2305). The melting/unwinding region of position −4 to −1 adjacent to the initial transcription region at position +1 is typically AT-rich (e.g., a TATA box for the T7 promoter).

The upstream (5′) promoter sequence of the polynucleotide template sense strand can be modified to incorporate the appropriate RNAP consensus sequence, or a variant thereof, by a person skilled in the art. The position of the first transcribed nucleotide is referred to as the +1 transcript nucleotide of the RNA, the second transcribed nucleotide as the +2 transcript nucleotide, and so on. The wild-type T7 consensus sequence comprises a GTP at position +1 of the sense strand. Moreover, for polynucleotide templates of the present disclosure comprising a promoter suitable for IVT using a T7 RNA polymerase, the TATA box in the antisense strand of the T7 consensus sequence corresponds to positions −4 to −1 of the polynucleotide template. For example, the consensus sequence for a wild-type T7 RNA polymerase is TAATACGACTCACTATAGGGAGA (SEQ ID NO: 7), wherein the nucleotide in bold corresponds to position +1 of the sense strand, and the “TATA” box in italics corresponds to positions −4 to −1. For polynucleotide templates of the present disclosure suitable for IVT using a different RNAP (e.g., an SP6 RNA polymerase), the TATA box in the antisense strand of the RNAP consensus sequence (e.g., an SP6 consensus sequence) corresponds to positions −4 to −1 of the polynucleotide template. For example, the consensus sequence for a wild-type SP6 RNA polymerase is ATTTAGGTGACACTATAGAAGAA (SEQ ID NO: 8), wherein the nucleotide in bold corresponds to position +1 of the sense strand, and the “TATA” box in italics corresponds to positions −4 to −1.

In the example shown in Table 1, the +1 transcript nucleotide is A and the +1 template nucleotide is T; the +2 transcript nucleotide is G and the +2 template nucleotide is C. The “TATA” sequence present in the sense strands is positions −4 to −1 of the template. The first step in transcriptional initiation is called de novo RNA synthesis, in which RNAP catalyzes formation of a phosphodiester bond between nucleoside 5′-triphosphates complementary to template residues at position +1 and +2 to form a dinucleotide which is subsequently elongated.

For the purpose of clarity, one of skill in the art would appreciate that the first transcribed nucleoside 5′-triphosphate forms complementary base pair with a nucleobase at position +1 of a polynucleotide template. The transcription starts from TSS between position −1 and position +1 of the polynucleotide template as shown in Table 1. Transcription occurs in the 5′ to 3′ direction for the nascent RNA strand and 3′ to 5′ for the antisense strand of the polynucleotide template.

TABLE 1
Exemplary DNA Template for T7 Polymerase (TC start)

| Position in transcript | | | | | | | | | | | |
|---|---|---|---|---|---|---|---|---|---|---|---|
| RNA transcript | | | | | | | | | | | |
| Coding (sense) strand | [symbol] | T | A | A | T | A | C | G | A | C | T |
| Template (antisense) strand | [symbol] | A | T | T | A | T | [symbol] | C | T | G | A |
| Position In template | | −17 | −16 | −1 [symbol] | −14 | −1 [symbol] | −12 | − [symbol] | −10 | − [symbol] | − [symbol] |

| Position in transcript | | | | | | | | +1 | +2 | +3 | +4 | | | | |
|---|---|---|---|---|---|---|---|---|---|---|---|---|---|---|---|
| RNA transcript | | | | | | [symbol] | [symbol] | A | G | G | G | A | A | [symbol] | [symbol] |
| Coding (sense) strand | C | A | C | T | A | T | A | A | G | G | G | A | A | [symbol] | [symbol] |
| Template (antisense) strand | [symbol] | T | [symbol] | A | T | A | T | T | C | C | C | T | T | [symbol] | [symbol] |
| Position In template | −7 | − [symbol] | − [symbol] | − [symbol] | −3 | −2 | −1 | [symbol] | [symbol] | [symbol] | [symbol] | [symbol] | [symbol] | | |

Exemplary T7 RNAP sequence for TC start (SEQ ID NO: 15) which corresponds to a sequence for T7 RNAP (positions −17 to +6)
TSS is indicated by a solid line between position −1 and position +1 of the DNA template [symbol]
indicates data missing or illegible when filed

As described herein, it was discovered that a ^{7m}G(5′)ppp(5′)A_{2′OMe}pA_{2′OMe}pG tetranucleotide capping reagent combined with a TC template in an IVT reaction generates Cap-2 mRNA with high capping efficiency (e.g., greater than about 70%, 75%, 80%, 85%, 90%, or 95%). The second, third and fourth nucleotides of the capping agent hybridize to positions −1, +1 and +2 of the TC template respectively. The high capping efficiency is achieved under equimolar GTP conditions (i.e., equimolar ratio of capping primer, NTP, and GTP), such that overall transcription yield is not sacrificed for the sake of increased capping efficiency. Moreover, it was found that a mismatch between the second nucleotide and the nucleobase at position −1 of the TC template did not reduce capping efficiency (e.g., if the third and the fourth nucleotides of the capping primer are complementary to positions +1 and +2 respectively of the TC template). It was demonstrated that capping primers having a second nucleotide with a non-natural nucleobase achieved efficient 5′-capping of mRNA in high yield.

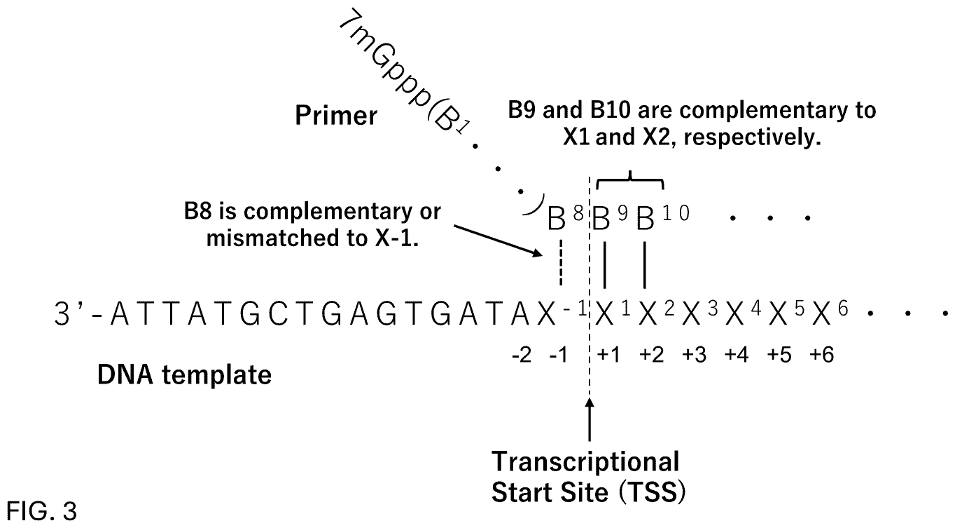

Without being bound by theory, it is believed that capping primers of 4 or more nucleotides in length (e.g., 4, 5, 6, 7, 8, 9, 10, 11, 12, 13, 14, 15, 16, 17, 18, 19, 20, 21, 22, or 23 nucleotides in length) facilitate efficient 5′-capping of RNA when hybridized to the polynucleotide template in the negative direction from the +2 position (i.e., position −1 to position +2 for a capping primer of 4 nucleotides in length; position −2 to position +2 for a capping primer of 5 nucleotides in length; position −3 to position +2 for a capping primer of 6 nucleotides in length, etc). As demonstrated in the Example section of the present disclosure, it was found that the Cap-3 mRNA having three 2′-O-methyl (OMe) groups at the first three transcribed nucleosides could be synthesized by using pentanucleotide capping primers designed to hybridize to positions −2 to +2. Further, synthesis is achieved regardless of whether the second nucleobase of the primer is complementary or mismatched to position −2 of the polynucleotide template. See FIG. 3.

Moreover, in some embodiments, it was found to be beneficial if the polynucleotide template comprises a nucleotide other than 2′-deoxycytidine at position +1. De novo RNA synthesis occurs from GTP when 2′-deoxycytidine is present at position +1, which competes with elongation from capping primer. Thus, by having a nucleotide other than 2′-deoxcytidine at position +1 of the polynucleotide template, the elongation reaction from the capping primer commences without competition with de novo RNA synthesis from GTP, which in turn improves capping efficiency. In exemplary embodiments of the disclosure, a tetranucleotide capping primer having a third and fourth nucleotide complementary to positions +1 and +2 of the polynucleotide template achieved efficient 5′-capping of mRNA in high yield with either a 2′-deoxyadenosine or 2′-deoxythymidine at position −1 of the polynucleotide template, and optionally with a mismatch and/or non-natural nucleobase at the second nucleotide of the capping primer.

Accordingly, the present disclosure provides capping primers, systems comprising the capping primers and polynucleotide templates, and methods of use thereof for synthesis of 5′-capped RNA (e.g., 5′-capped RNA comprising a Cap-1 or Cap-2 structure).

In some embodiments, in vitro transcription to synthesize an RNA of the disclosure is initiated using a transcription system described herein. In some embodiments, the transcription system comprises a polynucleotide template (e.g., a DNA template), an RNA polymerase (RNAP), a plurality of nucleoside 5′-triphosphates (NTPs), and a capping primer described herein. In some embodiments, the capping primer comprises a Cap-1 or a Cap-2 structure. As described herein, the capping primer is elongated by incorporation of NTP at the 3′-end, thereby resulting in an RNA comprising the capping primer at its 5′-end.

### Definitions

As used herein, the term “a” or “an” refers to one, or more than one, of that entity. In some embodiments, “a” refers to plural referents. As such, the terms “a” or “an”, “one or more” and “at least one” can be used interchangeably herein. In addition, reference to “an element” by the indefinite article “a” or “an” does not exclude the possibility that more than one of the elements is present, unless the context clearly requires that there is one and only one of the elements.

As used herein, the terms “approximately” or “about,” as applied to one or more values of interest, refers to a value that is similar to a stated reference value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). In some embodiments, the term “about” refers to ±10%, 9%, 8%, 7%, 6%, 5%, 4%, 3%, 2%, 1%, or less of the recited value.

As used herein, the term “nucleic acid” is used in its broadest sense and encompasses any compound and/or substance that includes a polymer of nucleotides or nucleosides joined together, e.g., by a phosphodiester linkage between 5′ and 3′ carbon atoms or a non-phosphate internucleoside linkage between 5′ and 3′ carbon atoms. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

When referring to a sequence of an oligonucleotide, reference is made to the sequence or order of nucleobase moieties, or modifications thereof, of the covalently linked nucleotides or nucleosides using the standard nucleotide nomenclature. “G,” “C,” “A,” “T” and “U,” which respectively corresponds to nucleotides or nucleosides comprising a guanine, cytosine, adenine, thymine and uracil as a nucleobase. In some embodiments, the nucleobase is a purine heterocyclic compound or pyrimidine heterocyclic compound found in a naturally occurring nucleic acid. In some embodiments, the nucleobase is a an analog or derivative of a purine heterocyclic compound or pyrimidine heterocyclic compound found in a naturally occurring nucleic acid. Guanine, cytosine, adenine, thymine and uracil are exemplary nucleobases found in naturally occurring nucleic acids.

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-limiting group consisting of small interfering RNA (siRNA), asymmetrical interfering RNA (aiRNA), microRNA (miRNA), Dicer-substrate RNA (dsRNA), small hairpin RNA (shRNA), mRNA, long non-coding RNA (lncRNA) and mixtures thereof.

As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule comprise a ORF, a 5′-UTR, a 3′-UTR, a 5′-cap and a polyA sequence.

The term “open reading frame,” also abbreviated as “ORF,” refers to a segment or region of an mRNA molecule that encodes a polypeptide. The ORF comprises a continuous stretch of non-overlapping, in-frame codons, beginning with the initiation codon and ending with a stop codon, and is translated by the ribosome.

The term “nucleobase” (alternatively “nucleotide base” or “nitrogenous base”) refers to a molecule comprising a purine or pyrimidine heterocyclic found in nucleic acids, including any derivatives or analogs of the natural and/or naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

The term “nucleoside” refers to a compound comprising a nucleobase (e.g., a purine or pyrimidine), covalently linked to a sugar molecule (e.g., a ribose in RNA or a deoxyribose in DNA), or a derivative or analog thereof, but lacking an internucleoside linkage group (e.g., a phosphate group).

The term “nucleotide” refers to a nucleoside covalently bonded to a phosphate-containing internucleoside linkage group, or a derivative or analog thereof that confers improved chemical or functional properties to a nucleic acid or a portion thereof (e.g., improved binding affinity, nuclease resistance, and/or chemical stability).

The term “natural” or “naturally-occurring” nucleobase refers to the purine bases adenine, N6-methyladenine, hypoxanthine and guanine, and the pyrimidine bases thymine, cytosine, 5-methylcytosine, 5-hydroxymethylcytosine, uracil, N1-methyluracil and 2-thiouracil. Natural, or naturally occurring nucleosides include, but are not limited to, ribonucleoside, 2′-O-methyl ribonucleoside, or 2′-deoxyribonucleoside derivatives of adenosine (A), guanosine (G), cytidine (C), uridine (U), inosine (I), N6-methyladenosine (m6A or ^{6m}A), 7-methylguanosine (^{7m}G), 5-methylcytidine (^{5m}C), 5-hydroxymethylcytidine (^{5hm}C), 5-methyluridine (^{5m}U), pseudouridine (W), N1-methyl-pseudouridine (^{1m}ψ), 2′-deoxyadenosine (A or dA), 2′-deoxyguanosine (G or dG), 2′-deoxycytidine (C or dC), and thymidine (T or dT).

The terms “analog” or “derivative” refers to a compound in which a functional group of a certain compound is substituted by another functional group. In some embodiments, hydrogen is substituted by alkyl group or halogen group in nucleobase derivatives. In some embodiments, oxygen atom of phosphate group is substituted by sulfur or boron atom in oligonucleotide derivative. In some embodiments, hydroxy group of 2′-position and carbon atom of 4′-position of ribose is bridged by methylene group in nucleic acid derivative.

As used herein, the terms “derivative or analog” of a nucleobase (e.g. adenine, guanine, thymine, uracil or cytosine), “modified nucleobases” or any variations such as “nucleobase derivatives,” include natural and naturally-occurring and artificially designed nucleobases which may form a base pair with a naturally-occurring nucleobase under appropriately stringent conditions. In some embodiments, a nucleobase derivative or analog of adenine comprises N6-methyladenine. In some embodiments, a nucleobase derivative or analog of cytosine comprises 5-methylcytosine. In some embodiments, a nucleobase derivative or analog of uracil comprises 5-methyluracil. Other natural, non-natural, and/or synthetic nucleobases, as known in the art and/or described herein, is included in nucleobase derivatives or analogs. In some embodiments, nucleobase derivatives or analogs of adenine comprising N-alkylated adenine synthesized by chemical reaction. Examples of derivative or analog of a nucleobase which may form a base pair with a naturally-occurring nucleobase under appropriately stringent conditions include, but not limited to, N6-methyladenine, 5-methylcytosine, 5-hydroxymethylcytosine, N1-methyl-uracil, and hypoxanthine. When a nucleobase analog or derivative is selected as part of a structural formula described herein, it will be appreciated that the nucleobase analog/derivative is expected to engage in a canonical Watson Crick base pairing with a corresponding nucleobase of a polynucleotide template. For example, pseudouridine can form a base pair with an adenosine nucleobase present in the polynucleotide template in a similar manner to a base pair formed between uridine and adenosine.

The terms “nucleoside analogs,” “nucleoside derivatives” or “modified nucleosides” include synthetic nucleosides. Nucleoside derivatives also include nucleosides having modified nucleobase or/and sugar moieties, with or without protecting groups and include, for example, 2′-deoxy-2′-fluorouridine, 5-fluorouridine and the like. The compounds and methods provided herein include such base rings and synthetic analogs thereof, as well as unnatural heterocycle-substituted base sugars, and acyclic substituted base sugars. Other nucleoside derivatives that may be utilized with the present disclosure include, for example, LNA nucleosides, halogen-substituted purines (e.g., 6-fluoropurine), halogen-substituted pyrimidines, N6-ethyladenine, N4-(alkyl)-cytosines, 5-ethylcytosine, and the like.

As used herein, the term “primer” refers to a oligonucleotide (e.g., RNA, DNA, or RNA/DNA hybrid) that hybridizes to a polynucleotide template and functions as a starting point for RNA synthesis.

As used herein, the term “modified capping primer” refers to a capping primer described herein comprising a modification. In some embodiments, the capping primer comprises a modified nucleoside/nucleotide or a modified internucleotide linkage. In some embodiments, a “modified nucleotide” or “modified nucleoside” is a non-standard nucleotide or nucleoside respectively, including non-natural ribonucleotides/ribonucleosides or deoxyribonucleotides/deoxyribonucleosides. For example, in some embodiments, a modified nucleotide/modified nucleoside comprise a substitution of, addition to, or removal of an internucleoside linkage, sugar moiety, and/or nucleobase. In some embodiments, the substitution, addition, or removal alters the chemical property of the nucleotide/nucleoside (e.g., to decrease susceptibility to enzymatic cleavage), but does not substantially impair its function (e.g., in base pairing).

In some embodiments, the capping primer comprises a modification at a sugar, a nucleobase, a triphosphate bridge, and/or an internucleoside phosphate. As used herein, the term “internucleotide linkage” refers to the bond or bonds that connect two nucleosides of a polynucleotide. In some embodiments, the internucleotide linkage comprises a phosphodiester linkage or a modified linkage (e.g., a phosphorothioate linkage). Examples of oligonucleotide internucleotide linkage modifications include, but are not limited to, phosphorothioate, boranophosphate, phosphonate, phosphoramidate, phosphotriester, and methylphosphonate derivatives. In some embodiments, the triphosphate bridge modifications include, but are not limited to, phosphorothioate, boranophosphate, phosphonate, phosphoramidate, phosphotriester, and methylphosphonate derivatives. As used herein, the term “marker” or “detectable marker” refers to a chemical moiety that can be detected directly or indirectly when operably linked to a molecule. For example, a detectable marker includes a radioisotope, a mass isotope, a dye, a fluorophore, an enzyme, or a hapten.

As used herein, the term “hybridize” or “specifically hybridize” refers to a process where a capping primer anneals to a polynucleotide template by forming base pairs under appropriately stringent conditions during a transcription reaction. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Those skilled in the art understand how to determine the appropriate stringency of hybridization/washing conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J., all of which are incorporated herein by reference in their entireties. In some embodiments, hybridization occurs between at least 2 contiguous nucleobases of the capping primer and the polynucleotide template. In some embodiments, hybridization occurs between 2-23 contiguous nucleobases of the capping primer and the polynucleotide template.

As used herein, a nucleobase of a primer that is “complementary,” or a “complement” to, or has “complementarity” to a nucleobase of a polynucleotide template refers to a nucleobase having a structure which forms Watson-Crick-type base pair with the nucleobase of polynucleotide template. As understood by the skilled artisan, Watson-Crick base pairing refers to the set of base pairing rules wherein a purine nucleobase binds to a pyrimidine nucleobase to form a complimentary base pair, e.g., the nucleobase adenine (A) binds to the nucleobase thymine (T) or uridine (U) to form a complementary base pair, e.g., the nucleobase guanine (G) binds to the nucleobase cytosine (C) to form a complementary base pair. The nature of the hydrogen bonding depends upon the particular base pair. For example, a guanine-cytosine base pair is formed by three hydrogen bonds and the adenine-thymine or adenine-uracil base pair is formed by two hydrogen bonds. It will be understood that modified nucleobases, for example 5-methylcytosine is often used in place of cytosine, and as such the term “complementary” base pair encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1). It is further appreciated by the skilled artisan that oligonucleotides comprising naturally-occurring nucleobases, or analogs or derivatives thereof, can form complimentary base pair interactions via conventional Watson-Crick base pair rules. In some embodiments, N6-methyladenine in the capping primer is a nucleobase which is complementary to thymine of the polynucleotide template. In some embodiments, 5-methylcytosine in the capping primer is a nucleobase which is complementary to guanine of the polynucleotide template. In some embodiments, 5-hydroxymethylcytosine in the capping primer is a nucleobase which is complementary to guanine of the polynucleotide template. In some embodiments, N1-methyl-uracil in the capping primer is a nucleobase which is complementary to adenine of the polynucleotide template. As used herein, nucleobases of a primer that are “fully complementary” to nucleobases of a polynucleotide template means that each nucleobase of such nucleobases is complementary to the relevant nucleobase of the polynucleotide template at indicated positions. When one or more of nucleobase(s) in particular contiguous nucleotides of a primer (e.g. B8, B9, and B10 in Formula II) is/are mismatched to nucleobase(s) of a polynucleotide template at indicated positions, such contiguous nucleobases in the primer are not fully complementary to the nucleobases of the polynucleotide template at indicated positions.

As used herein, a nucleobase in a capping primer that is “mismatching” to, or is “mismatched” to, or is “unmatched” to a nucleobase of a polynucleotide template refers to a nucleobase forming a non-canonical base pair other than Watson-Crick-type base pairing. As understood by the skilled artisan, some base pairs (e.g., Hoogsteen, reversed Hoogsteen hydrogen bonding, or wobble base pair) other than Watson-Crick type shows weaker affinity to the opposite nucleobase compared to Watson-Crick type base pair. In some embodiments, the nucleobase in the capping primer comprises a mismatched base with respect to the polynucleotide template. In some embodiments, adenine (A) in the capping primer is mismatched to adenine (A) of the polynucleotide template. In some embodiments, uracil (U) in the capping primer is mismatched to thymine (T) of the polynucleotide template. In some embodiments, hypoxanthine is mismatched to thymine.

The following provide definitions of various chemical terms.

The term “acyl,” as used herein, represents a hydrogen, amino group (e.g., —N(R^{N1})₂, wherein each R^{N1} is, independently, H, OH, NO₂, C1-20 alkyl, aryl, acyl), or an alkyl group (e.g., a haloalkyl group), as defined herein, that is attached to the parent molecular group through a carbonyl group, as defined herein, and is exemplified by formyl (i.e., a carboxyaldehyde group), acetyl, propionyl, butanoyl, carbamoyl, and the like. Exemplary unsubstituted acyl groups include from 1 to 7, from 1 to 11, or from 1 to 21 carbons. In some embodiments, the alkyl group is further substituted with 1, 2, 3, or 4 substituents as described herein. In some embodiments, a carbamoyl group is a carbamate group having the structure —OC(═O)N(R^{N1})₂, wherein each R^{N1} is, independently, H, OH, NO₂, C1-20 alkyl, aryl, acyl.

The term “carbonyl,” as used herein, represents a C(O) group, which can also be represented as C═O.

The term “halogen” as used herein refers to bromine, chlorine, iodine, or fluorine. In some embodiments, halogen is a fluorine.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl; an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

The term “alkenyl” refers to a type of alkyl group in which at least one carbon-carbon double bond is present. In one embodiment, an alkenyl group has the formula —C(R)═CR2, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂. Depending on the structure, an alkenyl group can be monovalent or divalent (i.e., an alkenylene group).

The term “alkynyl” refers to a type of alkyl group in which at least one carbon-carbon triple bond is present. Accordingly, “alkynylene” can refer to a divalent alkynyl group. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “benzyl” refers to a type of alkyl group possessing methylene group in which at least one hydrogen is substituted phenyl group. The phenyl group is optionally substituted. The methylene group is optionally substituted. Examples of benzyl group include, but are not limited to, benzyl, 4-methylbenzyl, 3-methylbenzyl, 2-methylbenzyl, 3,5-dimethylbenzyl, 4-methoxybenzyl, 4-nitrobenzyl, 4-fuluorobenzyl, 4-chlorobenzyl, 4-phenylbenzyl, and the like. In some embodiments, the benzyl is —CH₂-Ph, —CH₂-(4-Me-Ph), -4-Cl-Bn, or —CH(CH₃)-Ph.

The term “aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be monovalent or divalent (i.e., an “arylene” group). Unless stated otherwise specifically in the specification, the term “aryl” or the prefix “ar-” (such as in “aralkyl”) is meant to include aryl radicals that are optionally substituted. In some embodiments, an aryl group is partially reduced to form a cycloalkyl group defined herein. In some embodiments, an aryl group is fully reduced to form a cycloalkyl group defined herein. In some embodiments, an aryl group is a C₆-C₁₄ aryl. In some embodiments, an aryl group is a C₆-C₁₀ aryl.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted. Depending on the structure, a cycloalkyl group can be monovalent or divalent (i.e., a cycloalkylene group).

The term “haloalkyl” denotes an alkyl group wherein at least one of the hydrogen atoms of the alkyl group has been replaced by same or different halogen atoms, particularly fluoro atoms. Examples of haloalkyl include monofluoro-, difluoro-, or trifluoro-methyl, -ethyl or -propyl, for example 3,3,3-trifluoropropyl, 2-fluoroethyl, 2,2,2-trifluoroethyl, fluoromethyl, or trifluoromethyl. The term “perhaloalkyl” denotes an alkyl group where all hydrogen atoms of the alkyl group have been replaced by the same or different halogen atoms.

The term “heteroalkylene” refers to an alkyl radical as described above where one or more carbon atoms of the alkyl is replaced with a O, N or S atom. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —OCH₂CH₂O—, —OCH₂CH₂OCH₂CH₂O—, or —OCH₂CH₂OCH₂CH₂OCH₂CH₂O—.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted. As used herein, the term “heterocycloalkylene” can refer to a divalent heterocycloalkyl group.

The term “heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-10 atoms, 0-1 P atoms, and 0-1 S atoms in the ring. In some embodiments, a heteroaryl contains 1-4 N atoms, 0-10 atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group is partially reduced to form a heterocycloalkyl group defined herein. In some embodiments, a heteroaryl group is fully reduced to form a heterocycloalkyl group defined herein. Depending on the structure, a heteroaryl group can be monovalent or divalent (i.e., a “heteroarylene” group).

The term “substituted,” “substituent” or the like, unless otherwise indicated, can refer to the replacement of one or more hydrogen radicals in a given structure individually and independently with the radical of a specified substituent including, but not limited to: D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄ alkyl), —C(═O)NH₂, —C(═O)NH(C₁-C₄ alkyl), —C(═O)N(C₁-C₄ alkyl)₂, —S(═O)₂NH₂, —S(═O)₂NH(C₁-C₄ alkyl), —S(═O)₂N(C₁-C₄ alkyl)₂, C₁-C₄ alkyl, C₃-C₆ cycloalkyl, C₁-C₄ fluoroalkyl, C₁-C₄ heteroalkyl, C₁-C₄ alkoxy, C₁-C₄ fluoroalkoxy, —SC₁-C₄ alkyl, —S(═O)C₁-C₄ alkyl, and —S(═O)₂(C₁-C₄ alkyl). In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

The term “linked” when used with respect to two or more moieties, means that the moieties are physically associated or connected with one another, either directly or via one or more additional moieties that serves as a linker, to form a structure that is sufficiently stable so that the moieties remain physically associated under the conditions in which the structure is used, e.g., physiological conditions.

The term “linker,” as used herein, refers to a chemical group or molecule linking two adjacent molecules or moieties, e.g., adjacent nucleotides in a capping primer described herein. In some embodiments, the linker is a phosphate linker or a phosphoramidite linker. In some embodiments, the linker is an organic molecule, group, polymer, or chemical moiety. In some embodiments, the linker is a positioned between a point of attachment of capping primer described herein (e.g., any one of R1 to R12 if present, of a capping primer according to Formula II) and a second molecule (e.g., a detectable marker). In some embodiments, the linker is a cleavable unit. A “cleavable unit” refers to a linker comprising a cleavable linking structure. Exemplary cleavable linking structures include, but are not limited to, an acid-labile linker structure (e.g., hydrazone, e.g., carbamate), a protease-sensitive linker structure (e.g., a cathepsin B or lysosomal enzyme responsive linker, e.g., a pyrophosphatase and acid phosphatase enzyme responsive linker, e.g., a glucuronidase responsive linker) linker structure, a photolabile linker structure (e.g., 2-nitrobenzyl linking structure), a dimethyl linker structure or a reduction-sensitive linker structure (e.g., a disulfide bond). In some embodiments, the cleavable linking structure is one described in Inagaki, et al (2023) Nat Commun 14:2657.

### Transcription Systems

The present disclosure provides transcription systems comprising a capping primer and a polynucleotide template for use in synthesis of 5′-capped RNA (e.g., 5′-capped mRNA), each component of which is described in detail herein.

Generally, a capping primer of the disclosure is a primer comprising a 7-methylguanosine, or a derivative or analog thereof, at the 5′-end and a terminal nucleoside comprising an unmodified or open 3′—OH group at the 3′-end. In some embodiments, the 7-methylguanosine is operably linked to an adjacent nucleoside by a 5′-to-5′-triphosphate linkage. For clarity, for capping primers of the disclosure comprising 7-methylguanosine, or a derivative or analog thereof, the 7-methylguanosine is included in quantifying the total number of nucleoside units present in the capping primer. For example, a capping primer having the structure ^{7m}GpppA_{2′OMe}pA_{2′OMe} pG is considered to be a 4 mer (i.e., 4 nucleosides in length) with the 7-methyl guanosine and the remaining 3 nucleosides each counted as monomer units. In some embodiments, the capping primer is extended by an RNA polymerase when combined with a polynucleotide template under transcription conditions described herein (e.g., comprising nucleoside 5′-triphosphates (NTPs)) by addition of an NTP at the 3′-end. In some embodiments, the open 3′—OH group at the terminal nucleoside is a substrate for the RNA polymerase. In some embodiments, the reaction provides an RNA comprising the capping primer at its 5′-end. In some embodiments, the capping primer comprises a Cap-0, Cap-1, Cap-2, or trimethyl guanosine (TMG)-Cap structure. In some embodiments, the capping primer comprises a Cap-3 structure. In some embodiments, the capping primer is a modified capping primer (e.g., comprising a modified nucleoside/nucleotide, a modified sugar, and/or a modified internucleoside linkage). In some embodiments, the capping primer is a 4 mer, 5 mer, 6 mer, 7 mer, 8 mer, 9 mer, 10 mer, or 11 mer. In some embodiments, the capping primer is a 4 mer. In some embodiments, the capping primer is a 5 mer. In some embodiments, the capping primer is a 4 mer comprising a Cap-2 structure. In some embodiments, the capping primer is a 4 mer comprising a Cap-1 structure. In some embodiments, the capping primer is a 5 mer comprising a Cap-1 structure. In some embodiments, the capping primer is a 5 mer comprising a Cap-2 structure. In some embodiments, the capping primer is a 5 mer comprising a Cap-3 structure.

As provided herein, a polynucleotide template of the disclosure is a polynucleotide sequence used for IVT to produce an encoded RNA molecule. In some embodiments, the polynucleotide template comprises DNA, or is a DNA template. In some embodiments, the DNA template is single stranded or comprises single stranded DNA. In other embodiments, the DNA template is double stranded, or comprises double stranded DNA. Without being held to theory or mechanism, when a double-stranded DNA template is used for IVT, an encoded RNA molecule will be transcribed from the antisense strand of the DNA template (in the 3′ to 5′ direction of the antisense strand). In some embodiments, the DNA template comprises an RNA-coding region. In some embodiments, the RNA encoded by RNA-coding region is an mRNA. In some embodiments, the mRNA encoded by the RNA-coding region comprises a 5′-UTR, an ORF, a 3′-UTR, and a polyA sequence. In some embodiments, the mRNA molecule produced from the DNA template in an IVT reaction comprises a 5′-cap.

In some embodiments, the capping primer is designed to hybridize to the polynucleotide template at a region comprising the transcription start site (TSS). In some embodiments, the nucleoside at the 3′-end of the capping primer is designed to be complementary to the nucleoside of the polynucleotide template at position +2. In embodiments referring to position of the polynucleotide template, the reference is to a position of the antisense strand of the polynucleotide template. In embodiments referring to complementarity of the capping primer to one or more positions of the polynucleotide template, the reference is complementarity of the capping primer to one or more positions of the antisense strand of the polynucleotide template. In some embodiments, the capping primer is at least 4 nucleotides in length (e.g., 4, 5, 6, 7, 8, 9, 10, 11, 12, 13, 14, 15, 16, 17, 18, 19, 20, 21, 22, or 23 nucleotides in length).

In some embodiments, the nucleobases of the capping primer are fully complementary to the nucleobases of the polynucleotide template at the indicated positions. In some embodiments, the nucleobases of the capping primer are partially complementary to the nucleobases of the polynucleotide template at the indicated positions. For example, in some embodiments, the capping primer comprises a mismatch nucleobase with respect to the polynucleotide template. In some embodiments, at least two nucleobase(s) at the 3′-end of the capping primer are complementary to the nucleobases of the polynucleotide template (e.g., the nucleobases at position +1 and +2 of the polynucleotide template).

In some embodiments, the capping primer is 4 nucleotides in length, wherein the fourth nucleobase of the capping primer from the 5′-end is complimentary to position +2 of the polynucleotide template, wherein the third nucleobase of the capping primer from the 5′-end is complimentary to position +1 of the polynucleotide template, and wherein the second nucleobase of the capping primer from the 5′-end is complementary or mismatched to position −1 of the polynucleotide template.

In some embodiments, the capping primer is 5 nucleotides in length, wherein the fifth nucleobase of the capping primer from the 5′-end is complimentary to position +2 of the polynucleotide template, wherein the fourth nucleobase of the capping primer from the 5′-end is complimentary to position +1 of the polynucleotide template, wherein the third nucleobase of the capping primer from the 5′-end is complementary or mismatched to position −1 of the polynucleotide template, and wherein the second nucleobase of the capping primer from the 5′-end is complementary or mismatched to position −2 of the polynucleotide template. In some embodiments, the nucleobases of the capping primer are fully complementary to the polynucleotide template. In some embodiments, the capping primer comprises no more than one mismatch with respect to the polynucleotide template. In some embodiments, the capping primer comprises no more than two mismatches with respect to the polynucleotide template.

In some embodiments, the capping primer is 6 nucleotides in length, wherein the sixth nucleobase of the capping primer from the 5′-end is complimentary to position +2 of the polynucleotide template, wherein the fifth nucleobase of the capping primer from the 5′-end is complimentary to position +1 of the polynucleotide template, wherein the fourth nucleobase of the capping primer from the 5′-end is complementary or mismatched to position −1 of the polynucleotide template, wherein the third nucleobase of the capping primer from the 5′-end is complementary or mismatched to position −2 of the polynucleotide template, and wherein the second nucleobase of the capping primer from the 5′-end is complementary or mismatched to position −3 of the polynucleotide template. In some embodiments, the nucleobases of the capping primer are fully complementary to the polynucleotide template. In some embodiments, the capping primer comprises no more than one mismatch with respect to the polynucleotide template. In some embodiments, the capping primer comprises no more than two mismatches with respect to the polynucleotide template. In some embodiments, the capping primer comprises no more than three mismatches with respect to the polynucleotide template.

In some embodiments, the capping primer is 7 nucleotides in length, wherein the seventh nucleobase of the capping primer from the 5′-end is complimentary to position +2 of the polynucleotide template, wherein the sixth nucleobase of the capping primer from the 5′-end is complimentary to position +1 of the polynucleotide template, wherein the fifth nucleobase of the capping primer from the 5′-end is complementary or mismatched to position −1 of the polynucleotide template, wherein the fourth nucleobase of the capping primer from the 5′-end is complementary or mismatched to position −2 of the polynucleotide template, wherein the third nucleobase of the capping primer from the 5′-end is complementary or mismatched to position −3 of the polynucleotide template, and wherein the second nucleobase of the capping primer from the 5′-end is complementary or mismatched to position −4 of the polynucleotide template. In some embodiments, the nucleobases of the capping primer are fully complementary to the polynucleotide template. In some embodiments, the capping primer comprises no more than one mismatch with respect to the polynucleotide template. In some embodiments, the capping primer comprises no more than two mismatches with respect to the polynucleotide template. In some embodiments, the capping primer comprises no more than three mismatches with respect to the polynucleotide template. In some embodiments, the capping primer comprises no more than four mismatches with respect to the polynucleotide template.

In some embodiments, the capping primer is 8 nucleotides in length, wherein the eighth nucleobase of the capping primer from the 5′-end is complimentary to position +2 of the polynucleotide template, wherein the seventh nucleobase of the capping primer from the 5′-end is complimentary to position +1 of the polynucleotide template, wherein the sixth nucleobase of the capping primer from the 5′-end is complementary or mismatched to position −1 of the polynucleotide template, wherein the fifth nucleobase of the capping primer from the 5′-end is complementary or mismatched to position −2 of the polynucleotide template, wherein the fourth nucleobase of the capping primer from the 5′-end is complementary or mismatched to position −3 of the polynucleotide template, wherein the third nucleobase of the capping primer from the 5′-end is complementary or mismatched to position −4 of the polynucleotide template, and wherein the second nucleobase of the capping primer from the 5′-end is complementary or mismatched to position −5 of the polynucleotide template. In some embodiments, the nucleobases of the capping primer are fully complementary to the polynucleotide template. In some embodiments, the capping primer comprises no more than one mismatch with respect to the polynucleotide template. In some embodiments, the capping primer comprises no more than two mismatches with respect to the polynucleotide template. In some embodiments, the capping primer comprises no more than three mismatches with respect to the polynucleotide template. In some embodiments, the capping primer comprises no more than four mismatches with respect to the polynucleotide template. In some embodiments, the capping primer comprises no more than five mismatches with respect to the polynucleotide template.

In some embodiments, the capping primer is 9 nucleotides in length, wherein the ninth nucleobase of the capping primer from the 5′-end is complimentary to position +2 of the polynucleotide template, wherein the eighth nucleobase of the capping primer from the 5′-end is complimentary to position +1 of the polynucleotide template, wherein the seventh nucleobase of the capping primer from the 5′-end is complementary or mismatched to position −1 of the polynucleotide template, wherein the sixth nucleobase of the capping primer from the 5′-end is complementary or mismatched to position −2 of the polynucleotide template, wherein the fifth nucleobase of the capping primer from the 5′-end is complementary or mismatched to position −3 of the polynucleotide template, wherein the fourth nucleobase of the capping primer from the 5′-end is complementary or mismatched to position −4 of the polynucleotide template, wherein the third nucleobase of the capping primer from the 5′-end is complementary or mismatched to position −5 of the polynucleotide template, and wherein the second nucleobase of the capping primer from the 5′-end is complementary or mismatched to position −6 of the polynucleotide template. In some embodiments, the nucleobases of the capping primer are fully complementary to the polynucleotide template. In some embodiments, the capping primer comprises no more than one mismatch with respect to the polynucleotide template. In some embodiments, the capping primer comprises no more than two mismatches with respect to the polynucleotide template. In some embodiments, the capping primer comprises no more than three mismatches with respect to the polynucleotide template. In some embodiments, the capping primer comprises no more than four mismatches with respect to the polynucleotide template. In some embodiments, the capping primer comprises no more than five mismatches with respect to the polynucleotide template. In some embodiments, the capping primer comprises no more than six mismatches with respect to the polynucleotide template.

In some embodiments, the capping primer is 10 nucleotides in length, wherein the tenth nucleobase of the capping primer from the 5′-end is complimentary to position +2 of the polynucleotide template, wherein the ninth nucleobase of the capping primer from the 5′-end is complimentary to position +1 of the polynucleotide template, wherein the eighth nucleobase of the capping primer from the 5′-end is complementary or mismatched to position −1 of the polynucleotide template, wherein the seventh nucleobase of the capping primer from the 5′-end is complementary or mismatched to position −2 of the polynucleotide template, wherein the sixth nucleobase of the capping primer from the 5′-end is complementary or mismatched to position −3 of the polynucleotide template, wherein the fifth nucleobase of the capping primer from the 5′-end is complementary or mismatched to position −4 of the polynucleotide template, wherein the fourth nucleobase of the capping primer from the 5′-end is complementary or mismatched to position −5 of the polynucleotide template, wherein the third nucleobase of the capping primer from the 5′-end is complementary or mismatched to position −6 of the polynucleotide template, and wherein the second nucleobase of the capping primer from the 5′-end is complementary or mismatched to position −7 of the polynucleotide template. In some embodiments, the nucleobases of the capping primer are fully complementary to the polynucleotide template. In some embodiments, the capping primer comprises no more than one mismatch with respect to the polynucleotide template. In some embodiments, the capping primer comprises no more than two mismatches with respect to the polynucleotide template. In some embodiments, the capping primer comprises no more than three mismatches with respect to the polynucleotide template. In some embodiments, the capping primer comprises no more than four mismatches with respect to the polynucleotide template. In some embodiments, the capping primer comprises no more than five mismatches with respect to the polynucleotide template. In some embodiments, the capping primer comprises no more than six mismatches with respect to the polynucleotide template. In some embodiments, the capping primer comprises no more than seven mismatches with respect to the polynucleotide template.

In some embodiments, the capping primer is 11 nucleotides in length, wherein the eleventh nucleobase of the capping primer from the 5′-end is complimentary to position +2 of the polynucleotide template, wherein the tenth nucleobase of the capping primer from the 5′-end is complimentary to position +1 of the polynucleotide template, wherein the ninth nucleobase of the capping primer from the 5′-end is complementary or mismatched to position −1 of the polynucleotide template, wherein the eighth nucleobase of the capping primer from the 5′-end is complementary or mismatched to position −2 of the polynucleotide template, wherein the seventh nucleobase of the capping primer from the 5′-end is complementary or mismatched to position −3 of the polynucleotide template, wherein the sixth nucleobase of the capping primer from the 5′-end is complementary or mismatched to position −4 of the polynucleotide template, wherein the fifth nucleobase of the capping primer from the 5′-end is complementary or mismatched to position −5 of the polynucleotide template, wherein the fourth nucleobase of the capping primer from the 5′-end is complementary or mismatched to position −6 of the polynucleotide template, wherein the third nucleobase of the capping primer from the 5′-end is complementary or mismatched to position −7 of the polynucleotide template, and wherein the second nucleobase of the capping primer from the 5′-end is complementary or mismatched to position −8 of the polynucleotide template. In some embodiments, the nucleobases of the capping primer are fully complementary to the polynucleotide template. In some embodiments, the capping primer comprises no more than one mismatch with respect to the polynucleotide template. In some embodiments, the capping primer comprises no more than two mismatches with respect to the polynucleotide template. In some embodiments, the capping primer comprises no more than three mismatches with respect to the polynucleotide template. In some embodiments, the capping primer comprises no more than four mismatches with respect to the polynucleotide template. In some embodiments, the capping primer comprises no more than five mismatches with respect to the polynucleotide template. In some embodiments, the capping primer comprises no more than six mismatches with respect to the polynucleotide template. In some embodiments, the capping primer comprises no more than seven mismatches with respect to the polynucleotide template. In some embodiments, the capping primer comprises no more than eight mismatches with respect to the polynucleotide template.

In some embodiments, the polynucleotide template does not comprise a cytosine nucleobase at position +1 (i.e., at position +1 of the antisense strand). In some embodiments, the polynucleotide template comprises a thymine nucleobase, or a derivative or analog thereof, at position −1 (i.e., at position −1 of the antisense strand). In some embodiments, the polynucleotide template comprises an adenine nucleobase, or a derivative or analog thereof, at position −1 (i.e., at position −1 of the antisense strand). In some embodiments, the polynucleotide template comprises a thymine or an adenine nucleobase, or a derivative or analog thereof, at position +1 (i.e., at position +1 of the antisense strand). In some embodiments, the polynucleotide template comprises a thymine nucleobase, or a derivative or analog thereof, at position +1 (i.e., at position +1 of the antisense strand). In some embodiments, the polynucleotide template comprises an adenine nucleobase, or a derivative or analog thereof, at position +1 (i.e., at position +1 of the antisense strand). In some embodiments, the polynucleotide template comprises a guanine nucleobase, or a derivative or analog thereof, at position +1 (i.e., at position +1 of the antisense strand). In some embodiments, the polynucleotide template comprises a cytosine nucleobase, or a derivative or analog thereof, at position +2 (i.e., at position +2 of the antisense strand). In some embodiments, the polynucleotide template comprises a thymine nucleobase, or a derivative or analog thereof, at position +2 (i.e., at position +2 of the antisense strand). In some embodiments, the polynucleotide template comprises a guanine nucleobase, or a derivative or analog thereof, at position +2 (i.e., at position +2 of the antisense strand). In some embodiments, the polynucleotide template comprises an adenine nucleobase, or a derivative or analog thereof, at position +2 (i.e., at position +2 of the antisense strand).

### Exemplary Capping Primers

The present disclosure provides capping primers and compositions thereof. In some embodiments, the capping primers of the disclosure are designed for use in a transcription systems described herein for synthesis of 5′-capped RNA (e.g., 5′-capped mRNA).

In some embodiments, the capping primer comprises a compound comprising the structure according to Formula I:

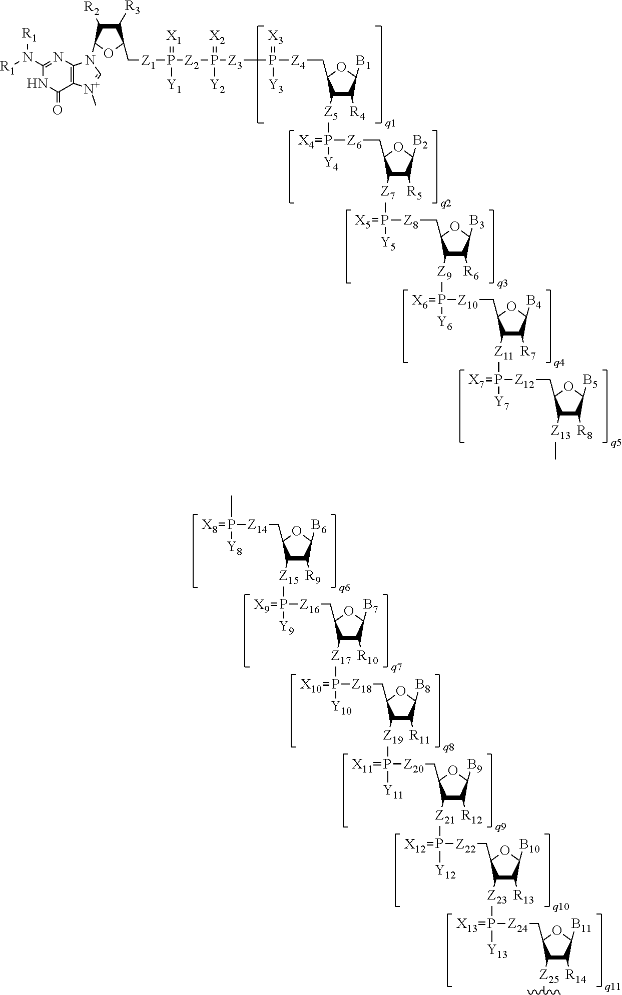

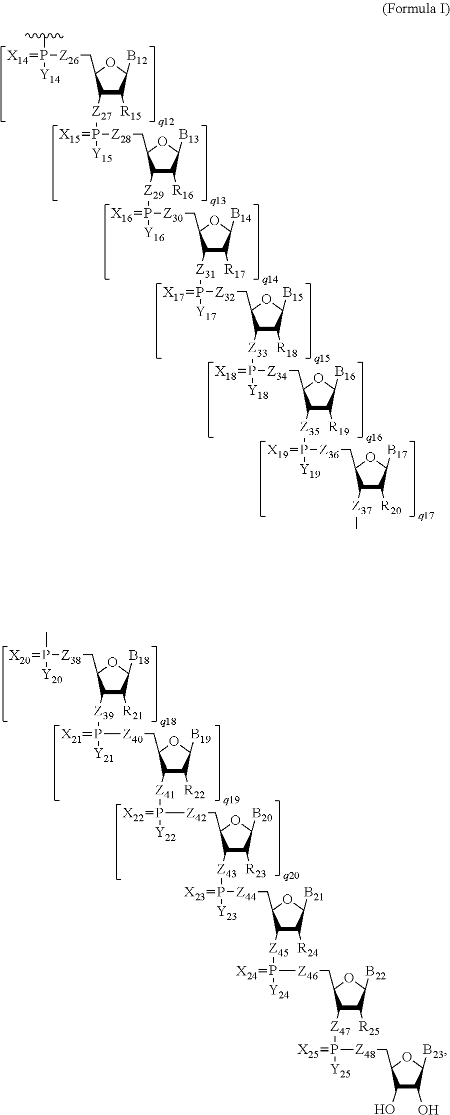

wherein q1 to q20, are each independently 0 or 1 (e.g., if q1=0, then it is absent from the capping primer, whereas if q1=1, then it is present in the capping primer); wherein B1 through B20, if present, are each a nucleobase independently selected from the group consisting of adenine, guanine, thymine, uracil, cytosine and any derivative or analog of the foregoing; wherein B21 is a nucleobase selected from the group consisting of adenine, guanine, thymine, uracil, cytosine and any derivative or analog of the foregoing; wherein B22 is a nucleobase selected from the group consisting of adenine, uracil, cytosine and any derivative or analog of the foregoing; wherein B23 is a nucleobase selected from the group consisting of adenine, guanine, uracil, cytosine and any derivative or analog of the foregoing; wherein R1 are each independently H, alkyl, acyl, benzyl, a cleavable unit; wherein R2 and R3 are each independently H, OH, alkyl, O-alkyl, halogen, LNA, a linker, or a detectable marker; wherein R4 to R25, if present, are each independently H, OH, alkyl, O-alkyl, halogen, LNA, a linker or a detectable marker; wherein X1 to X25, if present, are each independently O or S; wherein Y1 to Y25, if present, are each independently O⁻, S⁻, BH₃⁻, aryl, alkyl, O-alkyl or O-aryl; and wherein Z1 to Z48, if present, are each independently O, S, NH, CH₂, C(halogen)₂ or CH(halogen).

In some embodiments, the capping primer comprises a compound comprising the structure according to Formula I, wherein q1 to q20, are each independently 0 or 1 (e.g., if q1=0, then it is absent from the capping primer, whereas if q1=1, then it is present in the capping primer); wherein B1 through B20, if present, are each a nucleobase independently selected from the group consisting of adenine, guanine, thymine, uracil, cytosine, hypoxanthine, and any derivative or analog of the foregoing; wherein B21 is a nucleobase selected from the group consisting of adenine, guanine, thymine, uracil, cytosine, hypoxanthine, and any derivative or analog of the foregoing; wherein B22 is a nucleobase selected from the group consisting of adenine, uracil, cytosine and any derivative or analog of the foregoing; wherein B23 is a nucleobase selected from the group consisting of adenine, guanine, uracil, cytosine and any derivative or analog of the foregoing; wherein R1 are each independently H, alkyl, acyl, benzyl, or a cleavable unit; wherein R2 is H, OH, alkyl, O-alkyl, O-acyl, O-benzyl, O-methoxyethyl (MOE), halogen, LNA, a linker, a cleavable unit or a detectable marker; wherein R3 is H, OH, alkyl, O-alkyl, halogen, a linker, or a detectable marker; wherein R4 to R25, if present, are each independently H, OH, alkyl, O-alkyl, halogen, LNA, a linker or a detectable marker; wherein X1 to X25, if present, are each independently O or S; wherein Y1 to Y25, if present, are each independently O⁻, S⁻, BH₃⁻, aryl, alkyl, O-alkyl or O-aryl; and wherein Z1 to Z48, if present, are each independently O, S, NH, CH₂, C(halogen)₂ or CH(halogen). In some embodiments, the capping primer consists of the structure according to Formula I.

In some embodiments, the capping primer hybridizes to a polynucleotide template described herein, wherein the polynucleotide template comprises a nucleobase at position −1 immediately adjacent to and downstream (3′) of a transcriptional start site and a nucleobase at each of position +1, position +2 and position +3 immediately adjacent to and upstream (5′) of the transcriptional start site, and wherein the capping primer comprises a nucleobase complementary to the nucleobase at position +1 and position +2 of the polynucleotide template (each referring to a position of the antisense strand of the polynucleotide template). In some embodiments, B21 is complementary or mismatched to a nucleobase at positions −1 of the polynucleotide template. In some embodiments, B21 is complementary to a nucleobase at positions −1 of the polynucleotide template. In some embodiments, B21 is mismatched to a nucleobase at positions −1 of the polynucleotide template. In some embodiments, B22 is complementary to a nucleobase at positions +1 of the polynucleotide template. In some embodiments, B23 is complementary to a nucleobase at positions +2 of the polynucleotide template. In some embodiments, B21, B22, and B23 are not fully complementary to nucleobases at positions +1 to +3 of the polynucleotide template.

In some embodiments, the nucleobase at position +1 of the polynucleotide template is not cytosine. In some embodiments, the nucleobase at position −1 of the polynucleotide template comprises thymine, or a derivative or analog thereof. In some embodiments, the nucleobase at position −1 of the polynucleotide template comprises adenine, or a derivative or analog thereof. In some embodiments, the nucleobase at position +1 of the polynucleotide template comprises thymine, adenine, or a derivative or analog thereof. In some embodiments, the nucleobase at position +1 of the polynucleotide template comprises thymine, or a derivative or analog thereof. In some embodiments, the nucleobase at position +1 of the polynucleotide template comprises adenine, or a derivative or analog thereof. In some embodiments, the nucleobase at position +1 of the polynucleotide template comprises guanine, or a derivative or analog thereof. In some embodiments, the nucleobase at position +2 of the polynucleotide template comprises a cytosine nucleobase, or a derivative or analog thereof. In some embodiments, the nucleobase at position +2 of the polynucleotide template comprises a thymine nucleobase, or a derivative or analog thereof. In some embodiments, the nucleobase at position +2 of the polynucleotide template comprises a guanine nucleobase, or a derivative or analog thereof. In some embodiments, the nucleobase at position +2 of the polynucleotide template comprises an adenine nucleobase, or a derivative or analog thereof.

In some embodiments, B21 is complementary to the nucleobase at position −1 of the polynucleotide template; B22 is complementary to the nucleobase at position +1 of the polynucleotide template; and B23 is complementary to the nucleobase at positions +2 of the polynucleotide template. In some embodiments, B21 is mismatched to the nucleobase at position −1 of the polynucleotide template; B22 is complementary to the nucleobase at position +1 of the polynucleotide template; and B23 is complementary to the nucleobase at positions +2 of the polynucleotide template. In some embodiments, the nucleobase at position +1 of the polynucleotide template comprises thymine, or a derivative or analog thereof; and B22 comprises adenine, or a derivative or analog thereof. In some embodiments, the nucleobase at position +2 of the polynucleotide template comprises a cytosine nucleobase, or a derivative or analog thereof, and B23 comprises guanine, or a derivative or analog thereof.

In some embodiments, the nucleobase at position −1 of the polynucleotide template comprises thymine, or a derivative or analog thereof. In some embodiments, the nucleobase at position −1 of the polynucleotide template comprises adenine, or a derivative or analog thereof. In some embodiments, B21 is any nucleobase that is not N6-modified adenine (e.g. 6-methyladenine). In some embodiments, B21 is a nucleobase selected from the group consisting of adenine, uracil, thymine, cytosine, 5-methylcytosine, guanine, and hypoxanthine. In some embodiments, B21 is adenine or a derivative or analog thereof. In some embodiments, B21 is uracil or a derivative or analog thereof. In some embodiments, B21 comprises adenine or N6-methyladenosine and the nucleobase at position −1 of the polynucleotide template comprises thymine, or a derivative or analog thereof. In some embodiments, B21 comprises uracil and the nucleobase at position −1 of the polynucleotide template comprises thymine, or a derivative or analog thereof.

In some embodiments, B22 is selected from the group consisting of adenine, cytosine, 5-methylcytosine, thymine and uracil. In some embodiment, B22 is selected from the group consisting of adenine, thymine and uracil. In some embodiments, B23 is selected from the group consisting of adenine, guanine, cytosine, and uracil. In some embodiments, B22 is adenine, or a derivative or analog thereof, and B23 is guanine, or a derivative or analog thereof. In some embodiments, B22 is cytosine, or a derivative or analog thereof; and B23 is guanine, or a derivative or analog thereof. In some embodiments, B22 is uracil, or a derivative or analog thereof; and B23 is guanine, or a derivative or analog thereof. In some embodiments, B22 is adenine, or a derivative or analog thereof, and B23 is adenine, or a derivative or analog thereof. In some embodiments, B22 is adenine, or a derivative or analog thereof; and B23 is cytosine, or a derivative or analog thereof. In some embodiments, B22 is adenine, or a derivative or analog thereof; and B23 is uracil, or a derivative or analog thereof.

In some embodiments, B22 comprises adenine and the nucleobase at position +1 of the polynucleotide template comprises thymine, or a derivative or analog thereof. In some embodiments, B23 comprises guanine and the nucleobase at position +2 of the polynucleotide template comprises cytosine, or a derivative or analog thereof.

In some embodiments, R1 is H, alkyl, acyl, benzyl or a cleavable unit. In some embodiments, R1 is H. In some embodiments, R1 is a C1-C6 alkyl. In some embodiments, R1 is Me. In some embodiments, R2 is H, OH, alkyl, O-alkyl, O-acyl, O-benzyl, halogen, LNA, a linker, a cleavable unit or a detectable marker. In some embodiments, R2 is H, OH, O—(C1-C6 alkyl), O-MOE, fluoro, or LNA. In some embodiments, R2 is OH. In some embodiments, R3 is H, OH, alkyl, O-alkyl, O-acyl, O-benzyl, halogen, a linker, a cleavable unit or a detectable marker. In some embodiments, R3 is H, OH, O—(C1-C6 alkyl), O-MOE, or fluoro. In some embodiments R3 is OH. In some embodiments, R3 is OMe.

In some embodiments, q1 to q20 are 0; and R24 and R25 are each independently OH or O-alkyl. In some embodiments, R24 is OH or OMe and R25 is OH or OMe. In some embodiments, R24 is OMe and R25 is OH or OMe. In some embodiments, R24 is OMe and R25 is OMe. In some embodiments, q1 to q20 are 0; and R24 and R25 are each independently H, OH, O—(C1-C6 alkyl), O-MOE, fluoro, or LNA. In some embodiments, R24 is H, OH, OMe, O-MOE, or fluoro and R25 is OH or OMe. In some embodiments, Z1 to Z3 and Z44 to Z48 are each O. In some embodiments, X1, X2, and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 is 1; q2 to q20 are 0; and R4, R24, and R25 are each independently OH or O-alkyl. In some embodiments, R4 is OMe, R24 is OH or OMe, and R25 is OH or OMe. In some embodiments, R4 is OMe, R24 is OMe, and R25 is OH or OMe. In some embodiments, R4 is OMe, R24 is OMe, and R25 is OMe. In some embodiments, q1 is 1; q2 to q20 are 0; and R4, R24, and R25 are each independently H, OH, O—(C1-C6 alkyl), O-MOE, fluoro, or LNA. In some embodiments, Z1 to Z5 and Z44 to Z48 are each O. In some embodiments, X1 to X3 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q2 are 1; q3 to q20 are 0; and R4, R5, R24, and R25 are each independently OH or O-alkyl. In some embodiments, R4 is OMe, R5 is OH or OMe, R24 is OH or OMe, and R25 is OH or OMe. In some embodiments, R4 is OMe and R5, R24, and R25 are each OH. In some embodiments, R4 is OMe, R5 is OMe, R24 is OH or OMe, and R25 is OH or OMe. In some embodiments, R4 is OMe, R5 is OMe, and R24 and R25 are each OH. In some embodiments, R4 is OMe, R5 is OMe, and R24 and R25 are each OMe. In some embodiments, q1 to q2 are 1; q3 to q20 are 0; and R4, R5, R24, and R25 are each independently H, OH, O—(C1-C6 alkyl), O-MOE, fluoro, or LNA. In some embodiments, Z1 to Z7 and Z44 to Z48 are each O. In some embodiments, X1 to X4 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q3 are 1; q4 to q20 are 0; and R4 to R6, R24, and R25 are each independently OH or O-alkyl. In some embodiments, R4 is OMe, R5 is OH or OMe, R6 is OH or OMe, R24 is OH or OMe, and R25 is OH or OMe. In some embodiments, R4 is OMe and R5, R6, R24, and R25 are each OH. In some embodiments, R4 is OMe, R5 is OMe, R6 is OH or OMe, R24 is OH or OMe, and R25 is OH or OMe. In some embodiments, R4 is OMe, R5 is OMe and R6, R24, and R25 are each OH. In some embodiments, R4, R5 R6, R24, and R25 are each OMe. In some embodiments, q1 to q3 are 1; q4 to q20 are 0; and R4 to R6, R24, and R25 are each independently H, OH, O—(C1-C6 alkyl), O-MOE, fluoro, or LNA. In some embodiments, Z1 to Z9 and Z44 to Z48 are each O. In some embodiments, X1 to X5 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q4 are 1; q5 to q20 are 0; and R4 to R7, R24, and R25 are each independently OH or O-alkyl. In some embodiments, R4 is OMe, R5 is OH or OMe, R6 is OH or OMe, R7 is OH or OMe, R24 is OH or OMe, and R25 is OH or OMe. In some embodiments, R4 is OMe and R5 to R7, R24, and R25 are each OH. In some embodiments, R4 is OMe, R5 is OMe, R6 is OH or OMe, R7 is OH or OMe, R24 is OH or OMe, and R25 is OH or OMe. In some embodiments, R4 is OMe, R5 is OMe, and R6, R7, R24, and R25 are each OH. In some embodiments, R4, R5, R6, R7, R24, and R25 are each OMe. In some embodiments, q1 to q4 are 1; q5 to q20 are 0; and R4 to R7, R24, and R25 are each independently H, OH, O—(C1-C6 alkyl), O-MOE, fluoro, or LNA. In some embodiments, Z1 to Z11 and Z44 to Z48 are each O. In some embodiments, X1 to X6 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q5 are 1; q6 to q20 are 0; and R4 to R8, R24, and R25 are each independently OH or O-alkyl. In some embodiments, R4 is OMe, R5 is OH or OMe, R6 is OH or OMe, R7 is OH or OMe, R8 is OH or OMe, R24 is OH or OMe, and R25 is OH or OMe. In some embodiments, R4 is OMe and R5 to R8, R24, and R25 are each OH. In some embodiments, R4 is OMe, R5 is OMe, R6 is OH or OMe, R7 is OH or OMe, R8 is OH or OMe, R24 is OH or OMe, and R25 is OH or OMe. In some embodiments, R4 is OMe, R5 is OMe, and R6 to R8, R24, and R25 are each OH. In some embodiments, R4, R5, R6, R7, R8, R24, and R25 are each OMe. In some embodiments, q1 to q5 are 1; q6 to q20 are 0; and R4 to R8, R24, and R25 are each independently H, OH, O—(C1-C6 alkyl), O-MOE, fluoro, or LNA. In some embodiments, Z1 to Z13 and Z44 to Z48 are each O. In some embodiments, X1 to X7 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q6 are 1; q7 to q20 is 0; and R4 to R9, R24, and R25 are each independently OH or O-alkyl. In some embodiments, R4 is OMe, R5 is OH or OMe, R6 is OH or OMe, R7 is OH or OMe, R8 is OH or OMe, R9 is OH or OMe, R24 is OH or OMe, and R25 is OH or OMe. In some embodiments, R4 is OMe and R5 to R9, R24, and R25 are each OH. In some embodiments, R4 is OMe, R5 is OMe, R6 is OH or OMe, R7 is OH or OMe, R8 is OH or OMe, R9 is OH or OMe, R24 is OH or OMe, and R25 is OH or OMe. In some embodiments, R4 is OMe, R5 is OMe, and R6 to R9, R24, and R25 are each OH. In some embodiments, R4, R5, R6, R7, R8, R9, R24, and R25 are each OMe. In some embodiments, q1 to q6 are 1; q7 to q20 is 0; and R4 to R9, R24, and R25 are each independently H, OH, O—(C1-C6 alkyl), O-MOE, fluoro, or LNA. In some embodiments, Z1 to Z15 and Z44 to Z48 are each O. In some embodiments, X1 to X8 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q7 are 1; q8 to q20 are 0; and R4 to R10, R24, and R25 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R24 is OH or OMe, and R25 is OH or OMe. In some embodiments, R4 is OMe and R5 to R10, R24, and R25 are each OH. In some embodiments, R4 is OMe, R5 is OMe, R6 is OH or OMe, R7 is OH or OMe, R8 is OH or OMe, R9 is OH or OMe, R10 is OH or OMe, R24 is OH or OMe, and R25 is OH or OMe. In some embodiments, R4 is OMe, R5 is OMe, and R6 to R10, R24, and R25 are each OH. In some embodiments, R4, R5, R6, R7, R8, R9, R10, R24, and R25 are each OMe. In some embodiments, q1 to q7 are 1; q8 to q20 are 0; and R4 to R10, R24, and R25 are each independently H, OH, O—(C1-C6 alkyl), O-MOE, fluoro, or LNA. In some embodiments, Z1 to Z17 and Z44 to Z48 are each O. In some embodiments, X1 to X9 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q8 are 1; q9 to q20 are 0; and R4 to R11, R24, and R25 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R11 is OH or OMe, R24 is OH or OMe, and R25 is OH or OMe. In some embodiments, R4 is OMe and R5 to R11, R24, and R25 are each OH. In some embodiments, R4 is OMe, R5 is OMe, R6 is OH or OMe, R7 is OH or OMe, R8 is OH or OMe, R9 is OH or OMe, R10 is OH or OMe, R11 is OH or OMe, R24 is OH or OMe, and R25 is OH or OMe. In some embodiments, R4 is OMe, R5 is OMe, and R6 to R11, R24, and R25 are each OH. In some embodiments, R4, R5, R6, R7, R8, R9, R10, R11, R24, and R25 are each OMe. In some embodiments, q1 to q8 are 1; q9 to q20 are 0; and R4 to R11, R24, and R25 are each independently H, OH, O—(C1-C6 alkyl), O-MOE, fluoro, or LNA. In some embodiments, Z1 to Z19 and Z44 to Z48 are each O. In some embodiments, X1 to X10 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q9 are 1; q10 to q20 are 0; and R4 to R12, R24, and R25 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R11 is OH or OMe, R12 is OH or OMe, R24 is OH or OMe, and R25 is OH or OMe. In some embodiments, R4 is OMe and R5 to R12, R24, and R25 are each OH. In some embodiments, R4 is OMe, R5 is OMe, R6 is OH or OMe, R7 is OH or OMe, R8 is OH or OMe, R9 is OH or OMe, R10 is OH or OMe, R11 is OH or OMe, R12 is OH or OMe, R24 is OH or OMe, and R25 is OH or OMe. In some embodiments, R4 is OMe, R5 is OMe, and R6 to R12, R24, and R25 are each OH. In some embodiments, R4, R5, R6, R7, R8, R9, R10, R11, R12, R24, and R25 are each OMe. In some embodiments, q1 to q9 are 1; q10 to q20 are 0; and R4 to R12, R24, and R25 are each independently H, OH, O—(C1-C6 alkyl), O-MOE, fluoro, or LNA. In some embodiments, Z1 to Z21 and Z44 to Z48 are each O. In some embodiments, X1 to X11 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q10 are 1; q11 to q20 are 0; and R4 to R13, R24, and R25 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R24 is OH or OMe, and R25 is OH or OMe. In some embodiments, R4 is OMe and R5 to R13, R24, and R25 are each OH. In some embodiments, R4 is OMe, R5 is OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R24 is OH or OMe, and R25 is OH or OMe. In some embodiments, R4 is OMe, R5 is OMe, and R6 to R13, R24, and R25 are each OH. In some embodiments, R4, R5, R6, R7, R8, R9, R10, R11, R12, R13, R24, and R25 are each OMe. In some embodiments, q1 to q10 are 1; q11 to q20 are 0; and R4 to R13, R24, and R25 are each independently H, OH, O—(C1-C6 alkyl), O-MOE, fluoro, or LNA. In some embodiments, Z1 to Z23 and Z44 to Z48 are each O. In some embodiments, X1 to X12 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q11 are 1; q12 to q20 are 0; and R4 to R14, R24, and R25 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R24 is OH or OMe, and R25 is OH or OMe. In some embodiments, R4 is OMe and R5 to R14, R24, and R25 are each OH. In some embodiments, R4 is OMe, R5 is OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R24 is OH or OMe, and R25 is OH or OMe. In some embodiments, R4 is OMe, R5 is OMe, and R6 to R14, R24, and R25 are each OH. In some embodiments, R4, R5, R6, R7, R8, R9, R10, R11, R12, R13, R14, R24, and R25 are each OMe. In some embodiments, q1 to q11 are 1; q12 to q20 are 0; and R4 to R14, R24, and R25 are each independently H, OH, O—(C1-C6 alkyl), O-MOE, fluoro, or LNA. In some embodiments, Z1 to Z25 and Z44 to Z48 are each O. In some embodiments, X1 to X13 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q12 are 1; q13 to q20 are 0; and R4 to R15, R24, and R25 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R24 is OH or OMe, and R25 is OH or OMe. In some embodiments, R4 is OMe and R5 to R15, R24, and R25 are each OH. In some embodiments, R4 is OMe, R5 is OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R24 is OH or OMe, and R25 is OH or OMe. In some embodiments, R4 is OMe, R5 is OMe, and R6 to R15, R24, and R25 are each OH. In some embodiments, R4, R5, R6, R7, R8, R9, R10, R11, R12, R13, R14, R15, R24, and R25 are each OMe. In some embodiments, q1 to q12 are 1; q13 to q20 are 0; and R4 to R15, R24, and R25 are each independently H, OH, O—(C1-C6 alkyl), O-MOE, fluoro, or LNA. In some embodiments, Z1 to Z27 and Z44 to Z48 are each O. In some embodiments, X1 to X14 and X23 to X25 are each 0. In some embodiments, Y1, Y2, and Y23 to Y25 are each independently O⁻ or S⁻. In some embodiments, Y1, Y2, and Y23 to Y25 are each O⁻.

In some embodiments, q1 to q13 are 1; q14 to q20 are 0; and R4 to R16, R24, and R25 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R24 is OH or OMe, and R25 is OH or OMe. In some embodiments, R4 is OMe and R5 to R16, R24, and R25 are each OH. In some embodiments, R4 is OMe, R5 is OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R24 is OH or OMe, and R25 is OH or OMe. In some embodiments, R4 is OMe, R5 is OMe, and R6 to R16, R24, and R25 are each OH. In some embodiments, R4, R5, R6, R7, R8, R9, R10, R11, R12, R13, R14, R15, R16, R24, and R25 are each OMe. In some embodiments, q1 to q13 are 1; q14 to q20 are 0; and R4 to R16, R24, and R25 are each independently H, OH, O—(C1-C6 alkyl), O-MOE, fluoro, or LNA. In some embodiments, Z1 to Z29 and Z44 to Z48 are each O. In some embodiments, X1 to X15 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q14 are 1; q15 to q20 are 0; and R4 to R17, R24, and R25 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R17 is OH or OMe, R24 is OH or OMe, and R25 is OH or OMe. In some embodiments, R4 is OMe and R5 to R17, R24, and R25 are each OH. In some embodiments, R4 is OMe, R5 is OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R17 is OH or OMe, R24 is OH or OMe, and R25 is OH or OMe. In some embodiments, R4, R5, R6, R7, R8, R9, R10, R11, R12, R13, R14, R15, R16, R17, R24, and R25 are each OMe. In some embodiments, R4 is OMe, R5 is OMe, and R6 to R17, R24, and R25 are each OMe. In some embodiments, q1 to q14 are 1; q15 to q20 are 0; and R4 to R17, R24, and R25 are each independently H, OH, O—(C1-C6 alkyl), O-MOE, fluoro, or LNA. In some embodiments, Z1 to Z31 and Z44 to Z48 are each O. In some embodiments, X1 to X16 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q15 are 1; q16 to q20 are 0; and R4 to R18, R24, and R25 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R17 is OH or OMe, R18 is OH or OMe, R24 is OH or OMe, and R25 is OH or OMe. In some embodiments, R4 is OMe and R5 to R18, R24, and R25 are each OH. In some embodiments, R4 is OMe, R5 is OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R17 is OH or OMe, R18 is OH or OMe, R24 is OH or OMe, and R25 is OH or OMe. In some embodiments, R4 is OMe, R5 is OMe, and R6 to R18, R24, and R25 are each OH. In some embodiments, R4, R5, R6, R7, R8, R9, R10, R11, R12, R13, R14, R15, R16, R17, R18, R24, and R25 are each OMe. In some embodiments, q1 to q15 are 1; q16 to q20 are 0; and R4 to R18, R24, and R25 are each independently H, OH, O—(C1-C6 alkyl), O-MOE, fluoro, or LNA. In some embodiments, Z1 to Z33 and Z44 to Z48 are each O. In some embodiments, X1 to X17 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q16 are 1; q17 to q20 are 0; and R4 to R19, R24, and R25 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R17 is OH or OMe, R18 is OH or OMe, R19 is OH or OMe, R24 is OH or OMe, and R25 is OH or OMe. In some embodiments, R4 is OMe and R5 to R19, R24, and R25 are each OH. In some embodiments, R4 is OMe, R5 is OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R17 is OH or OMe, R18 is OH or OMe, R19 is OH or OMe, R24 is OH or OMe, and R25 is OH or OMe. In some embodiments, R4 is OMe, R5 is OMe, and R6 to R19, R24, and R25 are each OH. In some embodiments, R4, R5, R6, R7, R8, R9, R10, R11, R12, R13, R14, R15, R16, R17, R18, R19, R24, and R25 are each OMe. In some embodiments, q1 to q16 are 1; q17 to q20 are 0; and R4 to R19, R24, and R25 are each independently H, OH, O—(C1-C6 alkyl), O-MOE, fluoro, or LNA. In some embodiments, Z1 to Z35 and Z44 to Z48 are each O. In some embodiments, X1 to X18 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q17 are 1; q18 to q20 are 0; and R4 to R20, R24, and R25 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R17 is OH or OMe, R18 is OH or OMe, R19 is OH or OMe, R20 is OH or OMe, R24 is OH or OMe, and R25 is OH or OMe. In some embodiments, R4 is OMe and R5 to R20, R20, R24, and R25 are each OH. In some embodiments, R4 is OMe, R5 is OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R17 is OH or OMe, R18 is OH or OMe, R19 is OH or OMe, R20 is OH or OMe, R24 is OH or OMe, and R25 is OH or OMe. In some embodiments, R4 is OMe, R5 is OMe, and R6 to R20, R24, and R25 are each OH. In some embodiments, R4, R5, R6, R7, R8, R9, R10, R11, R12, R13, R14, R15, R16, R17, R18, R19, R20, R24, and R25 are each OMe. In some embodiments, q1 to q17 are 1; q18 to q20 are 0; and R4 to R20, R24, and R25 are each independently H, OH, O—(C1-C6 alkyl), O-MOE, fluoro, or LNA. In some embodiments, Z1 to Z37 and Z44 to Z48 are each O. In some embodiments, X1 to X19 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q18 are 1; q19 to q20 are 0; and R4 to R21, R24, and R25 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R17 is OH or OMe, R18 is OH or OMe, R19 is OH or OMe, R20 is OH or OMe, R21 is OH or OMe, R24 is OH or OMe, and R25 is OH or OMe. In some embodiments, R4 is OMe and R5 to R21, R20, R24, and R25 are each OH. In some embodiments, R4 is OMe, R5 is OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R17 is OH or OMe, R18 is OH or OMe, R19 is OH or OMe, R20 is OH or OMe, R21 is OH or OMe, R24 is OH or OMe, and R25 is OH or OMe. In some embodiments, R4 is OMe, R5 is OMe, and R6 to R21, R24, and R25 are each OH. In some embodiments, R4, R5, R6, R7, R8, R9, R10, R11, R12, R13, R14, R15, R16, R17, R18, R19, R20, R21, R24, and R25 are each OMe. In some embodiments, q1 to q18 are 1; q19 to q20 are 0; and R4 to R21, R24, and R25 are each independently H, OH, O—(C1-C6 alkyl), O-MOE, fluoro, or LNA. In some embodiments, Z1 to Z39 and Z44 to Z48 are each O. In some embodiments, X1 to X20 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q19 are 1; q20 is 0; and R4 to R22, R24, and R25 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R17 is OH or OMe, R18 is OH or OMe, R19 is OH or OMe, R20 is OH or OMe, R21 is OH or OMe, R22 is OH or OMe, R24 is OH or OMe, and R25 is OH or OMe. In some embodiments, R4 is OMe and R5 to R22, R24, and R25 are each OH. In some embodiments, R4 is OMe, R5 is OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R17 is OH or OMe, R18 is OH or OMe, R19 is OH or OMe, R20 is OH or OMe, R21 is OH or OMe, R22 is OH or OMe, R24 is OH or OMe, and R25 is OH or OMe. In some embodiments, R4 is OMe, R5 is OMe, and R6 to R22, R24, and R25 are each OH. In some embodiments, R4, R5, R6, R7, R8, R9, R10, R11, R12, R13, R14, R15, R16, R17, R18, R19, R20, R21, R22, R24, and R25 are each OMe. In some embodiments, q1 to q19 are 1; q20 is 0; and R4 to R22, R24, and R25 are each independently H, OH, O—(C1-C6 alkyl), O-MOE, fluoro, or LNA. In some embodiments, Z1 to Z41 and Z44 to Z48 are each O. In some embodiments, X1 to X21 and X23 to X25 are each O. In some embodiments, Y1, Y2, and Y23 to Y25 are each independently O⁻ or S⁻. In some embodiments, Y1, Y2, and Y23 to Y25 are each O⁻.

In some embodiments, q1 to q20 are 1; and R4 to R25 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R17 is OH or OMe, R18 is OH or OMe, R19 is OH or OMe, R20 is OH or OMe, R21 is OH or OMe, R22 is OH or OMe, R23 is OH or OMe, R24 is OH or OMe, and R25 is OH or OMe. In some embodiments, R4 is OMe and R5 to R25 are each OH. In some embodiments, R4 is OMe, R5 is OMe, R6 is OH or OMe, R7 is OH or OMe, R8 is OH or OMe, R9 is OH or OMe, R10 is OH or OMe, R11 is OH or OMe, R12 is OH or OMe, R13 is OH or OMe, R14 is OH or OMe, R15 is OH or OMe, R16 is OH or OMe, R17 is OH or OMe, R18 is OH or OMe, R19 is OH or OMe, R20 is OH or OMe, R21 is OH or OMe, R22 is OH or OMe, R23 is OH or OMe, R24 is OH or OMe, and R25 is OH or OMe. In some embodiments, R4 is OMe, R5 is OMe, and R6 to R25 are each OH. In some embodiments, R4 to R25 are each OMe. In some embodiments, q1 to q20 are 1; and R4 to R25 are each independently H, OH, O—(C1-C6 alkyl), O-MOE, fluoro, or LNA. In some embodiments, Z1 to Z48 are each O. In some embodiments, X1 to X25 are each O. In some embodiments, Y1, Y2, and Y23 to Y25 are each independently O⁻ or S⁻. In some embodiments, Y1, Y2, and Y23 to Y25 are each O⁻.

In some embodiments, the capping primer comprises a compound comprising a structure according to Formula II:

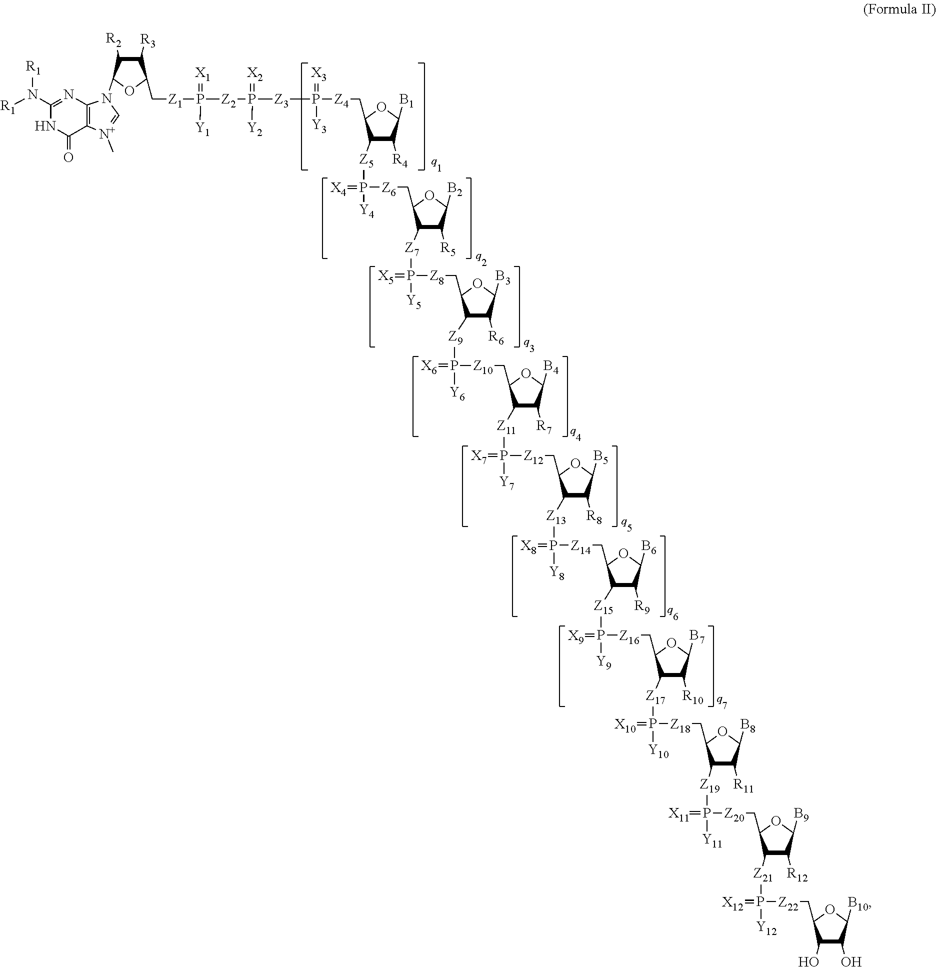

wherein q1 to q7, are each independently 0 or 1 (e.g., if q1=0, then it is absent from the capping primer, whereas if q1=1, then it is present in the capping primer); wherein B1 through B7, if present, are each a nucleobase independently selected from the group consisting of adenine, guanine, thymine, uracil, cytosine and any derivative or analog of the foregoing; wherein B8 is a nucleobase selected from the group consisting of adenine, guanine, thymine, uracil, cytosine and any derivative or analog of the foregoing; wherein B9 is selected from the group consisting of adenine, uracil, cytosine and any derivative or analog of the foregoing; wherein B10 is selected from the group consisting of adenine, guanine, uracil, cytosine and any derivative or analog of the foregoing; wherein R1 are each independently H, alkyl, acyl, benzyl or a cleavable unit; wherein R2 and R3 are each independently H, OH, alkyl, O-alkyl, O-acyl, O-benzyl, halogen, LNA, a linker, a cleavable unit or a detectable marker; wherein R4 to R12, if present, are each independently H, OH, alkyl, O-alkyl, halogen, LNA, a linker or a detectable marker; wherein X1 to X12, if present, are each independently O or S; wherein Y1 to Y12, if present, are each independently O⁻, S⁻, BH₃⁻, aryl, alkyl, O-alkyl or O-aryl; and wherein Z1 to Z22, if present, are each independently O, S, NH, CH₂, C(halogen)₂ or CH(halogen).

In some embodiments, the capping primer comprises a compound comprising a structure according to Formula II, wherein q1 to q7, are each independently 0 or 1 (e.g., if q1=0, then it is absent from the capping primer, whereas if q1=1, then it is present in the capping primer); wherein B1 through B7, if present, are each a nucleobase independently selected from the group consisting of adenine, guanine, thymine, uracil, hypoxanthine, cytosine and any derivative or analog of the foregoing; wherein B8 is a nucleobase selected from the group consisting of adenine, guanine, thymine, uracil, hypoxanthine, cytosine and any derivative or analog of the foregoing; wherein B9 is selected from the group consisting of adenine, uracil, cytosine and any derivative or analog of the foregoing; wherein B10 is selected from the group consisting of adenine, guanine, uracil, cytosine and any derivative or analog of the foregoing; wherein R1 are each independently H, alkyl, acyl, benzyl or a cleavable unit; wherein R2 is H, OH, alkyl, O-alkyl, O-acyl, O-benzyl, O-methoxyethyl (MOE), halogen, LNA, a linker, a cleavable unit or a detectable marker; wherein R3 is H, OH, alkyl, O-alkyl, halogen, LNA, a linker, or a detectable marker; wherein R4 to R12, if present, are each independently H, OH, alkyl, O-alkyl, halogen, LNA, a linker or a detectable marker; wherein X1 to X12, if present, are each independently O or S; wherein Y1 to Y12, if present, are each independently O⁻, S⁻, BH₃⁻, aryl, alkyl, O-alkyl or O-aryl; and wherein Z1 to Z22, if present, are each independently O, S, NH, CH₂, C(halogen)₂ or CH(halogen).

In some embodiments, the capping primer hybridizes to a polynucleotide template described herein, wherein the polynucleotide template comprises a nucleobase at position −1 immediately adjacent to and downstream (3′) of a transcriptional start site and a nucleobase at each of position +1, position +2 and position +3 immediately adjacent to and upstream (5′) of the transcriptional start site, and wherein the capping primer comprises a nucleobase complementary to the nucleobase at position +1 and position +2 of the polynucleotide template (each referring to a position of the antisense strand of the polynucleotide template). In some embodiments, B8 is complementary or mismatched to a nucleobase at position −1 of the polynucleotide template. In some embodiments, B8 is complementary to a nucleobase at position −1 of the polynucleotide template. In some embodiments, B8 is mismatched to a nucleobase at position −1 of the polynucleotide template. In some embodiments, B9 is complementary to a nucleobase at position +1 of the polynucleotide template. In some embodiments, B10 is complementary to a nucleobase at position +2 of the polynucleotide template. In some embodiments, B8, B9, and B10 are not fully complementary to nucleobases at positions +1 to +3 of the polynucleotide template.

In some embodiments, the nucleobase at position +1 of the polynucleotide template is not cytosine. In some embodiments, the nucleobase at position −1 of the polynucleotide template comprises thymine, or a derivative or analog thereof. In some embodiments, the nucleobase at position −1 of the polynucleotide template comprises adenine, or a derivative or analog thereof. In some embodiments, the nucleobase at position −1 of the polynucleotide template comprises adenine or thymine. In some embodiments, the nucleobase at position +1 of the polynucleotide template comprises thymine, or a derivative or analog thereof. In some embodiments, the nucleobase at position +1 of the polynucleotide template comprises adenine, or a derivative or analog thereof. In some embodiments, the nucleobase at position +1 of the polynucleotide template comprises guanine, or a derivative or analog thereof. In some embodiments, the nucleobase at position +1 of the polynucleotide template comprises thymine, guanine or adenine. In some embodiments, the nucleobase at position +1 of the polynucleotide template comprises thymine or adenine. In some embodiments, the nucleobase at position +2 of the polynucleotide template comprises a cytosine nucleobase, or a derivative or analog thereof. In some embodiments, the nucleobase at position +2 of the polynucleotide template comprises a thymine nucleobase, or a derivative or analog thereof. In some embodiments, the nucleobase at position +2 of the polynucleotide template comprises a guanine nucleobase, or a derivative or analog thereof. In some embodiments, the nucleobase at position +2 of the polynucleotide template comprises an adenine nucleobase, or a derivative or analog thereof. In some embodiments, the nucleobase at position +2 of the polynucleotide template comprises thymine, guanine, cytosine or adenine.

In some embodiments, B8 is complementary to the nucleobase at position −1 of the polynucleotide template; B9 is complementary to the nucleobase at position +1 of the polynucleotide template; and B10 is complementary to the nucleobase at positions +2 of the polynucleotide template. In some embodiments, B8 is mismatched to the nucleobase at position −1 of the polynucleotide template; B9 is complementary to the nucleobase at position +1 of the polynucleotide template; and B10 is complementary to the nucleobase at positions +2 of the polynucleotide template. In some embodiments, the nucleobase at position +1 of the polynucleotide template comprises thymine, or a derivative or analog thereof; and B9 comprises adenine, or a derivative or analog thereof. In some embodiments, the nucleobase at position +2 of the polynucleotide template comprises a cytosine nucleobase, or a derivative or analog thereof; and B10 comprises guanine, or a derivative or analog thereof.

In some embodiments, the nucleobase at position −1 of the polynucleotide template comprises thymine, or a derivative or analog thereof. In some embodiments, the nucleobase at position −1 of the polynucleotide template comprises adenine, or a derivative or analog thereof. In some embodiments, B8 is any nucleobase that is not N6-modified adenine (e.g. 6-methyladenine). In some embodiments, B8 is a nucleobase selected from the group consisting of adenine, uracil, thymine, cytosine, 5-methylcytosine, guanine, and hypoxanthine. In some embodiments, B8 is adenine or a derivative or analog thereof. In some embodiments, B8 is uracil or a derivative or analog thereof. In some embodiments, B8 comprises adenine or N6-methyladenosine and the nucleobase at position −1 of the polynucleotide template comprises thymine, or a derivative or analog thereof. In some embodiments, B8 comprises uracil and the nucleobase at position −1 of the polynucleotide template comprises thymine, or a derivative or analog thereof.

In some embodiments, B9 is selected from the group consisting of adenine, cytosine, and uracil. In some embodiments, B9 is selected from the group consisting of adenine and uracil. In some embodiments, B10 is selected from the group consisting of adenine, guanine, cytosine, and uracil. In some embodiments, B9 is adenine, or a derivative or analog thereof; and B10 is guanine, or a derivative or analog thereof. In some embodiments, B9 is cytosine, or a derivative or analog thereof, and B10 is guanine, or a derivative or analog thereof. In some embodiments, B9 is uracil, or a derivative or analog thereof; and B10 is guanine, or a derivative or analog thereof. In some embodiments, B9 is adenine, or a derivative or analog thereof; and B10 is adenine, or a derivative or analog thereof. In some embodiments, B9 is adenine, or a derivative or analog thereof; and B10 is cytosine, or a derivative or analog thereof. In some embodiments, B9 is adenine, or a derivative or analog thereof, and B10 is uracil, or a derivative or analog thereof.

In some embodiments, B9 comprises adenine and the nucleobase at position +1 of the polynucleotide template comprises thymine, or a derivative or analog thereof. In some embodiments, B10 comprises guanine and the nucleobase at position +2 of the polynucleotide template comprises cytosine, or a derivative or analog thereof.

In some embodiments, R1 are each independently H, alkyl, acyl, benzyl or a cleavable unit. In some embodiments, R1 is C1-C6 alkyl. In some embodiments, R1 is H. In some embodiments, R1 is Me. In some embodiments, R2 is H, OH, alkyl, O-alkyl, O-acyl, O-benzyl, halogen, LNA, a linker, a cleavable unit or a detectable marker. In some embodiments, R2 is H, OH, O—(C1-C6 alkyl), O-MOE, fluoro, or LNA. In some embodiments, R2 is OH. In some embodiments, R3 is H, OH, alkyl, O-alkyl, O-acyl, O-benzyl, halogen, a linker, a cleavable unit or a detectable marker. In some embodiments, R3 is H, OH, O—(C1-C6 alkyl), O-MOE, or fluoro. In some embodiments R3 is OH. In some embodiments, R3 is OMe.

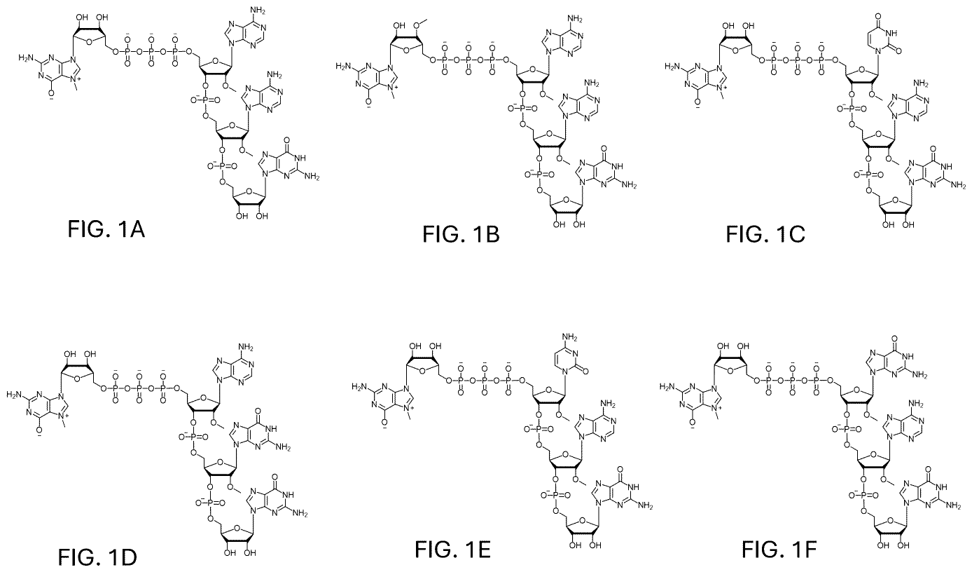

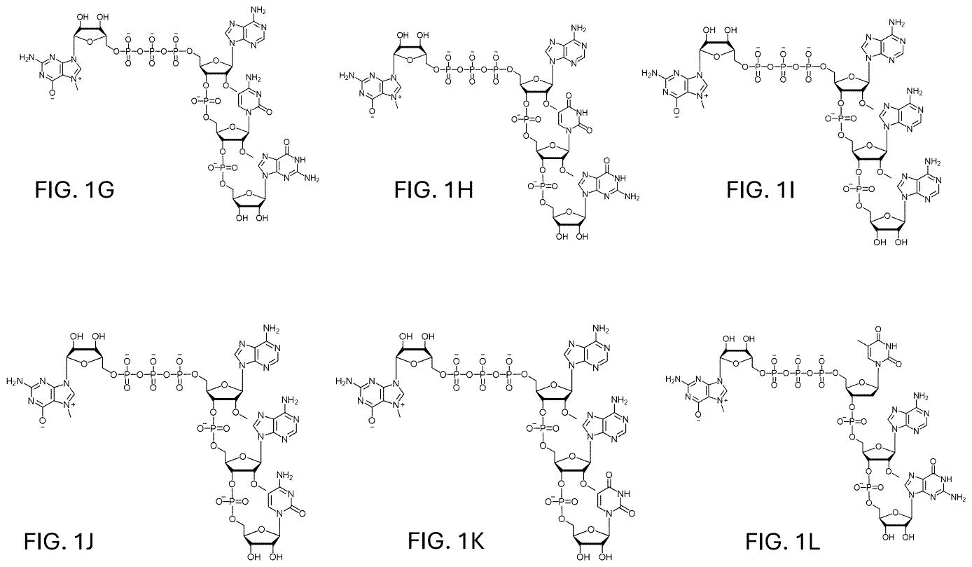

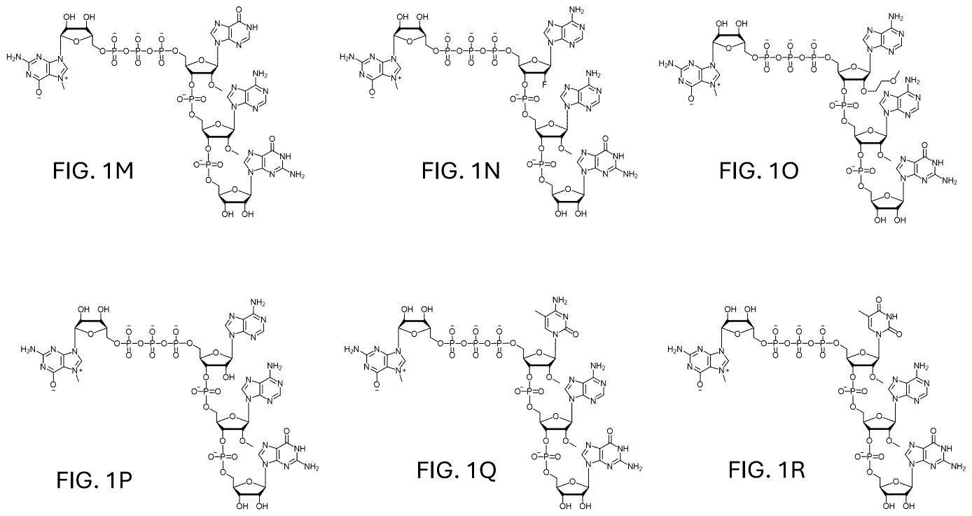

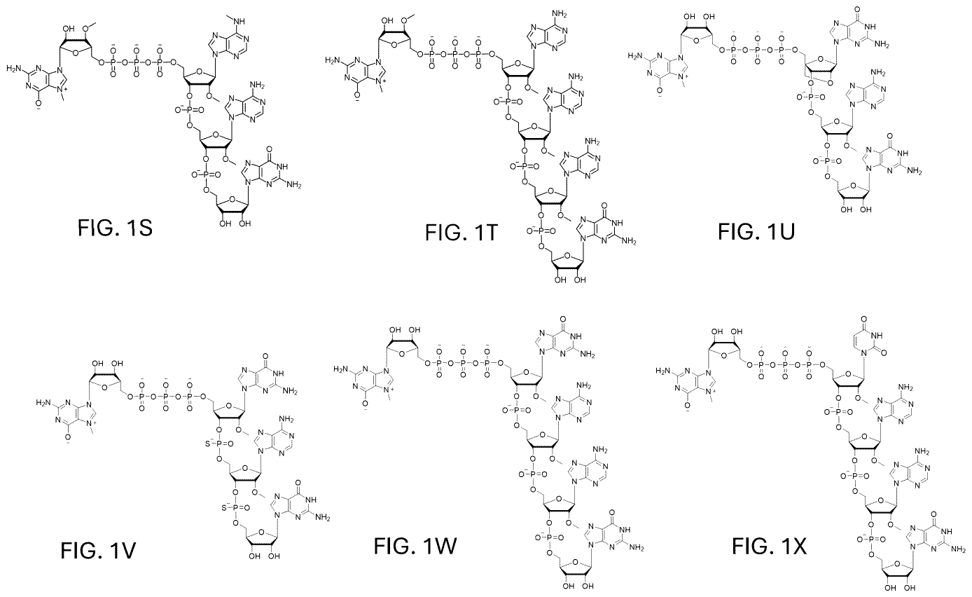

In some embodiments, q1 to q7 are 0; and R11 and R12 are each independently OH or O-alkyl. In some embodiments, R11 is OH or OMe and R12 is OH or OMe. In some embodiments, R11 is OMe and R12 is OH or OMe. In some embodiments, R11 is OMe and R12 is OMe. In some embodiments, q1 to q7 are 0; and R11 and R12 are each independently H, OH, O—(C1-C6 alkyl), O-MOE, fluoro, or LNA. In some embodiments, Z1 to Z3 and Z18 to Z22 are each O. In some embodiments, X1, X2, and X10 to X12 are each O. In some embodiments, Y1, Y2, and Y10 to Y12 are each independently S⁻ or O⁻. In some embodiments, Y1, Y2, and Y10 to Y12 are each O⁻. In some embodiments, the capping primer comprises or consists of a compound shown in any one of FIGS. 1A-1C, 1E-1S, 1U and 1V. In some embodiments, the capping primer comprises or consists of a compound shown in FIG. 1A. In some embodiments, the capping primer comprises or consists of a compound shown in FIG. 1B. In some embodiments, the capping primer comprises or consists of a compound shown in FIG. 1C. In some embodiments, the capping primer comprises or consists of a compound shown in FIG. 1E. In some embodiments, the capping primer comprises or consists of a compound shown in FIG. 1F. In some embodiments, the capping primer comprises or consists of a compound shown in FIG. 1G. In some embodiments, the capping primer comprises or consists of a compound shown in FIG. 1H. In some embodiments, the capping primer comprises or consists of a compound shown in FIG. 11. In some embodiments, the capping primer comprises or consists of a compound shown in FIG. 1J. In some embodiments, the capping primer comprises or consists of a compound shown in FIG. 1K. In some embodiments, the capping primer comprises or consists of a compound shown in FIG. 1L. In some embodiments, the capping primer comprises or consists of a compound shown in FIG. 1M. In some embodiments, the capping primer comprises or consists of a compound shown in FIG. 1N. In some embodiments, the capping primer comprises or consists of a compound shown in FIG. 1O. In some embodiments, the capping primer comprises or consists of a compound shown in FIG. 1P. In some embodiments, the capping primer comprises or consists of a compound shown in FIG. 1Q. In some embodiments, the capping primer comprises or consists of a compound shown in FIG. 1R. In some embodiments, the capping primer comprises or consists of a compound shown in FIG. 1S. In some embodiments, the capping primer comprises or consists of a compound shown in FIG. 1U. In some embodiments, the capping primer comprises or consists of a compound shown in FIG. 1V.

In some embodiments, q1 is 1; q2 to q7 are 0; and R4, R11, and R12 are each independently OH or O-alkyl. In some embodiments, R4 is OMe, R11 is OH or OMe, and R12 is OH or OMe. In some embodiments, R4 is OMe, R11 is OMe, and R12 is OH or OMe. In some embodiments, R4 is OMe, R11 is OMe, and R12 is OMe. In some embodiments, q1 is 1; q2 to q7 are 0; and R4, R11, and R12 are each independently H, OH, O—(C1-C6 alkyl), O-MOE, fluoro, or LNA. In some embodiments, Z1 to Z5 and Z18 to Z22 are each 0. In some embodiments, X1 to X3 and X10 to X12 are each 0. In some embodiments, Y1, Y2, and Y10 to Y12 are each independently S⁻ or O⁻. In some embodiments, Y1 to Y3 and Y10 to Y12 are each O⁻.

In some embodiments, B1 is complementary to a nucleobase at position −2 of the polynucleotide template. In some embodiments, B1 is mismatched to a nucleobase at position −2 of the polynucleotide template. In some embodiments, B8 is complementary to a nucleobase at position −1 of the polynucleotide template. In some embodiments, B8 is mismatched to a nucleobase at position −1 of the polynucleotide template. In some embodiments, B9 is complementary to a nucleobase at position +1 of the polynucleotide template. In some embodiments, B10 is complementary to a nucleobase at positions +2 of the polynucleotide template. In some embodiments, B8, B9, and B10 are not fully complementary to nucleobases at positions +1 to +3 of the polynucleotide template.

In some embodiments, the nucleobase at position +1 of the polynucleotide template is not cytosine. In some embodiments, the nucleobase at position −2 of the polynucleotide template comprises adenine, or a derivative or analog thereof. In some embodiments, the nucleobase at position −2 of the polynucleotide template is adenine. In some embodiments, the nucleobase at position −1 of the polynucleotide template comprises thymine, or a derivative or analog thereof. In some embodiments, the nucleobase at position −1 of the polynucleotide template comprises adenine, or a derivative or analog thereof. In some embodiments, the nucleobase at position −1 of the polynucleotide template comprises adenine or thymine. In some embodiments, the nucleobase at position +1 of the polynucleotide template comprises thymine, or a derivative or analog thereof. In some embodiments, the nucleobase at position +1 of the polynucleotide template comprises adenine, or a derivative or analog thereof. In some embodiments, the nucleobase at position +1 of the polynucleotide template comprises guanine, or a derivative or analog thereof. In some embodiments, the nucleobase at position +1 of the polynucleotide template comprises thymine, guanine or adenine. In some embodiments, the nucleobase at position +1 of the polynucleotide template comprises thymine or adenine. In some embodiments, the nucleobase at position +2 of the polynucleotide template comprises a cytosine nucleobase, or a derivative or analog thereof. In some embodiments, the nucleobase at position +2 of the polynucleotide template comprises a thymine nucleobase, or a derivative or analog thereof. In some embodiments, the nucleobase at position +2 of the polynucleotide template comprises a guanine nucleobase, or a derivative or analog thereof. In some embodiments, the nucleobase at position +2 of the polynucleotide template comprises an adenine nucleobase, or a derivative or analog thereof. In some embodiments, the nucleobase at position +2 of the polynucleotide template comprises thymine, guanine, cytosine or adenine.

In some embodiments, B1 is complementary to the nucleobase at position −2 of the polynucleotide template; B8 is complementary to the nucleobase at position −1 of the polynucleotide template; B9 is complementary to the nucleobase at position +1 of the polynucleotide template; and B10 is complementary to the nucleobase at position +2 of the polynucleotide template. In some embodiments, B1 is mismatched to the nucleobase at position −2 of the polynucleotide template; B8 is complimentary to the nucleobase at position −1 of the polynucleotide template; B9 is complementary to the nucleobase at position +1 of the polynucleotide template; and B10 is complementary to the nucleobase at positions +2 of the polynucleotide template. In some embodiments, B1 is mismatched to the nucleobase at position −2 of the polynucleotide template; B8 is mismatched to the nucleobase at position −1 of the polynucleotide template; B9 is complementary to the nucleobase at position +1 of the polynucleotide template; and B10 is complementary to the nucleobase at positions +2 of the polynucleotide template. In some embodiments, the nucleobase at position +1 of the polynucleotide template comprises thymine, or a derivative or analog thereof, and B9 comprises adenine, or a derivative or analog thereof. In some embodiments, the nucleobase at position +2 of the polynucleotide template comprises a cytosine nucleobase, or a derivative or analog thereof, and B10 comprises guanine, or a derivative or analog thereof.

In some embodiments, the nucleobase at position −2 of the polynucleotide template comprises adenine, or a derivative or analog thereof. In some embodiments, B1 is a nucleobase selected from the group consisting of adenine, uracil, thymine, cytosine, 5-methylcytosine, guanine, and hypoxanthine. In some embodiments, B1 is adenine, or a derivative or analog thereof. In some embodiments, B1 is uracil, or a derivative or analog thereof. In some embodiments, B1 comprises uracil, and the nucleobase at position −2 of the polynucleotide template comprises adenine, or a derivative or analog thereof. In some embodiments, q1 is 1 and q2 to q7 are 0, wherein B1 comprises guanine, and wherein the nucleobase at position −2 of the polynucleotide template comprises adenine, or a derivative or analog thereof.

In some embodiments, the capping primer comprises or consists of a compound shown in FIG. 1T. In some embodiments, the capping primer comprises or consists of a compound shown in FIG. 1W. In some embodiments, the capping primer comprises or consists of a compound shown in FIG. 1X.

In some embodiments, q1 to q2 are 1; q3 to q7 are 0; and R4, R5, R11, and R12 are each independently OH or O-alkyl. In some embodiments, R4 is OMe, R5 is OH or OMe, R11 is OH or OMe, and R12 is OH or OMe. In some embodiments, R4 is OMe and R5, R11, and R12 are each OH, R11 is OH or OMe, and R12 is OH or OMe. In some embodiments, R4 is OMe, R5 is OMe, R11 is OH or OMe, and R12 is OH or OMe. In some embodiments, R4 is OMe, R5 is OMe, and R11 and R12 are each OH. In some embodiments, R4 is OMe, R5 is OMe, and R11 and R12 are each OMe. In some embodiments, q1 to q2 are 1; q3 to q7 are 0; and R4, R5, R11, and R12 are each independently H, OH, O—(C1-C6 alkyl), O-MOE, fluoro, or LNA. In some embodiments, Z1 to Z7 and Z18 to Z22 are each O. In some embodiments, X1 to X4 and X10 to X12 are each O. In some embodiments, Y1, Y2, and Y10 to Y12 are each independently S⁻ or O⁻. In some embodiments, Y1 to Y4 and Y10 to Y12 are each O⁻.

In some embodiments, q1 to q3 are 1; q4 to q7 are 0; and R4 to R6, R11, and R12 are each independently OH or O-alkyl. In some embodiments, R4 is OMe, R5 is OH or OMe, R6 is OH or OMe, R11 is OH or OMe, and R12 is OH or OMe. In some embodiments, R4 is OMe and R5, R6, and R11 to R12 are each OH. In some embodiments, R4 is OMe, R5 is OMe, R6 is OH or OMe, R11 is OH or OMe, and R12 is OH or OMe. In some embodiments, R4 is OMe, R5 is OMe and R6 and R11 to R12 are each OH. In some embodiments, R4 is OMe, R5 is OMe and R6 and R11 to R12 are each OMe. In some embodiments, q1 to q3 are 1; q4 to q7 are 0; and R4 to R6, R11, and R12 are each independently H, OH, O—(C1-C6 alkyl), O-MOE, fluoro, or LNA. In some embodiments, Z1 to Z9 and Z18 to Z22 are each O. In some embodiments, X1 to X5 and X10 to X12 are each O. In some embodiments, Y1, Y2, and Y10 to Y12 are each independently S⁻ or O⁻. In some embodiments, Y1 to Y5 and Y10 to Y12 are each O⁻.

In some embodiments, q1 to q4 are 1; q5 to q7 are 0; and R4 to R7, R11, and R12 are each independently OH or O-alkyl. In some embodiments, R4 is OMe, R5 is OH or OMe, R6 is OH or OMe, R7 is OH or OMe, R11 is OH or OMe, and R12 is OH or OMe. In some embodiments, R4 is OMe and R5 to R7, R11, and R12 are each OH. In some embodiments, R4 is OMe, R5 is OMe, R6 is OH or OMe, R7 is OH or OMe, R11 is OH or OMe, and R12 is OH or OMe. In some embodiments, R4 is OMe, R5 is OMe, and R6, R7, R11, and R12 are each OH. In some embodiments, R4 is OMe, R5 is OMe, and R6, R7, R11, and R12 are each OMe. In some embodiments, q1 to q4 are 1; q5 to q7 are 0; and R4 to R7, R11, and R12 are each independently H, OH, O—(C1-C6 alkyl), O-MOE, fluoro, or LNA. In some embodiments, Z1 to Z11 and Z18 to Z22 are each O. In some embodiments, X1 to X6 and X10 to X12 are each O. In some embodiments, Y1, Y2, and Y10 to Y12 are each independently S⁻ or O⁻. In some embodiments, Y1 to Y6 and Y10 to Y12 are each O⁻.

In some embodiments, q1 to q5 are 1; q6 to q7 are 0; and R4 to R8, R11, and R12 are each independently OH or O-alkyl. In some embodiments, R4 is OMe, R5 is OH or OMe, R6 is OH or OMe, R7 is OH or OMe, R8 is OH or OMe, R11 is OH or OMe, and R12 is OH or OMe. In some embodiments, R4 is OMe and R5 to R8, R11, and R12 are each OH. In some embodiments, R4 is OMe, R5 is OMe, R6 is OH or OMe, R7 is OH or OMe, R8 is OH or OMe, R11 is OH or OMe, and R12 is OH or OMe. In some embodiments, R4 is OMe, R5 is OMe, and R6 to R8, R11, and R12 are each OH. In some embodiments, R4 is OMe, R5 is OMe, and R6 to R8, R11, and R12 are each OMe. In some embodiments, q1 to q5 are 1; q6 to q7 are 0; and R4 to R8, R11, and R12 are each independently H, OH, O—(C1-C6 alkyl), O-MOE, fluoro, or LNA. In some embodiments, Z1 to Z13 and Z18 to Z22 are each O. In some embodiments, X1 to X7 and X10 to X12 are each O. In some embodiments, Y1, Y2, and Y10 to Y12 are each independently S⁻ or O⁻. In some embodiments, Y1 to Y7 and Y10 to Y12 are each O⁻.

In some embodiments, q1 to q6 are 1; q7 is 0; and R4 to R9, R11, and R12 are each independently OH or O-alkyl. In some embodiments, R4 is OMe, R5 is OH or OMe, R6 is OH or OMe, R7 is OH or OMe, R8 is OH or OMe, R9 is OH or OMe, R11 is OH or OMe, and R12 is OH or OMe. In some embodiments, R4 is OMe and R5 to R9, R11, and R12 are each OH. In some embodiments, R4 is OMe, R5 is OMe, R6 is OH or OMe, R7 is OH or OMe, R8 is OH or OMe, R9 is OH or OMe, R11 is OH or OMe, and R12 is OH or OMe. In some embodiments, R4 is OMe, R5 is OMe, and R6 to R9, R11, and R12 are each OH. In some embodiments, R4 is OMe, R5 is OMe, and R6 to R9, R11, and R12 are each OMe. In some embodiments, q1 to q6 are 1; q7 is 0; and R4 to R9, R11, and R12 are each independently H, OH, O—(C1-C6 alkyl), O-MOE, fluoro, or LNA. In some embodiments, Z1 to Z15 and Z18 to Z22 are each O. In some embodiments, X1 to X8 and X10 to X12 are each O. In some embodiments, Y1, Y2, and Y10 to Y12 are each independently S⁻ or O⁻. In some embodiments, Y1 to Y8 and Y10 to Y12 are each O⁻.

In some embodiments, q1 to q7 are 1; and R4 to R12 are each independently OH or O-alkyl. In some embodiments, R4 is OMe, R5 is OH or OMe, R6 is OH or OMe, R7 is OH or OMe, R8 is OH or OMe, R9 is OH or OMe, R10 is OH or OMe, R11 is OH or OMe, and R12 is OH or OMe. In some embodiments, R4 is OMe and R5 to R12 are each OH. In some embodiments, R4 is OMe, R5 is OMe, R6 is OH or OMe, R7 is OH or OMe, R8 is OH or OMe, R9 is OH or OMe, R10 is OH or OMe, R11 is OH or OMe, and R12 is OH or OMe. In some embodiments, R4 is OMe, R5 is OMe, and R6 to R12 are each OH. In some embodiments, R4 is OMe, R5 is OMe, and R6 to R12 are each OMe. In some embodiments, q1 to q7 are 1; and R4 to R12 are each independently H, OH, O—(C1-C6 alkyl), O-MOE, fluoro, or LNA. In some embodiments, Z1 to Z22 are each O. In some embodiments, X1 to X12 are each O. In some embodiments, Y1, Y2, and Y10 to Y12 are each independently S⁻ or O⁻. In some embodiments, Y1 to Y12 are each O⁻.

In some embodiments, the structure according to Formula II is any one shown in FIGS. 1A-1X. In some embodiments, the structure according to Formula II is any one shown in 1A-1C, 1E-1S, and 1U-1X.

In some embodiments, the capping primer comprises a compound comprising the structure according to Formula III:

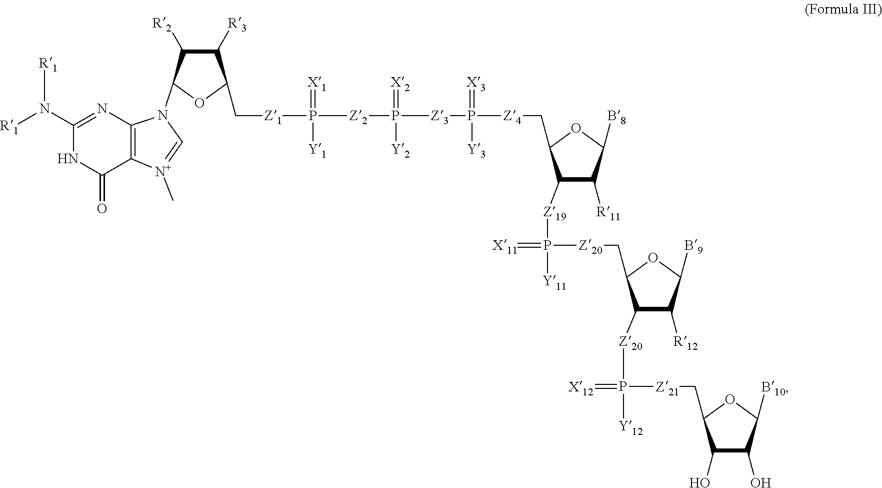

B′8 is a nucleobase selected from the group consisting of adenine, guanine, thymine, uracil, cytosine, and hypoxanthine and any derivative or analog of the foregoing; B′9 is a nucleobase selected from the group consisting of adenine, uracil, cytosine, and any derivative or analog of the foregoing; B′10 is a nucleobase selected from the group consisting of adenine, guanine, uracil, cytosine, and any derivative or analog of the foregoing; R′1 are each independently H, alkyl, acyl, benzyl or a cleavable unit; R′2 is H, OH, alkyl, O-alkyl, O-acyl, O-benzyl, O-MOE, halogen, LNA, a linker, a cleavable unit or a detectable marker; R′3 is H, OH, alkyl, O-alkyl, O-acyl, O-benzyl, O-MOE, halogen, a linker, a cleavable unit or a detectable marker; R′11 and R′12 are each independently H, OH, alkyl, O-alkyl, O-MOE, halogen, LNA, a linker or a detectable marker; X′1 to X′3, X′11, and X′12 are each independently O or S; Y′1 to Y′3, Y′11, and Y′12 are each independently O⁻, S⁻, BH₃⁻, aryl, alkyl, O-alkyl or O-aryl; and Z′1 to Z′4 and Z′19 to Z′21 are each independently O or S, NH, CH₂, C(halogen)₂ or CH(halogen).

In some embodiments, the capping primer hybridizes to a polynucleotide template described herein, wherein the polynucleotide template comprises a nucleobase at position −1 immediately adjacent to and downstream (3′) of a transcriptional start site and a nucleobase at each of position +1, position +2 and position +3 immediately adjacent to and upstream (5′) of the transcriptional start site, and wherein the capping primer comprises a nucleobase complementary to the nucleobase at position +1 and position +2 of the polynucleotide template (each referring to a position of the antisense strand of the polynucleotide template). In some embodiments, B′8 is complementary or mismatched to a nucleobase at positions −1 of the polynucleotide template. In some embodiments, B′9 is complementary to a nucleobase at positions +1 of the polynucleotide template. In some embodiments, B′10 is complementary to a nucleobase at positions +2 of the polynucleotide template.

In some embodiments, the nucleobase at position +1 of the polynucleotide template is not cytosine. In some embodiments, the nucleobase at position −1 of the polynucleotide template comprises thymine, or a derivative or analog thereof. In some embodiments, the nucleobase at position −1 of the polynucleotide template comprises adenine, or a derivative or analog thereof. In some embodiments, the nucleobase at position −1 of the polynucleotide template comprises adenine or thymine. In some embodiments, the nucleobase at position +1 of the polynucleotide template comprises thymine, or a derivative or analog thereof. In some embodiments, the nucleobase at position +1 of the polynucleotide template comprises adenine, or a derivative or analog thereof. In some embodiments, the nucleobase at position +1 of the polynucleotide template comprises guanine, or a derivative or analog thereof. In some embodiments, the nucleobase at position +1 of the polynucleotide template comprises thymine, guanine or adenine. In some embodiments, the nucleobase at position +1 of the polynucleotide template comprises thymine or adenine. In some embodiments, the nucleobase at position +2 of the polynucleotide template comprises a cytosine nucleobase, or a derivative or analog thereof. In some embodiments, the nucleobase at position +2 of the polynucleotide template comprises a thymine nucleobase, or a derivative or analog thereof. In some embodiments, the nucleobase at position +2 of the polynucleotide template comprises a guanine nucleobase, or a derivative or analog thereof. In some embodiments, the nucleobase at position +2 of the polynucleotide template comprises an adenine nucleobase, or a derivative or analog thereof. In some embodiments, the nucleobase at position +2 of the polynucleotide template comprises adenine, guanine, cytosine or thymine.

In some embodiments, B′8 is complementary to the nucleobase at position −1 of the polynucleotide template; B′9 is complementary to the nucleobase at position +1 of the polynucleotide template; and B′10 is complementary to the nucleobase at position +2 of the polynucleotide template. In some embodiments, B′8 is mismatched to the nucleobase at position −1 of the polynucleotide template; B′9 is complementary to the nucleobase at position +1 of the polynucleotide template; and B′10 is complementary to the nucleobase at position +2 of the polynucleotide template. In some embodiments, the nucleobase at position +1 of the polynucleotide template comprises thymine, or a derivative or analog thereof; and B′9 comprises adenine, or a derivative or analog thereof. In some embodiments, the nucleobase at position +2 of the polynucleotide template comprises a cytosine nucleobase, or a derivative or analog thereof; and B′10 comprises guanine, or a derivative or analog thereof.

In some embodiments, the nucleobase at position −1 of the polynucleotide template comprises thymine, or a derivative or analog thereof. In some embodiments, the nucleobase at position −1 of the polynucleotide template comprises adenine, or a derivative or analog thereof. In some embodiments, the nucleobase at position −1 of the polynucleotide template comprises thymine or adenine. In some embodiments, B′8 is any nucleobase that is not N6-modified adenine (e.g. 6-methyladenine). In some embodiments, B′8 is a nucleobase selected from the group consisting of adenine, uracil, thymine, cytosine, 5-methylcytosine, guanine, and hypoxanthine. In some embodiments, B′8 is adenine or a derivative or analog thereof. In some embodiments, B′8 is uracil or a derivative or analog thereof. In some embodiments, B′8 comprises adenine or N6-methyladenosine and the nucleobase at position −1 of the polynucleotide template comprises thymine, or a derivative or analog thereof. In some embodiments, B′8 comprises uracil and the nucleobase at position −1 of the polynucleotide template comprises thymine, or a derivative or analog thereof.

In some embodiments, B′9 is selected from the group consisting of adenine, cytosine, and uracil. In some embodiments, B′10 is selected from the group consisting of adenine, guanine, cytosine, and uracil. In some embodiments, B′9 is adenine, or a derivative or analog thereof; and B′10 is guanine, or a derivative or analog thereof. In some embodiments, B′9 is cytosine, or a derivative or analog thereof; and B′10 is guanine, or a derivative or analog thereof. In some embodiments, B′9 is uracil, or a derivative or analog thereof, and B′10 is guanine, or a derivative or analog thereof. In some embodiments, B′9 is adenine, or a derivative or analog thereof; and B′10 is adenine, or a derivative or analog thereof. In some embodiments, B′9 is adenine, or a derivative or analog thereof, and B′10 is cytosine, or a derivative or analog thereof. In some embodiments, B′9 is adenine, or a derivative or analog thereof; and B′10 is uracil, or a derivative or analog thereof.

In some embodiments, B′9 comprises adenine and the nucleobase at position +1 of the polynucleotide template comprises thymine, or a derivative or analog thereof. In some embodiments, B′10 comprises guanine and the nucleobase at position +2 of the polynucleotide template comprises cytosine, or a derivative or analog thereof.

In some embodiments, R′1 are each independently H, alkyl, acyl, benzyl or a cleavable unit. In some embodiments, R′1 are each C1-C6 alkyl. In some embodiments, R′1 are each H. In some embodiments, R′1 are each Me. In some embodiments, R′2 is H, OH, alkyl, O-alkyl, O-acyl, O-benzyl, halogen, LNA, a linker, a cleavable unit or a detectable marker. In some embodiments, R′2 is H, OH, O—(C1-C6 alkyl), O-MOE, fluoro, or LNA. In some embodiments, R′2 is OH. In some embodiments, R′3 is H, OH, alkyl, O-alkyl, O-acyl, O-benzyl, halogen, a linker, a cleavable unit or a detectable marker. In some embodiments, R′3 is H, OH, O—(C1-C6 alkyl), O-MOE, or fluoro. In some embodiments R′3 is OH. In some embodiments, R′3 is OMe.

In some embodiments, R′11 and R′12 are each independently OH or O-alkyl. In some embodiments, R′11 is OH or OMe and R′12 is OH or OMe. In some embodiments, R′11 is OMe and R′12 is OH or OMe. In some embodiments, R′11 is OMe and R′12 is OMe. In some embodiments, R′11 and R′12 are each independently H, OH, O—(C1-C6 alkyl), O-MOE, fluoro, or LNA. In some embodiments, Z′1 to Z′3 and Z′18 to Z′22 are each O. In some embodiments, X′1, X′2, and X′10 to X′12 are each O. In some embodiments, Y1, Y2, and Y10 to Y12 are each independently S⁻ or O⁻. In some embodiments, Y′1, Y′2, and Y′10 to Y′12 are each O⁻.

In some embodiments, the structure according to Formula III is any one shown in FIGS. 1A-1C, 1E-1S, 1U and 1V. In some embodiments, the capping primer consists of the structure according to Formula III, wherein the structure is any one shown in FIGS. 1A-1C, 1E-1S, 1U and 1V. In some embodiments, the capping primer consists of the structure shown in any one of FIGS. 1A-1C, 1E-1S, 1U and 1V.

In some embodiments, the capping primer comprises a compound comprising the structure according to Formula IV:

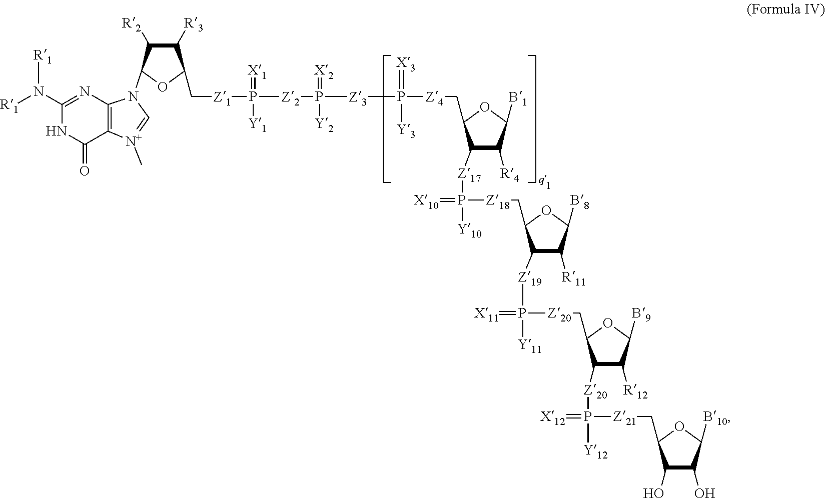

wherein q′1 is 0 or 1; B′1, if present, is a nucleobase independently selected from the group consisting of adenine, guanine, thymine, uracil, cytosine, and hypoxanthine and any derivative or analog of the foregoing; B′8 is a nucleobase selected from the group consisting of adenine, guanine, thymine, uracil, cytosine, and hypoxanthine and any derivative or analog of the foregoing; B′9 is a nucleobase selected from the group consisting of adenine, uracil, cytosine, and any derivative or analog of the foregoing; B′10 is a nucleobase selected from the group consisting of adenine, guanine, uracil, cytosine, and any derivative or analog of the foregoing; R′1 are each independently H, alkyl, acyl, benzyl or a cleavable unit; R′2 is H, OH, alkyl, O-alkyl, O-acyl, O-benzyl, O-MOE, halogen, LNA, a linker, a cleavable unit or a detectable marker; R′3 is H, OH, alkyl, O-alkyl, O-acyl, O-benzyl, O-MOE, halogen, a linker, a cleavable unit or a detectable marker; R′4, if present, is H, OH, alkyl, O-alkyl, O-methoxyethyl (MOE), halogen, LNA, a linker, or a detectable marker; R′4, if present, R′11 and R′12 are each independently H, OH, alkyl, O-alkyl, O-MOE, halogen, LNA, a linker or a detectable marker; X′1 to X′2, X′3, if present, and X′10 to X′12 are each independently O or S; Y′1 to Y′2, Y′3, if present, and Y′10 to Y′12 are each independently O⁻, S⁻, BH₃⁻, aryl, alkyl, O-alkyl or O-aryl; and Z′1 to Z′3, Z′4 if present, Z′17, if present, and Z′18 to Z′21 are each independently O or S, NH, CH₂, C(halogen)₂ or CH(halogen).

In some embodiments, the capping primer hybridizes to a polynucleotide template described herein, wherein the polynucleotide template comprises a nucleobase at position −2 and position −1 immediately adjacent to and downstream (3′) of a transcriptional start site and a nucleobase at each of position +1, position +2 and position +3 immediately adjacent to and upstream (5′) of the transcriptional start site, and wherein the capping primer comprises a nucleobase complementary to the nucleobase at position +1 and position +2 of the polynucleotide template (each referring to a position of the antisense strand of the polynucleotide template). In some embodiments, B′8 is complementary or mismatched to a nucleobase at position −1 of the polynucleotide template. In some embodiments, B′9 is complementary to a nucleobase at position +1 of the polynucleotide template. In some embodiments, B′10 is complementary to a nucleobase at position +2 of the polynucleotide template.

In some embodiments, q′1 is 1, wherein B′1 is complementary or mismatched to a nucleobase at position −2 of the polynucleotide template. In some embodiments, B′1 is complementary to a nucleobase at position −2 of the polynucleotide template. In some embodiments, B′1 is mismatched to a nucleobase at position −2 of the polynucleotide template. In some embodiments, B′8 is complementary to a nucleobase at position −1 of the polynucleotide template. In some embodiments, B′8 is mismatched to a nucleobase at position −1 of the polynucleotide template. In some embodiments, B′1 is complementary to the nucleobase at position −2 of the polynucleotide template; B′8 is complementary to the nucleobase at position −1 of the polynucleotide template; B′9 is complementary to the nucleobase at position +1 of the polynucleotide template; and B′10 is complementary to the nucleobase at position +2 of the polynucleotide template. In some embodiments, B′1 is complementary to the nucleobase at position −2 of the polynucleotide template; B′8 is mismatched to the nucleobase at position −1 of the polynucleotide template; B′9 is complementary to the nucleobase at position +1 of the polynucleotide template; and B′10 is complementary to the nucleobase at position +2 of the polynucleotide template. In some embodiments, B′1 is mismatched to the nucleobase at position −2 of the polynucleotide template; B′8 is complementary to the nucleobase at position −1 of the polynucleotide template; B′9 is complementary to the nucleobase at position +1 of the polynucleotide template; and B′10 is complementary to the nucleobase at position +2 of the polynucleotide template. In some embodiments, B′1 is mismatched to the nucleobase at position −2 of the polynucleotide template; B′8 is mismatched to the nucleobase at position −1 of the polynucleotide template; B′9 is complementary to the nucleobase at position +1 of the polynucleotide template; and B′10 is complementary to the nucleobase at position +2 of the polynucleotide template. In some embodiments, the nucleobase at position +1 of the polynucleotide template comprises thymine, or a derivative or analog thereof; and B′9 comprises adenine, or a derivative or analog thereof. In some embodiments, the nucleobase at position +2 of the polynucleotide template comprises a cytosine nucleobase, or a derivative or analog thereof; and B′10 comprises guanine, or a derivative or analog thereof.

In some embodiments, the nucleobase at position +1 of the polynucleotide template is not cytosine. In some embodiments, the nucleobase at position −2 of the polynucleotide template comprises adenine, or a derivative or analog thereof. In some embodiments, the nucleobase at position −2 of the polynucleotide template is adenine. In some embodiments, the nucleobase at position −1 of the polynucleotide template comprises thymine, or a derivative or analog thereof. In some embodiments, the nucleobase at position −1 of the polynucleotide template comprises adenine, or a derivative or analog thereof. In some embodiments, the nucleobase at position −1 of the polynucleotide template comprises adenine or thymine. In some embodiments, the nucleobase at position +1 of the polynucleotide template comprises thymine, or a derivative or analog thereof. In some embodiments, the nucleobase at position +1 of the polynucleotide template comprises adenine, or a derivative or analog thereof. In some embodiments, the nucleobase at position +1 of the polynucleotide template comprises guanine, or a derivative or analog thereof. In some embodiments, the nucleobase at position +1 of the polynucleotide template comprises thymine, guanine or adenine. In some embodiments, the nucleobase at position +1 of the polynucleotide template comprises thymine or adenine. In some embodiments, the nucleobase at position +2 of the polynucleotide template comprises a cytosine nucleobase, or a derivative or analog thereof. In some embodiments, the nucleobase at position +2 of the polynucleotide template comprises a thymine nucleobase, or a derivative or analog thereof. In some embodiments, the nucleobase at position +2 of the polynucleotide template comprises a guanine nucleobase, or a derivative or analog thereof. In some embodiments, the nucleobase at position +2 of the polynucleotide template comprises an adenine nucleobase, or a derivative or analog thereof. In some embodiments, the nucleobase at position +2 of the polynucleotide template comprises thymine, guanine, cytosine or adenine.

In some embodiments, B′1 is a nucleobase selected from the group consisting of adenine, uracil, thymine, cytosine, 5-methylcytosine, guanine, and hypoxanthine. In some embodiments, B′1 is adenine, or a derivative or analog thereof. In some embodiments, B′1 is uracil, or a derivative or analog thereof. In some embodiments, B′1 comprises uracil, wherein the nucleobase at position −2 of the polynucleotide template comprises adenine, or a derivative or analog thereof. In some embodiments, B′1 comprises guanine, wherein the nucleobase at position −2 of the polynucleotide template comprises adenine, or a derivative or analog thereof.

In some embodiments, the capping primer comprises or consists of a compound shown in FIG. 1T. In some embodiments, the capping primer comprises or consists of a compound shown in FIG. 1W. In some embodiments, the capping primer comprises or consists of a compound shown in FIG. 1X.

In some embodiments, the disclosure provides a stereoisomer, tautomer, or salt form of a capping primer described herein. In some embodiments, the disclosure provides a pharmaceutically acceptable salt form of a capping primer described herein.

### Polynucleotide Templates

The present disclosure provides polynucleotide templates for use in the transcription systems described herein.

In some embodiments, the polynucleotide template is a DNA template. In some embodiments, the DNA template is a linear molecule. In some embodiments, the DNA template is a circular molecule. In some embodiments, the DNA template is double-stranded. In some embodiments, the DNA template is partially double-stranded. In some embodiments, the DNA template is a double-stranded linear DNA, a partially double-stranded linear DNA, a circular double-stranded DNA, a DNA plasmid, a PCR amplicon, or a modified DNA. In some embodiments, the DNA template comprises at least one chemically modified deoxynucleotide. Methods to generate DNA are known in the art, and include, e.g., gene synthesis, recombinant DNA technology, or a combination thereof. In some embodiments, the DNA template is obtained by cloning of a nucleic acid (e.g. cDNA) into a vector for IVT.

In some embodiments, the polynucleotide template is an RNA template. In some embodiments, the RNA is a single-stranded RNA. In some embodiments, the RNA template is an alphaviral polynucleotide template. In some embodiments, the RNA template comprises at least one chemically modified ribonucleotide.

In some embodiments, the polynucleotide template comprises DNA and RNA.

In some embodiments, the polynucleotide template provides an RNA when transcribed. In some embodiments, the RNA transcribed from the polynucleotide template is a protein-coding RNA or a non-coding RNA. In some embodiments, the protein-coding RNA is an mRNA. In some embodiments, the non-coding RNA is a guide RNA (e.g., for a CRISPR system), a small nuclear RNA (snRNA), a small nucleolar RNA (snoRNA), a ribosomal RNA, an antisense RNA, a short hairpin RNA (shRNA), a long non-coding RNA, a ribozyme, an aptamer, or a small cajal body-specific RNA (scaRNA).

In some embodiments, the polynucleotide template (e.g., DNA template) comprises a nucleotide sequence coding for a transcribed region of interest (e.g., coding for an RNA described herein) and a promoter sequence recognized by an RNAP described herein for use in IVT (e.g., a T7 RNAP).

In some embodiments, the transcribed region codes for an mRNA, a self-amplifying RNA, or a non-coding RNA. In some embodiments, the non-coding RNA is a spliceosomal RNA, a small nucleolar RNA, or an enhancer RNA. In some embodiments, the transcribed region codes for an mRNA or a self-amplifying RNA. In some embodiments, the sense strand comprises from 5′ to 3′: an RNAP promoter, a TSS, and a transcribed region. In some embodiments, the sense strand comprises from 5′ to 3′: an RNAP promoter, a TSS, and a transcribed region coding for an mRNA, wherein the transcribed region comprises a 5′-UTR, an ORF, a 3′-UTR, and a polyA sequence. In some embodiments, the sense strand comprises from 5′ to 3′: an RNAP promoter, a TSS, and a transcribed region coding for a self-amplifying RNA, wherein the transcribed region comprises a 5′-UTR, a replicase sequence (e.g., an alphavirus replicase sequence, such as one described in Ballesteros-Briones, et al (2020) Curr Opin Virol 44:145), an ORF, a 3′-UTR, and a polyA sequence.

In some embodiments, the consensus sequence for the RNAP spans the RNAP promoter, the TSS and the transcribed region. In some embodiments, the consensus sequence for the RNAP spans a region of the sense strand, wherein the region extends from about 17, 16, 15, or 14 nucleotides upstream of the TSS to about 2, 3, 4, 5, or 6 nucleotides downstream of the TSS.

In some embodiments, the consensus sequence for the RNAP comprises a wild-type promoter or a variant thereof. In some embodiments, the consensus sequence for the RNAP comprises a wild-type T7 promoter. An exemplary consensus sequence comprising a wild-type T7 promoter, as present in the sense strand, is TAATACGACTCACTATAGGGAGA (SEQ ID NO: 7), wherein the nucleotide at position +1 is indicated in bold. In some embodiments, the consensus sequence for the RNAP comprises a promoter comprising one or more substitutions with respect to a wild-type T7 promoter.

In some embodiments, the consensus sequence for the RNAP comprises a wild-type SP6 promoter. An exemplary consensus sequence comprising a wild-type SP6 promoter, as present in the sense strand, is ATTTAGGTGACACTATAGAAGAA (SEQ ID NO: 8), wherein the nucleotide at position +1 is indicated in bold. In some embodiments, the consensus sequence for the RNAP comprises a promoter comprising one or more substitutions with respect to a wild-type SP6 promoter.

In some embodiments, the consensus sequence for the RNAP comprises a wild-type T3 promoter. An exemplary consensus sequence comprising a wild-type T3 promoter, as present in the sense strand, is AATTAACCCTCACTAAAGGGAGA (SEQ ID NO: 9), wherein the TSS is indicated in bold. In some embodiments, the consensus sequence for the RNAP comprises a promoter comprising one or more substitutions with respect to a wild-type T3 promoter.

In some embodiments, the RNAP promoter, as present in the sense strand, comprises a sequence of SEQ ID NO: 1. In some embodiments, the RNAP promoter, as present in the sense strand, consists of a sequence of SEQ ID NO: 1.

In some embodiments, the consensus sequence for the RNAP, as present in the sense strand of the polynucleotide template, comprises an RNAP promoter in positions −17 to −1, dA in position +1, and dG in position +2, wherein the RNAP promoter is SEQ ID NO: 1.

In some embodiments, the consensus sequence for the RNAP, as present in the sense strand of the polynucleotide template, comprises an RNAP promoter in positions −17 to −1, dA in position +1, and dG in position +2, wherein the RNAP promoter is SEQ ID NO: 4.

In some embodiments, the consensus sequence for the RNAP, as present in the sense strand of the polynucleotide template, comprises an RNAP promoter in positions −17 to −1, dC in position +1, and dG in position +2, wherein the RNAP promoter is SEQ ID NO: 1.

In some embodiments, the consensus sequence for the RNAP, as present in the sense strand of the polynucleotide template, comprises an RNAP promoter in positions −17 to −1, dT in position +1, and dG in position +2, wherein the RNAP promoter is SEQ ID NO: 1.

In some embodiments, the consensus sequence for the RNAP, as present in the sense strand of the polynucleotide template, comprises an RNAP promoter in positions −17 to −1, dA in position +1, and dA in position +2, wherein the RNAP promoter is SEQ ID NO: 1.

In some embodiments, the consensus sequence for the RNAP, as present in the sense strand of the polynucleotide template, comprises an RNAP promoter in positions −17 to −1, dA in position +1, and dC in position +2, wherein the RNAP promoter is SEQ ID NO: 1.

In some embodiments, the consensus sequence for the RNAP, as present in the sense strand of the polynucleotide template, comprises an RNAP promoter in positions −17 to −1, dA in position +1, and dT in position +2, wherein the RNAP promoter is SEQ ID NO: 1.

In some embodiments, the transcribed region encodes a non-coding RNA. In some embodiments, the transcribed region (e.g., the ORF of the transcribed region) encodes a protein. In some embodiments, the encoded protein comprises an antigen (e.g., a cancer antigen or an infectious antigen), a cytokine, a chemokine, an enzyme (e.g., a gene editing nuclease, e.g., a CRISPR enzyme), a hormone, a signaling receptor or portion thereof, an antibody (e.g., a monospecific or multispecific antibody) or antigen binding fragment thereof (e.g., an scFv, a Fab), a transcription factor, a structural protein, an immunoglobulin, or a combination thereof. In some embodiments, the protein comprises a secretory peptide. In some embodiments, the protein comprises a nuclear localization signal.

In some embodiments, the antisense strand comprises a nucleobase at position −1, wherein the nucleobase is adenine or a derivative or analog thereof. In some embodiments, the nucleobase at position −1 is thymine, or a derivative or analog thereof.

In some embodiments, the antisense strand comprises a nucleobase at position +1, wherein the nucleobase is adenine or a derivative or analog thereof. In some embodiments, the nucleobase at position +1 is thymine, or a derivative or analog thereof. In some embodiments, the nucleobase at position +1 is guanine, or a derivative or analog thereof. In some embodiments, the nucleobase at position +1 is thymine.

In some embodiments, the antisense strand comprises a nucleobase at position +2, wherein the nucleobase is adenine or a derivative or analog thereof. In some embodiments, the nucleobase at position +2 is thymine, or a derivative or analog thereof. In some embodiments, the nucleobase at position +2 is guanine, or a derivative or analog thereof. In some embodiments, the nucleobase at position +2 is cytosine, or a derivative or analog thereof. In some embodiments, the nucleobase at position +2 is cytosine.

In some embodiments, the consensus sequence for the RNAP, as present in the antisense strand of the polynucleotide template, comprises 5′-X⁶X⁵X⁴X³X²X¹X⁻¹ATAGTGAGTCGTATTA-3′ (SEQ ID NO: 6). The sequence of the polynucleotide template adjacent to TSS (e.g. the nucleotides X⁻¹ to X⁶ at positions −1 to +6 of the antisense strand of the polynucleotide template) may be modified by a person skilled in the art using methods known in the art, as appropriate for the intended design. In some embodiments, X⁻¹ corresponds to position −1 and is any nucleotide. In some embodiments, X⁻¹ is a deoxyribonucleotide (e.g., dA, dT, dC, or dG). In some embodiments, X⁻¹ is A or T (e.g., dA or dT). In some embodiments, X¹ corresponds to position +1 and comprises a nucleobase other than cytosine. In some embodiments, X¹ is a deoxyribonucleotide that is not dC (e.g., dA, dT, or dG). In some embodiments, X¹ is dG, dA, or dT. In some embodiments, X¹ is dA or dT. In some embodiments, X¹ is T (e.g., dT). In some embodiments, X² corresponds to position +2 and comprises any nucleotide. In some embodiments, X² is a deoxyribonucleotide (e.g., dA, dT, dC, or dG). In some embodiments, X² is C (e.g., dC). In some embodiments, X² is dC, dT, dG, or dA. In some embodiments, X⁻¹ is dT or dA, X¹ is dA, dG or dT, and X² is dC, dT, dG, or dA. In some embodiments, X⁻¹ is dT, X¹ is dT, and X² is dC. In some embodiments, X⁻¹ is dA, X¹ is dT, and X² is dC. In some embodiments, X⁻¹ is dT, X¹ is dA, and X² is dC. In some embodiments, X⁻¹ is dT, X¹ is dT, and X² is dT. In some embodiments, X⁻¹ is dT, X¹ is dT, and X² is dG. In some embodiments, X⁻¹ is dT, X¹ is dT, and X² is dA.

### Exemplary Transcription Systems

Exemplary transcription systems of the disclosure are set forth by the following enumerated embodiments. “E” corresponds to “embodiment.”

E1. A system comprising a capping primer and a polynucleotide template, wherein the capping primer comprises a structure according to Formula I, or a diastereomer, salt, or solvated form thereof, wherein q1 to q20, are each independently 0 or 1; B1 through B20, if present, are each a nucleobase independently selected from the group consisting of adenine, guanine, thymine, uracil, cytosine, hypoxanthine, and any derivative or analog of the foregoing; B21 is a nucleobase selected from the group consisting of adenine, guanine, thymine, uracil, cytosine, hypoxanthine, and any derivative or analog of the foregoing; B22 is selected from the group consisting of adenine, uracil, cytosine and any derivative or analog of the foregoing; B23 is selected from the group consisting of adenine, guanine, uracil, cytosine and any derivative or analog of the foregoing; R1 are each independently H, alkyl, acyl, benzyl or a cleavable unit; R2 is H, OH, alkyl, O-alkyl, O-acyl, O-benzyl, O-methoxyethyl (MOE), halogen, LNA, a linker, a cleavable unit or a detectable marker; and R3 is H, OH, alkyl, O-alkyl, O-acyl, O-benzyl, halogen, a linker, a cleavable unit or a detectable marker; R4 to R25, if present, are each independently H, OH, O-MOE, alkyl, O-alkyl, halogen, a linker, a cleavable unit, or a detectable marker; X1 to X25, if present, are each independently O or S; Y1 to Y25, if present, are each independently O⁻, S⁻, BH₃⁻, aryl, alkyl, O-alkyl or O-aryl; and Z1 to Z48, if present, are each independently O, S, NH, CH₂, C(halogen)₂ or CH(halogen); wherein the polynucleotide template comprises an antisense strand comprising a nucleobase at position −1 immediately adjacent to and downstream (3′) of a transcriptional start site and a nucleobase at each of position +1, position +2 and position +3 immediately adjacent to and upstream (5′) of the transcriptional start site, wherein B21 is complementary or mismatched to a nucleobase at positions −1 of the polynucleotide template, wherein B22 is complementary to a nucleobase at positions +1 of the polynucleotide template, wherein B23 is complementary to a nucleobase at positions +2 of the polynucleotide template.

E2. The system of E1, wherein B21 to B23 are not fully complementary to positions +1 to +3 of the polynucleotide template.

E3. The system of any of E1-E2, wherein R4 to R25, if present, are OH or O-alkyl; X1-X25, if present, are O; Y1-Y25, if present, are O; and Z1 to Z48, if present, are O.

E4. The system of any of E1-E2, wherein R4 to R23, if present, are OH or OMe; R24 is OMe; R25 is OMe; X1-X25, if present, are O; Y1-Y25, if present, are O or S⁻; and Z1 to Z48, if present, are O.

E5. The system of any of E1-E4, wherein q1 to q20 is 0.

E6. The system of E5, wherein R24 and R25 are each independently H, OH, O—(C1-C6 alkyl), O-MOE, fluoro, or LNA.

E7. The system of E5, wherein R24 and R25 are each independently OH or OMe.

E8. The system of any of E1-E4, wherein at least one of q1 to q20 is 1.

E9. The system of E8, wherein q1 is 1 and q2 to q20 are 0, wherein R4, R24 and R25 are each independently OH or OMe.

E10. The system of E9, wherein R4, R24, and R25 are each OMe.

E11. The system of E9 or E10, wherein B1 is complementary to a nucleobase at position −2 of the polynucleotide template.

E12. The system of E9 or E10, wherein B1 is mismatched to a nucleobase at position −2 of the polynucleotide template.

E13. The system of any of E1-E4, wherein at least two of q1 to q20 are 1.

E14. The system of E8, wherein q1 to q2 are 1 and q3 to q20 are 0, wherein each of R4, R5, R24 and R25 are each independently OH or OMe.

E15. The system of any of E1-E4, wherein at least three of q1 to q20 is 1.

E16. The system of E15, wherein q1 to q3 are 1 and q4 to q20 are 0, wherein R4 to R6, R24 and R25 are each independently OH or OMe.

E17. The system of any of E1-E4, wherein at least four of q1 to q20 are 1.

E18. The system of E17, wherein q1 to q4 are 1 and q5 to q20 are 0, wherein R4 to R7, R24 and R25 are each independently OH or OMe.

E19. The system of any of E1-E4, wherein at least five of q1 to q20 are 1.

E20. The system of E19, wherein q1 to q5 are 1 and q6 to q20 are 0, wherein R4 to R8, R24, and R25 are each independently OH or OMe.

E21. The system of any of E1-E4, wherein at least six of q1 to q20 are 1.

E22. The system of E21, wherein q1 to q6 are 1 and q7 to q20 are 0, wherein R4 to R9, R24, and R25 are each independently OH or OMe.

E23. The system of any of E1-E4, wherein at least seven of q1 to q20 are 1.

E24. The system of E23, wherein q1 to q7 are 1 and q8 to q20 are 0, wherein R4 to R10, R24, and R25 are each independently OH or OMe.

E25. The system of any of E1-E4, wherein at least eight of q1 to q20 are 1.

E26. The system of E25, wherein q1 to q8 are 1 and q9 to q20 are 0, wherein R4 to R11, R24, and R25 are each independently OH or OMe.

E27. The system of any of E1-E4, wherein at least nine of q1 to q20 are 1.

E28. The system of E27, wherein q1 to q9 are 1 and q10 to q20 are 0, wherein R4 to R12, R24, and R25 are each independently OH or OMe.

E29. The system of any of E1-E4, wherein at least ten of q1 to q20 are 1.

E30. The system of E29, wherein q1 to q10 are 1 and q11 to q20 are 0, wherein R4 to R13, R24, and R25 are each independently OH or OMe.

E31. The system of any of E1-E4, wherein at least eleven of q1 to q20 are 1.

E32. The system of E31, wherein q1 to q11 are 1 and q12 to q20 are 0, wherein R4 to R14, R24, and R25 are each independently OH or OMe.

E33. The system of any of E1-E4, wherein at least twelve of q1 to q20 are 1.

E34. The system of E33, wherein q1 to q12 are 1 and q13 to q20 are 0, wherein R4 to R15, R24, and R25 are each independently OH or OMe.

E35. The system of any of E1-E4, wherein at least thirteen of q1 to q20 are 1.

E36. The system of E35, wherein q1 to q13 are 1 and q14 to q20 are 0, wherein R4 to R16, R24, and R25 are each independently OH or OMe.

E37. The system of any of E1-E4, wherein at least fourteen of q1 to q20 are 1.

E38. The system of E37, wherein q1 to q14 are 1 and q15 to q20 are 0, wherein R4 to R17, R24, and R25 are each independently OH or OMe.

E39. The system of any of E1-E4, wherein at least fifteen of q1 to q20 are 1.

E40. The system of E39, wherein q1 to q15 are 1 and q16 to q20 are 0, wherein R4 to R18, R24, and R25 are each independently OH or OMe.

E41. The system of any of E1-E4, wherein at least sixteen of q1 to q20 are 1.

E42. The system of E41, wherein q1 to q16 are 1 and q17 to q20 are 0, wherein R4 to R19, R24, and R25 are each independently OH or OMe.

E43. The system of any of E1-E4, wherein at least seventeen of q1 to q20 are 1.

E44. The system of E43, wherein q1 to q17 are 1 and q18 to q20 are 0, wherein R4 to R20, R24, and R25 are each independently OH or OMe.

E45. The system of any of E1-E4, wherein at least eighteen of q1 to q20 are 1.

E46. The system of E45, wherein q1 to q18 are 1 and q19 to q20 are 0, wherein R4 to R21, R24, and R25 are each independently OH or OMe.

E47. The system of any of E1-E4, wherein at least nineteen of q1 to q20 are 1.

E48. The system of E47, wherein q1 to q19 are 1 and q20 is 0, wherein R4 to R22, R24, and R25 are each independently OH or OMe.

E49. The system of any of E1-E4, wherein each of q1 to q20 are 1.

E50. The system of E18, wherein q1 to q20 are 1, and wherein R4 to R25 are each independently OH or OMe.

E51. The system of any of E1-E50, wherein B21 is not N6-modified adenine.

E52. The system of any of E1-E51, wherein B21 is a nucleobase selected from the group consisting of adenine, uracil, thymine, cytosine, 5-methylcytosine, guanine, and hypoxanthine.

E53. The system of E1-E50, wherein B21 is adenine or N6-methyladenosine.

E54. The system of any of E1-E52, wherein B21 is uracil.

E55. The system of any of E1-E54, wherein B22 is selected from the group consisting of adenine, cytosine, 5-methylcytosine, thymine and uracil.

E56. The system of any one of E1-E54, wherein B22 is selected from the group consisting of adenine, cytosine, and uracil.

E57. The system of any one of E1-E54, wherein B22 is adenine or uracil.

E58. The system of E57, wherein B22 is adenine.

E59. The system of any one of E1-E58, wherein B23 is selected from the group consisting of adenine, guanine, cytosine, and uracil.

E60. The system of E59, wherein B23 is guanine.

E61. The system of any one of E1-E54, wherein B22 is adenine, or a derivative or analog thereof, and B23 is guanine, or a derivative or analog thereof.

E62. The system of any one of E1-E54, wherein B22 is cytosine, or a derivative or analog thereof, and B23 is guanine, or a derivative or analog thereof.

E63. The system of any one of E1-E54, wherein B22 is uracil, or a derivative or analog thereof, and B23 is guanine, or a derivative or analog thereof.

E64. The system of any one of E1-E54, wherein B22 is adenine, or a derivative or analog thereof, and B23 is adenine, or a derivative or analog thereof.

E65. The system of any one of E1-E42, wherein B22 is adenine, or a derivative or analog thereof, and B23 is uracil, or a derivative or analog thereof.

E66. The system of any one of E1-E65, wherein R1 are each independently H or a C1-C6 alkyl.

E67. The system of any one of E1-E65, wherein R1 are each H.

E68. The system of any one of E1-E67, wherein R2 is H, OH, O—(C1-C6 alkyl), O-MOE, fluoro, or LNA, and wherein R3 is H, OH, O—(C1-C6 alkyl), O-MOE, or fluoro.

E69. The system of any one of E1-E67, wherein R2 is OH and R3 is OH or OMe.

E70. The system of any one of E1-E69, wherein B21 is complementary to the nucleobase at position −1 of the polynucleotide template.

E71. The system of any one of E1-E69, wherein B21 is mismatched to the nucleobase at position −1 of the polynucleotide template.

E72. The system of any one of E1-E69, wherein the nucleobase at position −1 of the polynucleotide template is selected from adenine, thymine and any analog or derivative thereof.

E73. The system of any one of E1-E69, wherein the nucleobase at position −1 of the polynucleotide template is thymine, or any analog or derivative thereof.

E74. The system of any one of E1-E73, wherein the nucleobase at position +1 of the polynucleotide template is selected from adenine, guanine, thymine, and any analog or derivative thereof.

E75. The system of any one of E1-E73, wherein the nucleobase at position +1 of the polynucleotide template is thymine, or any analog or derivative thereof.

E76. The system of any one of E1-E75, wherein the nucleobase at position +2 of the polynucleotide template is selected from adenine, guanine, cytosine, thymine, and any analog or derivative thereof.

E77. The system of any one of E1-E75, wherein the nucleobase at position +2 of the polynucleotide template is cytosine, or any analog or derivative thereof.

E78. The system of any one of E1-E77, wherein contacting the system with an RNA polymerase under transcription conditions results in synthesis of an RNA.

E79. The system of E78, wherein the transcription conditions comprise nucleoside 5′-triphosphates (NTPs), wherein the capping primer and NTPs are present in approximately equimolar concentrations.

E80. The system of E79, wherein the NTPs comprise adenosine triphosphate (ATP) or a derivative or analog thereof, guanosine triphosphate (GTP) or a derivative or analog thereof, cytidine triphosphate (CTP) or a derivative or analog thereof, and uridine triphosphate (UTP) or a derivative or analog thereof, each at an approximately equimolar concentration, optionally wherein the derivative or analog of UTP is pseudouridine triphosphate (pseudo-UTP) or N1-methylpseudouridine triphosphate (N1-pseudo-UTP).

E81. The system of E79 or E80, wherein the NTPs comprise ATP, GTP, CTP, and UTP at an approximately equimolar concentration, optionally wherein UTP is pseudo-UTP or N1-pseudo-UTP, and wherein (a) each NTP is present in the IVT reaction at a molar concentration that is about 1.1-fold, 1.2-fold, 1.3-fold, 1.4-fold, 1.5-fold, 2-fold, 2.5-fold, 3-fold, 4-fold, 5-fold, or 10-fold higher than a molar concentration of the capping primer, or (b) the capping primer is present in the IVT reaction at a molar concentration that is about 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, or 5-fold higher than a molar concentration of each NTP.

E82. The system of any of E78-E81, wherein the synthesis is characterized by an increased capping efficiency as compared to contacting a control system contacted with the RNA polymerase, optionally wherein capping efficiency is measured by densitometry.

E83. The system of any of E78-E82, wherein the synthesis is characterized by an increased output of RNA comprising a 5′cap as compared to contacting a control system with the RNA polymerase, optionally wherein output of RNA is measured by densitometry.

E84. The system of E82 or E83, wherein the control system comprises a capping primer and a polynucleotide template comprising a transcriptional start site, wherein (i) the capping primer is fully complementary to position +1 to position +3 of the polynucleotide template; (ii) the polynucleotide template comprises a 2′-deoxycytidine at each of position +1 and position +2; or (iii) both (i) and (ii).

E85. The system of any of E78-E84, wherein at least about 65%, about 70%, about 75%, about 80%, about 85%, about 90%, about 91%, about 92%, about 93%, about 94%, about 95%, about 96%, about 97%, about 98%, or about 99% of the RNA molecules produced by IVT reaction are 5′-capped.

E86. The system of any of E78-E85, wherein the synthesis results in a yield of RNA molecules that is at least about 60%, about 70%, about 75%, about 80%, about 85%, about 90%, about 91%, about 92%, about 93%, about 94%, about 95%, about 96%, about 97%, about 98%, about 99% higher relative to the RNA yield obtained in the reaction without using a primer.

E87. The system of any of E78-E86, wherein the synthesis results in a yield of RNA molecule that is at least about 1.1-fold, 1.5-fold, 2-fold, 3-fold, 4-fold, 5-fold, 6-fold, 7-fold, 8-fold, 9-fold, or 10-fold higher relative to the RNA yield obtained in the reaction without using a primer.

E88. The system of any of E78-E87, wherein the synthesis results in (a) a yield of RNA molecules that is at least about 30% relative to the RNA yield obtained in the reaction without using a primer; and (b) at least about 30% of the RNA molecules produced are 5′-capped.

E89. The system of any of E78-E87, wherein the synthesis results in (a) a yield of RNA molecules that is at least about 50% relative to the RNA yield obtained in the reaction without using a primer; and (b) at least about 50% of the RNA molecules produced are 5′-capped.

E90. The system of any of E78-E87, wherein the synthesis results in (a) a yield of RNA molecules that is at least about 70% relative to the RNA yield obtained in the reaction without using a primer; and (b) at least about 70% of the RNA molecules produced are 5′-capped.

E91. The system of any of E78-E90, wherein the RNA polymerase is a T7 RNA polymerase or a variant thereof, a T3 RNA polymerase or a variant thereof, or a SP6 RNA polymerase or a variant thereof.

E92. A system comprising a capping primer and a polynucleotide template, wherein the capping primer comprises a structure according to Formula II, or a diastereomer, salt, or solvated form thereof, wherein q1 to q7, are each independently 0 or 1; B1 through B7, if present, are each a nucleobase independently selected from the group consisting of adenine, guanine, thymine, uracil, cytosine, hypoxanthine, and any derivative or analog of the foregoing; B8 is a nucleobase selected from the group consisting of adenine, guanine, thymine, uracil, cytosine, hypoxanthine, and any derivative or analog of the foregoing; B9 is selected from the group consisting of adenine, uracil, cytosine and any derivative or analog of the foregoing; B10 is selected from the group consisting of adenine, guanine, uracil, cytosine and any derivative or analog of the foregoing; R1 are each independently H, alkyl, acyl, benzyl or a cleavable unit; R2 is H, OH, alkyl, O-alkyl, O-acyl, O-benzyl, O-methoxyethyl (MOE), halogen, LNA, a linker, a cleavable unit or a detectable marker, R3 is H, OH, alkyl, O-alkyl, O-acyl, O-benzyl, halogen, LNA, a linker, a cleavable unit or a detectable marker; R4 to R12, if present, are each independently H, OH, alkyl, O-alkyl, O-MOE, halogen, LNA, a linker or a detectable marker; X1 to X12, if present, are each independently O or S; Y1 to Y12, if present, are each independently O—, S—, BH3-, aryl, alkyl, O-alkyl or O-aryl; and Z1 to Z22, if present, are each independently O, S, NH, CH2, C(halogen)2 or CH(halogen); wherein the polynucleotide template comprises an antisense strand comprising a nucleobase at position −1 immediately adjacent to and downstream (3′) of a transcriptional start site and a nucleobase at each of position +1, position +2 and position +3 immediately adjacent to and upstream (5′) of the transcriptional start site, wherein B8 is complementary or mismatched to a nucleobase at positions −1 of the polynucleotide template, wherein B9 is complementary to a nucleobase at positions +1 of the polynucleotide template, wherein B10 is complementary to a nucleobase at positions +2 of the polynucleotide template.

E93. The system of E92, wherein B8 to B10 is not fully complementary to positions +1 to +3 of the polynucleotide template.

E94. The system of any of E92 or E93, wherein R4 to R12, if present, are OH or O-alkyl; X1-X12, if present, are O; Y1-Y12, if present, are O⁻; and Z1 to Z22, if present, are O.

E95. The system of any of E92-E94, wherein q1 to q7 is 0.

E96. The system of E95, wherein R11 and R12 are each independently H, OH, O—(C1-C6 alkyl), O-MOE, fluoro, or LNA.

E97. The system of E95, wherein R11 and R12 are each independently OH or OMe.

E98. The system of E95, wherein R11 and R12 are each OMe.

E99. The system of any of E92-E94, wherein at least one of q1 to q7 is 1.

E100. The system of E99, wherein q1 is 1 and q2 to q7 are 0, wherein R4, R11 and R12 are each independently OH or OMe.

E101. The system of E100, wherein R4, R11, and R12 are each OMe.

E102. The system of E99-E101, wherein B1 is complementary to a nucleobase at position −2 of the polynucleotide template.

E103. The system of E99-E101, wherein B1 is mismatched to a nucleobase at position −2 of the polynucleotide template.

E104. The system of any of E92-E94, wherein at least two of q1 to q7 is 1.

E105. The system of E104, wherein q1 to q2 are 1 and q3 to q7 are 0, wherein each of R4, R5, R11 and R12 are each independently OH or OMe.

E106. The system of any of E92-E94, wherein at least three of q1 to q7 is 1.

E107. The system of E106, wherein q1 to q3 are 1 and q4 to q7 are 0, wherein R4 to R6, R11 and R12 are each independently OH or OMe.

E108. The system of any of E92-E94, wherein at least four of q1 to q7 is 1.

E109. The system of E108, wherein q1 to q4 are 1 and q5 to q7 are 0, wherein R4 to R7, R11 and R12 are each independently OH or OMe.

E110. The system of any of E92-E94, wherein at least five of q1 to q7 is 1.

E111. The system of E110, wherein q1 to q5 are 1 and q6 to q7 are 0, wherein R4 to R8, R11, and R12 are each independently OH or OMe.

E112. The system of any of E92-E94, wherein at least six of q1 to q7 is 1.

E113. The system of E112, wherein q1 to q6 are 1 and q7 is 0, wherein R4 to R9, R11, and R12 are each independently OH or OMe.

E114. The system of any of E92-E94, wherein each of q1 to q7 is 1.

E115. The system of E114, wherein q1 to q7 are 1, wherein R4 to R12 are each independently OH or OMe.

E116. The system of E92-E115, wherein B8 is not N6-modified adenine.

E117. The system of E92-E116, wherein B8 is a nucleobase selected from the group consisting of adenine, uracil, thymine, cytosine, 5-methylcytosine, guanine, and hypoxanthine.

E118. The system of any of E92-E115, wherein B8 is adenine or N6-methyladenosine.

E119. The system of any of E92-E116, wherein B8 is uracil.

E120. The system of E92-E119, wherein B9 is selected from the group consisting of adenine, cytosine, 5-methylcytosine, thymine and uracil.

E121. The system of any of E92-E119, wherein B9 is selected from the group consisting of adenine, cytosine, and uracil.

E122. The system of E92-E119, wherein B9 is adenine or uracil.

E123. The system of any one of E92-E119, wherein B9 is adenine.

E124. The system of any one of E92-E123, wherein B10 is selected from the group consisting of adenine, guanine, cytosine, and uracil.

E125. The system of any one of E97-E123, wherein B10 is guanine.

E126. The system of E92-E119, wherein B9 is adenine, or a derivative or analog thereof, and B10 is guanine, or a derivative or analog thereof.

E127. The system of E92-E119, wherein B9 is cytosine, or a derivative or analog thereof, and B10 is guanine, or a derivative or analog thereof.

E128. The system of E92-E119, wherein B9 is uracil, or a derivative or analog thereof, and B10 is guanine, or a derivative or analog thereof.

E129. The system of E92-E119, wherein B9 is adenine, or a derivative or analog thereof, and B10 is adenine, or a derivative or analog thereof.

E130. The system of E92-E119, wherein B9 is adenine, or a derivative or analog thereof, and B10 is uracil, or a derivative or analog thereof.

E131. The system of E92-E130, wherein R1 are each H or a C1-C6 alkyl.

E132. The system of E92-E130, wherein R1 are each H.

E133. The system of E92-E132, wherein R2 is H, OH, O—(C1-C6 alkyl), O-MOE, fluoro, or LNA, and wherein R3 is H, OH, O—(C1-C6 alkyl), O-MOE, or fluoro.

E134. The system of any one of E92-E132, wherein R2 is OH and R3 is OH or OMe.

E135. The system of any one of E92-E134, wherein B8 is complementary to the nucleobase at position −1 of the polynucleotide template.

E136. The system of any one of E92-E134, wherein B8 is mismatched to the nucleobase at position −1 of the polynucleotide template.

E137. The system of any one of E92-E134, wherein the nucleobase at position −1 of the polynucleotide template is selected from adenine, thymine and any analog or derivative thereof.

E138. The system of any one of E92-E134, wherein the nucleobase at position −1 of the polynucleotide template is thymine, or any analog or derivative thereof.

E139. The system of any one of E92-E134, wherein the nucleobase at position +1 of the polynucleotide template is selected from adenine, guanine, thymine, and any analog or derivative thereof.

E140. The system of any one of E92-E134, wherein the nucleobase at position +1 of the polynucleotide template is thymine, or any analog or derivative thereof.

E141. The system of any one of E92-E134, wherein the nucleobase at position +2 of the polynucleotide template is selected from adenine, guanine, cytosine, thymine, and any analog or derivative thereof.

E142. The system of any one of E92-E134, wherein the nucleobase at position +2 of the polynucleotide template is cytosine, or any analog or derivative thereof.

E143. A system comprising a capping primer and a polynucleotide template, wherein the capping primer is a compound, or a diastereomer, salt, or solvated form thereof, wherein the polynucleotide template comprises an antisense strand comprising a nucleobase at position −1 immediately adjacent to and downstream (3′) of a transcriptional start site and a nucleobase at each of position +1, position +2 and position +3 immediately adjacent to and upstream (5′) of the transcriptional start site, wherein (i) the compound identified in FIG. 11, the nucleobase at position +1 of the polynucleotide template is thymine, and the nucleobase at position +2 of the polynucleotide template is thymine; (ii) the compound is identified in FIG. 1G, the nucleobase at position +1 of the polynucleotide template is guanine, and the nucleobase at position +2 of the polynucleotide template is cytosine; (iii) the compound is identified in FIG. 1H, the nucleobase at position +1 of the polynucleotide template is adenine, and the nucleobase at position +2 of the polynucleotide template is cytosine; (iv) the compound is identified in FIG. 1J, the nucleobase at position +1 of the polynucleotide template is thymine, and the nucleobase at position +2 of the polynucleotide template is guanine; (v) the compound is identified in FIG. 1K, the nucleobase at position +1 of the polynucleotide template is thymine, and the nucleobase at position +2 of the polynucleotide template is adenine; or (vi) the compound is any one identified in FIGS. 1A, 1B, 1C, 1E, 1F, 1L, 1M, 1N, 1O, 1P, 1Q, 1R, 1S, 1T, 1U, 1V, 1W, and 1X, the nucleobase at position +1 of the polynucleotide template is thymine, and the nucleobase at position +2 of the polynucleotide template is cytosine.

E144. The system of E143, wherein the compound, or a diastereomer, salt, or solvated form thereof, is not fully complementary to nucleobases at positions +1 to +3 of the polynucleotide template.

E145. The system of E143 or E144, wherein the compound, or a diastereomer, salt, or solvated form thereof, is complementary to the nucleobase at position −1 of the polynucleotide template.

E146. The system of E143 or E144, wherein the compound, or a diastereomer, salt, or solvated form thereof, is mismatched to the nucleobase at position −1 of the polynucleotide template.

E147. The system of any one of E92-E146, wherein contacting the system with an RNA polymerase under transcription conditions results in synthesis of an RNA.

E148. The system of E147, wherein the transcription conditions comprise nucleoside 5′-triphosphates (NTPs), wherein the capping primer and NTPs are present in approximately equimolar concentrations.

E149. The system of E148, wherein the NTPs comprise adenosine triphosphate (ATP) or a derivative or analog thereof, guanosine triphosphate (GTP) or a derivative or analog thereof, cytidine triphosphate (CTP) or a derivative or analog thereof, and uridine triphosphate (UTP) or a derivative or analog thereof, each at an approximately equimolar concentration, optionally wherein the derivative or analog of UTP is pseudouridine triphosphate (pseudo-UTP) or N1-methylpseudouridine triphosphate (N1-pseudo-UTP).

E150. The system of E148 or E149, wherein the NTPs comprise ATP, GTP, CTP, and UTP at an approximately equimolar concentration, optionally wherein UTP is pseudo-UTP or N1-pseudo-UTP, and wherein (a) each NTP is present in the IVT reaction at a molar concentration that is about 1.1-fold, 1.2-fold, 1.3-fold, 1.4-fold, 1.5-fold, 2-fold, 2.5-fold, 3-fold, 4-fold, 5-fold, or 10-fold higher than a molar concentration of the capping primer, or (b) the capping primer is present in the IVT reaction at a molar concentration that is about 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, or 5-fold higher than a molar concentration of each NTP.

E151. The system of any of E147-E150, wherein the synthesis is characterized by an increased capping efficiency as compared to contacting a control system contacted with the RNA polymerase, optionally wherein capping efficiency is measured by densitometry.

E152. The system of any of E147-E150, wherein the synthesis is characterized by an increased output of RNA comprising a 5′cap as compared to contacting a control system with the RNA polymerase, optionally wherein output of RNA is measured by densitometry.

E153. The system of any of E147-E152, wherein the control system comprises a capping primer and a polynucleotide template comprising a transcriptional start site, wherein (i) the capping primer is fully complementary to position +1 to position +3 of the polynucleotide template; (ii) the polynucleotide template comprises a 2′-deoxycytidine at each of position +1 and position +2; or (iii) both (i) and (ii).

E154. The system of any of E147-E153, wherein at least about 65%, about 70%, about 75%, about 80%, about 85%, about 90%, about 91%, about 92%, about 93%, about 94%, about 95%, about 96%, about 97%, about 98%, or about 99% of the RNA molecules produced by IVT reaction is 5′-capped.

E155. The system of any of E147-E154, wherein the synthesis results in a yield of RNA molecules that is at least about 60%, about 70%, about 75%, about 80%, about 85%, about 90%, about 91%, about 92%, about 93%, about 94%, about 95%, about 96%, about 97%, about 98%, about 99% relative to the RNA yield obtained in the reaction without using a primer.

E156. The system of any of E147-E154, wherein the synthesis results in a yield of RNA molecule that is at least about 1.1-fold, 1.5-fold, 2-fold, 3-fold, 4-fold, 5-fold, 6-fold, 7-fold, 8-fold, 9-fold, or 10-fold higher relative to the RNA yield obtained in the reaction without using a primer.

E157. The system of any of E147-E156, wherein the synthesis results in (a) a yield of RNA molecules that is at least about 30% relative to the RNA yield obtained in the reaction without using a primer; and (b) at least about 30% of the RNA molecules produced are 5′-capped.

E158. The system of any of E147-E156, wherein the synthesis results in (a) a yield of RNA molecules that is at least about 50% relative to the RNA yield obtained in the reaction without using a primer; and (b) at least about 50% of the RNA molecules produced are 5′-capped.

E159. The system of any of E147-E156, wherein the synthesis results in (a) a yield of RNA molecules that is at least about 70% relative to the RNA yield obtained in the reaction without using a primer; and (b) at least about 70% of the RNA molecules produced are 5′-capped.

E160. The system of any of E147-E159, wherein the RNA polymerase is a T7 RNA polymerase or a variant thereof, a T3 RNA polymerase or a variant thereof, or a SP6 RNA polymerase or a variant thereof.

### Methods of Making 5′-Capped RNA

The present disclosure provides methods for synthesis of 5′-capped RNA using the transcription systems described herein.

In some embodiments, the method comprises mixing a transcription system described herein (e.g., a transcription system comprising a capping primer and a polynucleotide template described herein) with an RNAP and nucleoside 5′-triphosphates, and incubating the mixture under transcription conditions, thereby synthesizing the 5′-capped RNA. In some embodiments, the method further comprises purifying the 5′-capped RNA.

Various RNA polymerases suitable for transcription reactions are known in the art, including, but not limited to, DNA dependent RNA polymerases (e.g., T7 RNAP, T3 RNAP, SP6 RNAP, N4 virion RNAP, or a variant or functional fragment thereof).

In some embodiments, the RNAP is a T7 RNA polymerase or a variant thereof, a T3 polymerase or a variant thereof, a T3 RNA polymerase or a variant thereof, or a SP6 RNA polymerase or a variant thereof.

In some embodiments, the RNAP is a T7 RNAP, the origin of which is non-limiting. Methods to clone and express T7 RNAP are known in the art (see, e.g., U.S. Pat. No. 4,952,496, herein incorporated by reference). Moreover, variants of T7 RNAP having improved stability are also known (see, e.g., U.S. Pat. Nos. 9,193,959; 8,551,752; and 7,507,567, each of which are herein incorporated by reference). In some embodiments, the RNAP is a variant of a variant T7 RNAP having improved properties, such as one described in U.S. Pat. No. 10,738,286, which is herein incorporated by reference.

In some embodiments, the nucleoside 5′-triphosphates are unmodified ribonucleoside 5′-triphosphates. Unmodified ribonucleoside 5′-triphosphates include the purine bases adenine (A) and guanine (G), and the pyrimidine bases cytosine (C) and uracil (U). In some embodiments, all four unmodified ribonucleoside 5′-triphosphates are present in the transcription conditions.

In some embodiments, the nucleoside triphosphates comprise modified ribonucleoside 5′-triphosphates. Modified ribonucleoside triphosphates include a modification selected from (i) an end modification, (ii) a nucleobase modification, (iii) a sugar modification, and (iv) a modified phosphodiester linkage. Exemplary modifications are known in the art, see, e.g., WO2022122689, herein incorporated by reference. In some embodiments, the ribonucleoside 5′-triphosphates comprise a derivative or analog of ATP, a derivative or analog of CTP, a derivative or analog of GTP, and/or a derivative or analog of UTP. In some embodiments, modified nucleoside 5′-triphosphates comprising pseudouridine 5′-triphosphates (WTP), N1-methyl-pseudouridine 5′-triphosphate (^{1m}ψTP) instead of UTP is included in the transcription conditions.

Conventional methods for in vitro synthesis of 5′-capped RNA incorporate a cap structure during IVT. T7 RNA polymerase naively initiates transcription with incorporation of guanosine opposite a cytosine residue at position +1 of the polynucleotide template. Typically, the 7-methyloguanosine-containing capping reagent is present in the IVT reaction at a high concentration relative to GTP to achieve high efficiency of capping. The transcription systems of the disclosure achieve high efficiency capping (e.g., greater than 90% 5′-capped RNA relative to uncapped RNA) with an approximately equimolar ratio of capping primer to NTP (including GTP), providing a more cost-effective and scalable production process to generate 5′-capped RNA.

In some embodiments, the ribonucleoside 5′-triphosphates comprise an approximately equimolar ratio of ATP, CTP, GTP, and UTP, or derivatives or analogs thereof (e.g., a molar ratio of 1:1:1:1 ATP:CTP:GTP:UTP). In some embodiments, the transcription conditions comprise an approximately equimolar ratio of capping primer and ribonucleoside 5′-triphosphates (e.g., ATP, CTP, GTP, and UTP, or derivatives or analogs thereof). In some embodiments, the transcription conditions comprise a molar ratio of 1:1:1:1:1 capping primer:ATP:CTP:GTP:UTP. In some embodiments, the transcription conditions comprise a molar ratio of 1:1:1:1:1 capping primer:ATP:CTP:GTP:ψTP. In some embodiments, the transcription conditions comprise a molar ratio of 1:1:1:1:1 capping primer:ATP:CTP:GTP:^{1m}ψTP.

In some embodiments, the method results in a greater amount of RNA transcription occurring from the capping primer rather than NTP, thereby resulting in greater production of 5′capped RNA. In some embodiments, at least about 60%, about 70%, about 80%, about 90%, or about 100% of RNA synthesized from the method initiates from the capping primer. In some embodiments, at least about 65% of RNA synthesized from the method initiates from the capping primer. In some embodiments, at least about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% of RNA synthesized from the method initiates from the capping primer. In some embodiments, at least about 65% about 70%, about 75%, about 80%, about 85%, about 90%, about 91%, about 92%, about 93%, about 94%, about 95%, about 96%, about 97%, about 98%, or about 99% of RNA synthesized from the method initiates from the capping primer.

In some embodiments, the method results in yield of RNA synthesized that is at least about 60%, about 65%, about 70%, about 75%, about 80%, about 85%, about 90%, about 91%, about 92%, about 93%, about 94%, about 95%, about 96%, about 97%, about 98%, or about 99% of RNA yield from a reaction performed without a capping primer

In some embodiments, the transcription conditions further comprise a buffer system. In some embodiments, the pH range of the buffer system is 6.0 to 10.0. In some embodiments, the pH value of the buffer system is 6.5 to 9.0. In some embodiments, the pH value of the buffer system is 7.0 to 8.5. In some embodiments, the pH value of the buffer system is 7.5 to 8.0. In some embodiments, the transcription conditions further comprise an Rnase inhibitor, a pyrophosphatase (e.g., an inorganic pyrophosphatase), a salt (e.g., monovalent salt and/or divalent salt, e.g., Li+, Na+, K+, NH⁴⁺, tris(hydroxymethyl)aminomethane cation, Mg²⁺, Ba²⁺, or Mn²⁺), a reducing agent (e.g, dithithreitol, 2-mercaptoethanol, etc.), spermidine, or combinations thereof. In some embodiments, the reaction components are combined simultaneously. In some embodiments, the reaction components are combined stepwise, e.g., in a specific order.

In some embodiments, the reaction is mixed by agitation.

In some embodiments, the reaction generates about 200 g to about 600 g RNA in a 100 μL scale IVT reaction. In some embodiments, the reaction generates about 1 mg to about 3 mg RNA in a 500 μL scale IVT reaction. In some embodiments, the reaction generates about 2 mg to about 6 mg RNA in a 1 mL scale IVT reaction. In some embodiments, the reaction generates about 10 mg to about 30 mg RNA in a 5 mL scale IVT reaction. In some embodiments, the reaction generates about 20 mg to about 60 mg RNA in a 10 mL scale IVT reaction. In some embodiments, the reaction generates about 100 mg to about 300 mg RNA in a 50 mL scale IVT reaction. In some embodiments, the reaction generates about 200 mg to about 600 mg RNA in a 100 mL scale IVT reaction. In some embodiments, the reaction generates about 1 g to about 3 g RNA in a 500 mL scale IVT reaction. In some embodiments, the reaction generates about 2 g to about 6 g RNA in a 1 L scale IVT reaction. In some embodiments, the reaction generates about 10 g to about 30 g RNA in a 5 L scale IVT reaction. In some embodiments, the reaction generates about 20 g to about 60 g RNA in a 10 L scale IVT reaction. In some embodiments, the reaction generates about 100 g to about 300 g RNA in a 50 L scale IVT reaction. In some embodiments, the reaction generates about 200 g to about 600 g RNA in a 100 L scale IVT reaction. In some embodiments, the reaction generates about 400 g to about 1.2 kg RNA in a 200 L scale IVT reaction.

In some embodiments, the reaction generates about 100 μg to about 500 μg RNA per reaction. In some embodiments, the reaction generates about 100 μg to about 1 mg RNA per reaction. In some embodiments, the reaction generates about 1 mg to about 10 mg RNA per reaction. In some embodiments, the reaction generates about 1 mg to about 100 mg RNA per reaction. In some embodiments, the reaction generates about 1 mg to about 1 g RNA per reaction. In some embodiments, the reaction generates about 100 mg to about 500 mg RNA per reaction. In some embodiments, the reaction generates about 100 mg to about 1 g RNA per reaction. In some embodiments, the reaction generates about 100 mg to about 10 g RNA per reaction. In some embodiments, the reaction generates about 1 g to about 10 g RNA per reaction. In some embodiments, the reaction generates about 1 g to about 100 g RNA per reaction. In some embodiments, the reaction generates about 10 g to about 100 g RNA per reaction. In some embodiments, the reaction generates about 10 g to about 200 g RNA per reaction. In some embodiments, the reaction generates about 10 g to about 300 g RNA per reaction. In some embodiments, the reaction generates about 10 g to about 500 g RNA per reaction. In some embodiments, the reaction generates about 10 g to about 1 kg RNA per reaction.

In some embodiments, at least about 30% of total RNA generated by the reaction comprises a 5′-cap. In some embodiments, at least about 35% of total RNA generated by the reaction comprises a 5′-cap. In some embodiments, at least about 40% of total RNA generated by the reaction comprises a 5′-cap. In some embodiments, at least about 45% of total RNA generated by the reaction comprises a 5′-cap. In some embodiments, at least about 50% of total RNA generated by the reaction comprises a 5′-cap. In some embodiments, at least about 55% of total RNA generated by the reaction comprises a 5′-cap. In some embodiments, at least about 60% of total RNA generated by the reaction comprises a 5′-cap. In some embodiments, at least about 65% of total RNA generated by the reaction comprises a 5′-cap. In some embodiments, at least about 70% of total RNA generated by the reaction comprises a 5′-cap. In some embodiments, at least about 75% of total RNA generated by the reaction comprises a 5′-cap. In some embodiments, at least about 80% of total RNA generated by the reaction comprises a 5′-cap. In some embodiments, at least about 85% of total RNA generated by the reaction comprises a 5′-cap. In some embodiments, at least about 90% of total RNA generated by the reaction comprises a 5′-cap. In some embodiments, at least about 91% of total RNA generated by the reaction comprises a 5′-cap. In some embodiments, at least about 92% of total RNA generated by the reaction comprises a 5′-cap. In some embodiments, at least about 93% of total RNA generated by the reaction comprises a 5′-cap. In some embodiments, at least about 94% of total RNA generated by the reaction comprises a 5′-cap. In some embodiments, at least about 95% of total RNA generated by the reaction comprises a 5′-cap.

In some embodiments, the yield of RNA is about 30% relative to the RNA yield obtained in the reaction without using a primer. In some embodiments, the yield of RNA is about 35% relative to the RNA yield obtained in the reaction without using a primer. In some embodiments, the yield of RNA is about 40% relative to the RNA yield obtained in the reaction without using a primer. In some embodiments, the yield of RNA is about 45% relative to the RNA yield obtained in the reaction without using a primer. In some embodiments, the yield of RNA is about 50% relative to the RNA yield obtained in the reaction without using a primer. In some embodiments, the yield of RNA is about 60% relative to the RNA yield obtained in the reaction without using a primer. In some embodiments, the yield of RNA is about 65% relative to the RNA yield obtained in the reaction without using a primer. In some embodiments, the yield of RNA is about 70% relative to the RNA yield obtained in the reaction without using a primer. In some embodiments, the yield of RNA is about 75% relative to the RNA yield obtained in the reaction without using a primer. In some embodiments, the yield of RNA is about 80% relative to the RNA yield obtained in the reaction without using a primer. In some embodiments, the yield of RNA is about 85% relative to the RNA yield obtained in the reaction without using a primer. In some embodiments, the yield of RNA is about 90% relative to the RNA yield obtained in the reaction without using a primer. In some embodiments, the yield of RNA is about 95% relative to the RNA yield obtained in the reaction without using a primer. In some embodiments, the yield of RNA is about 96% relative to the RNA yield obtained in the reaction without using a primer. In some embodiments, the yield of RNA is about 97% relative to the RNA yield obtained in the reaction without using a primer. In some embodiments, the yield of RNA is about 98% relative to the RNA yield obtained in the reaction without using a primer. In some embodiments, the yield of RNA is or about 99% relative to the RNA yield obtained in the reaction without using a primer.

In some embodiments, the capping primer comprises a detectable marker, such that a 5′-capped RNA transcribed from the capping primer can be identified using the detectable marker. For example, in some embodiments, the detectable marker is a fluorescent dye, and the RNA is detectable by fluorescence spectroscopy. In some embodiments, the detectable marker is an affinity marker (e.g., biotin), and the RNA is detectable by affinity capture.

Methods for detection and/or measurement of polypeptides in biological material are well known in the art and include, but are not limited to, Western-blotting, flow cytometry, ELISAs, RIAs, and various proteomics techniques. An exemplary method to measure or detect a polypeptide is an immunoassay, such as an ELISA. This type of protein quantitation can be based on an antibody capable of capturing a specific antigen, and a second antibody capable of detecting the captured antigen. Exemplary assays for detection and/or measurement of polypeptides are described in Harlow, E. and Lane, D. Antibodies: A Laboratory Manual, (1988), Cold Spring Harbor Laboratory Press.

Methods for detection and/or measurement of RNA in biological material are well known in the art and include, but are not limited to, Northern-blotting, RNA protection assay, RT PCR. Suitable methods are described in Molecular Cloning: A Laboratory Manual (Fourth Edition) By Michael R. Green, Joseph Sambrook, Peter MacCallum 2012, 2,028 pp, ISBN 978193611342-2.

### 5′-Capped RNA

The present disclosure provides 5′-capped RNA synthesized using a method and/or transcription system described herein.

In some embodiments, the 5′-capped RNA comprises a length of at least about 200 nucleotides, about 300 nucleotides, about 400 nucleotides, about 500 nucleotides, about 700 nucleotides, about 900 nucleotides, about 1100 nucleotides, about 1300 nucleotides about 1500 nucleotides, about 2000 nucleotides, about 3000 nucleotides, about 4000 nucleotides, about 5000 nucleotides, about 6000 nucleotides, about 7000 nucleotides, about 8000 nucleotides, about 9000 nucleotides, about 10000 nucleotides, about 30000 nucleotides, about 50000 nucleotides, about 50000 nucleotides, about 70000 nucleotides, about 100000 nucleotides, about 150000 nucleotides, about 200000 nucleotides, about 250000 nucleotides, or about 300000 nucleotides.

In some embodiments, the 5′-capped RNA is an mRNA. In some embodiments, the mRNA comprises ORF encoding a protein. In some embodiments, the mRNA comprises a 5′-UTR, an ORF, a 3′-UTR, polyA.

In some embodiments, the 5′-capped RNA is a non-coding RNA. In some embodiments, the 5′-capped RNA is a self-amplifying RNA.

In some embodiments, the protein comprises an antigen (e.g., a cancer antigen or an infectious antigen), a cytokine, a chemokine, an enzyme, a hormone, a signaling receptor or portion thereof, an antibody or antigen binding fragment thereof, a transcription factor, a structural protein, an immunoglobulin, or a combination thereof. In some embodiments, the protein comprises a secretory peptide. In some embodiments, the enzyme is a gene editing enzyme, e.g., a CRISPR enzyme. In some embodiments, the enzyme is a retrotransposase.

One of skill in the art will appreciate 5′-UTR and/or 3′-UTR sequence suitable for use in the mRNAs of the disclosure. In some embodiments, the 5′-UTR and/or 3′-UTR sequences are selected for improving mRNA stability, directing mRNA intracellular localization, and or improving mRNA translation efficiency.

In some embodiments, the ORF comprises a Kozak consensus sequence (see, e.g., Kozak (1987) Nucleic Acids Res., 15:8125).

In some embodiments, the mRNA comprises a polyA sequence.

In some embodiments, the mRNA comprises from 5′ to 3′: a 5′-cap, a 5′-UTR, an ORF (e.g., an ORF comprising a Kozak consensus sequence), a 3′-UTR, and a polyA sequence. In some embodiments, the 5′-cap is a capping primer described herein, wherein the 3′-OH is operably linked to the 5′-end of the 5′-UTR, e.g., by phosphate linkage.

In some embodiments, the RNA comprises at least one modified ribonucleotide. In some embodiments, at least one of A, U, C, and G present in the RNA is a modified ribonucleotide. In some embodiments, each of the uridines in the RNA are modified uridines (e.g., pseudouridine, e.g., N1-methylpseudouridine). In some embodiments, each of the cytidines in the RNA are modified cytidines (e.g., 5-methylcytidine).

### Pharmaceutical Compositions

The present disclosure provides pharmaceutical compositions comprising a 5′-capped RNA, or a delivery vehicle comprising the 5′-capped RNA, described herein, and a pharmaceutically acceptable carrier.

Methods for preparing pharmaceutical compositions are known to those skilled in the art, see, e.g., Remington's Pharmaceutical Sciences (19th Ed., Williams & Wilkins, 1995). In some embodiments, the pharmaceutical composition comprises a quantity of a 5′-capped RNA determined to be in a pharmaceutically safe and effective amount. In some embodiments, the 5′-capped RNA is a 5′-capped mRNA comprising a ORF, wherein the effective amount results in increased expression of the encoded protein in a target cell population or tissue upon in vivo administration.

For treatment of a subject, such as a mammal or a human, dosages are determined based on factors such as the weight and overall health of the subject, the condition treated, severity of symptoms, etc. Dosages and concentrations are determined to produce the desired benefit while avoiding any undesirable side effects. Typical dosages of the subject compounds are in the range of about 0.0005 to 500 mg/day for a human patient, and ranging in some embodiments between about 1-100 mg/day.

### Methods of Use

The present disclosure provides methods of use of a 5′-capped RNA described herein, a delivery vehicle comprising the 5′-capped RNA described herein, or a pharmaceutical composition comprising the 5′-capped RNA or the delivery vehicle, e.g., as a therapeutic agent.

In some embodiments, the disclosure provides a method for treating a subject having a disease or disorder, comprising administering to the subject a 5′-capped RNA described herein, a delivery vehicle comprising the 5′-capped RNA described herein, or a pharmaceutical composition comprising the 5′-capped RNA or the delivery vehicle described herein. In some embodiments, the 5′-capped RNA is a 5′-capped mRNA comprising a ORF, wherein expression of the protein encoded by the ORF provides a therapeutic benefit to the subject.

In some embodiments, the 5′-capped RNA is formulated in a delivery vehicle. In some embodiments, the delivery vehicle comprises a nanoparticle. In some embodiments, the nanoparticle is a polymeric nanoparticle. In some embodiments, the nanoparticle is a lipid nanoparticle. In some embodiments, the delivery vehicle comprises a liposome. Accordingly provided are nanoparticles, polymeric nanoparticles, lipid nanoparticles, and liposomes comprising the 5′-capped RNAs (e.g. 5′-capped mRNAs) produced by the methods and systems of the disclosure.

In some embodiments, the 5′-capped RNA is formulated in a pharmaceutical composition described herein. In some embodiments, a delivery vehicle (e.g., lipid nanoparticle) comprising the 5′-capped RNA is formulated in a pharmaceutical composition described herein.

In some embodiments, the method comprises administering an effective dose of the 5′-capped RNA.

### Kits

The present disclosure provides kits comprising a capping primer, or a transcription system comprising the capping primer, described herein.

In some embodiments, the kit comprises a capping primer with instructions for performing RNA synthesis. In some embodiments, the kit further comprises a polynucleotide template, one or more unmodified NTPs, one or more modified NTPs (e.g., pseudouridine 5′-triphosphate), an RNA polymerase, other transcription enzymes, a reaction buffer, and/or magnesium. In some embodiments, the RNA polymerase is a T7 RNAP or variant thereof described herein.

In some embodiments, the disclosure provides a kit comprising a 5′-capped RNA described herein, and instructions for administering the 5′-capped RNA to a subject for treating a disease or disorder.

In some embodiments, the disclosure provides a kit comprising a 5′-capped mRNA described herein, wherein the 5′-capped mRNA comprises an ORF encoding a protein, and instructions for expressing protein from the 5′-capped mRNA. In some embodiments, the instructions provide for protein expression in vitro or in vivo.

### OTHER EMBODIMENTS

The present disclosure provides the following enumerated embodiments.

Embodiment I-1. A capping primer comprising a structure according to Formula II, wherein, q1 to q7, are each independently 0 or 1; B1 through B7, if present, are each a nucleobase independently selected from the group consisting of adenine, guanine, thymine, uracil, cytosine and any derivative or analog of the foregoing; B8 is a nucleobase selected from the group consisting of adenine, guanine, thymine, uracil, cytosine and any derivative or analog of the foregoing; B9 is a nucleobase selected from the group consisting of adenine, uracil, cytosine and any derivative or analog of the foregoing; B10 is a nucleobase selected from the group consisting of adenine, guanine, uracil, cytosine and any derivative or analog of the foregoing; R1 are each independently H, alkyl, acyl, benzyl, a cleavable unit; R2 and R3 are each independently H, OH, alkyl, O-alkyl, O-acyl, O-benzyl, halogen, LNA, a linker, a cleavable unit or a detectable marker; R4 to R12, if present, are each independently H, OH, alkyl, O-alkyl, halogen, LNA, a linker or a detectable marker; X1 to X12, if present, are each independently O or S; Y1 to Y12, if present, are each independently O, S, BH₃, aryl, alkyl, O-alkyl or O-aryl; and Z1 to Z22, if present, are each independently O, S, NH, CH₂, C(halogen)₂ or CH(halogen); wherein, the capping primer hybridizes to a polynucleotide template, wherein said polynucleotide template comprises a nucleobase at position −1 immediately adjacent to and downstream (3′) of a transcriptional start site and a nucleobase at each of position +1, position +2 and position +3 immediately adjacent to and upstream (5′) of the transcriptional start site; and wherein, B8 is complementary or mismatched to a nucleobase at positions −1 of the polynucleotide template; B9 is complementary to a nucleobase at position +1 of the polynucleotide template; and B10 is complementary to a nucleobase at position +2 of the polynucleotide template; wherein B8, B9, and B10 are not fully complementary to nucleobases at positions +1 to +3 of the polynucleotide template.

Embodiment I-2. The capping primer of embodiment I-1, wherein the capping primer consists of the structure according to Formula II.

Embodiment I-3. The capping primer of embodiment I-1 or I-2, wherein, q1 to q7 are O; R1 are each independently H, alkyl, acyl, benzyl or a cleavable unit; R2 and R3 are each independently H, OH, alkyl, O-alkyl, O-acyl, O-benzyl, halogen, LNA, a linker, a cleavable unit or a detectable marker; and R11 and R12 are O-alkyl.

Embodiment I-4. The capping primer of embodiment I-1 or I-2, wherein at least one of q1 to q7 is 1.

Embodiment I-5. The capping primer of embodiment I-1 or I-2, wherein at least two of q1 to q7 is 1, or wherein at least three of q1 to q7 is 1.

Embodiment I-6. The capping primer of embodiment I-1 or I-2, wherein at least four of q1 to q7 is 1.

Embodiment I-7. The capping primer of embodiment I-1 or I-2, wherein at least five of q1 to q7 is 1.

Embodiment I-8. The capping primer of embodiment I-1 or I-2, wherein at least six of q1 to q7 is 1.

Embodiment I-9. The capping primer of embodiment I-1 or I-2, wherein q1 to q7 is 1.

Embodiment I-10. The capping primer of any of embodiments I-1 to I-9, wherein B8 is adenine or N6-methyladenosine.

Embodiment I-11. The capping primer of any of embodiments I-1 to I-9, wherein B8 is uracil, or a derivative or analog thereof.

Embodiment I-12. The capping primer of any of embodiments I-1 to I-11, wherein B9 is adenine, or a derivative or analog thereof.

Embodiment I-13. The capping primer of any of embodiments I-1 to I-12, wherein B10 is guanine, or a derivative or analog thereof.

Embodiment I-14. The capping primer of any of embodiments I-1 to I-13, wherein R2 is OH.

Embodiment I-15. The capping primer of any of embodiments I-1 to I-14, wherein R3 is OMe.

Embodiment I-16. The capping primer of any of embodiments I-1 to I-15, wherein R4 to R10, if present, and R11 and R12 are OMe.

Embodiment I-17. The capping primer of any of embodiments I-1 to I-16, wherein B8 is complementary to a nucleobase at position −1 of the polynucleotide template.

Embodiment I-18. The capping primer of any of embodiments I-1 to I-16, wherein B8 is mismatched to a nucleobase at positions −1 of the polynucleotide template.

Embodiment I-19. A stereoisomer, tautomer or salt form of the capping primer of any of embodiments I-1 to I-18.

Embodiment I-20. A system comprising a capping primer and a polynucleotide template, wherein the capping primer comprises a structure according to Formula II, wherein q1 to q7, are each independently 0 or 1; B1 through B7, if present, are each a nucleobase independently selected from the group consisting of adenine, guanine, thymine, uracil, cytosine and any derivative or analog of the foregoing; B8 is a nucleobase selected from the group consisting of adenine, guanine, thymine, uracil, cytosine and any derivative or analog of the foregoing; B9 is a nucleobase selected from the group consisting of adenine, uracil, cytosine and any derivative or analog of the foregoing; B10 is a nucleobase selected from the group consisting of adenine, guanine, uracil, cytosine and any derivative or analog of the foregoing; R1 are each independently H, alkyl, acyl, benzyl or a cleavable unit; R2 and R3 are each independently H, OH, alkyl, O-alkyl, O-acyl, O-benzyl, halogen, LNA, a linker, a cleavable unit or a detectable marker; R4 to R12, if present, are each independently H, OH, alkyl, O-alkyl, halogen, LNA, a linker or a detectable marker; X1 to X12, if present, are each independently O or S; Y1 to Y12, if present, are each independently O⁻, S⁻, BH₃⁻, aryl, alkyl, O-alkyl or O-aryl; and Z1 to Z22, if present, are each independently O, S, NH, CH₂, C(halogen)₂ or CH(halogen); wherein the polynucleotide template comprises a nucleobase at position −1 immediately adjacent to and downstream (3′) of a transcriptional start site and a nucleobase at each of position +1, position +2 and position +3 immediately adjacent to and upstream (5′) of the transcriptional start site, and wherein B8 is complementary or mismatched to a nucleobase at positions −1 of the polynucleotide template; B9 is complementary to a nucleobase at position +1 of the polynucleotide template; and B10 is complementary to a nucleobase at position +2 of the polynucleotide template; and wherein B8, B9, and B10 are not fully complementary to nucleobases at positions +1 to +3 of the polynucleotide template.

Embodiment I-21. The system of embodiment I-20, wherein the capping primer consists of the structure according to Formula II.

Embodiment I-22. The system of embodiment I-20 or I-21, wherein q1 to q7 are O; R1 are each independently H, alkyl, acyl, benzyl or a cleavable unit; R2 and R3 are each independently H, OH, alkyl, O-alkyl, O-acyl, O-benzyl, halogen, LNA, a linker, a cleavable unit or a detectable marker; and R11 and R12 are O-alkyl.

Embodiment I-23. The system of embodiment I-20 or I-21, wherein at least one of q1 to q7 is 1.

Embodiment I-24. The system of embodiment I-20 or I-21, wherein at least two of q1 to q7 is 1.

Embodiment I-25. The system of embodiment I-20 or I-21, wherein at least three of q1 to q7 is 1.

Embodiment I-26. The system of embodiment I-20 or I-21, wherein at least four of q1 to q7 is 1.

Embodiment I-27. The system of embodiment I-20 or I-21, wherein at least five of q1 to q7 is 1.

Embodiment I-28. The system of embodiment I-20 or I-21, wherein at least six of q1 to q7 is 1.

Embodiment I-29. The system of embodiment I-20 or I-21, wherein q1 to q7 is 1.

Embodiment I-30. The system of any of embodiments I-20 to I-29, wherein B8 is adenine or N6-methyladenosine.

Embodiment I-31. The system of any of embodiments I-20 to I-29, wherein B8 is uracil.

Embodiment I-32. The system of any of embodiments I-20 to I-31, wherein B9 is adenine.

Embodiment I-33. The system of any of embodiments I-20 to I-32, wherein B10 is guanine.

Embodiment I-34. The system of any of embodiments I-20 to I-33, wherein R2 is OH.

Embodiment I-35. The system of any of embodiments I-20 to I-34, wherein R3 is OMe.

Embodiment I-36. The system of any of embodiments I-20 to I-35, wherein R4 to R10, if present, and R11 and R12 are OMe.

Embodiment I-37. The system of any of embodiments I-20 to I-36, wherein the polynucleotide template is a DNA template.

Embodiment I-38. The system of embodiment I-37, wherein the DNA template comprises a sequence encoding a 5′-untranslated region (UTR), an open-reading frame (ORF), a 3′-UTR, and a polyA sequence.

Embodiment I-39. A method of producing 5′-capped RNA molecules in an in vitro transcription (IVT) reaction comprising the steps of: mixing the capping primer of any of embodiments I-1 to I-18, or the stereoisomer, tautomer, or salt form of embodiment I-19, with a polynucleotide template, nucleoside 5′-triphosphates and a RNA polymerase; and incubating the mixture under transcription conditions, thereby resulting in synthesis of a 5′-capped RNA molecule.

Embodiment I-40. A method of producing a 5′-capped RNA molecule in an in vitro transcription (IVT) reaction comprising the steps of: mixing the system of any of embodiments I-20 to I-38 with nucleoside 5′-triphosphates and a RNA polymerase; and incubating the mixture under transcription conditions, thereby resulting in synthesis of a 5′-capped RNA molecule.

Embodiment I-41. The method of embodiment I-39 or I-40, wherein the RNA polymerase is a T7 RNA polymerase or a variant thereof, a T3 RNA polymerase or a variant thereof, or a SP6 RNA polymerase or a variant thereof.

Embodiment I-42. The method of embodiment I-39 or I-40, wherein the RNA polymerase is a T7 RNA polymerase or a variant thereof.

Embodiment I-43. The method of any of embodiments I-39 to I-42, wherein the capping primer and the nucleoside 5′-triphosphates are present in the IVT reaction at approximately equimolar concentrations.

Embodiment I-44. The method of any of embodiments I-39 to I-43, wherein at least about 70%, about 75%, about 80%, about 85%, about 90%, about 91%, about 92%, about 93%, about 94%, about 95%, about 96%, about 97%, about 98%, or about 99% of the RNA molecules produced by the IVT reaction are 5′-capped.

Embodiment I-45. The method of any of embodiments I-39 to I-44, wherein the method further comprises a step of purifying the 5′-capped RNA molecules.

Embodiment I-46. 5′-capped RNA molecules produced by the method of any of embodiments I-39 to I-45.

Embodiment I-47. The 5′-capped RNA molecules of embodiment I-46, comprising 5′-capped messenger RNAs (mRNAs).

Embodiment I-48. A cell comprising the 5′-capped RNA molecules of embodiment I-46 or I-47.

Embodiment I-49. A pharmaceutical composition comprising the 5′-capped RNA molecules of embodiment I-46 or I-47.

Embodiment I-50. The pharmaceutical composition comprising the 5′-capped RNA molecules of embodiment I-49, wherein the pharmaceutical composition is formulated as a lipid nanoparticle (LNP).

Embodiment I-51. An LNP comprising the 5′-capped RNA molecule of embodiment I-46 or I-47.

Embodiment I-52. A kit comprising the capping primer of any of embodiments I-1 to I-18, the stereoisomer, tautomer, or salt form of embodiment I-19, or the system of any of embodiments I-21 to I-38, and instructions for use in an IVT reaction to synthesize 5′-capped RNA molecules.

Embodiment II-1. A capping primer comprising a structure according to Formula II, or a pharmaceutically acceptable salt thereof, wherein, q1 to q7, are each independently 0 or 1; B1 through B7, if present, are each a nucleobase independently selected from the group consisting of adenine, guanine, thymine, uracil, cytosine, hypoxanthine, and any derivative or analog of the foregoing; B8 is a nucleobase selected from the group consisting of adenine, guanine, thymine, uracil, cytosine, hypoxanthine, and any derivative or analog of the foregoing; B9 is a nucleobase selected from the group consisting of adenine, uracil, cytosine, and any derivative or analog of the foregoing; B10 is a nucleobase selected from the group consisting of adenine, guanine, uracil, cytosine, and any derivative or analog of the foregoing; R1 are each independently H, alkyl, acyl, benzyl, or a cleavable unit; R2 is H, OH, alkyl, O-alkyl, O-acyl, O-benzyl, O-methoxyethyl (MOE), halogen, LNA, a linker, a cleavable unit, or a detectable marker; R3 is H, OH, alkyl, O-alkyl, O-acyl, O-benzyl, O-methoxyethyl (MOE), halogen, a linker, a cleavable unit, or a detectable marker; R4 to R12, if present, are each independently H, OH, alkyl, O-alkyl, O-methoxyethyl (MOE), halogen, LNA, a linker, or a detectable marker; X1 to X12, if present, are each independently O or S; Y1 to Y12, if present, are each independently O⁻, S⁻, BH₃⁻, aryl, alkyl, O-alkyl, or O-aryl; and Z1 to Z22, if present, are each independently O, S, NH, CH₂, C(halogen)₂, or CH(halogen); wherein, the capping primer hybridizes to a polynucleotide template, wherein said polynucleotide template comprises an antisense strand comprising a nucleobase at position −1 immediately adjacent to and downstream (3′) of a transcriptional start site, and a nucleobase at each of position +1, position +2 and position +3 immediately adjacent to and upstream (5′) of the transcriptional start site; and wherein, B8 is complementary or mismatched to a nucleobase at position −1 of the polynucleotide template; B9 is complementary to a nucleobase at position +1 of the polynucleotide template; and B10 is complementary to a nucleobase at position +2 of the polynucleotide template; and B8, B9, and B10 are not fully complementary to nucleobases at positions +1 to +3 of the polynucleotide template.

Embodiment II-2. The capping primer of embodiment II-1, wherein the capping primer consists of the structure according to Formula II.

Embodiment II-3. The capping primer of embodiment II-1 or II-2, wherein q1 to q7 are each 0.

Embodiment II-4. The capping primer of embodiment II-1 or II-2, wherein at least one of q1 to q7 is 1.

Embodiment II-5. The capping primer of embodiment II-1 or II-2, wherein at least two of q to q7 are 1.

Embodiment II-6. The capping primer of embodiment II-1 or II-2, wherein at least three of q to q7 are 1.

Embodiment II-7. The capping primer of embodiment II-1 or II-2, wherein at least four of q to q7 are 1.

Embodiment II-8. The capping primer of embodiment II-1 or II-2, wherein at least five of q to q7 are 1.

Embodiment II-9. The capping primer of embodiment II-1 or II-2, wherein at least six of q to q7 are 1.

Embodiment II-10. The capping primer of embodiment II-1 or II-2, wherein q1 to q7 are each 1.

Embodiment II-11. The capping primer of any one of embodiments II-1 to II-10, wherein B8 is a nucleobase selected from the group consisting of adenine, uracil, thymine, cytosine, 5-methylcytosine, guanine, and hypoxanthine.

Embodiment II-12. The capping primer of any of embodiments II-1 to II-11, wherein B9 is selected from the group consisting of adenine, cytosine, 5-methylcytosine, thymine and uracil.

Embodiment II-13. The capping primer of any of embodiments II-1 to II-11, wherein B9 is selected from the group consisting of adenine, cytosine, and uracil.

Embodiment II-14. The capping primer of any of embodiments II-1 to II-13, wherein B10 is selected from the group consisting of adenine, guanine, cytosine, 5-methylcytosine, thymine and uracil.

Embodiment II-15. The capping primer of any of embodiments II-1 to II-13, wherein B10 is selected from the group consisting of adenine, guanine, cytosine, and uracil.

Embodiment II-16. The capping primer of any one of embodiments II-1 to II-11, wherein B9 is adenine, or a derivative or analog thereof, and B10 is guanine, or a derivative or analog thereof.

Embodiment II-17. The capping primer of any one of embodiments II-1 to II-11, wherein B9 is cytosine, or a derivative or analog thereof, and B10 is guanine, or a derivative or analog thereof.

Embodiment II-18. The capping primer of any one of embodiments II-1 to II-11, wherein B9 is uracil, or a derivative or analog thereof, and B10 is guanine, or a derivative or analog thereof.

Embodiment II-19. The capping primer of any one of embodiments II-1 to II-11, wherein B9 is adenine, or a derivative or analog thereof, and B10 is adenine, or a derivative or analog thereof.

Embodiment II-20. The capping primer of any one of embodiments II-1 to II-11, wherein B9 is adenine, or a derivative or analog thereof, and B10 is cytosine, or a derivative or analog thereof.

Embodiment II-21. The capping primer of any one of embodiments II-1 to II-11, wherein B9 is adenine, or a derivative or analog thereof, and B10 is uracil, or a derivative or analog thereof.

Embodiment II-22. The capping primer of any of embodiments II-1 to II-21, wherein R1 are each independently H or C1-C6 alkyl.

Embodiment II-23. The capping primer of any of embodiments II-1 to II-21, wherein R1 are each H.

Embodiment II-24. The capping primer of any of embodiments II-1 to II-23, wherein R2 is H, OH, O—(C1-C6 alkyl), O-MOE, fluoro, or LNA.

Embodiment II-25. The capping primer of any of embodiments II-1 to II-23, wherein R2 is OH.

Embodiment II-26. The capping primer of any of embodiments II-1 to II-25, wherein R3 is H, OH, O—(C1-C6 alkyl), O-MOE, or fluoro.

Embodiment II-27. The capping primer of any of embodiments II-1 to II-25, wherein R3 is OH or OMe.

Embodiment II-28. The capping primer of any of embodiments II-1 to II-27, wherein R4 to R10, if present, and R11 and R12 are each independently H, OH, O—(C1-C6 alkyl), O-MOE, fluoro, or LNA.

Embodiment II-29. The capping primer of any of embodiments II-1 to II-27, wherein R11 is H, OH, OMe, O-MOE, or fluoro, and R12 is OH or OMe.

Embodiment II-30. The capping primer of any of embodiments II-1 to II-29, wherein B8 is complementary to a nucleobase at position −1 of the polynucleotide template.

Embodiment II-31. The capping primer of any of embodiments II-1 to II-29, wherein B8 is mismatched to a nucleobase at position −1 of the polynucleotide template.

Embodiment II-32. A stereoisomer, tautomer or salt form of the capping primer of any of embodiments II-1 to II-31.

Embodiment II-33. A system comprising a capping primer and a polynucleotide template, wherein the capping primer comprises a structure according to Formula II, or a pharmaceutically acceptable salt thereof, wherein q1 to q7, are each independently 0 or 1; B1 through B7, if present, are each a nucleobase independently selected from the group consisting of adenine, guanine, thymine, uracil, cytosine, hypoxanthine, and any derivative or analog of the foregoing; B8 is a nucleobase selected from the group consisting of adenine, guanine, thymine, uracil, cytosine, hypoxanthine, and any derivative or analog of the foregoing; B9 is a nucleobase selected from the group consisting of adenine, uracil, cytosine, and any derivative or analog of the foregoing; B10 is a nucleobase selected from the group consisting of adenine, guanine, uracil, cytosine, and any derivative or analog of the foregoing; R1 are each independently H, alkyl, acyl, benzyl, or a cleavable unit; R2 is H, OH, alkyl, O-alkyl, O-acyl, O-benzyl, O-MOE, halogen, LNA, a linker, a cleavable unit, or a detectable marker; R3 is H, OH, alkyl, O-alkyl, O-acyl, O-benzyl, O-MOE, halogen, a linker, a cleavable unit, or a detectable marker; R4 to R12, if present, are each independently H, OH, alkyl, O-alkyl, O-MOE, halogen, LNA, a linker, or a detectable marker; X1 to X12, if present, are each independently O or S; Y1 to Y12, if present, are each independently O⁻, S⁻, BH₃⁻, aryl, alkyl, O-alkyl, or O-aryl; and Z1 to Z22, if present, are each independently O, S, NH, CH₂, C(halogen)₂, or CH(halogen); wherein the polynucleotide template comprises an antisense strand comprising a nucleobase at position −1 immediately adjacent to and downstream (3′) of a transcriptional start site, and a nucleobase at each of position +1, position +2 and position +3 immediately adjacent to and upstream (5′) of the transcriptional start site, and wherein B8 is complementary or mismatched to a nucleobase at positions −1 of the polynucleotide template; B9 is complementary to a nucleobase at position +1 of the polynucleotide template; B10 is complementary to a nucleobase at position +2 of the polynucleotide template; and B8, B9, and B10 are not fully complementary to nucleobases at positions +1 to +3 of the polynucleotide template.

Embodiment II-34. The system of embodiment II-33, wherein the capping primer consists of the structure according to Formula II.

Embodiment II-35. The system of embodiment II-33 or II-34, wherein q1 to q7 are each 0.

Embodiment II-36. The system of embodiment II-33 or II-34, wherein at least one of q1 to q7 is 1.

Embodiment II-37. The system of embodiment II-33 or II-34, wherein at least two of q1 to q7 are 1.

Embodiment II-38. The system of embodiment II-33 or II-34, wherein at least three of q1 to q7 are 1.

Embodiment II-39. The system of embodiment II-33 or II-34, wherein at least four of q1 to q7 are 1.

Embodiment II-40. The system of embodiment II-33 or II-34, wherein at least five of q1 to q7 are 1.

Embodiment II-41. The system of embodiment II-33 or II-34, wherein at least six of q1 to q7 are 1.

Embodiment II-42. The system of embodiment II-33 or II-34, wherein q1 to q7 are each 1.

Embodiment II-43. The system of any of embodiments II-33 to II-42, wherein B8 is a nucleobase selected from the group consisting of adenine, uracil, thymine, cytosine, 5-methylcytosine, guanine, and hypoxanthine.

Embodiment II-44. The system of any of embodiments II-33 to II-43, wherein B9 is selected from the group consisting of adenine, cytosine, 5-methylcytosine, thymine and uracil.

Embodiment II-45. The system of any of embodiments II-33 to II-43, wherein B9 is selected from the group consisting of adenine, cytosine and uracil.

Embodiment II-46. The system of any of embodiments II-33 to II-45, wherein B10 is selected from the group consisting of adenine, guanine, cytosine, 5-methylcytosine, thymine and uracil.

Embodiment II-47. The system of any of embodiments II-33 to II-45, wherein B10 is selected from the group consisting of adenine, guanine, cytosine and uracil.

Embodiment II-48. The system of any of embodiments II-33 to II-43, wherein B9 is adenine, or a derivative or analog thereof; and B10 is guanine, or a derivative or analog thereof.

Embodiment II-49. The system of embodiment II-48, wherein (i) the nucleobase at position +1 of the polynucleotide template is thymine, and (ii) the nucleobase at position +2 of the polynucleotide template is cytosine.

Embodiment II-50. The system of any of embodiments II-33 to II-43, wherein B9 is cytosine, or a derivative or analog thereof; and B10 is guanine, or a derivative or analog thereof.

Embodiment II-51. The system of embodiment II-50, wherein (i) the nucleobase at position +1 of the polynucleotide template is guanine, and (ii) the nucleobase at position +2 of the polynucleotide template is cytosine.

Embodiment II-52. The system of any of embodiments II-33 to II-43, wherein B9 is uracil, or a derivative or analog thereof, and B10 is guanine, or a derivative or analog thereof.

Embodiment II-53. The system of embodiment II-52, wherein (i) the nucleobase at position +1 of the polynucleotide template is adenine, and (ii) the nucleobase at position +2 of the polynucleotide template is cytosine.

Embodiment II-54. The system of any of embodiments II-33 to II-43, wherein B9 is adenine, or a derivative or analog thereof; and B10 is adenine, or a derivative or analog thereof.

Embodiment II-55. The system of embodiment II-54, wherein (i) the nucleobase at position +1 of the polynucleotide template is thymine, and (ii) the nucleobase at position +2 of the polynucleotide template is thymine.

Embodiment II-56. The system of any of embodiments II-33 to II-43, wherein B9 is adenine, or a derivative or analog thereof; and B10 is cytosine, or a derivative or analog thereof.

Embodiment II-57. The system of embodiment II-56, wherein (i) the nucleobase at position +1 of the polynucleotide template is thymine, and (ii) the nucleobase at position +2 of the polynucleotide template is guanine.

Embodiment II-58. The system of any of embodiments II-33 to II-43, wherein B9 is adenine, or a derivative or analog thereof; and B10 is uracil, or a derivative or analog thereof.

Embodiment II-59. The system of embodiment II-58, wherein (i) the nucleobase at position +1 of the polynucleotide template is thymine, and (ii) the nucleobase at position +2 of the polynucleotide template is adenine.

Embodiment II-60. The system of any of embodiments II-33 to II-59, wherein the nucleobase at position −1 of the polynucleotide template is thymine, or adenine.

Embodiment II-61. The system of any of embodiments II-33 to II-60, wherein R1 are each independently H or C1-C6 alkyl.

Embodiment II-62. The system of any of embodiments II-33 to II-60, wherein R1 are each H.

Embodiment II-63. The system of any of embodiments II-33 to II-62, wherein R2 is H, OH, O—(C1-C6 alkyl), O-MOE, fluoro, or LNA.

Embodiment II-64. The system of any one of embodiments II-33 to II-62, wherein R2 is OH.

Embodiment II-65. The system of any of embodiments II-33 to II-64, wherein R3 is H, OH, O—(C1-C6 alkyl), O-MOE, or fluoro.

Embodiment II-66. The system of any of embodiments II-33 to II-64, wherein R3 is OH or OMe.

Embodiment II-67. The system of any of embodiments II-33 to II-66, wherein R4 to R10, if present, and R11 and R12 are each independently H, OH, O—(C1-C6 alkyl), O-MOE, fluoro, or LNA.

Embodiment II-68. The system of any of embodiments II-33 to II-66, wherein R11 is H, OH, OMe, O-MOE, or fluoro and R12 is OH or OMe.

Embodiment II-69. The system of any of embodiments II-33 to II-68, wherein B8 is complementary to a nucleobase at position −1 of the polynucleotide template.

Embodiment II-70. The system of any of embodiments II-33 to II-68, wherein B8 is mismatched to a nucleobase at position −1 of the polynucleotide template.

Embodiment II-71. The system of any of embodiments II-33 to II-70, wherein the polynucleotide template is a DNA template.

Embodiment II-72. The system of any of embodiments II-33 to II-71, wherein the polynucleotide template is linear DNA.

Embodiment II-73. The system of any of embodiments II-33 to II-72, wherein the polynucleotide template comprises a sequence encoding a 5′-untranslated region (UTR), an open-reading frame (ORF), a 3′-UTR, and a polyA sequence.

Embodiment II-74. A method of producing 5′-capped RNA molecules in an in vitro transcription (IVT) reaction comprising the steps of: mixing the capping primer of any of embodiments II-1 to II-31, or the stereoisomer, tautomer, or salt form of embodiment II-32, with the polynucleotide template, nucleoside 5′-triphosphates (NTPs), and a RNA polymerase; and incubating the mixture under transcription conditions, thereby resulting in synthesis of a 5′-capped RNA molecule.

Embodiment II-75. A method of producing a 5′-capped RNA molecule in an in vitro transcription (IVT) reaction comprising the steps of: mixing the system of any of embodiments II-33 to II-73 with nucleoside 5′-triphosphates (NTPs), and an RNA polymerase; and incubating the mixture under transcription conditions, thereby resulting in synthesis of a 5′-capped RNA molecule.

Embodiment II-76. The method of embodiment II-74 or II-75, wherein the RNA polymerase is a T7 RNA polymerase or a variant thereof, a T3 RNA polymerase or a variant thereof, or a SP6 RNA polymerase or a variant thereof.

Embodiment II-77. The method of embodiment II-74 or II-75, wherein the RNA polymerase is a T7 RNA polymerase or a variant thereof.

Embodiment II-78. The method of any of embodiments II-74 to II-77, wherein the capping primer and the NTPs are present in the IVT reaction at approximately equimolar concentrations.

Embodiment II-79. The method of any of embodiments II-74 to II-78, wherein the NTPs comprise adenosine triphosphate (ATP) or a derivative or analog thereof, guanosine triphosphate (GTP) or a derivative or analog thereof, cytosine triphosphate (CTP) or a derivative or analog thereof, and uridine triphosphate (UTP) or a derivative or analog thereof, each at an approximately equimolar concentration, optionally wherein the derivative or analog of UTP is pseudouridine triphosphate (pseudo-UTP) or N1-methylpseudouridine triphosphate (N1-pseudo-UTP).

Embodiment II-80. The method of any of embodiments II-74 to II-79, wherein the NTPs comprise ATP, GTP, CTP, and UTP at an approximately equimolar concentration, optionally wherein UTP is pseudo-UTP or N1-pseudo-UTP, and wherein (i) each NTP is present in the IVT reaction at a molar concentration that is about 1.1-fold, 1.2-fold, 1.3-fold, 1.4-fold, 1.5-fold, 2-fold, 2.5-fold, 3-fold, 4-fold, 5-fold, or 10-fold higher than a molar concentration of the capping primer, or (ii) the capping primer is present in the IVT reaction at a molar concentration that is about 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, or 5-fold higher than a molar concentration of each NTP.

Embodiment II-81. The method of any of embodiments II-74 to II-80, wherein at least about 65%, about 70%, about 75%, about 80%, about 85%, about 90%, about 91%, about 92%, about 93%, about 94%, about 95%, about 96%, about 97%, about 98%, or about 99% of the RNA molecules produced by the IVT reaction are 5′-capped.

Embodiment II-82. The method of any of embodiments II-74 to II-80, wherein the synthesis results in a yield of RNA molecules that is at least about 60%, about 70%, about 75%, about 80%, about 85%, about 90%, about 91%, about 92%, about 93%, about 94%, about 95%, about 96%, about 97%, about 98%, or about 99% relative to the RNA yield obtained in the reaction without using a primer.

Embodiment II-83. The method of any of embodiments II-74 to II-80, wherein the synthesis results in a yield of RNA molecules that is at least about 1.1-fold, 1.5-fold, 2-fold, 3-fold, 4-fold, 5-fold, 6-fold, 7-fold, 8-fold, 9-fold, or 10-fold higher relative to the RNA yield obtained in the reaction without using a primer.

Embodiment II-84. The method of any of embodiments II-74 to II-80, wherein the synthesis results in (a) a yield of RNA molecules that is at least about 30% relative to the RNA yield obtained in the reaction without using a primer; and (b) at least about 30% of the RNA molecules produced are 5′-capped.

Embodiment II-85. The method of any of embodiments II-74 to II-80, wherein the synthesis results in (a) a yield of RNA molecules that is at least about 50% relative to the RNA yield obtained in the reaction without using a primer; and (b) at least about 50% of the RNA molecules produced are 5′-capped.

Embodiment II-86. The method of any of embodiments II-74 to II-80, wherein the synthesis results in (a) a yield of RNA molecules that is at least about 70% relative to the RNA yield obtained in the reaction without using a primer; and (b) at least about 70% of the RNA molecules produced are 5′-capped.

Embodiment II-87. The method of any of embodiments II-74 to II-86, wherein the method further comprises a step of purifying the 5′-capped RNA molecules.

Embodiment II-88. 5′-capped RNA molecules produced by the method of any of embodiments II-74 to II-87.

Embodiment II-89. The 5′-capped RNA molecules of embodiment II-88, comprising 5′-capped messenger RNAs (mRNAs).

Embodiment II-90. A cell comprising the 5′-capped RNA molecules of embodiment II-88 or II-89.

Embodiment II-91. A pharmaceutical composition comprising the 5′-capped RNA molecules of embodiment II-88 or II-89.

Embodiment II-92. The pharmaceutical composition comprising the 5′-capped RNA molecules of embodiment II-91, wherein the pharmaceutical composition is formulated as a lipid nanoparticle (LNP).

Embodiment II-93. An LNP comprising the 5′-capped RNA molecule of embodiment II-88 or II-89.

Embodiment II-94. A kit comprising the capping primer of any of embodiments II-1 to II-33, the stereoisomer, tautomer, or salt form of embodiment II-32, or the system of any of embodiments II-33 to II-73, and instructions for use in an IVT reaction to synthesize 5′-capped RNA molecules.

Embodiment II-95. A kit for producing a 5′-capped RNA molecule, comprising: (a) a capping primer comprising a structure according to Formula III, or a pharmaceutically acceptable salt thereof, wherein, B′8 is a nucleobase selected from the group consisting of adenine, guanine, thymine, uracil, cytosine, and hypoxanthine and any derivative or analog of the foregoing; B′9 is a nucleobase selected from the group consisting of adenine, uracil, cytosine, and any derivative or analog of the foregoing; B′10 is a nucleobase selected from the group consisting of adenine, guanine, uracil, cytosine, and any derivative or analog of the foregoing; R′1 are each independently H, alkyl, acyl, benzyl, or a cleavable unit; R′2 is H, OH, alkyl, O-alkyl, O-acyl, O-benzyl, O-MOE, halogen, LNA, a linker, a cleavable unit, or a detectable marker; R′3 is H, OH, alkyl, O-alkyl, O-acyl, O-benzyl, O-MOE, halogen, a linker, a cleavable unit, or a detectable marker; R′11 and R′12 are each independently H, OH, alkyl, O-alkyl, O-MOE, halogen, LNA, a linker, or a detectable marker; X′1 to X′3, X′11, and X′12 are each independently O or S; Y′1 to Y′3, Y′11, and Y′12 are each independently O⁻, S⁻, BH₃⁻, aryl, alkyl, O-alkyl, or O-aryl; and Z′1 to Z′4 and Z′19 to Z′21 are each independently O or S, NH, CH₂, C(halogen)₂ or CH(halogen); and (b) instructions for use of the capping primer in an IVT reaction with a polynucleotide template to synthesize 5′-capped RNA molecules, wherein: the polynucleotide template comprises an antisense strand comprising a nucleobase at position −1 immediately adjacent to and downstream (3′) of a transcriptional start site, and a nucleobase at each of position +1, position +2 and position +3 immediately adjacent to and upstream (5′) of the transcriptional start site; B′8 is complementary or mismatched to a nucleobase at positions −1 of the polynucleotide template; B′9 is complementary to a nucleobase at position +1 of the polynucleotide template; B′10 is complementary to a nucleobase at position +2 of the polynucleotide template; and B′8, B′9, and B′10 are not fully complementary to nucleobases at positions +1 to +3 of the polynucleotide template.

Embodiment II-96. The kit of embodiment II-95, wherein the capping primer consists of the structure according to Formula III.

Embodiment II-97. The kit of embodiment II-95 or II-96, wherein B′8 is a nucleobase selected from the group consisting of adenine, uracil, thymine, cytosine, 5-methylcytosine, guanine, and hypoxanthine.

Embodiment II-98. The kit of any of embodiments II-95 to II-97, wherein B′9 is selected from the group consisting of adenine, cytosine, 5-methylcytosine, thymine and uracil.

Embodiment II-99. The kit of any of embodiments II-95 to II-98, wherein B′9 is selected from the group consisting of adenine, cytosine and uracil.

Embodiment II-100. The kit of any of embodiments II-95 to II-99, wherein B′10 is selected from the group consisting of adenine, guanine, cytosine, 5-methylcytosine, thymine and uracil.

Embodiment II-101. The kit of any of embodiments II-95 to II-99, wherein B′10 is selected from the group consisting of adenine, guanine, cytosine and uracil.

Embodiment II-102. The kit of any one of embodiments II-95 to II-97, wherein B′9 is adenine, or a derivative or analog thereof; and B10 is guanine, or a derivative or analog thereof,

Embodiment II-103. The kit of embodiment II-102, wherein (i) the nucleobase at position +1 of the polynucleotide template is thymine, and (ii) the nucleobase at position +2 of the polynucleotide template is cytosine.

Embodiment II-104. The kit of any of embodiments II-95 to II-97, wherein B′9 is cytosine, or a derivative or analog thereof; and B′10 is guanine, or a derivative or analog thereof.

Embodiment II-105. The kit of embodiment II-104, wherein (i) the nucleobase at position +1 of the polynucleotide template is guanine, and (ii) the nucleobase at position +2 of the polynucleotide template is cytosine.

Embodiment II-106. The kit of any of embodiments II-95 to II-97, wherein B′9 is uracil, or a derivative or analog thereof; and B′10 is guanine, or a derivative or analog thereof.

Embodiment II-107. The kit of embodiment II-106, wherein (i) the nucleobase at position +1 of the polynucleotide template is adenine, and (ii) the nucleobase at position +2 of the polynucleotide template is cytosine.

Embodiment II-108. The kit of any of embodiments II-95 to II-97, wherein B′9 is adenine, or a derivative or analog thereof; and B′10 is adenine, or a derivative or analog thereof.

Embodiment II-109. The kit of embodiment II-108, wherein (i) the nucleobase at position +1 of the polynucleotide template is thymine, and (ii) the nucleobase at position +2 of the polynucleotide template is thymine.

Embodiment II-110. The kit of any of embodiments II-95 to II-97, wherein B′9 is adenine, or a derivative or analog thereof; and B′10 is cytosine, or a derivative or analog thereof.

Embodiment II-111. The kit of embodiment II-110, wherein (i) the nucleobase at position +1 of the polynucleotide template is thymine, and (ii) the nucleobase at position +2 of the polynucleotide template is guanine.

Embodiment II-112. The kit of any of embodiments II-95 to II-97, wherein B′9 is adenine, or a derivative or analog thereof; and B′10 is uracil, or a derivative or analog thereof.

Embodiment II-113. The kit of embodiment II-112, wherein (i) the nucleobase at position +1 of the polynucleotide template is thymine, and (ii) the nucleobase at position +2 of the polynucleotide template is adenine.

Embodiment II-114. The kit of any one of embodiments II-95 to II-97, wherein the nucleobase at position −1 of the polynucleotide template is thymine or adenine.

Embodiment II-115. The kit of any of embodiments II-95 to II-114, wherein R′1 are each independently H or C1-C6 alkyl.

Embodiment II-116. The kit of any of embodiments II-95 to II-114, wherein R′ are each H.

Embodiment II-117. The kit of any of embodiments II-95 to II-116, wherein R′2 is H, OH, O—(C1-C6 alkyl), O-MOE, fluoro, or LNA.

Embodiment II-118. The kit of any of embodiments II-95 to II-116, wherein R′2 is OH.

Embodiment II-119. The kit of any of embodiments II-95 to II-118, wherein R′3 is H, OH, O—(C1-C6 alkyl), O-MOE, or fluoro.

Embodiment II-120. The kit of any of embodiments II-95 to II-118, wherein R′3 is OH or OMe.

Embodiment II-121. The kit of any of embodiments II-95 to II-120, wherein R′11 and R′12 are each independently H, OH, O—(C1-C6 alkyl), O-MOE, fluoro, or LNA.

Embodiment II-122. The kit of any of embodiments II-95 to II-120, wherein R′11 is H, OH, OMe, O-MOE, or fluoro; and R′12 is OH or OMe.

Embodiment II-123. The kit of any of embodiments II-95 to II-122, wherein B′8 is complementary to a nucleobase at position −1 of the polynucleotide template.

Embodiment II-124. The kit of any of embodiments II-95 to II-122, wherein B′8 is mismatched to a nucleobase at position −1 of the polynucleotide template.

Embodiment II-125. The kit of any of embodiments II-95 to II-124, wherein the polynucleotide template is a DNA template.

Embodiment II-126. The kit of any of embodiments II-95 to II-124, wherein the polynucleotide template is linear DNA.

Embodiment II-127. The kit of any of embodiments II-95 to II-126, wherein the polynucleotide template comprises a sequence encoding a 5′-untranslated region (UTR), an open-reading frame (ORF), a 3′-UTR, and a polyA sequence.

Embodiment III-1. A capping primer comprising a structure according to Formula II, or a pharmaceutically acceptable salt thereof, wherein, q1 to q7, are each independently 0 or 1; B1 through B7, if present, are each a nucleobase independently selected from the group consisting of adenine, guanine, thymine, uracil, cytosine, hypoxanthine, and any derivative or analog of the foregoing; B8 is a nucleobase selected from the group consisting of adenine, guanine, thymine, uracil, cytosine, hypoxanthine, and any derivative or analog of the foregoing; B9 is a nucleobase selected from the group consisting of adenine, uracil, cytosine, and any derivative or analog of the foregoing; B10 is a nucleobase selected from the group consisting of adenine, guanine, uracil, cytosine, and any derivative or analog of the foregoing; R1 are each independently H, alkyl, acyl, benzyl, or a cleavable unit; R2 is H, OH, alkyl, O-alkyl, O-acyl, O-benzyl, O-methoxyethyl (MOE), halogen, LNA, a linker, a cleavable unit, or a detectable marker; R3 is H, OH, alkyl, O-alkyl, O-acyl, O-benzyl, O-methoxyethyl (MOE), halogen, a linker, a cleavable unit, or a detectable marker; R4 to R12, if present, are each independently H, OH, alkyl, O-alkyl, O-methoxyethyl (MOE), halogen, LNA, a linker, or a detectable marker; X1 to X12, if present, are each independently O or S; Y1 to Y12, if present, are each independently O⁻, S⁻, BH₃⁻, aryl, alkyl, O-alkyl, or O-aryl; and Z1 to Z22, if present, are each independently O, S, NH, CH₂, C(halogen)₂, or CH(halogen); wherein, the capping primer hybridizes to a polynucleotide template, wherein said polynucleotide template comprises a nucleobase at position −1 immediately adjacent to and downstream (3′) of a transcriptional start site, and a nucleobase at each of position +1, position +2 and position +3 immediately adjacent to and upstream (5′) of the transcriptional start site; and wherein, B8 is complementary or mismatched to a nucleobase at position −1 of the polynucleotide template; B9 is complementary to a nucleobase at position +1 of the polynucleotide template; and B10 is complementary to a nucleobase at position +2 of the polynucleotide template; and B8, B9, and B10 are not fully complementary to nucleobases at positions +1 to +3 of the polynucleotide template.

Embodiment III-2. The capping primer of embodiment III-1, wherein the capping primer consists of the structure according to Formula II.

Embodiment III-3. The capping primer of embodiment III-1 or III-2, wherein q1 to q7 are each 0.

Embodiment III-4. The capping primer of embodiment III-1 or III-2, wherein, q1 to q7 are 0; R1 is each independently H, alkyl, acyl, benzyl or a cleavable unit; R2 and R3 are each independently H, OH, alkyl, O-alkyl, O-acyl, O-benzyl, halogen, LNA, a linker, a cleavable unit or a detectable marker; and R11 and R12 are O-alkyl

Embodiment III-5. The capping primer of embodiment III-1 or III-2, wherein at least one of q1 to q7 is 1.

Embodiment III-6. The capping primer of embodiment III-1 or III-2, wherein at least two of q to q7 are 1.

Embodiment III-7. The capping primer of embodiment III-1 or III-2, wherein at least three of q to q7 are 1.

Embodiment III-8. The capping primer of embodiment III-1 or III-2, wherein at least four of q to q7 are 1.

Embodiment III-9. The capping primer of embodiment III-1 or III-2, wherein at least five of q to q7 are 1.

Embodiment III-10. The capping primer of embodiment III-1 or III-2, wherein at least six of q to q7 are 1.

Embodiment III-11. The capping primer of embodiment III-1 or III-2, wherein q1 to q7 are each 1.

Embodiment III-12. The capping primer of any one of embodiments III-1 to III-11, wherein B8 is not N6-modified adenine.

Embodiment III-13. The capping primer of any one of embodiments III-1 to III-11, wherein B8 is a nucleobase selected from the group consisting of adenine, uracil, thymine, cytosine, 5-methylcytosine, guanine, and hypoxanthine.

Embodiment III-14. The capping primer of any of embodiments III-1 to III-13, wherein B9 is selected from the group consisting of adenine, cytosine, 5-methylcytosine, thymine and uracil.

Embodiment III-15. The capping primer of any of embodiments III-1 to III-13, wherein B9 is selected from the group consisting of adenine, cytosine, and uracil.

Embodiment III-16. The capping primer of any of embodiments III-1 to III-13, wherein B9 is adenine or uracil.

Embodiment III-17. The capping primer of any of embodiments III-1 to III-16, wherein B10 is selected from the group consisting of adenine, guanine, cytosine, 5-methylcytosine, thymine and uracil.

Embodiment III-18. The capping primer of any of embodiments III-1 to III-16, wherein B10 is selected from the group consisting of adenine, guanine, cytosine, and uracil.

Embodiment III-19. The capping primer of any one of embodiments III-1 to III-13, wherein B9 is adenine, or a derivative or analog thereof, and B10 is guanine, or a derivative or analog thereof.

Embodiment III-20. The capping primer of any one of embodiments III-1 to III-13, wherein B9 is cytosine, or a derivative or analog thereof; and B10 is guanine, or a derivative or analog thereof.

Embodiment III-21. The capping primer of any one of embodiments III-1 to III-13, wherein B9 is uracil, or a derivative or analog thereof, and B10 is guanine, or a derivative or analog thereof.

Embodiment III-22. The capping primer of any one of embodiments III-1 to III-13, wherein B9 is adenine, or a derivative or analog thereof, and B10 is adenine, or a derivative or analog thereof.

Embodiment III-23. The capping primer of any one of embodiments III-1 to III-13, wherein B9 is adenine, or a derivative or analog thereof, and B10 is cytosine, or a derivative or analog thereof.

Embodiment III-24. The capping primer of any one of embodiments III-1 to III-13, wherein B9 is adenine, or a derivative or analog thereof, and B10 is uracil, or a derivative or analog thereof.

Embodiment III-25. The capping primer of embodiment III-1 or III-2, wherein q1 is 1 and q2 to q7 are each 0.

Embodiment III-26. The capping primer of embodiment III-25, wherein B1 is mismatched to a nucleobase at position −2 of the polynucleotide template.

Embodiment III-27. The capping primer of embodiment III-25, wherein B1 is complimentary to a nucleobase at position −2 of the polynucleotide template.

Embodiment III-28. The capping primer of any of embodiments III-25 to III-27, wherein B1 is selected from the group consisting of adenine, uracil, thymine, cytosine, 5-methylcytosine, guanine, and hypoxanthine.

Embodiment III-29. The capping primer of any of embodiments III-25 to III-27, wherein B1 is adenine, or a derivative or analog thereof.

Embodiment III-30. The capping primer of any of embodiments III-25 to III-27, wherein B1 is uracil, or a derivative or analog thereof.

Embodiment III-31. The capping primer of any of embodiments III-25 to III-27, wherein B1 is guanine, or a derivative or analog thereof.

Embodiment III-32. The capping primer of any of embodiments III-25 to III-27, wherein B1 is cytosine, or a derivative or analog thereof.

Embodiment III-33. The capping primer of any one of embodiments III-25 to III-32, B8 is not N6-modified adenine.

Embodiment III-34. The capping primer of any one of embodiments III-25 to III-32, wherein B8 is a nucleobase selected from the group consisting of adenine, uracil, thymine, cytosine, 5-methylcytosine, guanine, and hypoxanthine.

Embodiment III-35. The capping primer of any of embodiments III-25 to III-34, wherein B9 is selected from the group consisting of adenine, cytosine, 5-methylcytosine, thymine and uracil.

Embodiment III-36. The capping primer of any of embodiments III-25 to III-34, wherein B9 is selected from the group consisting of adenine, cytosine, and uracil.

Embodiment III-37. The capping primer of any of embodiments III-25 to III-34, wherein B9 is adenine or uracil.

Embodiment III-38. The capping primer of any of embodiments III-25 to III-37, wherein B10 is selected from the group consisting of adenine, guanine, cytosine, 5-methylcytosine, thymine and uracil.

Embodiment III-39. The capping primer of any of embodiments III-25 to III-37, wherein B10 is selected from the group consisting of adenine, guanine, cytosine, and uracil.

Embodiment III-40. The capping primer of any one of embodiments III-25 to III-34, wherein B9 is adenine, or a derivative or analog thereof, and B10 is guanine, or a derivative or analog thereof.

Embodiment III-41. The capping primer of any one of embodiments III-25 to III-34, wherein B9 is cytosine, or a derivative or analog thereof; and B10 is guanine, or a derivative or analog thereof.

Embodiment III-42. The capping primer of any one of embodiments III-25 to III-34, wherein B9 is uracil, or a derivative or analog thereof, and B10 is guanine, or a derivative or analog thereof.

Embodiment III-43. The capping primer of any one of embodiments III-25 to III-34, wherein B9 is adenine, or a derivative or analog thereof, and B10 is adenine, or a derivative or analog thereof.

Embodiment III-44. The capping primer of any one of embodiments III-25 to III-34, wherein B9 is adenine, or a derivative or analog thereof, and B10 is cytosine, or a derivative or analog thereof.

Embodiment III-45. The capping primer of any one of embodiments III-25 to III-34, wherein B9 is adenine, or a derivative or analog thereof, and B10 is uracil, or a derivative or analog thereof.

Embodiment III-46. The capping primer of any of embodiments III-1 to III-45, wherein R1 are each independently H or C1-C6 alkyl.

Embodiment III-47. The capping primer of any of embodiments III-1 to III-45, wherein R1 are each H.

Embodiment III-48. The capping primer of any of embodiments III-1 to III-47, wherein R2 is H, OH, O—(C1-C6 alkyl), O-MOE, fluoro, or LNA.

Embodiment III-49. The capping primer of any of embodiments III-1 to III-47, wherein R2 is OH.

Embodiment III-50. The capping primer of any of embodiments III-1 to III-49, wherein R3 is H, OH, O—(C1-C6 alkyl), O-MOE, or fluoro.

Embodiment III-51. The capping primer of any of embodiments III-1 to III-49, wherein R3 is OH or OMe.

Embodiment III-52. The capping primer of any of embodiments III-1 to III-51, wherein R4 to R10, if present, and R11 and R12 are each independently H, OH, O—(C1-C6 alkyl), O-MOE, fluoro, or LNA.

Embodiment III-53. The capping primer of any of embodiments III-1 to III-51, wherein R4 to R10, if present, are each independently H, OH, OMe, O-MOE, fluoro, or LNA, R11 is H, OH, OMe, O-MOE, fluoro, or LNA, and R12 is OH or OMe.

Embodiment III-54. The capping primer of any of embodiments III-1 to III-51, wherein R4 to R10, if present, are each OMe, R11 is OMe, and R12 is OMe.

Embodiment III-55. The capping primer of any of embodiments III-1 to III-54, wherein X1 to X12, if present, are each independently 0; Y1 to Y12, if present, are each independently O⁻ or S⁻; and Z1 to Z22, if present, are each independently O.

Embodiment III-56. The capping primer of any of embodiments III-1 to III-55, wherein B8 is complementary to a nucleobase at position −1 of the polynucleotide template.

Embodiment III-57. The capping primer of any of embodiments III-1 to III-55, wherein B8 is mismatched to a nucleobase at position −1 of the polynucleotide template.

Embodiment III-58. A stereoisomer, tautomer or salt form of the capping primer of any of embodiments III-1 to III-57.

Embodiment III-59. A system comprising a capping primer and a polynucleotide template, wherein the capping primer comprises a structure according to Formula II, or a pharmaceutically acceptable salt thereof, wherein q1 to q7, are each independently 0 or 1; B1 through B7, if present, are each a nucleobase independently selected from the group consisting of adenine, guanine, thymine, uracil, cytosine, hypoxanthine, and any derivative or analog of the foregoing; B8 is a nucleobase selected from the group consisting of adenine, guanine, thymine, uracil, cytosine, hypoxanthine, and any derivative or analog of the foregoing; B9 is a nucleobase selected from the group consisting of adenine, uracil, cytosine, and any derivative or analog of the foregoing; B10 is a nucleobase selected from the group consisting of adenine, guanine, uracil, cytosine, and any derivative or analog of the foregoing; R1 are each independently H, alkyl, acyl, benzyl, or a cleavable unit; R2 is H, OH, alkyl, O-alkyl, O-acyl, O-benzyl, O-MOE, halogen, LNA, a linker, a cleavable unit, or a detectable marker; R3 is H, OH, alkyl, O-alkyl, O-acyl, O-benzyl, O-MOE, halogen, a linker, a cleavable unit, or a detectable marker; R4 to R12, if present, are each independently H, OH, alkyl, O-alkyl, O-MOE, halogen, LNA, a linker, or a detectable marker; X1 to X12, if present, are each independently O or S; Y1 to Y12, if present, are each independently O⁻, S⁻, BH₃⁻, aryl, alkyl, O-alkyl, or O-aryl; and Z1 to Z22, if present, are each independently O, S, NH, CH₂, C(halogen)₂, or CH(halogen); wherein the polynucleotide template comprises a nucleobase at position −1 immediately adjacent to and downstream (3′) of a transcriptional start site, and a nucleobase at each of position +1, position +2 and position +3 immediately adjacent to and upstream (5′) of the transcriptional start site, and wherein B8 is complementary or mismatched to a nucleobase at positions −1 of the polynucleotide template; B9 is complementary to a nucleobase at position +1 of the polynucleotide template; B10 is complementary to a nucleobase at position +2 of the polynucleotide template; and B8, B9, and B10 are not fully complementary to nucleobases at positions +1 to +3 of the polynucleotide template.

Embodiment III-60. The system of embodiment III-59, wherein the capping primer consists of the structure according to Formula II.

Embodiment III-61. The system of embodiment III-59 or III-60, wherein q1 to q7 are each 0.

Embodiment III-62. The system of embodiment III-59 or III-60, wherein, q1 to q7 are O; R1 is each independently H, alkyl, acyl, benzyl or a cleavable unit; R2 and R3 are each independently H, OH, alkyl, O-alkyl, O-acyl, O-benzyl, halogen, LNA, a linker, a cleavable unit or a detectable marker; and R11 and R12 are O-alkyl.

Embodiment III-63. The system of embodiment III-59 or III-60, wherein at least one of q1 to q7 is 1.

Embodiment III-64. The system of embodiment III-59 or III-60, wherein at least two of q to q7 are 1.

Embodiment III-65. The system of embodiment III-59 or III-60, wherein at least three of q to q7 are 1.

Embodiment III-66. The system of embodiment III-59 or III-60, wherein at least four of q to q7 are 1.

Embodiment III-67. The system of embodiment III-59 or III-60, wherein at least five of q to q7 are 1.

Embodiment III-68. The system of embodiment III-59 or III-60, wherein at least six of q1 to q7 are 1.

Embodiment III-69. The system of embodiment III-59 or III-60, wherein q1 to q7 are each 1.

Embodiment III-70. The system of any of embodiments III-59 to III-69, wherein B8 is not N6-modified adenine.

Embodiment III-71. The system of any of embodiments III-59 to III-70, wherein B8 is a nucleobase selected from the group consisting of adenine, uracil, thymine, cytosine, 5-methylcytosine, guanine, and hypoxanthine.

Embodiment III-72. The system of any of embodiments III-59 to III-71, wherein B9 is selected from the group consisting of adenine, cytosine, 5-methylcytosine, thymine and uracil.

Embodiment III-73. The system of any of embodiments III-59 to III-71, wherein B9 is selected from the group consisting of adenine, cytosine and uracil.

Embodiment III-74. The system of any of embodiments III-59 to III-71, wherein B9 is adenine or uracil.

Embodiment III-75. The system of any of embodiments III-59 to III-74, wherein B10 is selected from the group consisting of adenine, guanine, cytosine, 5-methylcytosine, thymine and uracil.

Embodiment III-76. The system of any of embodiments III-59 to III-74, wherein B10 is selected from the group consisting of adenine, guanine, cytosine and uracil.

Embodiment III-77. The system of any of embodiments III-59 to III-71, wherein B9 is adenine, or a derivative or analog thereof; and B10 is guanine, or a derivative or analog thereof.

Embodiment III-78. The system of embodiment III-77, wherein (i) the nucleobase at position +1 of the polynucleotide template is thymine, and (ii) the nucleobase at position +2 of the polynucleotide template is cytosine.

Embodiment III-79. The system of any of embodiments III-59 to III-71, wherein B9 is cytosine, or a derivative or analog thereof; and B10 is guanine, or a derivative or analog thereof.

Embodiment III-80. The system of embodiment III-79, wherein (i) the nucleobase at position +1 of the polynucleotide template is guanine, and (ii) the nucleobase at position +2 of the polynucleotide template is cytosine.

Embodiment III-81. The system of any of embodiments III-59 to III-71, wherein B9 is uracil, or a derivative or analog thereof, and B10 is guanine, or a derivative or analog thereof.

Embodiment III-82. The system of embodiment III-81, wherein (i) the nucleobase at position +1 of the polynucleotide template is adenine, and (ii) the nucleobase at position +2 of the polynucleotide template is cytosine.

Embodiment III-83. The system of any of embodiments III-59 to III-71, wherein B9 is adenine, or a derivative or analog thereof; and B10 is adenine, or a derivative or analog thereof.

Embodiment III-84. The system of embodiment III-83, wherein (i) the nucleobase at position +1 of the polynucleotide template is thymine, and (ii) the nucleobase at position +2 of the polynucleotide template is thymine.

Embodiment III-85. The system of any of embodiments III-59 to III-71, wherein B9 is adenine, or a derivative or analog thereof; and B10 is cytosine, or a derivative or analog thereof.

Embodiment III-86. The system of embodiment III-85, wherein (i) the nucleobase at position +1 of the polynucleotide template is thymine, and (ii) the nucleobase at position +2 of the polynucleotide template is guanine.

Embodiment III-87. The system of any of embodiments III-59 to III-71, wherein B9 is adenine, or a derivative or analog thereof; and B10 is uracil, or a derivative or analog thereof.

Embodiment III-88. The system of embodiment III-87, wherein (i) the nucleobase at position +1 of the polynucleotide template is thymine, and (ii) the nucleobase at position +2 of the polynucleotide template is adenine.

Embodiment III-89. The system of any of embodiments III-59 to III-88, wherein the nucleobase at position −1 of the polynucleotide template is thymine or adenine.

Embodiment III-90. The system of embodiment III-59 or III-60, wherein q1 is 1 and q2 to q7 are each 0.

Embodiment III-91. The system of embodiment III-90, wherein B1 is mismatched to a nucleobase at position −2 of the polynucleotide template.

Embodiment III-92. The system of embodiment III-90, wherein B1 is complimentary to a nucleobase at position −2 of the polynucleotide template.

Embodiment III-93. The system of any one of embodiments III-90 to III-92, wherein B1 is selected from the group consisting of adenine, uracil, thymine, cytosine, 5-methylcytosine, guanine, and hypoxanthine.

Embodiment III-94. The system of any of embodiments III-90 to III-92, wherein B1 is adenine, or a derivative or analog thereof.

Embodiment III-95. The system of any of embodiments III-90 to III-92, wherein B1 is uracil, or a derivative or analog thereof.

Embodiment III-96. The system of any of embodiments III-90 to III-92, wherein B1 is guanine, or a derivative or analog thereof.

Embodiment III-97. The system of any of embodiments III-90 to III-92, wherein B1 is cytosine, or a derivative or analog thereof.

Embodiment III-98. The system of any one of embodiments III-90 to III-97, B8 is not N6-modified adenine.

Embodiment III-99. The system of any one of embodiments III-90 to III-98, wherein B8 is a nucleobase selected from the group consisting of adenine, uracil, thymine, cytosine, 5-methylcytosine, guanine, and hypoxanthine.

Embodiment III-100. The system of any of embodiments III-90 to III-99, wherein B9 is selected from the group consisting of adenine, cytosine, 5-methylcytosine, thymine and uracil.

Embodiment III-101. The system of any of embodiments III-90 to III-99, wherein B9 is selected from the group consisting of adenine, cytosine, and uracil.

Embodiment III-102. The system of any of embodiments III-90 to III-99, wherein B9 is adenine or uracil.

Embodiment III-103. The system of any of embodiments III-90 to III-102, wherein B10 is selected from the group consisting of adenine, guanine, cytosine, 5-methylcytosine, thymine and uracil.

Embodiment III-104. The system of any of embodiments III-90 to III-102, wherein B10 is selected from the group consisting of adenine, guanine, cytosine, and uracil.

Embodiment III-105. The system of any one of embodiments III-90 to III-99, wherein B9 is adenine, or a derivative or analog thereof, and B10 is guanine, or a derivative or analog thereof.

Embodiment III-106. The system of embodiment III-105, wherein (i) the nucleobase at position +1 of the polynucleotide template is thymine, and (ii) the nucleobase at position +2 of the polynucleotide template is thymine.

Embodiment III-107. The system of any one of embodiments III-90 to III-99, wherein B9 is cytosine, or a derivative or analog thereof, and B10 is guanine, or a derivative or analog thereof.

Embodiment III-108. The system of embodiment III-107, wherein (i) the nucleobase at position +1 of the polynucleotide template is guanine, and (ii) the nucleobase at position +2 of the polynucleotide template is cytosine.

Embodiment III-109. The system of any one of embodiments III-90 to III-99, wherein B9 is uracil, or a derivative or analog thereof; and B10 is guanine, or a derivative or analog thereof.

Embodiment III-110. The system of embodiment III-109, wherein (i) the nucleobase at position +1 of the polynucleotide template is adenine, and (ii) the nucleobase at position +2 of the polynucleotide template is cytosine.

Embodiment III-111. The system of any one of embodiments III-90 to III-99, wherein B9 is adenine, or a derivative or analog thereof, and B10 is adenine, or a derivative or analog thereof.

Embodiment III-112. The system of embodiment III-111, wherein (i) the nucleobase at position +1 of the polynucleotide template is thymine, and (ii) the nucleobase at position +2 of the polynucleotide template is thymine.

Embodiment III-113. The system of any one of embodiments III-90 to III-99, wherein B9 is adenine, or a derivative or analog thereof, and B10 is cytosine, or a derivative or analog thereof.

Embodiment III-114. The system of embodiment III-113, wherein (i) the nucleobase at position +1 of the polynucleotide template is thymine, and (ii) the nucleobase at position +2 of the polynucleotide template is guanine.

Embodiment III-115. The system of any one of embodiments III-90 to III-99, wherein B9 is adenine, or a derivative or analog thereof, and B10 is uracil, or a derivative or analog thereof.

Embodiment III-116. The system of embodiment III-115, wherein (i) the nucleobase at position +1 of the polynucleotide template is thymine, and (ii) the nucleobase at position +2 of the polynucleotide template is adenine.

Embodiment III-117. The system of any of embodiments III-90 to III-116, wherein the nucleobase at position −1 of the polynucleotide template is thymine or adenine.

Embodiment III-118. The system of any of embodiments III-59 to III-117, wherein R1 are each independently H or C1-C6 alkyl.

Embodiment III-119. The system of any of embodiments III-59 to III-117, wherein R1 are each H.

Embodiment III-120. The system of any of embodiments III-59 to III-119, wherein R2 is H, OH, O—(C1-C6 alkyl), O-MOE, fluoro, or LNA.

Embodiment III-121. The system of any one of embodiments III-59 to III-119, wherein R2 is OH.

Embodiment III-122. The system of any of embodiments III-59 to III-121, wherein R3 is H, OH, O—(C1-C6 alkyl), O-MOE, or fluoro.

Embodiment III-123. The system of any of embodiments III-59 to III-121, wherein R3 is OH or OMe.

Embodiment III-124. The system of any of embodiments III-59 to III-123, wherein R4 to R10, if present, and R11 and R12 are each independently H, OH, O—(C1-C6 alkyl), O-MOE, fluoro, or LNA.

Embodiment III-125. The system of any of embodiments III-59 to III-123, wherein R4 to R10, if present, are each independently H, OH, OMe, O-MOE, fluoro, R11 is H, OH, OMe, O-MOE, fluoro, or LNA and R12 is OH or OMe.

Embodiment III-126. The system of any of embodiments III-59 to III-123, wherein R4 to R10, if present, are each OMe, R11 is OMe, and R12 is OMe.

Embodiment III-127. The system of any of embodiments III-59 to III-126, wherein B8 is complementary to a nucleobase at position −1 of the polynucleotide template.

Embodiment III-128. The system of any of embodiments III-59 to III-126, wherein B8 is mismatched to a nucleobase at position −1 of the polynucleotide template.

Embodiment III-129. The system of any of embodiments III-59- to III-128, wherein the polynucleotide template is a DNA template.

Embodiment III-130. The system of any of embodiments III-59 to III-129, wherein the polynucleotide template is linear DNA.

Embodiment III-131. The system of any of embodiments III-59 to III-130, wherein the polynucleotide template comprises a sequence encoding a 5′-untranslated region (UTR), an open-reading frame (ORF), a 3′-UTR, and a polyA sequence.

Embodiment III-132. A method of producing 5′-capped RNA molecules in an in vitro transcription (IVT) reaction comprising the steps of: mixing the capping primer of any of embodiments III-1 to III-57, or the stereoisomer, tautomer, or salt form of embodiment III-58, with the polynucleotide template, nucleoside 5′-triphosphates (NTPs), and a RNA polymerase; and incubating the mixture under transcription conditions, thereby resulting in synthesis of a 5′-capped RNA molecule.

Embodiment III-133. A method of producing a 5′-capped RNA molecule in an in vitro transcription (IVT) reaction comprising the steps of: mixing the system of any of embodiments III-59 to III-131 with nucleoside 5′-triphosphates (NTPs), and an RNA polymerase; and incubating the mixture under transcription conditions, thereby resulting in synthesis of a 5′-capped RNA molecule.

Embodiment III-134. The method of embodiment III-132 or III-133, wherein the RNA polymerase is a T7 RNA polymerase or a variant thereof, a T3 RNA polymerase or a variant thereof, or a SP6 RNA polymerase or a variant thereof.

Embodiment III-135. The method of embodiment III-132 or III-133, wherein the RNA polymerase is a T7 RNA polymerase or a variant thereof.

Embodiment III-136. The method of any of embodiments III-132 to III-135, wherein the capping primer and the NTPs are present in the IVT reaction at approximately equimolar concentrations.

Embodiment III-137. The method of any of embodiments III-132 to III-136, wherein the NTPs comprise adenosine triphosphate (ATP) or a derivative or analog thereof, guanosine triphosphate (GTP) or a derivative or analog thereof, cytosine triphosphate (CTP) or a derivative or analog thereof, and uridine triphosphate (UTP) or a derivative or analog thereof, each at an approximately equimolar concentration, optionally wherein the derivative or analog of UTP is pseudouridine triphosphate (pseudo-UTP) or N1-methylpseudouridine triphosphate (N1-pseudo-UTP).

Embodiment III-138. The method of any of embodiments III-132 to III-137, wherein the NTPs comprise ATP, GTP, CTP, and UTP at an approximately equimolar concentration, optionally wherein UTP is pseudo-UTP or N1-pseudo-UTP, and wherein (i) each NTP is present in the IVT reaction at a molar concentration that is about 1.1-fold, 1.2-fold, 1.3-fold, 1.4-fold, 1.5-fold, 2-fold, 2.5-fold, 3-fold, 4-fold, 5-fold, or 10-fold higher than a molar concentration of the capping primer, or (ii) the capping primer is present in the IVT reaction at a molar concentration that is about 1.1-fold, 1.2-fold, 1.3-fold, 1.4-fold, 1.5-fold, 1.6-fold, 1.7-fold, 1.8-fold, 1.9-fold, 2-fold, 2.5-fold, 3-fold, 4-fold, or 5-fold higher than a molar concentration of each NTP.

Embodiment III-139. The method of any of embodiments III-132 to III-137, wherein at least about 65%, about 70%, about 75%, about 80%, about 85%, about 90%, about 91%, about 92%, about 93%, about 94%, about 95%, about 96%, about 97%, about 98%, or about 99% of the RNA molecules produced by the IVT reaction are 5′-capped.

Embodiment III-140. The method of any of embodiments III-132 to III-137, wherein the synthesis results in a yield of RNA molecules that is at least about 60%, about 70%, about 75%, about 80%, about 85%, about 90%, about 91%, about 92%, about 93%, about 94%, about 95%, about 96%, about 97%, about 98%, or about 99% relative to the RNA yield obtained in the reaction without using a primer.

Embodiment III-141. The method of any of embodiments III-132 to III-137, wherein the synthesis results in a yield of RNA molecules that is at least about 1.1-fold, 1.5-fold, 2-fold, 3-fold, 4-fold, 5-fold, 6-fold, 7-fold, 8-fold, 9-fold, or 10-fold higher relative to the RNA yield obtained in the reaction without using a primer.

Embodiment III-142. The method of any of embodiments III-132 to III-137, wherein the synthesis results in (a) a yield of RNA molecules that is at least about 30% relative to the RNA yield obtained in the reaction without using a primer; and (b) at least about 30% of the RNA molecules produced are 5′-capped.

Embodiment III-143. The method of any of embodiments III-132 to III-137, wherein the synthesis results in (a) a yield of RNA molecules that is at least about 50% relative to the RNA yield obtained in the reaction without using a primer; and (b) at least about 50% of the RNA molecules produced are 5′-capped.

Embodiment III-144. The method of any of embodiments III-132 to III-137, wherein the synthesis results in (a) a yield of RNA molecules that is at least about 70% relative to the RNA yield obtained in the reaction without using a primer; and (b) at least about 70% of the RNA molecules produced are 5′-capped.

Embodiment III-145. The method of any of embodiments III-132 to III-144, wherein the method further comprises a step of purifying the 5′-capped RNA molecules.

Embodiment III-146. 5′-capped RNA molecules produced by the method of any of embodiments III-132 to III-145.

Embodiment III-147. The 5′-capped RNA molecules of embodiment III-146, comprising 5′-capped messenger RNAs (mRNAs).

Embodiment III-148. A cell comprising the 5′-capped RNA molecules of embodiment III-146 or III-147.

Embodiment III-149. A pharmaceutical composition comprising the 5′-capped RNA molecules of embodiment III-146 or III-147.

Embodiment III-150. The pharmaceutical composition comprising the 5′-capped RNA molecules of embodiment III-149, wherein the pharmaceutical composition is formulated as a lipid nanoparticle (LNP).

Embodiment III-151. An LNP comprising the 5′-capped RNA molecule of embodiment III-146 or III-147.

Embodiment III-152. A kit comprising the capping primer of any of embodiments III-1 to III-57, the stereoisomer, tautomer, or salt form of embodiment III-58, or the system of any of embodiments III-59 to III-131, and instructions for use in an IVT reaction to synthesize 5′-capped RNA molecules.

Embodiment III-153. A kit for producing a 5′-capped RNA molecule, comprising: (a) a capping primer comprising a structure according to Formula IV, or a pharmaceutically acceptable salt thereof, wherein, q′1 is 0 or 1; B′1, if present, is a nucleobase independently selected from the group consisting of adenine, guanine, thymine, uracil, cytosine, and hypoxanthine, and any derivative or analog of the foregoing; B′8 is a nucleobase selected from the group consisting of adenine, guanine, thymine, uracil, cytosine, and hypoxanthine and any derivative or analog of the foregoing; B′9 is a nucleobase selected from the group consisting of adenine, uracil, cytosine, and any derivative or analog of the foregoing; B′10 is a nucleobase selected from the group consisting of adenine, guanine, uracil, cytosine, and any derivative or analog of the foregoing; R′1 are each independently H, alkyl, acyl, benzyl, or a cleavable unit; R′2 is H, OH, alkyl, O-alkyl, O-acyl, O-benzyl, O-MOE, halogen, LNA, a linker, a cleavable unit, or a detectable marker; R′3 is H, OH, alkyl, O-alkyl, O-acyl, O-benzyl, O-MOE, halogen, a linker, a cleavable unit, or a detectable marker; R′4, if present, R′11 and R′12 are each independently H, OH, alkyl, O-alkyl, O-MOE, halogen, LNA, a linker, or a detectable marker; X′1 to X′2, X′3, if present, and X′10 to X′12 are each independently O or S; Y′1 to Y′2, Y′3, if present, and Y′10 to Y′12 are each independently O⁻, S⁻, BH₃⁻, aryl, alkyl, O-alkyl, or O-aryl; and Z′1 to Z′3, Z′4 if present, Z′17, if present, and Z′18 to Z′21 are each independently O or S, NH, CH₂, C(halogen)₂ or CH(halogen); and (b) instructions for use of the capping primer in an IVT reaction with a polynucleotide template to synthesize 5′-capped RNA molecules, wherein: the polynucleotide template comprises a nucleobase at position −2 and position −1 immediately adjacent to and downstream (3′) of a transcriptional start site, and a nucleobase at each of position +1, position +2 and position +3 immediately adjacent to and upstream (5′) of the transcriptional start site; B′1, if present, is complementary or mismatched to a nucleobase at position −2 of the polynucleotide template; B′8 is complementary or mismatched to a nucleobase at position −1 of the polynucleotide template; B′9 is complementary to a nucleobase at position +1 of the polynucleotide template; B′10 is complementary to a nucleobase at position +2 of the polynucleotide template; and B′8, B′9, and B′10 are not fully complementary to nucleobases at positions +1 to +3 of the polynucleotide template.

Embodiment III-154. The kit of embodiment III-153, wherein the capping primer consists of the structure according to Formula IV.

Embodiment III-155. The kit of embodiment III-153 or III-154, wherein q′1 is 0.

Embodiment III-156. The kit of embodiment III-153 or III-154, wherein, q′1 is 0; R1 is each independently H, alkyl, acyl, benzyl or a cleavable unit; R2 and R3 are each independently H, OH, alkyl, O-alkyl, O-acyl, O-benzyl, halogen, LNA, a linker, a cleavable unit or a detectable marker; and R11 and R12 are O-alkyl.

Embodiment III-157. The kit of embodiment III-153 or III-154, wherein q′1 is 1.

Embodiment III-158. The kit of embodiment III-157, wherein B′1 is mismatched to the nucleobase at position −2 of the polynucleotide template.

Embodiment III-159. The kit of embodiment III-157, wherein B′1 is complimentary to the nucleobase at position −2 of the polynucleotide template.

Embodiment III-160. The kit of any one of embodiments III-157 to III-159, wherein B′1 is selected from the group consisting of adenine, uracil, thymine, cytosine, 5-methylcytosine, guanine, and hypoxanthine.

Embodiment III-161. The kit of any one of embodiments III-157 to III-159, wherein B′1 is adenine, or a derivative or analog thereof.

Embodiment III-162. The kit of any one of embodiments III-157 to III-159, wherein B′1 is uracil, or a derivative or analog thereof.

Embodiment III-163. The kit of any one of embodiments III-157 to III-159, wherein B′1 is guanine, or a derivative or analog thereof.

Embodiment III-164. The kit of any one of embodiments III-157 to III-159, wherein B′1 is cytosine, or a derivative or analog thereof.

Embodiment III-165. The kit of any one of embodiments III-153 to III-164, wherein B′8 is not N6-modified adenine.

Embodiment III-166. The kit of embodiment III-153 to III-164, wherein B′8 is a nucleobase selected from the group consisting of adenine, uracil, thymine, cytosine, 5-methylcytosine, guanine, and hypoxanthine.

Embodiment III-167. The kit of any of embodiments III-153 to III-166, wherein B′9 is selected from the group consisting of adenine, cytosine, 5-methylcytosine, thymine and uracil.

Embodiment III-168. The kit of any of embodiments III-153 to III-166, wherein B′9 is selected from the group consisting of adenine, cytosine and uracil.

Embodiment III-169. The kit of any of embodiments III-153 to III-166, wherein B′9 is adenine or uracil.

Embodiment III-170. The kit of any of embodiments III-153 to III-169, wherein B′10 is selected from the group consisting of adenine, guanine, cytosine, 5-methylcytosine, thymine and uracil.

Embodiment III-171. The kit of any of embodiments III-153 to III-169, wherein B′10 is selected from the group consisting of adenine, guanine, cytosine and uracil.

Embodiment III-172. The kit of any one of embodiments III-153 to III-166, wherein B′9 is adenine, or a derivative or analog thereof, and B10 is guanine, or a derivative or analog thereof.

Embodiment III-173. The kit of embodiment III-172, wherein (i) the nucleobase at position +1 of the polynucleotide template is thymine, and (ii) the nucleobase at position +2 of the polynucleotide template is cytosine.

Embodiment III-174. The kit of any of embodiments III-153 to III-166, wherein B′9 is cytosine, or a derivative or analog thereof; and B′10 is guanine, or a derivative or analog thereof.

Embodiment III-175. The kit of embodiment III-174, wherein (i) the nucleobase at position +1 of the polynucleotide template is guanine, and (ii) the nucleobase at position +2 of the polynucleotide template is cytosine.

Embodiment III-176. The kit of any of embodiments III-153 to III-166, wherein B′9 is uracil, or a derivative or analog thereof, and B′10 is guanine, or a derivative or analog thereof.

Embodiment III-177. The kit of embodiment III-176, wherein (i) the nucleobase at position +1 of the polynucleotide template is adenine, and (ii) the nucleobase at position +2 of the polynucleotide template is cytosine.

Embodiment III-178. The kit of any of embodiments III-153 to III-166, wherein B′9 is adenine, or a derivative or analog thereof; and B′10 is adenine, or a derivative or analog thereof.

Embodiment III-179. The kit of embodiment III-178, wherein (i) the nucleobase at position +1 of the polynucleotide template is thymine, and (ii) the nucleobase at position +2 of the polynucleotide template is thymine.

Embodiment III-180. The kit of any of embodiments III-153 to III-166, wherein B′9 is adenine, or a derivative or analog thereof; and B′10 is cytosine, or a derivative or analog thereof.

Embodiment III-181. The kit of embodiment III-180, wherein (i) the nucleobase at position +1 of the polynucleotide template is thymine, and (ii) the nucleobase at position +2 of the polynucleotide template is guanine.

Embodiment III-182. The kit of any of embodiments III-153 to III-166, wherein B′9 is adenine, or a derivative or analog thereof; and B′10 is uracil, or a derivative or analog thereof.

Embodiment III-183. The kit of embodiment III-182, wherein (i) the nucleobase at position +1 of the polynucleotide template is thymine, and (ii) the nucleobase at position +2 of the polynucleotide template is adenine.

Embodiment III-184. The kit of any one of embodiments III-153 to III-183, wherein the nucleobase at position −1 of the polynucleotide template is thymine or adenine.

Embodiment III-185. The kit of any of embodiments III-153 to III-184, wherein R′1 are each independently H or C1-C6 alkyl.

Embodiment III-186. The kit of any of embodiments III-153 to III-184, wherein R′1 are each H.

Embodiment III-187. The kit of any of embodiments III-153 to III-186, wherein R′2 is H, OH, O—(C1-C6 alkyl), O-MOE, fluoro, or LNA.

Embodiment III-188. The kit of any of embodiments III-153 to III-186, wherein R′2 is OH.

Embodiment III-189. The kit of any of embodiments III-153 to III-188, wherein R′3 is H, OH, O—(C1-C6 alkyl), O-MOE, or fluoro.

Embodiment III-190. The kit of any of embodiments III-153 to III-188, wherein R′3 is OH or OMe.

Embodiment III-191. The kit of any of embodiments III-153 to III-190, wherein R′4, if present, and R′11 and R′12 are each independently H, OH, O—(C1-C6 alkyl), O-MOE, fluoro, or LNA.

Embodiment III-192. The kit of any of embodiments III-153 to III-190, wherein R′4, if present, is H, OH, OMe, O-MOE, or fluoro; R′11 is H, OH, OMe, O-MOE, or fluoro; and R′12 is OH or OMe.

Embodiment III-193. The kit of any of embodiments III-153 to III-190, wherein R′4, if present, is OMe; R′11 is OMe; and R′12 is OMe.

Embodiment III-194. The kit of any of embodiments III-153 to III-193, wherein B′8 is complementary to a nucleobase at position −1 of the polynucleotide template.

Embodiment III-195. The kit of any of embodiments III-153 to III-193, wherein B′8 is mismatched to a nucleobase at position −1 of the polynucleotide template.

Embodiment III-196. The kit of any of embodiments III-153 to III-195, wherein the polynucleotide template is a DNA template.

Embodiment III-197. The kit of any of embodiments III-153 to III-195, wherein the polynucleotide template is linear DNA.

Embodiment III-198. The kit of any of embodiments III-153 to III-197, wherein the polynucleotide template comprises a sequence encoding a 5′-untranslated region (UTR), an open-reading frame (ORF), a 3′-UTR, and a polyA sequence.

### EXAMPLES

### Example 1: Synthesis of 5′-Capping Primers

This Examples describes exemplary tetranucleotide 5′-capping primers having a Cap-2 structure. The capping primers were designed to anneal to positions −1 to +2 of a DNA template having a thymine nucleobase at position +1 and a cytosine nucleobase at position +2 (“TC template”).

### General Synthesis of Trinucleotide 5′-Monophosphates (Procedure 1)

The trinucleotide 5′-monophosphates were synthesized by solid-phase synthesis following the phosphoramidite method. Each sequence was synthesized from 3′ to 5′ by incorporating 2′-O-methyl (2′-OMe) phosphoramidite. Primer Support 5G ribo G 300 (100 μmol) was used as a solid-support, and the 5′-phosphorylation was performed by Bis(2-cyanoethyl)-N,N-diisopropyl phosphoramidite. After deprotecting the trinucleotide 5′-monophosphates according to standard procedures, they were purified as triethylammonium (TEA) salt by anion-exchange chromatography on DEAE-Sephadex (linear gradient of 0.05-0.8 M TEAB over 50 min) or reverse-phased chromatography on ODS column (Yamazen Flush ODS Premium M size, Buffer A: 0.1 M TEAA, Buffer B: ACN, linear gradient 1%-20% of Buffer B over 20 min). The structures of the synthesized trinucleotide 5′-monophosphate were confirmed by ESI-MS system (Waters Xevo G2-XS QTof) and summarized in Table 2.

TABLE 2
Characterization of exemplary trinucleotide
5′-monophosphates produced by Procedure 1

| | Calculated. | Observed |
|---|---|---|
| Compound | MS [M − H]⁻ | MS [M − H]⁻ |
| PA_{2′OMe}pA_{2′OMe}pG | 1048.1871 | 1048.1858 |
| pU_{2′OMe}PA_{2′OMe}pG | 1025.1598 | 1025.1603 |

### General Synthesis of Trinucleotide 5′-Monophosphates (Procedure 2)

The trinucleotide 5′-monophosphates were synthesized by solid-phase synthesis following the phosphoramidite method. Each sequence was synthesized from 3′ to 5′ by incorporating 2′-O-methyl (2′-OMe) phosphoramidite or 2′-deoxy phosphoramidite. Primer Support 5G ribo G 300 (100 μmol), Primer Support 5G ribo A 300 (100 μmol), Primer Support 5G ribo C 300 (100 μmol) and Primer Support 5G ribo U 300 (100 μmol) were used as a solid-support, and the 5′-phosphorylation was performed by Bis(2-cyanoethyl)-N,N-diisopropyl phosphoramidite. After deprotecting the trinucleotide 5′-monophosphates according to standard procedures, they were purified as triethylammonium (TEA) salt by reverse-phased chromatography on ODS column (Yamazen Flush ODS Premium M size, Buffer A: 0.1 M TEAB, Buffer B: ACN, isocratic 1% of Buffer B for 5 min then linear gradient 1%-20% of Buffer B over 20 min). The structures of the synthesized trinucleotide 5′-monophosphate were confirmed by ESI-MS system (Thermo Fisher SCIENTIFIC LTQ Orbitrap™ XL) and summarized in Table 3.

TABLE 3
Characterization of exemplary trinucleotide
5′-monophosphates produced by Procedure 2

| | Calculated | Observed |
|---|---|---|
| Compound | MS [M-H]⁻ | MS [M-H]⁻ |
| pA_{2′OMe}pC_{2′OMe}pG | 1024.1758 | 1024.1755 |
| pA_{2′OMe}pU_{2′OMe}pG | 1025.1598 | 1025.1606 |
| pA_{2′OMe}pA_{2′OMe}pA | 1032.1921 | 1032.1938 |
| pA_{2′OMe}pA_{2′OMe}pC | 1008.1809 | 1008.1829 |
| pA_{2′OMe}pA_{2′OMe}pU | 1009.1649 | 1009.1668 |
| pI_{2′OMe}pA_{2′OMe}pG | 1049.1711 | 1049.1721 |
| pdTpA_{2′OMe}pG | 1009.1649 | 1009.1672 |

### General Synthesis of Trinucleotide 5′-Monophosphates (Procedure 3)

The trinucleotide 5′-monophosphates were synthesized by solid-phase synthesis following the phosphoramidite method. Each sequence was synthesized from 3′ to 5′ by incorporating 2′-O-methyl (2′-OMe) phosphoramidite, 2′-fluoro (2′-F) phosphoramidite, 2′-O-triisopropylsilyloxymethyl (2′-TOM) phosphoramidite or 2′-O-methoxyethyl (2′-MOE) phosphoramidite. Primer Support 5G ribo G 300 (100 μmol) was used as a solid-support, and the 5′-phosphorylation was performed by Bis(2-cyanoethyl)-N,N-diisopropyl phosphoramidite. After deprotecting the trinucleotide 5′-monophosphates according to standard procedures, they were purified as triethylammonium (TEA) salt by reverse-phased chromatography on ODS column (Yamazen Flush ODS Premium M size, Buffer A: 0.1 M TEAB, Buffer B: ACN, isocratic 1% of Buffer B for 5 min then linear gradient 1%-20% of Buffer B over 20 min). The structures of the synthesized trinucleotide 5′-monophosphate were confirmed by ESI-MS system (Waters Xevo G2-XS QTof) and summarized in Table 4 and 5.

TABLE 4
Characterization of exemplary trinucleotide
5′-monophosphates produced by Procedure 3

| | Calculated | Observed |
|---|---|---|
| Compound | MS [M − H]⁻ | MS [M − H]⁻ |
| pC_{2′OMe}pA_{2′OMe}pG | 1024.1758 | 1024.1781 |
| pG_{2′OMe}pA_{2′OMe}pG | 1064.1820 | 1064.1826 |
| p^{m5}C_{2′OMe}pA_{2′OMe}pG | 1038.1915 | 1038.1904 |
| p^{m5}U_{2′OMe}pA_{2′OMe}pG | 1039.1755 | 1039.1733 |
| pA_{2′F}pA_{2′OMe}pG | 1036.1671 | 1036.1603 |
| prApA_{2′OMe}pG | 1034.1714 | 1034.1715 |
| pA_{2′MOE}pA_{2′OMe}pG | 1092.2133 | 1092.2136 |
| p^{m6}A_{2′OMe}pA_{2′OMe}pG | 1062.2027 | 1062.1938 |
| pA_{2′OMe}pA_{2′OMe}pA_{2′OMe}pG | 1391.2552 | 1391.2515 |

TABLE 5
Characterization of additional exemplary trinucleotide
5′-monophosphates produced by Procedure 2

| | Calculated | Observed |
|---|---|---|
| Compound | MS [M − H]⁻ | MS [M − H]⁻ |
| pGₗₙₐₚA_{2′OMe}pG | 1062.1663 | 1061.9755 |
| pG_{2′OMe}psA_{2′OMe}psG | 1096.1335 | 1096.1363 |
| pG_{2′OMe}pA_{2′OMe}pA_{2′OMe}pG | 1407.2501 | 1407.2449 |
| pU_{2′OMe}pA_{2′OMe}pA_{2′OMe}pG | 1368.2280 | 1368.2312 |

Synthesis of ^{m7}GpppA_{2′OMe}pA_{2′OMe}pG

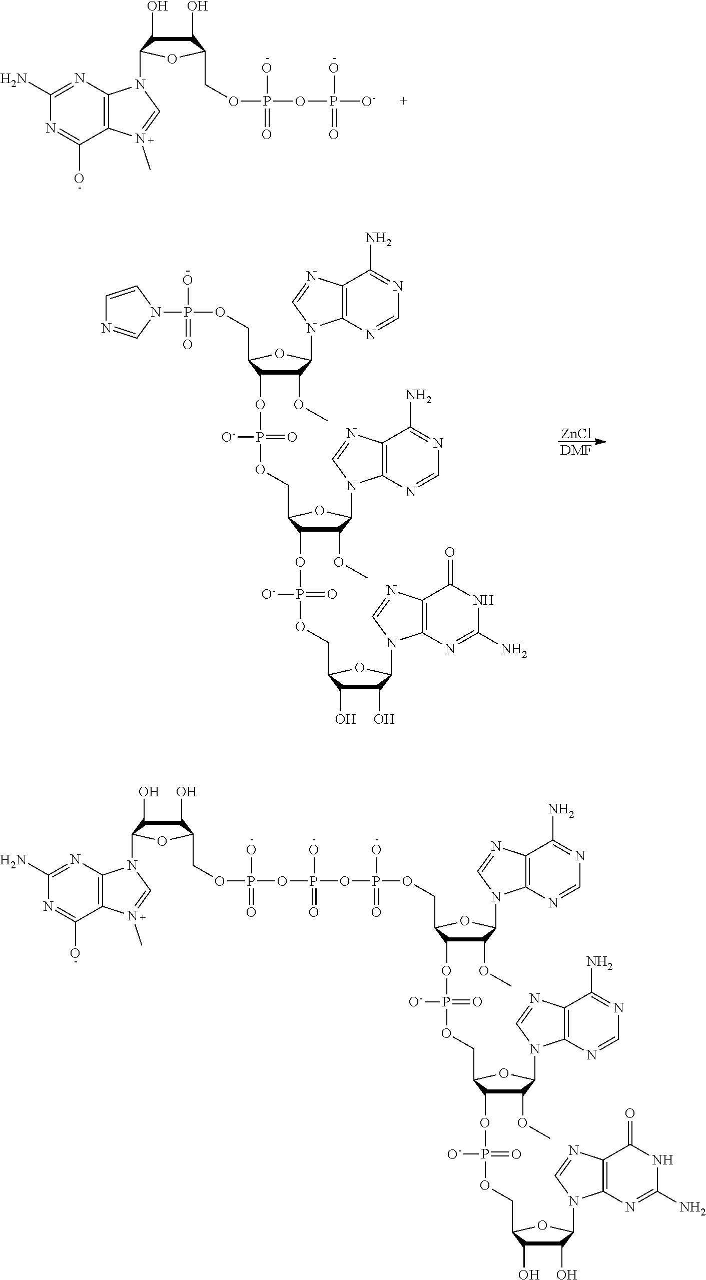

To a solution of pA_{2′OMe}pA_{2′OMe}pG (42 mg, 29 μmol) in DMF (1 mL), imidazole (31.5 mg, 462 μmol), 2,2′-dithiodipyridine (38.2 mg, 173 μmol), TEA (24 μL, 173 μmol) and triphenylphosphine (45.4 mg, 173 μmol) was added and the mixture was stirred at room temperature for 1 hour. The phosphorimidazolide of pA_{2′OMe}pA_{2′OMe}pG was precipitated by addition of sodium perchlorate (35.5 mg, 290 μmol) in acetone (10 mL). The solution was centrifuged at 4° C. washed with chilled acetone 3 times and dried under reduced pressure to give pA_{2′OMe}pA_{2′OMe}pG-Im. pA_{2′OMe}pA_{2′OMe}pG-Im was then dissolved in DMF (0.5 mL), and ^{m7}GDP·TEA salt (23.4 mg, 31 mol) and ZnCl₂ (4.2 mg, 31 μmol) were added. The mixture was stirred at room temperature for 12 hours, and the reaction was quenched by addition of 0.5 M EDTA aq. (0.6 mL) and 0.1 M NaHCO₃ aq. (10 mL). The tetranucleotide product was purified by anion-exchanging chromatography on DEAE Sephadex (linear gradient of 0.05-0.8 M TEAB over 50 min) and RP-HPLC (Biotage Sfür C18, 6 g, Buffer A: 0.1% ammonium formate buffer, Buffer B: ACN, linear gradient 1%-12% of Buffer B over 25 min). The isolated product was concentrated by evaporator and precipitated by sodium perchlorate (10 mg) in acetone (10 mL). The solution was centrifuged at 4° C. washed with chilled acetone 3 times and dried under reduced pressure to obtain ^{m7}GpppA_{2′OMe}pA_{2′OMe}pG·Na salt (8 mg, 5 μmol, 24% yield).

¹H NMR (700 MHz, D₂O) δ 3.46 (s, 3H), 3.58 (s, 3H), 3.97 (s, 3H), 4.10-4.12 (m, 1H), 4.16-4.20 (m, 7H), 4.25-4.29 (m, 5H), 4.35-4.38 (m, 2H), 4.41 (dd, J=4.3 Hz, 4.5 Hz, 2H), 4.45-4.47 (m, 2H), 4.66 (t, J=5.3 Hz, 1H), 5.78 (d, J=5.4 Hz, 1H), 5.81 (d, J=4.0 Hz, 1H), 5.87 (d, J=5.5 Hz, 1H), 6.01 (d, J=4.2 Hz, 1H), 7.86 (s, 1H), 7.87 (s, 1H), 8.09 (s, 1H), 8.22 (s, 1H), 8.33 (s, 1H). ³¹P NMR (162 MHz, D₂O) δ −22.90 (dd, J=17.8 Hz, 19.4 Hz, 1P), −11.54 (d, J=17.8 Hz, 1P), −11.42 (d, J=19.4 Hz, 1P), −1.09 (s, 1P), −0.89 (s, 1P). ESI-HRMS calculated for C₄₃H₅₆N₂₀O₃₀P₅ 1487.2164 [M-H]⁻, found 1487.2168 [M-H]⁻.

Synthesis of ^{m7}G_{3′OMe}pppA_{2′OMe}pA_{2′OMe}pG

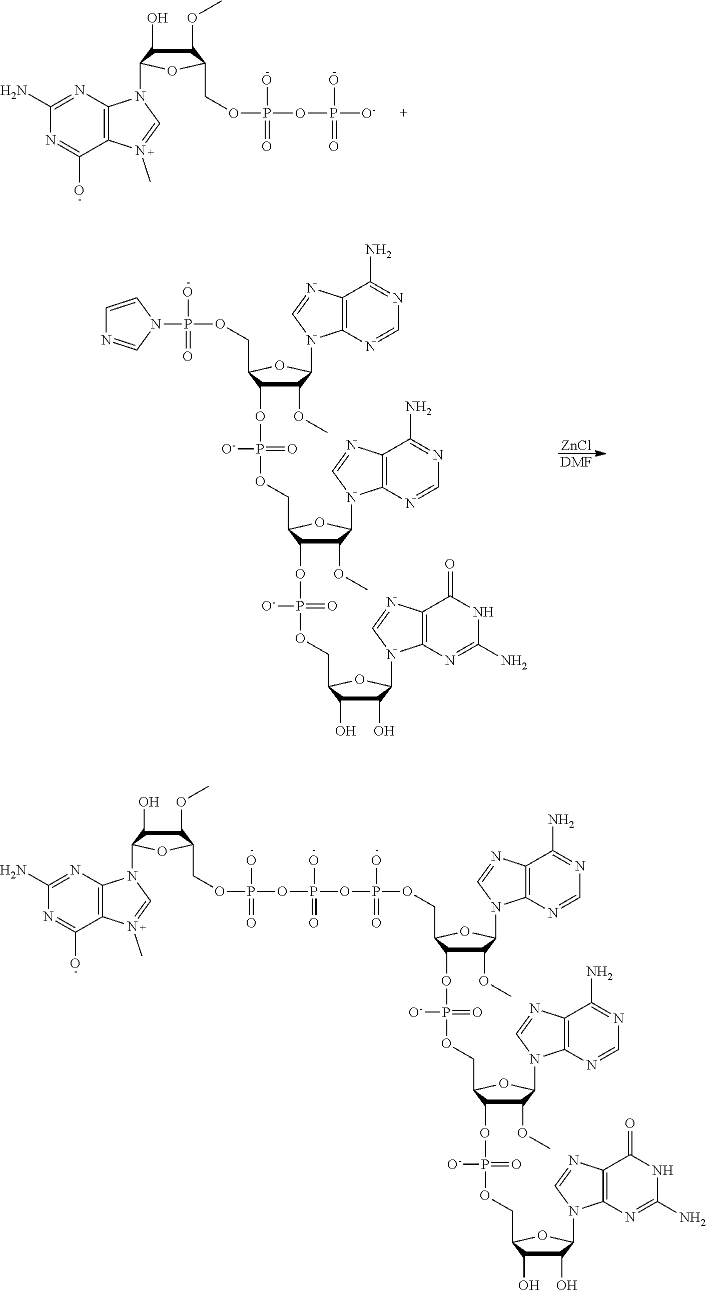

To a solution of pA_{2′OMe}pA_{2′OMe}pG (87 mg, 60 μmol) in DMF (0.5 mL), imidazole (40.7 mg, 597 μmol), 2,2′-dithiodipyridine (79 mg, 358 μmol), TEA (50 μL, 358 μmol) and triphenylphosphine (94 mg, 358 μmol) was added and the mixture was stirred at room temperature for 1 hour. The phosphorimidazolide of pA_{2′OMe}pA_{2′OMe}pG was precipitated by addition of sodium perchlorate (35.5 mg, 290 μmol) in acetone (10 mL). The solution was centrifuged at 4° C. washed with chilled acetone 3 times and dried under reduced pressure to give pA_{2′OMe}pA_{2′OMe}pG-Im.

pA_{2′OMe}pA_{2′OMe}pG-Im was then dissolved in DMF (0.5 mL), and ^{m7}G_{3′OMe}DP·TEA salt (92 mg, 119 μmol) and ZnCl₂ (40.7 mg, 299 μmol) were added. The mixture was stirred at room temperature for 12 hours, and the reaction was quenched by addition of 0.5 M EDTA aq. (0.6 mL) and 0.1 M NaHCO₃ aq. (10 mL). The tetranucleotide product was purified by RP-HPLC (Yamazen Flush ODS Premium M size, Buffer A: 0.1 M TEAA, Buffer B: ACN, linear gradient 2%-20% of Buffer B over 30 min). The isolated product was concentrated by evaporator and precipitated by sodium perchlorate (122 mg) in acetone (10 mL). The solution was centrifuged at 4° C. washed with chilled acetone 3 times and dried under reduced pressure to obtain m7G_{3′OMe}pppA_{2′OMe}pA_{2′OMe}pG·Na salt (27 mg, 17 μmol, 28% yield).

¹H NMR (700 MHz, D₂O) δ 3.42 (s, 3H), 3.49 (s, 3H), 3.50 (s, 3H), 4.01-4.03 (m, 4H), 4.11-4.23 (m, 8H), 4.27-4.31 (m, 5H), 4.38-4.41 (m, 2H), 4.44 (dd, J=4.7 Hz, 4.8 Hz, 1H), 4.48 (m, 1H), 4.55 (dd, J=4.6 Hz, 4.7 Hz, 1H), 4.70 (dd, J=4.8 Hz, 5.8 Hz, 1H), 5.70 (d, J=4.5 Hz, 1H), 5.77 (d, J=5.2 Hz, 1H), 5.83 (d, J=5.5 Hz, 1H), 5.97 (d, J=4.3 Hz, 1H), 7.85 (s, 1H), 7.86 (s, 1H), 8.09 (s, 1H), 8.21 (s, 1H), 8.33 (s, 1H). ³¹P NMR (162 MHz, D₂O) δ −22.90 (t, J=17.8 Hz, 1P), −11.47 (t, J=17.8 Hz, 2P), −0.99 (s, 1P), −0.90 (s, 1P). ESI-HRMS calculated for C₄₄H₅₈N₂₀O₃₀P₅ 1501.2321 [M-H]⁻, found 1501.2327 [M-H]⁻.

Synthesis of ^{m7}GpppU_{2′OMe}pA_{2′OMe}pG

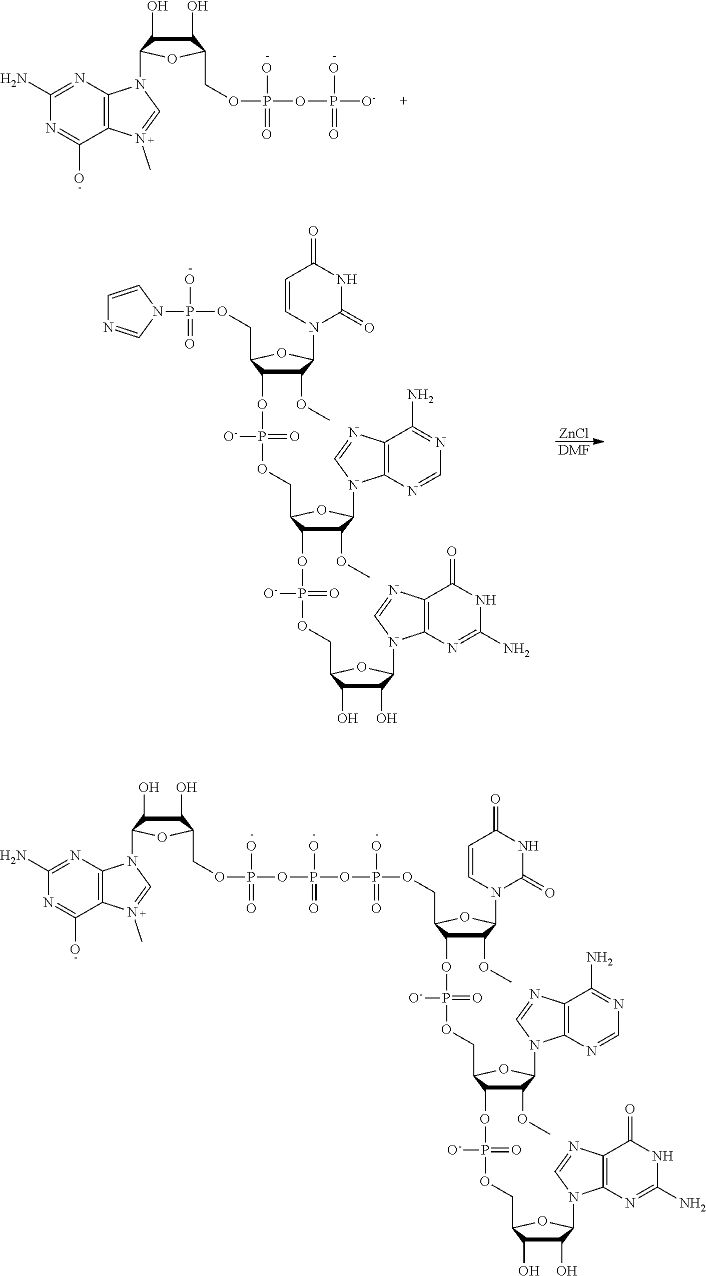

To a solution of pU_{2′OMe}pA_{2′OMe}pG (50 mg, 35 μmol) in DMF (1 mL), imidazole (38 mg, 559 μmol), 2,2′-dithiodipyridine (46.2 mg, 210 μmol), TEA (29 μL, 210 μmol) and triphenylphosphine (55 mg, 210 μmol) was added and the mixture was stirred at room temperature for 1 hour. The phosphorimidazolide of pU_{2′OMe}pA_{2′OMe}pG was precipitated by addition of sodium perchlorate (42.9 mg, 350 μmol) in acetone (10 mL). The solution was centrifuged at 4° C. washed with chilled acetone 3 times and dried under reduced pressure to give pU_{2′OMe}pA_{2′OMe}pG-Im.

pU_{2′OMe}pA_{2′OMe}pG-Im was then dissolved in DMF (0.5 mL), and ^{m7}GDP·TEA salt (58.3 mg, 105 μmol) and ZnCl₂ (28.6 mg, 210 μmol) were added. The mixture was stirred at room temperature for 12 hours, and the reaction was quenched by addition of 0.5 M EDTA aq. (0.6 mL) and 0.1 M TEAA buffer (8 mL). The tetranucleotide product was purified by anion-exchanging chromatography on DEAE Sephadex (linear gradient of 0.05-0.8 M TEAB over 50 min) and RP-HPLC (Biotage Sfür C18, 6 g, Buffer A: 0.1% ammonium formate, Buffer B: ACN, linear gradient 1%-12% of Buffer B over 25 min). The isolated product was concentrated by evaporator and precipitated by sodium perchlorate (43 mg) in acetone (10 mL). The solution was centrifuged at 4° C. washed with chilled acetone 3 times and dried under reduced pressure to obtain ^{m7}GpppU_{2′OMe}pA_{2′OMe}pG·Na salt (18 mg, 11 μmol, 33% yield).

¹H NMR (700 MHz, D₂O) δ 3.48 (s, 3H), 3.36 (s, 3H), 3.98-4.00 (m, 1H), 4.05 (s, 3H), 4.09-4.10 (m, 3H), 4.13-4.21 (m, 5H), 4.29-4.34 (m, 5H), 4.40 (s, 1H), 4.43-4.47 (m, 4H), 4.61 (dd, J=4.1 Hz, 4.5 Hz, 1H), 4.68-4.71 (m, 1H), 4.73 (t, J=5.3 Hz, 1H), 4.90-4.92 (m, 1H), 5.76 (d, J=5.7 Hz, 1H), 5.79 (d, J=5.5 Hz, 1H), 5.89 (d, J=8.1 Hz, 1H), 5.94 (d, J=3.7 Hz, 1H), 6.05 (d, J=4.9 Hz, 1H), 7.79 (d, J=8.1 Hz, 1H), 7.90 (s, 1H), 8.11 (s, 1H), 8.33 (s, 1H). ³¹P NMR (162 MHz, D₂O) δ −22.82 (t, J=18.2 Hz, 1P), −11.55 (d, J=17.9 Hz, 1P) 11.37 (d, J=19.2 Hz, 1P), −1.13 (s, 1P), −0.87 (s, 1P).

ESI-HRMS calculated for C₄₂H₅₅N₁₇O₃₂P₅ 1464.1893 [M-H]⁻, found 1464.1936 [M-H]⁻.

Synthesis of ^{m7}GpppA_{2′OMe}pC_{2′OMe}pG

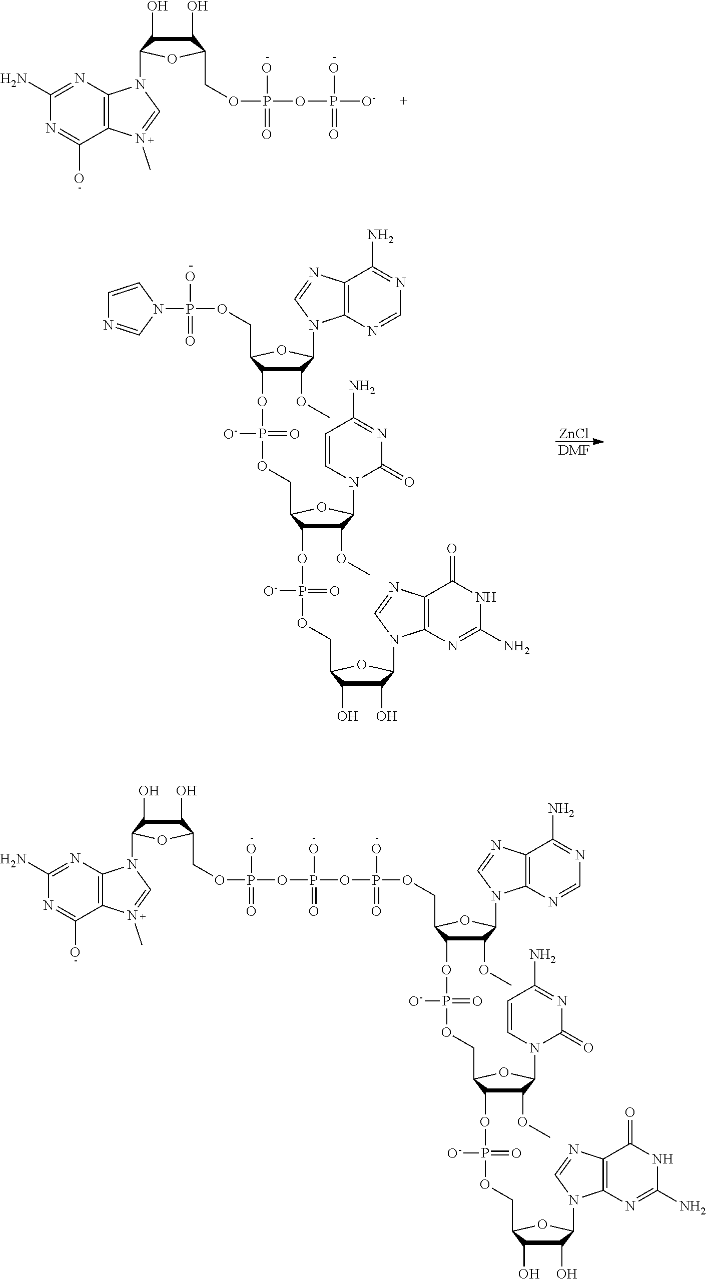

To a solution of pA_{2′OMe}pC_{2′OMe}pG (42 mg, 29 μmol) in DMF (1 mL), imidazole (20 mg, 295 μmol), 2,2′-dithiodipyridine (39 mg, 177 μmol), TEA (25 μL, 177 μmol) and triphenylphosphine (46 mg, 177 μmol) was added and the mixture was stirred at room temperature for 1 hour. The phosphorimidazolide of pA_{2′OMe}pC_{2′OMe}pG was precipitated by addition of sodium perchlorate (36 mg, 290 μmol) in acetone (10 mL). The solution was centrifuged at 4° C. washed with chilled acetone 3 times and dried under reduced pressure to give pA_{2′OMe}pC_{2′OMe}pG-Im.

pA_{2′OMe}pC_{2′OMe}pG-Im was dissolved in DMF (1 mL), followed by addition of ^{m7}GDP·TEA salt (45 mg, 59 μmol) and ZnCl₂ (20 mg, 147 μmol). The mixture was stirred at room temperature for 12 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (36 mg) in acetone (10 mL). The solution was centrifuged at 4° C. washed with chilled acetone 3 times and dried under reduced pressure to obtain ^{m7}GpppA_{2′OMe}pC_{2′OMe}pG·Na salt (31 mg, 20 μmol, 67% yield).

¹H NMR (700 MHz, D₂O) δ 3.53 (s, 3H), 3.56 (s, 3H), 3.92-3.93 (m, 1H), 4.04 (s, 3H), 4.11-4.14 (m, 3H), 4.21-4.25 (m, 3H), 4.30-4.32 (m, 3H), 4.36-4.47 (m, 7H), 4.50-4.52 (m, 2H), 4.56-4.57 (m, 1H), 5.60 (d, J=7.0 Hz, 1H), 5.77 (d, J=2.1 Hz, 1H), 5.79 (d, J=7.0 Hz, 1H), 5.85 (d, J=4.2 Hz, 1H), 5.97 (d, J=4.2 Hz, 1H), 7.62 (d, J=7.7 Hz, 1H), 7.92 (s, 1H), 8.04 (s, 1H), 8.36 (s, 1H). ³¹P NMR (162 MHz, D₂O) δ −22.87 (dd, J=19.4 Hz, 17.8 Hz 1P), −11.56 (d, J=17.8 Hz, 1P), −11.44 (d, J=19.4 Hz, 1P), −1.37 (s, 1P), −1.00 (s, 1P). ESI-HRMS calculated for C₄₂H₅₆N₁₈O₃₁P₅ 1463.2052 [M-H]⁻, found 1463.2018 [M-H]⁻.

Synthesis of ^{m7}GpppA_{2′OMe}pU_{2′OMe}pG

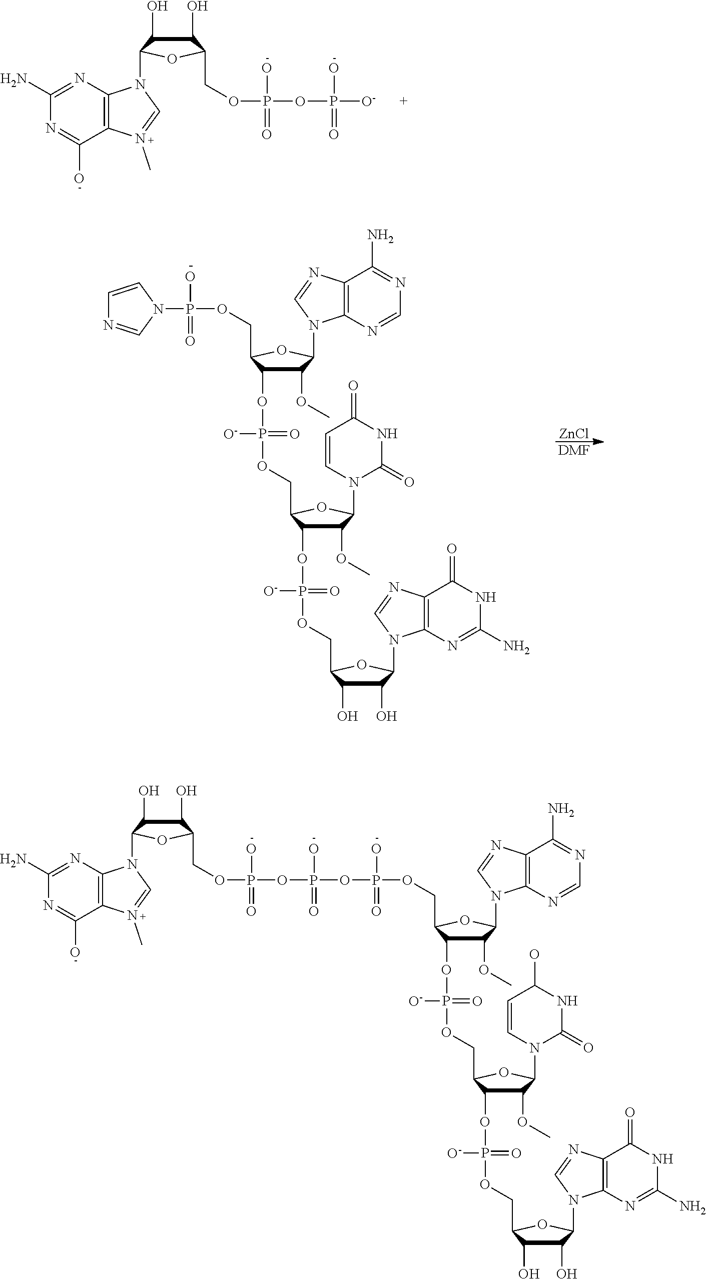

To a solution of pA_{2′OMe}pU_{2′OMe}pG (94 mg, 61 μmol) in DMF (1 mL), imidazole (42 mg, 615 μmol), 2,2′-dithiodipyridine (81 mg, 369 μmol), TEA (51 μL, 369 μmol) and triphenylphosphine (97 mg, 369 μmol) was added and the mixture was stirred at room temperature for 1 hour. The phosphorimidazolide of pA_{2′OMe}pU_{2′OMe}pG was precipitated by addition of sodium perchlorate (75 mg, 610 μmol) in acetone (10 mL). The solution was centrifuged at 4° C. washed with chilled acetone 3 times and dried under reduced pressure to give pA_{2′OMe}pU_{2′OMe}pG-Im.

pA_{2′OMe}pU_{2′OMe}pG-Im was dissolved in DMF (1 mL), followed by addition of ⁷GDP·TEA salt (93 mg, 123 μmol) and ZnCl₂ (42 mg, 308 μmol)e. The mixture was stirred at room temperature for 12 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (75 mg) in acetone (10 mL). The solution was centrifuged at 4° C. washed with chilled acetone 3 times and dried under reduced pressure to obtain ^{m7}GpppA_{2′OMe}pU_{2′OMe}pG·Na salt (85 mg, 54 μmol, 88% yield).

¹H NMR (700 MHz, D₂O) δ 3.44 (s, 3H), 3.45 (s, 3H), 4.00 (d, J=4.7 Hz, 1H), 4.01 (s, 3H), 4.07-4.14 (m, 5H), 4.17-4.23 (m, 3H), 4.29-4.30 (m, 5H), 4.39 (d, J=4.9 Hz, 1H), 4.40 (d, J=4.9 Hz, 1H), 4.43 (d, J=4.9 Hz, 1H), 4.44 (d, J=4.2 Hz, 1H), 4.48 (t, J=4.2 Hz, 1H), 4.53 (s, 1H), 4.87-4.88 (m, 1H), 5.66 (d, J=8.4 Hz, 1H), 5.78 (d, J=4.2 Hz, 1H), 5.80-5.81 (m, 2H), 6.00 (d, J=6.3 Hz, 1H), 7.72 (d, J=8.4 Hz, 1H), 7.94 (s, 1H), 8.09 (s, 1H), 8.40 (s, 1H).

³¹P NMR (162 MHz, D₂O) δ −22.95 (dd, J=19.4 Hz, 17.8 Hz, 1P), −11.51 (d, J=17.8 Hz, 1P), −11.45 (d, J=17.8 Hz, 1P), −1.04 (s, 1P), −0.98 (s, 1P). ESI-HRMS calculated for C₄₂H₅₅N₁₇O₃₂P₅ 1464.1893 [M-H]⁻, found 1464.1884 [M-H]⁻.

Synthesis of ^{m7}GpppA_{2′OMe}pA_{2′OMe}pA

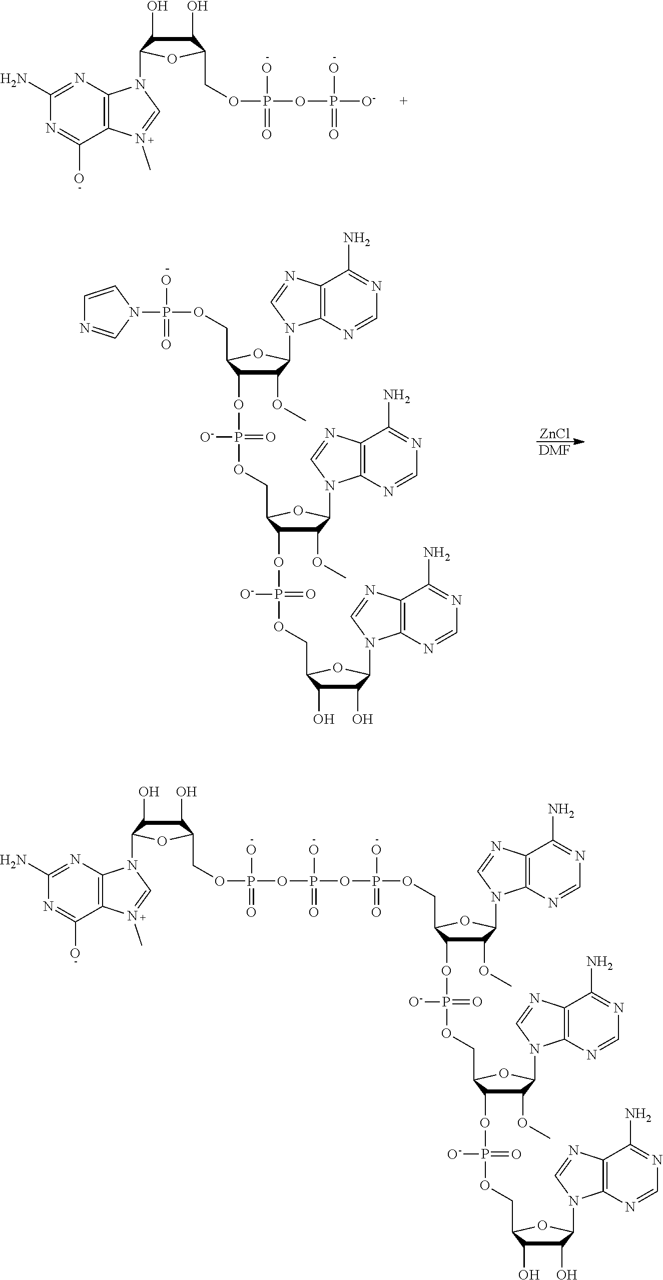

To a solution of pA_{2′OMe}pA_{2′OMe}pA (90 mg, 63 μmol) in DMF (1 mL), imidazole (43 mg, 626 μmol), 2,2′-dithiodipyridine (83 mg, 376 μmol), TEA (52 μL, 376 μmol) and triphenylphosphine (99 mg, 376 μmol) was added and the mixture was stirred at room temperature for 1 hour. The phosphorimidazolide of pA_{2′OMe}pA_{2′OMe}pA was precipitated by addition of sodium perchlorate (77 mg, 630 μmol) in acetone (10 mL). The solution was centrifuged at 4° C. washed with chilled acetone 3 times and dried under reduced pressure to give pA_{2′OMe}pA_{2′OMe}pA-Im.

pA_{2′OMe}pA_{2′OMe}pA-Im was dissolved in DMF (1 mL), followed by addition of ^{m7}GDP·TEA salt (95 mg, 125 μmol) and ZnCl₂ (43 mg, 313 μmol). The mixture was stirred at room temperature for 12 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (77 mg) in acetone (10 mL). The solution was centrifuged at 4° C. washed with chilled acetone 3 times and dried under reduced pressure to obtain ^{m7}GpppA_{2′OMe}pA_{2′OMe}pA·Na salt (78 mg, 49 μmol, 78% yield).

¹H NMR (700 MHz, D₂O) δ 3.50 (s, 3H), 3.52 (s, 3H), 3.99 (s, 3H), 4.16-4.36 (m, 12H), 4.39 (t, J=4.9 Hz, 1H), 4.41 (t, J=4.9 Hz, 1H), 4.45-4.52 (m, 6H), 4.48 (t, J=4.4 Hz, 1H), 4.53 (s, 1H), 4.87-4.88 (m, 1H), 5.80 (d, J=3.5 Hz, 1H), 5.84 (d, J=5.6 Hz, 1H), 5.99 (d, J=5.6 Hz, 1H), 5.99 (d, J=4.9 Hz, 1H), 7.80 (s, 1H), 8.00 (s, 1H), 8.13 (s, 1H), 8.20 (s, 1H). 8.24 (s, 1H), 8.31 (s, 1H). ³¹P NMR (162 MHz, D₂O) δ −22.98 (t, J=17.8 Hz, 1P), −11.48 (d, J=17.8 Hz, 2P), −1.05 (s, 2P). ESI-HRMS calculated for C₄₃H₅₆N₂₀O₃₀P₅ 1471.2216 [M-H]⁻, found 1471.2219 [M-H]⁻.

Synthesis of ^{m7}GpppA_{2′OMe}pA_{2′OMe}pC

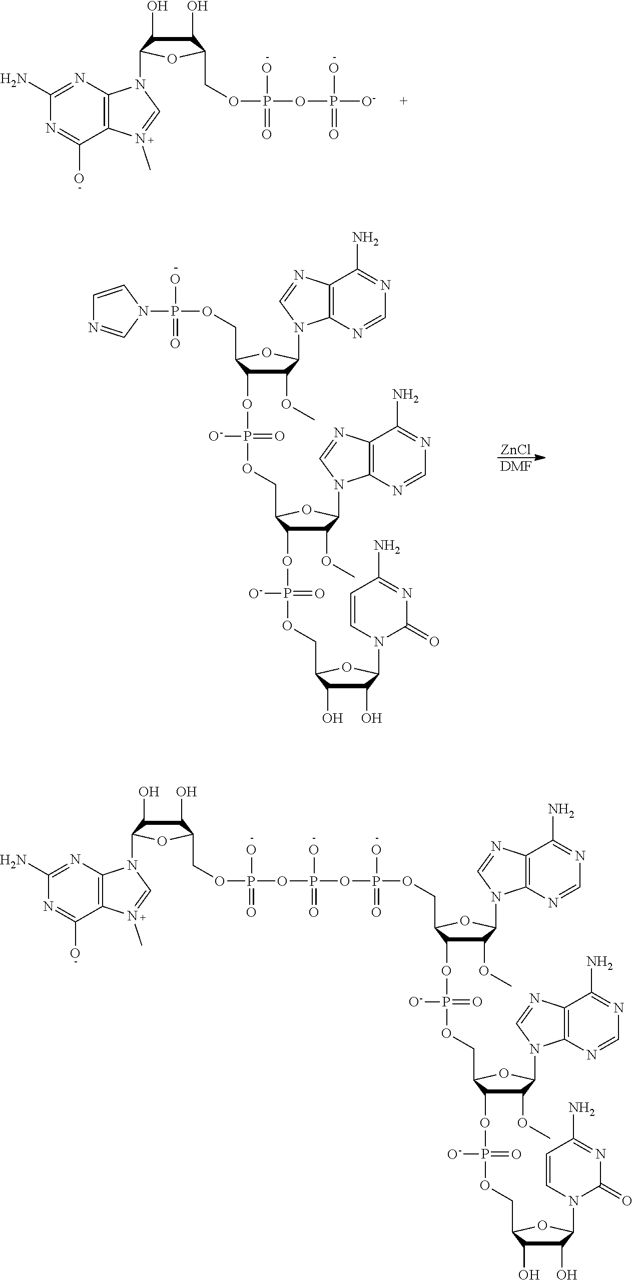

To a solution of pA_{2′OMe}pA_{2′OMe}pC (84 mg, 60 μmol) in DMF (1 mL), imidazole (41 mg, 595 μmol), 2,2′-dithiodipyridine (79 mg, 357 μmol), TEA (50 μL, 357 μmol) and triphenylphosphine (94 mg, 357 μmol) was added and the mixture was stirred at room temperature for 1 hour. The phosphorimidazolide of pA_{2′OMe}pA_{2′OMe}pC was precipitated by addition of sodium perchlorate (73 mg, 600 μmol) in acetone (10 mL). The solution was centrifuged at 4° C. washed with chilled acetone 3 times and dried under reduced pressure to give pA_{2′OMe}pA_{2′OMe}pC-Im.

pA_{2′OMe}pA_{2′OMe}pC-Im was dissolved in DMF (1 mL), followed by addition of ^{m7}GDP·TEA salt (90 mg, 119 μmol) and ZnCl₂ (41 mg, 298 μmol). The mixture was stirred at room temperature for 12 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (73 mg) in acetone (10 mL). The solution was centrifuged at 4° C. washed with chilled acetone 3 times and dried under reduced pressure to obtain ^{m7}GpppA_{2′OMe}pA_{2′OMe}pC·Na salt (78 mg, 50 μmol, 84% yield).

¹H NMR (700 MHz, D₂O) δ 3.56 (s, 3H), 3.61 (s, 3H), 4.00 (s, 3H), 4.11 (t, J=3.5 Hz, 2H), 4.21-4.36 (m, 13H), 4.41 (t, J=4.9 Hz, 1H), 4.44 (dd, J=4.9 Hz, 4.2 Hz, 1H), 4.50-4.54 (m, 3H), 5.67 (d, J=7.0 Hz, 1H), 5.75 (d, J=2.1 Hz, 1H), 5.82 (d, J=4.2 Hz, 1H), 5.90 (d, J=4.9 Hz, 1H), 6.10 (d, J=2.8 Hz, 1H), 7.67 (d, J=7.7 Hz, 1H), 7.81 (s, 1H), 8.11 (s, 1H), 8.24 (s, 1H), 8.33 (s, 1H). ³¹P NMR (162 MHz, D₂O) δ −22.96 (dd, J=19.4 Hz, 17.8 Hz, 1P), −11.47 (d, J=17.8 Hz, 2P), −1.16 (s, 1P), −1.06 (s, 1P). ESI-HRMS calculated for C₄₂H₅₆N₁₈O₃₀P₅ 1447.2103 [M-H]⁻, found 1447.2118 [M-H]⁻.

Synthesis of ^{m7}GpppA_{2′OMe}pA_{2′OMe}PU

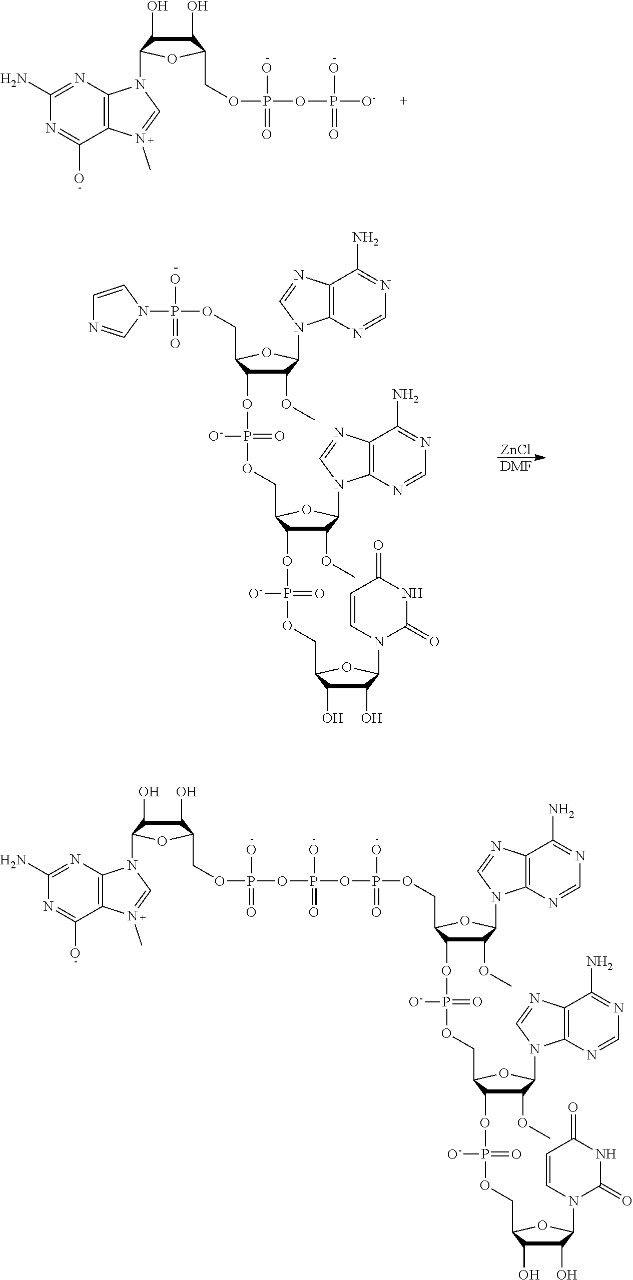

To a solution of pA_{2′OMe}pA_{2′OMe}pU (68 mg, 48 μmol) in DMF (1 mL), imidazole (33 mg, 481 μmol), 2,2′-dithiodipyridine (64 mg, 289 μmol), TEA (40 μL, 289 μmol) and triphenylphosphine (76 mg, 289 μmol) was added and the mixture was stirred at room temperature for 1 hour. The phosphorimidazolide of pA_{2′OMe}pA_{2′OMe}pU was precipitated by addition of sodium perchlorate (59 mg, 481 μmol) in acetone (10 mL). The solution was centrifuged at 4° C. washed with chilled acetone 3 times and dried under reduced pressure to give pA_{2′OMe}pA_{2′OMe}pU-Im.

pA_{2′OMe}pA_{2′OMe}pU-Im was dissolved in DMF (1 mL), followed by addition of ^{m7}GDP·TEA salt (73 mg, 96 μmol) and ZnCl₂ (33 mg, 241 μmol). The mixture was stirred at room temperature for 18 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (59 mg) in acetone (10 mL). The solution was centrifuged at 4° C. washed with chilled acetone 3 times and dried under reduced pressure to obtain ^{m7}GpppA_{2′OMe}pA_{2′OMe}pU·Na salt (53 mg, 34 μmol, 70% yield).

¹H NMR (700 MHz, D₂O) δ 3.52 (s, 3H), 3.56 (s, 3H), 4.00 (s, 3H), 4.11-4.13 (m, 1H), 4.20-4.35 (m, 14H), 4.38 (dd, J=4.9 Hz, 4.2 Hz, 1H), 4.41 (t, J=4.9 Hz, 1H), 4.49-4.50 (m, 2H), 4.55 (d, J=1.6 Hz, 1H), 5.61 (d, J=8.4 Hz, 1H), 5.82 (dd, J=4.9 Hz, 4.2 Hz, 1H), 5.85 (d, J=4.2 Hz, 1H), 6.12 (d, J=4.2 Hz, 1H), 7.74 (d, J=8.4 Hz, 1H), 7.83 (s, 1H), 8.14 (s, 1H), 8.26 (s, 1H), 8.31 (s, 1H). ³¹P NMR (162 MHz, D₂O) δ −22.95 (dd, J=19.4 Hz, 17.8 Hz, 1P), −11.46 (d, J=17.8 Hz, 2P), −1.10 (s, 2P). ESI-HRMS calculated for C₄₂H₅₆N₁₈O₃₀P₅ 1448.1943 [M-H]⁻, found 1448.2008 [M-H]⁻.

Synthesis of ^{m7}GpppC_{2′OMe}pA_{2′OMe}pG

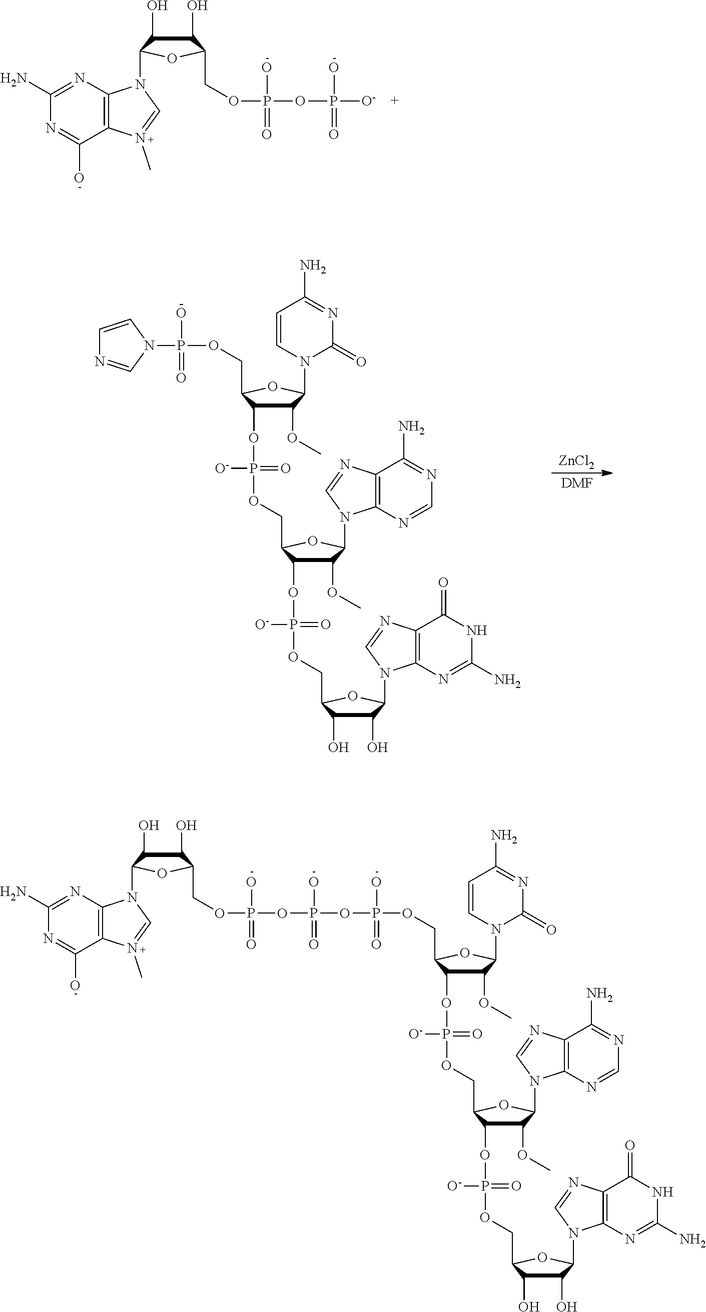

To a solution of pC_{2′OMe}pA_{2′OMe}pG (101 mg, 71 μmol) in DMF (0.5 mL), imidazole (48 mg, 706 μmol), 2,2′-dithiodipyridine (93 mg, 424 μmol), TEA (59 μL, 424 μmol) and triphenylphosphine (111 mg, 424 μmol) was added and the mixture was stirred at room temperature for 1 hour. The phosphorimidazolide of pC_{2′OMe}pA_{2′OMe}pG was precipitated by addition of sodium perchlorate (86 mg, 706 μmol) in acetone (10 mL). The solution was centrifuged at 4° C. washed with chilled acetone 3 times and dried under reduced pressure to give pC_{2′OMe}pA_{2′OMe}pG-Im.

pC_{2′OMe}pA_{2′OMe}pG-Im was dissolved in DMF (0.5 mL), followed by addition of ^{m7}GDP·TEA salt (80 mg, 106 μmol) and ZnCl₂ (48 mg, 353 μmol). The mixture was stirred at room temperature for 18 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (86 mg) in acetone (10 mL). The solution was centrifuged at 4° C. washed with chilled acetone 3 times and dried under reduced pressure to obtain ^{m7}GpppC_{2′OMe}pA_{2′OMe}pG·Na salt (20 mg, 13 μmol, 18% yield).

¹H NMR (700 MHz, D₂O) δ 3.42 (s, 3H), 3.51 (s, 3H), 4.04 (dd, J=4.9 Hz, 4.2 Hz, 1H), 4.09 (s, 3H), 4.11 (s, 2H), 4.15-4.24 (m, 5H), 4.30-4.36 (m, 3H), 4.42 (s, 2H), 4.46 (dd, J=4.9 Hz, 4.2 Hz, 1H), 4.50 (t, J=4.9 Hz, 1H), 4.65 (t, J=4.2 Hz, 1H), 4.90-4.93 (m, 1H), 5.74 (s, 1H), 5.80 (d, J=5.6 Hz, 1H), 5.98 (d, J=3.5 Hz, 1H), 6.10 (d, J=4.2 Hz, 1H), 6.14 (d, J=7.7 Hz, 1H), 7.92 (d, J=6.3 Hz, 1H), 8.38 (s, 1H), 8.43 (s, 1H), 9.17 (s, 1H). ³¹P NMR (162 MHz, D₂O) δ −22.84 (t, J=17.8 Hz, 1P), −11.52 (d, J=17.8 Hz, 1P), −11.36 (d, J=17.8 Hz, 1P), −1.19 (s, 1P), −0.87 (s, 1P). ESI-HRMS calculated for C₄₃H₅₆N₂₀O₃₀P₅ 1463.2052 [M-H]⁻, found 1463.2018 [M-H]⁻.

Synthesis of ^{m7}GpppG_{2′OMe}pA_{2′OMe}pG

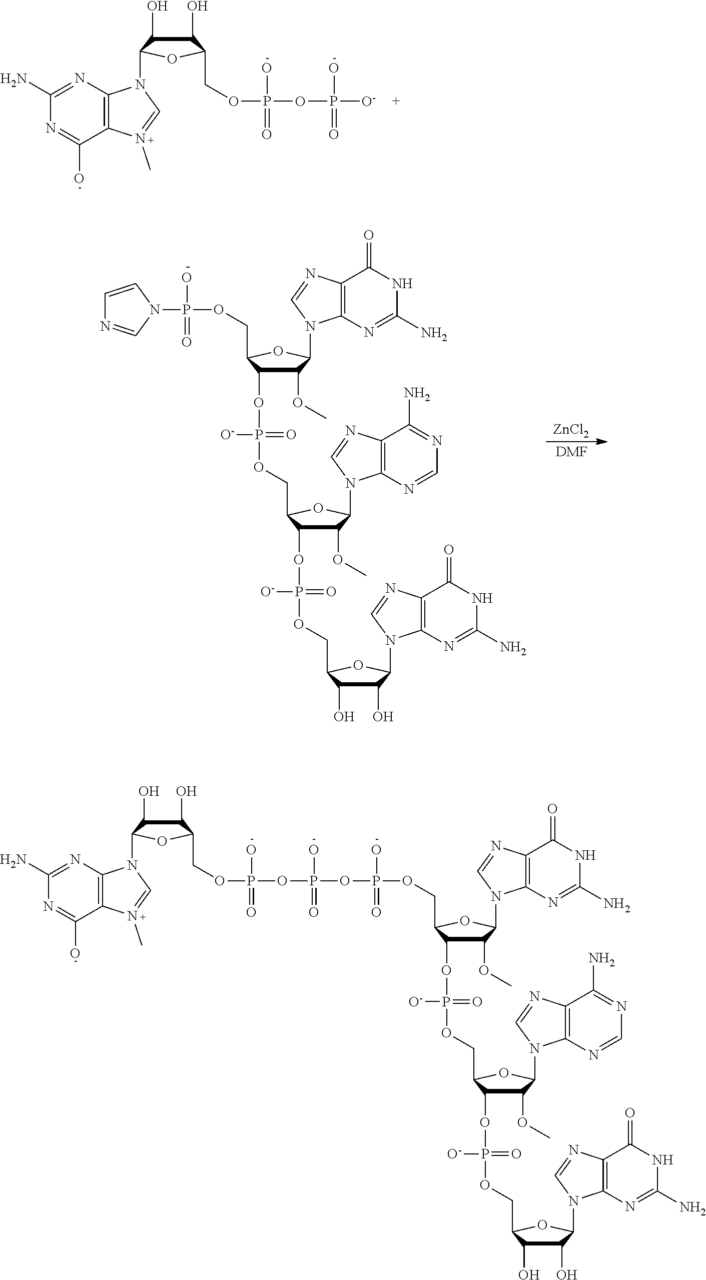

To a solution of pG_{2′OMe}pA_{2′OMe}pG (87 mg, 59 μmol) in DMF (7 mL) and DMSO (1 mL), imidazole (40 mg, 592 μmol), 2,2′-dithiodipyridine (78 mg, 355 μmol), TEA (50 μL, 355 μmol) and triphenylphosphine (93 mg, 355 μmol) was added and the mixture was stirred at room temperature for 1 hour. The phosphorimidazolide of pG_{2′OMe}pA_{2′OMe}pG was precipitated by addition of sodium perchlorate (72 mg, 590 μmol) in acetone (10 mL). The solution was centrifuged at 4° C. washed with chilled acetone 3 times and dried under reduced pressure to give pG_{2′OMe}pA_{2′OMe}pG-Im.

pG_{2′OMe}pA_{2′OMe}pG-Im was dissolved in DMF (0.6 mL), followed by addition of ^{m7}GDP·TEA salt (90 mg, 118 μmol) and ZnCl₂ (40 mg, 118 μmol). The mixture was stirred at room temperature for 18 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (72 mg) in acetone (10 mL). The solution was centrifuged at 4° C. washed with chilled acetone 3 times and dried under reduced pressure to obtain ^{m7}GpppG_{2′OMe}pA_{2′OMe}pG·Na salt (52 mg, 32 μmol, 54% yield).

¹H NMR (700 MHz, D₂O) δ 3.42 (s, 3H), 3.48 (s, 3H), 4.03 (s, 3H), 4.14-4.21 (m, 7H), 4.23-4.24 (m, 1H), 4.26-4.29 (m, 1H), 4.30-4.31 (m, 1H), 4.33-4.34 (m, 3H), 4.38-4.42 (m, 5H), 4.47 (dd, J=4.9 Hz, 4.2 Hz, 1H), 4.51 (t, J=4.2 Hz, 1H), 5.63 (d, J=5.6 Hz, 1H), 5.81 (d, J=5.6 Hz, 1H), 5.83 (d, J=3.5 Hz, 1H), 6.04 (d, J=5.6 Hz, 1H), 7.92 (s, 1H), 7.95 (s, 1H), 8.14 (s, 1H), 8.29 (s, 1H). ³¹P NMR (162 MHz, D₂O) δ −22.95 (t, J=17.8 Hz, 1P), −11.44 (d, J=17.8 Hz, 1P), −11.40 (d, J=17.8 Hz, 1P), −0.97 (s, 1P), −0.80 (s, 1P). ESI-HRMS calculated for C₄₃H₅₆N₂₀O₃₁P₅ 1503.2114 [M-H]⁻, found 1503.2119 [M-H]⁻.

Synthesis of ^{m7}GpppdTpA_{2′OMe}pG

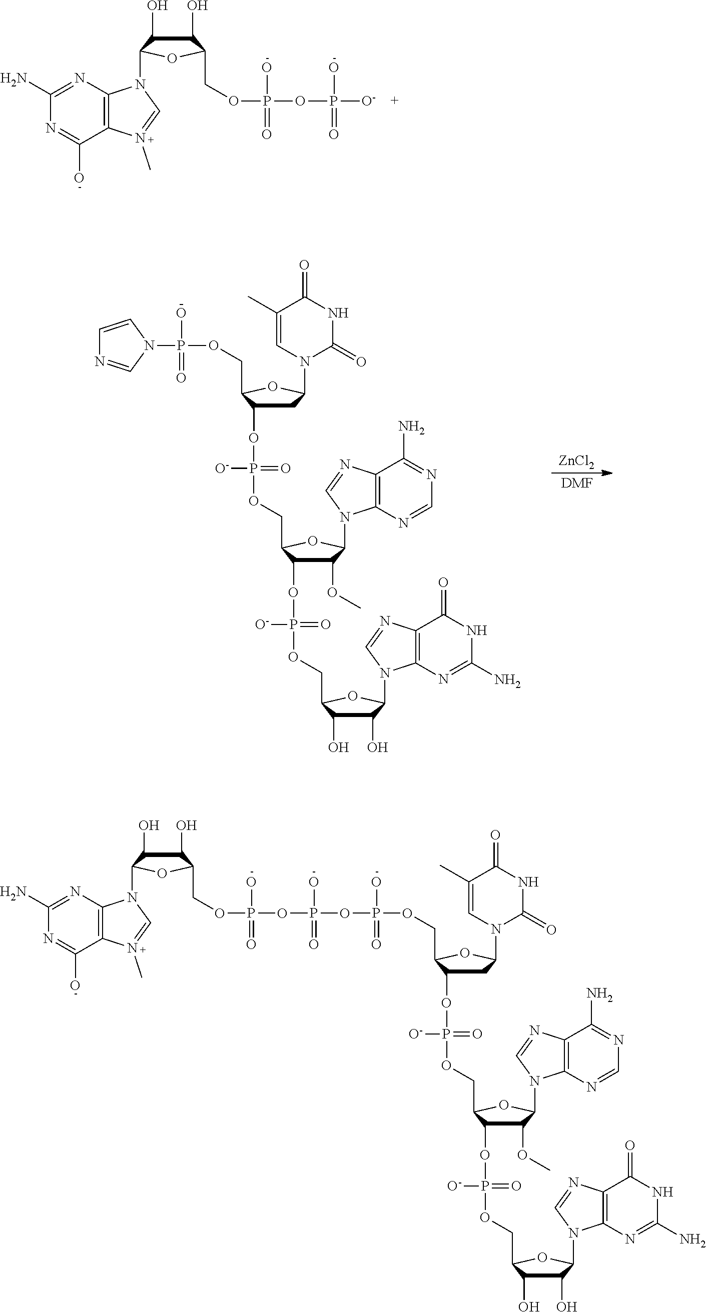

To a solution of pdTA_{2′OMe}pG (64 mg, 45 μmol) in DMF (1 mL), imidazole (31 mg, 454 mol), 2,2′-dithiodipyridine (60 mg, 272 μmol), TEA (38 μL, 272 μmol) and triphenylphosphine (71 mg, 272 μmol) was added and the mixture was stirred at room temperature for 1 hour. The phosphorimidazolide of pdTpA_{2′OMe}pG was precipitated by addition of sodium perchlorate (56 mg, 454 μmol) in acetone (10 mL). The solution was centrifuged at 4° C. washed with chilled acetone 3 times and dried under reduced pressure to give pdTA_{2′OMe}pG-Im.

pdTpA_{2′OMe}pG-Im was dissolved in DMF (1 mL), followed by addition of ^{m7}GDP·TEA salt (69 mg, 91 μmol) and ZnCl₂ (31 mg, 227 μmol). The mixture was stirred at room temperature for 18 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (56 mg) in acetone (10 mL). The solution was centrifuged at 4° C. washed with chilled acetone 3 times and dried under reduced pressure to obtain ^{m7}GpppdTpA_{2′OMe}pG·Na salt (49 mg, 31 μmol, 69% yield).

¹H NMR (700 MHz, D₂O) δ 1.88 (s, 3H), 3.49 (s, 3H), 3.99 (d, J=11.9 Hz, 1H), 4.09-4.10 (m, 5H), 4.19-4.25 (m, 4H), 4.32-4.35 (m, 4H), 4.40 (s, 1H), 4.50 (t, J=4.9 Hz, 1H), 4.52 (dd, J=4.9 Hz, 4.2 Hz, 1H), 4.55 (t, J=5.6 Hz, 2H), 4.63-4.64 (m, 2H), 4.98-5.01 (m, 2H), 5.88 (d, J=5.6 Hz, 1H), 5.94 (dd, J=9.1 Hz, 5.6 Hz, 1H), 5.96 (d, J=4.2 Hz, 1H), 6.00 (d, J=6.3 Hz, 1H), 7.38 (s, 1H), 8.00 (s, 1H), 8.15 (s, 1H), 8.40 (s, 1H). ³¹P NMR (162 MHz, D₂O) δ −23.05 (dd, J=19.4 Hz, 17.8 Hz, 1P), −11.74 (d, J=19.4 Hz, 1P), −11.47 (d, J=17.8 Hz, 1P), −1.42 (s, 1P), −0.75 (s, 1P). ESI-HRMS calculated for C₄₂H₅₄N₁₇O₃₁P₅ 1448.1943 [M-H]⁻, found 1448.1930 [M-H]⁻.

Synthesis of ^{m7}GpppI_{2′OMe}pA_{2′OMe}pG

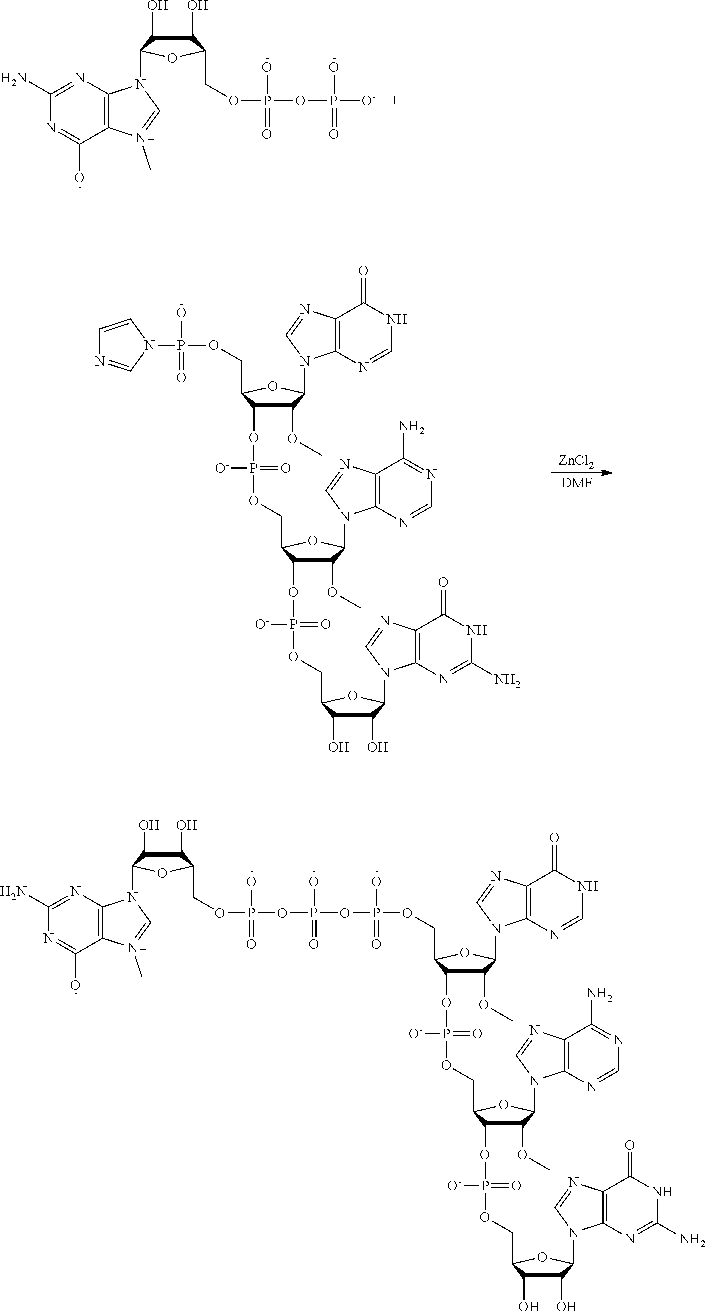

To a solution of pI_{2′OMe}A_{2′OMe}pG (78 mg, 54 μmol) in DMF (1 mL), imidazole (37 mg, 537 mol), 2,2′-dithiodipyridine (71 mg, 322 μmol), TEA (45 μL, 322 μmol) and triphenylphosphine (84 mg, 322 μmol) was added and the mixture was stirred at room temperature for 1 hour. The phosphorimidazolide of pI_{2′OMe}pA_{2′OMe}pG was precipitated by addition of sodium perchlorate (66 mg, 537 μmol) in acetone (10 mL). The solution was centrifuged at 4° C. washed with chilled acetone 3 times and dried under reduced pressure to give pI_{2′OMe}pA_{2′OMe}pG-Im.

pI_{2′OMe}pA_{2′OMe}pG-Im was dissolved in DMF (1 mL), followed by addition of ⁷GDP·TEA salt (81 mg, 107 μmol) and ZnCl₂ (37 mg, 268 μmol). The mixture was stirred at room temperature for 18 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (66 mg) in acetone (10 mL). The solution was centrifuged at 4° C. washed with chilled acetone 3 times and dried under reduced pressure to obtain ^{m7}GpppI_{2′OMe}pA_{2′OMe}pG·Na salt (56 mg, 35 μmol, 65% yield).

¹H NMR (700 MHz, D₂O) δ 3.39 (s, 3H), 3.46 (s, 3H), 4.03 (s, 3H), 4.15-5.23 (m, 8H), 4.29-4.32 (m, 2H), 4.38-4.43 (m, 4H), 4.46-4.47 (m, 2H), 4.51-4.53 (m, 1H), 4.88-4.92 (m, 3H), 5.81 (d, J=5.6 Hz, 1H), 5.83 (d, J=3.5 Hz, 1H), 5.85 (d, J=6.3 Hz, 1H), 6.02 (d, J=5.6 Hz, 1H), 7.90 (s, 1H), 7.91 (s, 1H), 8.13 (s, 1H), 8.31 (s, 1H), 8.34 (s, 1H). ³¹P NMR (162 MHz, D₂O) δ −22.98 (t, J=19.4 Hz, 1P), −11.53 (d, J=17.8 Hz, 1P), −11.49 (d, J=17.8 Hz, 1P), −1.06 (s, 1P), −0.91 (s, 1P). ESI-HRMS calculated for C₄₃H₅₅N₁₉O₃₁P₅ 1488.2005 [M-H]⁻, found 1488.1962 [M-H]⁻.

Synthesis of ^{m7}Gppp^{m5}C_{2′OMe}pA_{2′OMe}pG

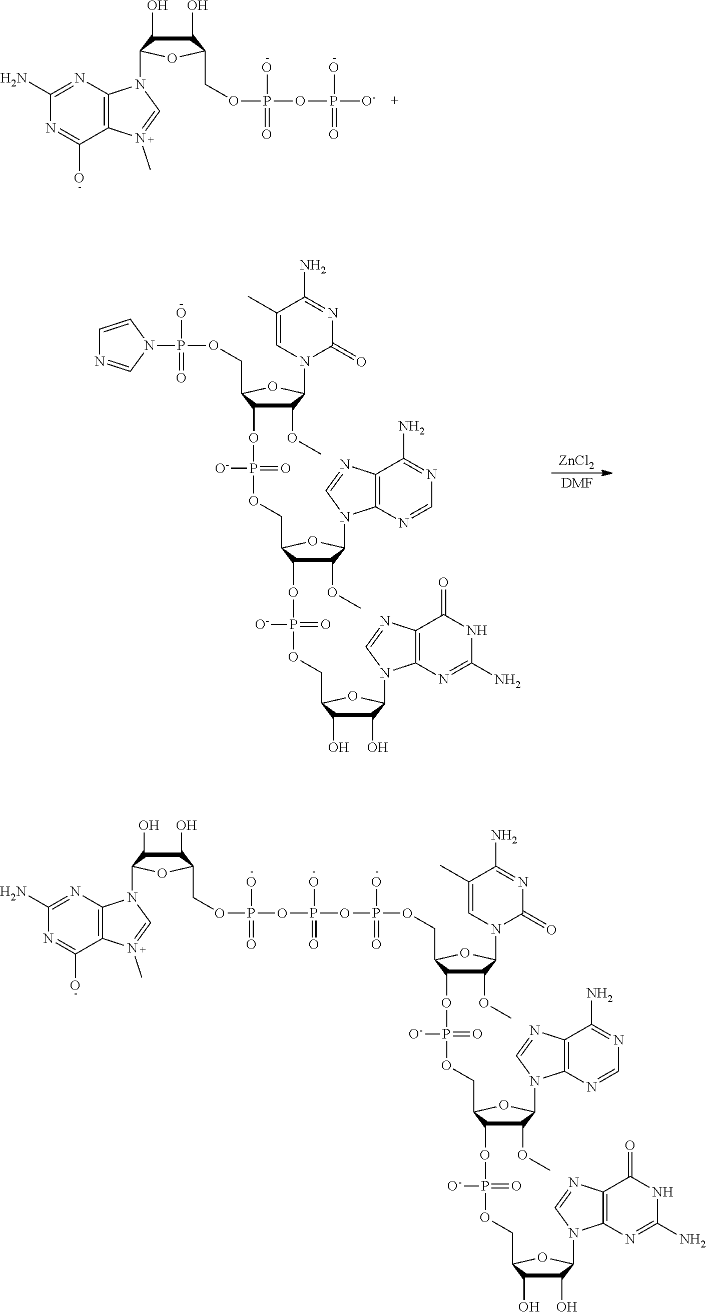

To a solution of p^{m5}C_{2′OMe}A_{2′OMe}pG (31 mg, 21 μmol) in DMF (0.5 mL), imidazole (15 mg, 215 μmol), 2,2′-dithiodipyridine (28 mg, 129 μmol), TEA (18 μL, 129 μmol) and triphenylphosphine (34 mg, 129 μmol) was added and the mixture was stirred at room temperature for 1 hour. The phosphorimidazolide of p^{m5}C_{2′OMe}pA_{2′OMe}pG was precipitated by addition of sodium perchlorate (26 mg, 215 μmol) in acetone (10 mL). The solution was centrifuged at 4° C. washed with chilled acetone 3 times and dried under reduced pressure to give p^{m5}C_{2′OMe}pA_{2′OMe}pG-Im.

p^{m5}C_{2′OMe}pA_{2′OMe}pG-Im was dissolved in DMF (0.5 mL), followed by addition of ^{m7}GDP·TEA salt (33 mg, 43 μmol) and ZnCl₂ (15 mg, 107 μmol). The mixture was stirred at room temperature for 18 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (26 mg) in acetone (10 mL). The solution was centrifuged at 4° C. washed with chilled acetone 3 times and dried under reduced pressure to obtain ^{m7}Gppp^{m5}C_{2′OMe}pA_{2′OMe}pG·Na salt (4.3 mg, 2.7 μmol, 12% yield).

¹H NMR (700 MHz, D₂O) δ 3.44 (s, 3H), 3.54 (s, 3H), 4.04 (dd, J=4.9 Hz, 3.5 Hz, 1H), 4.06 (s, 3H), 4.10-4.20 (m, 7H), 4.24-4.28 (m, 7H), 4.38-4.43 (m, 4H), 4.49-4.53 (m, 4H), 5.60 (br s, 1H), 5.73 (d, J=4.9 Hz, 1H), 5.90 (s, 1H), 6.08 (d, J=3.5 Hz, 1H), 7.46 (s, 1H), 7.81 (s, 1H), 8.08 (br s, 1H), 8.26 (br s, 1H). ³¹P NMR (162 MHz, D₂O) δ −22.89 (t, J=17.8 Hz, 1P), −11.56 (d, J=17.8 Hz, 1P), −11.30 (d, J=17.8 Hz, 1P), −1.21 (s, 1P), −1.00 (s, 1P). ESI-HRMS calculated for C₄₃H₅₈N₁₈O₃₁P₅ 1477.2209 [M-H]⁻, found 1477.2179 [M-H]⁻.

Synthesis of ^{m7}Gppp^{m5}U_{2′OMe}pA_{2′OMe}pG

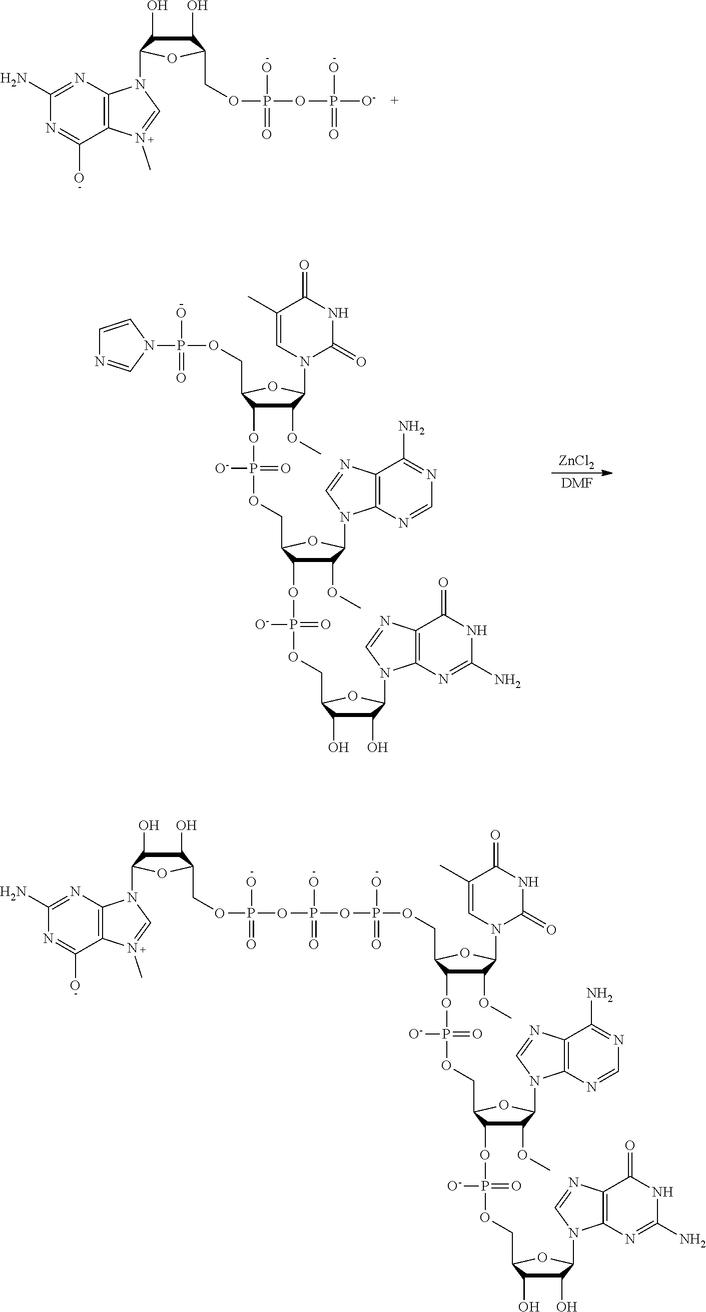

To a solution of p^{m5}U_{2′OMe}A_{2′OMe}pG (50 mg, 35 μmol) in DMF (1 mL), imidazole (24 mg, 346 μmol), 2,2′-dithiodipyridine (46 mg, 208 μmol), TEA (29 μL, 208 μmol) and triphenylphosphine (54 mg, 208 μmol) was added and the mixture was stirred at room temperature for 1 hour. The phosphorimidazolide of p^{m5}U_{2′OMe}pA_{2′OMe}pG was precipitated by addition of sodium perchlorate (42 mg, 346 μmol) in acetone (10 mL). The solution was centrifuged at 4° C. washed with chilled acetone 3 times and dried under reduced pressure to give p^{m5}U_{2′OMe}pA_{2′OMe}pG-Im.

p^{m5}U_{2′OMe}pA_{2′OMe}pG-Im was dissolved in DMF (1 mL), followed by addition of ^{m7}GDP·TEA salt (53 mg, 69 μmol) and ZnCl₂ (24 mg, 173 μmol). The mixture was stirred at room temperature for 18 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (42 mg) in acetone (10 mL). The solution was centrifuged at 4° C. washed with chilled acetone 3 times and dried under reduced pressure to obtain ^{m7}Gppp^{m5}U_{2′OMe}pA_{2′OMe}pG·Na salt (30 mg, 19 μmol, 55% yield).

¹H NMR (700 MHz, D₂O) δ 3.35 (s, 3H), 3.49 (s, 3H), 4.01 (t, J=4.9 Hz, 1H), 4.07 (s, 3H), 4.10-4.24 (m, 9H), 4.31-4.32 (m, 5H), 4.41 (s, 1H), 4.45-4.48 (m, 4H), 4.58 (t, J=4.2 Hz, 1H), 4.70-4.72 (m, 1H), 4.91-4.92 (m, 1H), 5.72 (d, J=5.6 Hz, 1H), 5.81 (d, J=5.6 Hz, 1H), 5.94 (d, J=4.2 Hz, 1H), 6.06 (d, J=4.9 Hz, 1H), 7.58 (s, 1H), 7.92 (s, 1H), 8.15 (s, 1H), 8.35 (s, 1H). ³¹P NMR (162 MHz, D₂O) δ −22.91 (dd, J=19.4 Hz, 17.8 Hz, 1P), −11.78 (d, J=17.8 Hz, 1P), −11.41 (d, J=17.8 Hz, 1P), −1.14 (s, 1P), −0.88 (s, 1P). ESI-HRMS calculated for C₄₃H₅₇N17032P₅ 1478.2049 [M-H]—, found 1478.2014 [M-H]⁻.

Synthesis of ^{m7}GpppA_{2′F}pA_{2′OMe}pG

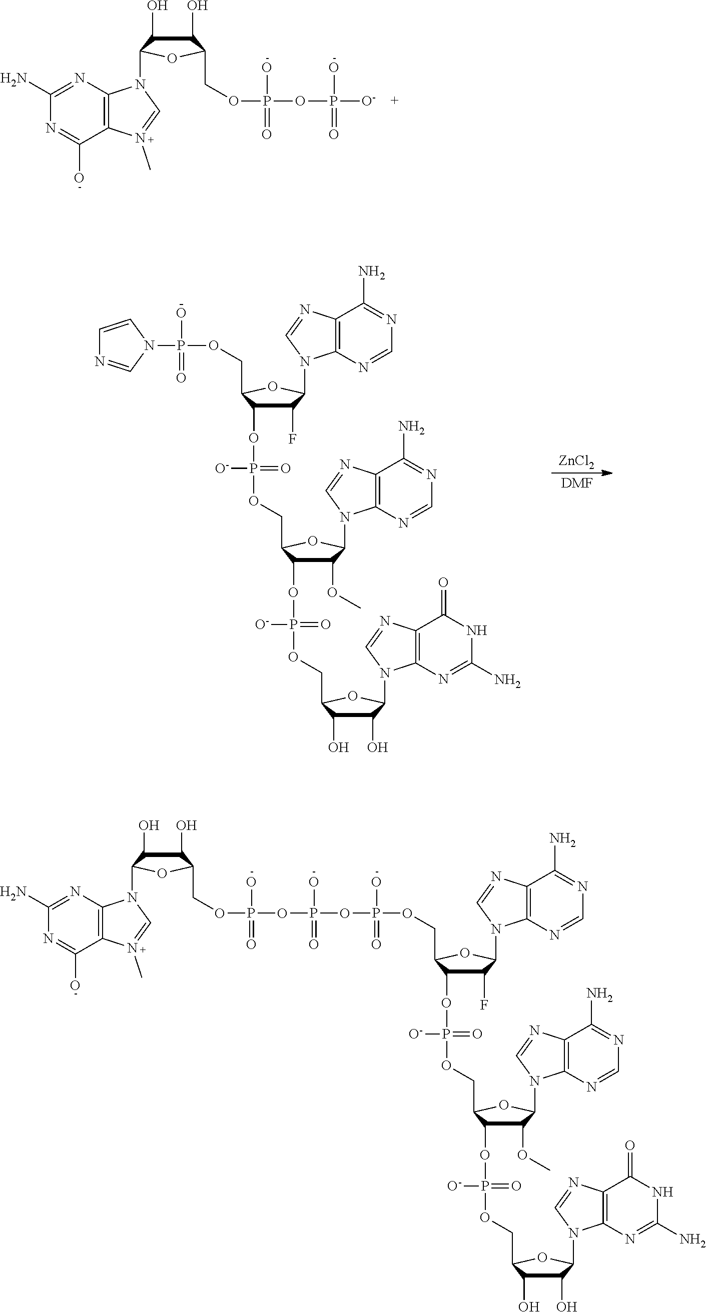

To a solution of pA_{2′F}pA_{2′OMe}pG (38 mg, 26 μmol) in DMF (1 mL), imidazole (18 mg, 263 mol), 2,2′-dithiodipyridine (35 mg, 158 μmol), TEA (22 μL, 158 μmol) and triphenylphosphine (42 mg, 158 μmol) was added and the mixture was stirred at room temperature for 1 hour. The phosphorimidazolide of pA_{2′F}pA_{2′OMe}pG was precipitated by addition of sodium perchlorate (32 mg, 263 μmol) in acetone (10 mL). The solution was centrifuged at 4° C. washed with chilled acetone 3 times and dried under reduced pressure to give pA_{2′F}pA_{2′OMe}pG-Im.

pA_{2′F}pA_{2′OMe}pG-Im was dissolved in DMF (1 mL), followed by addition of ^{m7}GDP·TEA salt (40 mg, 53 μmol) and ZnCl₂ (18 mg, 132 μmol). The mixture was stirred at room temperature for 18 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (32 mg) in acetone (10 mL). The solution was centrifuged at 4° C. washed with chilled acetone 3 times and dried under reduced pressure to obtain ^{m7}GpppA_{2′F}pA_{2′OMe}pG·Na salt (18 mg, 11 μmol, 42% yield).

¹H NMR (700 MHz, D₂O) δ 3.51 (s, 3H), 3.98 (s, 3H), 4.06-4.29 (m, 9H), 4.29-4.30 (m, 1H), 4.35-4.40 (m, 5H), 4.43-4.50 (m, 4H), 5.31 (d, J=53.9 Hz, 1H), 5.66 (d, J=4.9 Hz, 1H), 5.79 (d, J=3.5 Hz, 1H), 5.90 (d, J=4.2 Hz, 1H), 5.92 (d, J=14.7 Hz, 1H), 7.68 (s, 1H), 7.76 (s, 1H), 7.97 (s, 1H), 7.98 (s, 1H), 8.12 (s, 1H). ³¹P NMR (162 MHz, D₂O) δ −22.83 (dd, J=19.4 Hz, 17.8 Hz, 1P), −11.39 (d, J=17.8 Hz, 2P), −1.62 (s, 1P), −0.98 (s, 1P). ESI-HRMS calculated for C₄₃H₅₇N₁₇O₃₂P₅ 1475.1965 [M-H]⁻, found 1475.1969 [M-H]⁻.

Synthesis of ^{m7}GpppA_{2′MOE}A_{2′OMe}pG

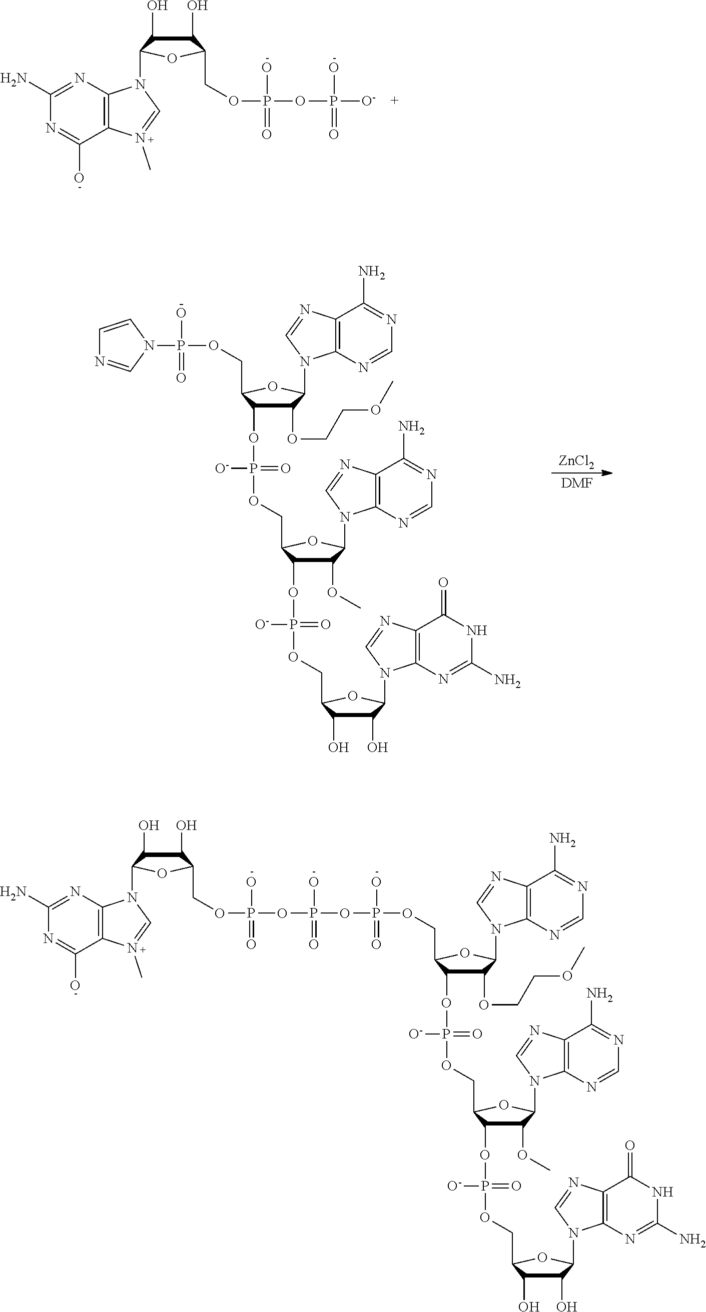

To a solution of pA_{2′MOE}pA_{2′OMe}pG (50 mg, 34 μmol) in DMF (1 mL), imidazole (23 mg, 334 μmol), 2,2′-dithiodipyridine (44 mg, 201 μmol), TEA (28 μL, 201 μmol) and triphenylphosphine (53 mg, 201 μmol) was added and the mixture was stirred at room temperature for 1 hour. The phosphorimidazolide of pA_{2′MOE}pA_{2′OMe}pG was precipitated by addition of sodium perchlorate (41 mg, 334 μmol) in acetone (10 mL). The solution was centrifuged at 4° C. washed with chilled acetone 3 times and dried under reduced pressure to give pA_{2′MOE}pA_{2′OMe}pG-Im.

pA_{2′MOE}pA_{2′OMe}pG-Im was dissolved in DMF (1 mL), followed by addition of ^{m7}GDP·TEA salt (51 mg, 67 μmol) and ZnCl₂ (23 mg, 167 μmol). The mixture was stirred at room temperature for 18 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (32 mg) in acetone (10 mL). The solution was centrifuged at 4° C. washed with chilled acetone 3 times and dried under reduced pressure to obtain ^{m7}GpppA_{2′MOE}pA_{2′OMe}pG·Na salt (29 mg, 18 μmol, 53% yield).

¹H NMR (700 MHz, D₂O) δ 3.13 (s, 3H), 3.42-3.47 (m, 2H), 3.52 (s, 3H), 3.65-3.68 (m, 1H), 3.82-3.84 (m, 1H), 4.14-4.17 (m, 1H), 4.21-4.25 (m, 6H), 4.29-4.36 (m, 5H), 4.41-4.50 (m, 6H), 4.53 (br s, 1H), 4.72 (t, J=4.9 Hz, 2H), 5.80 (dd, J=6.3 Hz, 4.2 Hz, 1H), 5.90 (d, J=4.2 Hz, 1H), 6.01 (d, J=54.9 Hz, 1H), 7.88 (s, 1H), 7.91 (s, 1H), 8.11 (s, 1H), 8.27 (s, 1H), 8.34 (s, 1H). ³¹P NMR (162 MHz, D₂O) δ −22.95 (dd, J=19.4 Hz, 17.8 Hz, 1P), −11.51 (d, J=17.8 Hz, 1P), −11.46 (d, J=19.4 Hz, 1P), −1.01 (s, 1P), −0.91 (s, 1P). ESI-HRMS calculated for C₄₅H₆₀N₂₀O₃₁P₅ 1531.2427 [M-H]⁻, found 1531.2422 [M-H]⁻.

Synthesis of ^{m7}GppprApA_{2′OMe}pG

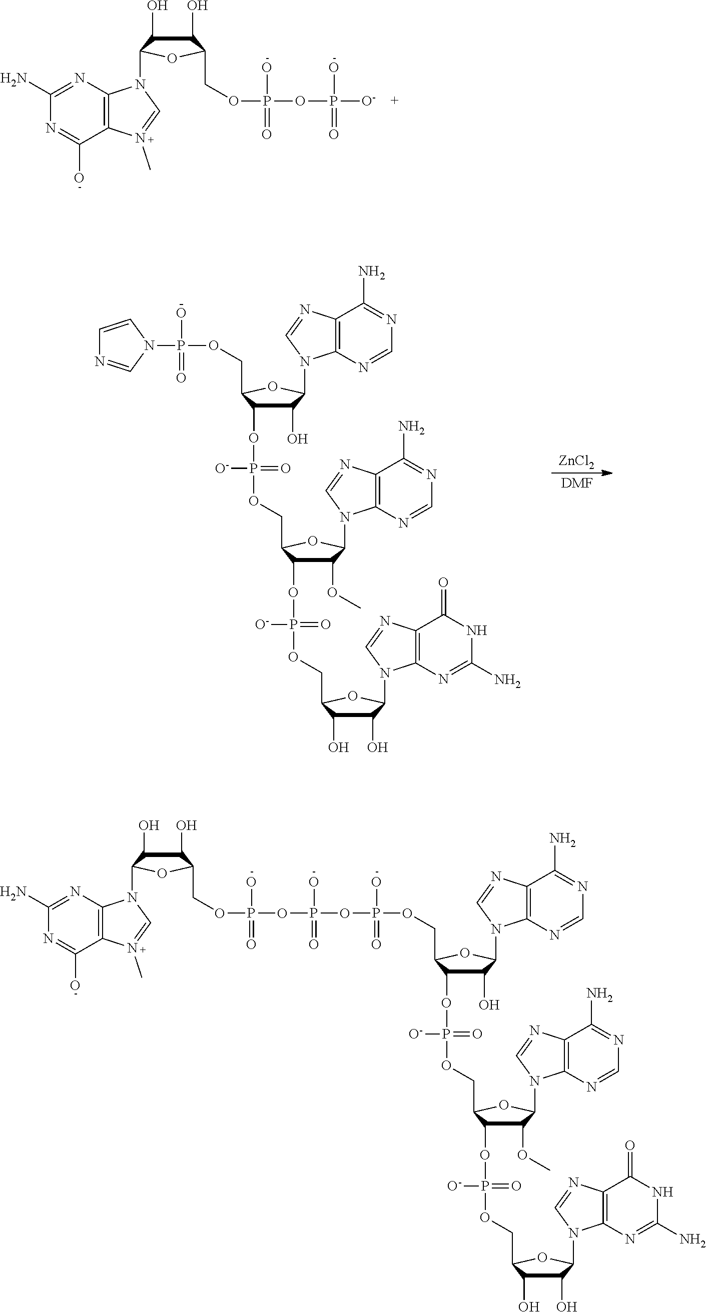

To a solution of prApA_{2′OMe}pG (58 mg, 40 μmol) in DMF (1 mL), imidazole (27 mg, 403 mol), 2,2′-dithiodipyridine (53 mg, 242 μmol), TEA (34 μL, 242 μmol) and triphenylphosphine (33 mg, 242 μmol) was added and the mixture was stirred at room temperature for 1 hour. The phosphorimidazolide of prApA_{2′OMe}pG was precipitated by addition of sodium perchlorate (49 mg, 403 μmol) in acetone (10 mL). The solution was centrifuged at 4° C. washed with chilled acetone 3 times and dried under reduced pressure to give prApA_{2′OMe}pG-Im.

prApA_{2′OMe}pG-Im was dissolved in DMF (1 mL), followed by the addition of ^{m7}GDP·TEA salt (61 mg, 81 μmol) and ZnCl₂ (27 mg, 201 μmol). The mixture was stirred at room temperature for 18 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (32 mg) in acetone (10 mL). The solution was centrifuged at 4° C. washed with chilled acetone 3 times and dried under reduced pressure to obtain ^{m7}GppprApA_{2′OMe}pG·Na salt (27 mg, 17 μmol, 43% yield).

¹H NMR (700 MHz, D₂O) δ 3.50 (s, 3H), 3.99 (s, 3H), 4.12-4.14 (m, 1H), 4.17-4.22 (m, 5H), 4.25 (t, J=4.2 Hz, 1H), 4.28-4.35 (m, 6H), 4.40-4.42 (m, 3H), 4.49-4.51 (m, 2H), 4.64-4.66 (m, 2H), 5.74 (dd, J=5.6 Hz, 4.2 Hz, 2H), 5.81 (d, J=4.2 Hz, 1H), 5.97 (d, J=4.2 Hz, 1H), 7.84 (s, 1H), 7.86 (s, 1H), 8.07 (s, 1H), 8.19 (s, 1H), 8.26 (s, 1H). ³¹P NMR (162 MHz, D₂O) δ −22.87 (dd, J=19.4 Hz, 17.8 Hz, 1P), −11.41 (d, J=19.4 Hz, 1P), −11.36 (d, J=17.8 Hz, 1P), −0.95 (s, 1P), −0.90 (s, 1P). ESI-HRMS calculated for C₄₂H₅₄N₂₀O₃₀P₅ 1473.2008 [M-H]⁻, found 1473.1930 [M-H]—.

Synthesis of ^{m7}G_{3′OMe} ppp^{m6}A_{2′OMe} pA_{2′OMe}pG

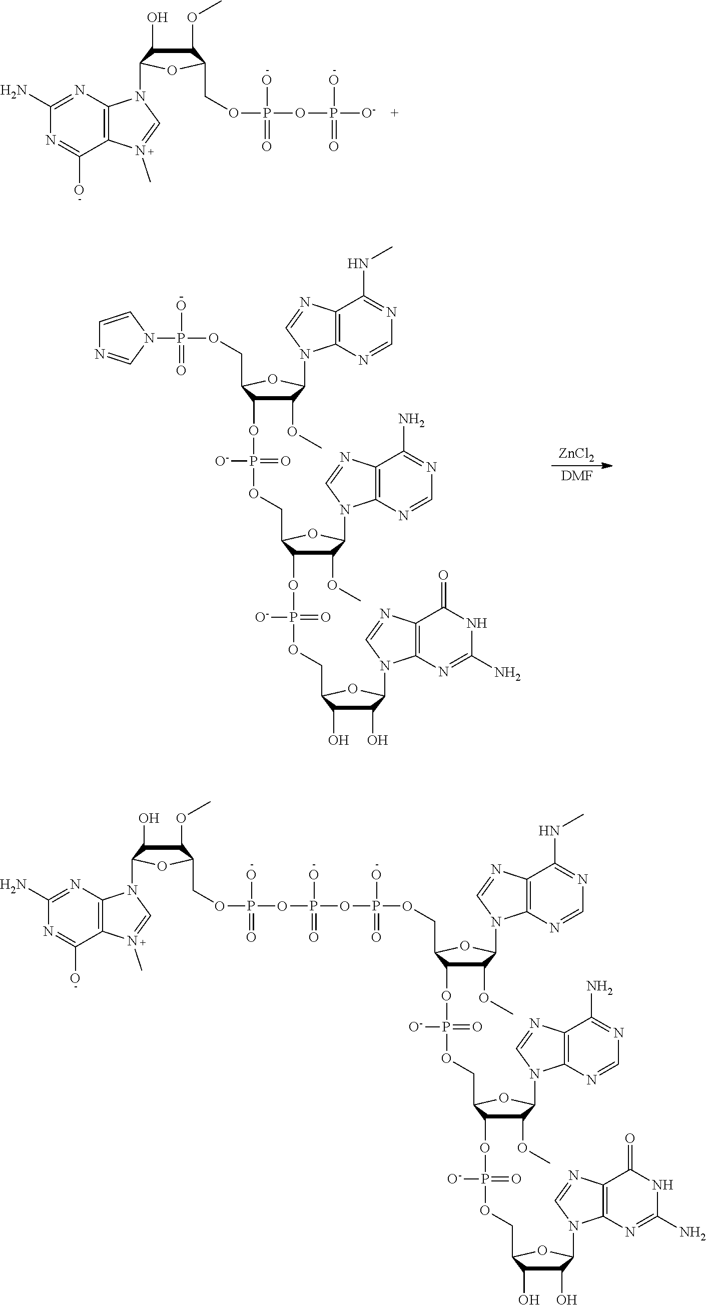

To a solution of p^{m6}A_{2′OMe}pA_{2′OMe}pG (8 mg, 5.5 μmol) in DMF (0.1 mL), imidazole (3.7 mg, 54 μmol), 2,2′-dithiodipyridine (7.2 mg, 33 μmol), TEA (4.6 μL, 33 μmol) and triphenylphosphine (8.6 mg, 33 μmol) was added and the mixture was stirred at room temperature for 1 hour. The phosphorimidazolide of p^{m6}A_{2′OMe}pA_{2′OMe}pG was precipitated by addition of sodium perchlorate (6.7 mg, 55 μmol) in acetone (10 mL). The solution was centrifuged at 4° C. washed with chilled acetone 3 times and dried under reduced pressure to give p^{m6}A_{2′OMe}pA_{2′OMe}pG-Im.

p^{m6}A_{2′OMe}pA_{2′OMe}pG-Im was dissolved in DMF (0.1 mL) and DMSO (50 L), followed by the addon of ^{m7}G_{3′OMe} DP·TEA salt (12.6 mg, 16 μmol) and ZnCl₂ (3.7 mg, 27 μmol). The mixture was stirred at room temperature for 18 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (6.7 mg) in acetone (10 mL). The solution was centrifuged at 4° C. washed with chilled acetone 3 times and dried under reduced pressure to obtain ^{m7}G_{3′OMe} ppp^{m6}A_{2′OMe}pA_{2′OMe}pG·Na salt (5.0 mg, 3.1 μmol, 56% yield).

¹H NMR (700 MHz, D₂O) δ 3.35 (s, 3H), 3.43 (s, 3H), 3.52 (s, 3H), 3.56 (s, 3H), 3.97 (s, 3H), 4.02 (t, J=4.9 Hz, 1H), 4.10-4.15 (m, 2H), 4.18-4.26 (m, 6H), 4.30-4.34 (m, 3H), 4.41-4.45 (m, 3H), 4.49 (br d, J=3.5 Hz, 1H), 4.56 (br s, 1H), 4.61-4.62 (m, 1H), 4.65 (dd, J=5.6 Hz, 4.9 Hz, 1H), 5.70 (d, J=4.2 Hz, 1H), 5.74 (d, J=4.9 Hz, 1H), 5.83 (d, J=4.9 Hz, 1H), 5.96 (d, J=3.5 Hz, 1H), 7.82 (s, 1H), 7.83 (s, 1H), 8.06 (s, 1H), 8.14 (s, 1H), 8.26 (s, 1H). ³¹P NMR (162 MHz, D₂O) δ −22.78 (dd, J=19.4 Hz, 17.8 Hz, 1P), −11.49 (d, J=19.4 Hz, 1P), −11.42 (d, J=17.8 Hz, 1P), −1.08 (s, 1P), −0.98 (s, 1P). ESI-HRMS calculated for C₄₅H₆₀N₂₀O₃₀P₅ 1515.2478 [M-H]⁻, found 1515.2501 [M-H]⁻.

Synthesis of ^{m7}G_{3′OMe}pppA_{2′OMe}pA_{2′OMe}pA_{2′OMe}pG

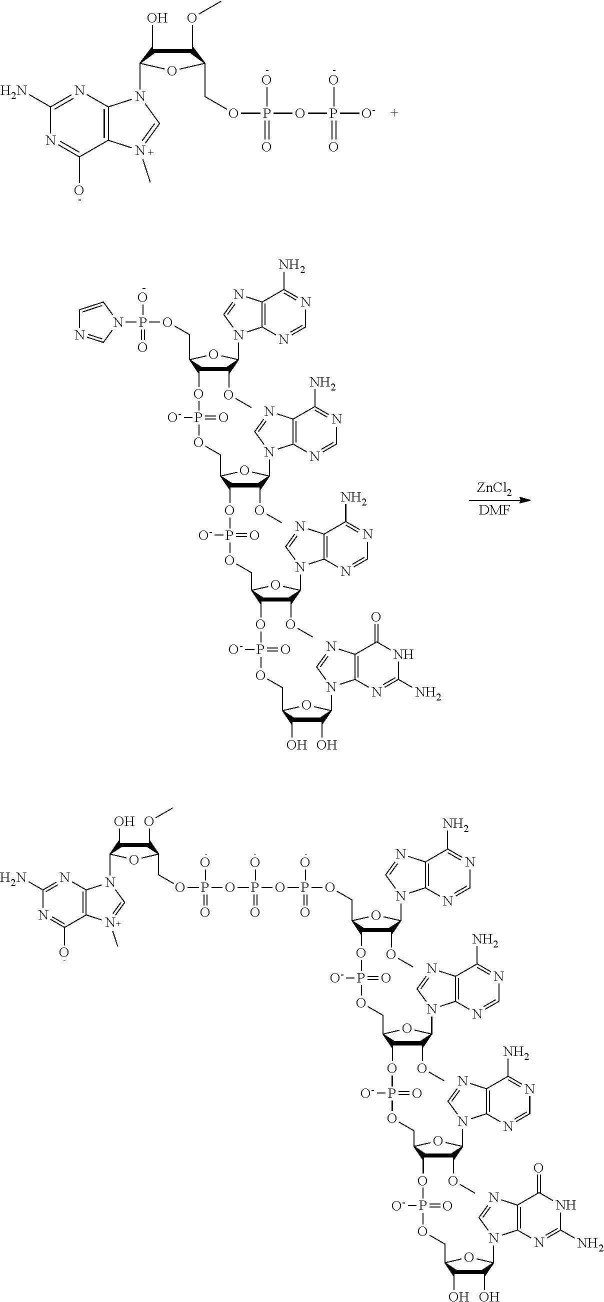

To a solution of pA_{2′OMe}pA_{2′OMe}pA_{2′OMe}pG (143 mg, 75 μmol) in DMF (0.5 mL), imidazole (51 mg, 753 μmol), 2,2′-dithiodipyridine (100 mg, 452 μmol), TEA (63 μL, 452 μmol) and triphenylphosphine (119 mg, 452 μmol) was added and the mixture was stirred at room temperature for 1 hour. The phosphorimidazolide of pA_{2′OMe}pA_{2′OMe}pA_{2′OMe}pG was precipitated by addition of sodium perchlorate (92 mg, 753 μmol) in acetone (10 mL). The solution was centrifuged at 4° C. washed with chilled acetone 3 times and dried under reduced pressure to give pA_{2′OMe}pA_{2′OMe}pA_{2′OMe}pG-Im.

pA_{2′OMe}pA_{2′OMe}pA_{2′OMe}pG-Im was dissolved in DMF (0.5 mL) and DMSO (1 mL), followed by addition of ^{m7}G_{3′OMe}DP·TEA salt (116 mg, 151 μmol) and ZnCl₂ (51 mg, 377 μmol). The mixture was stirred at room temperature for 18 hours, and the reaction was quenched by addition of 0.5 M EDTA aq. and 0.1 M TEAA. The tetranucleotide product was purified by RP-HPLC (Yamazen Flush ODS Premium L size, Buffer A: 0.1M TEAA, Buffer B: ACN, linear gradient 1-30% of Buffer B over 20 min.). The isolated product was concentrated by evaporator and precipitated by sodium perchlorate (92 mg) in acetone (10 mL). The solution was centrifuged at 4° C. washed with chilled acetone 3 times and dried under reduced pressure to obtain ^{m7}G_{3′OMe}pppA_{2′OMe}pA_{2′OMe}pA_{2′OMe}pG·Na salt (67 mg, 34 μmol, 45% yield).

¹H NMR (700 MHz, D₂O) δ 3.46 (s, 3H), 3.49 (s, 3H), 3.56 (s, 3H), 3.59 (s, 3H), 3.89 (s, 3H), 4.10-4.30 (m, 12H), 4.35-4.36 (m, 3H), 4.40 (t, J=4.9 Hz, 1H), 4.43-4.45 (m, 2H), 4.48-4.49 (m, 1H), 4.49-4.51 (m, 2H), 4.64-4.66 (m, 2H), 4.54 (br s, 1H), 4.57 (dd, J=5.6 Hz, 4.9 Hz, 1H), 4.67 (dd, J=5.6 Hz, 4.9 Hz, 1H), 4.95-4.97 (m, 1H), 5.72 (d, J=4.9 Hz, 1H), 5.85 (d, J=4.2 Hz, 1H), 5.94 (d, J=2.1 Hz, 1H), 6.00 (d, J=5.6 Hz, 1H), 6.01 (br s, 1H), 7.82 (s, 1H), 7.86 (s, 1H), 7.92 (s, 1H), 8.01 (s, 2H), 8.31 (s, 1H), 8.36 (s, 1H). ³¹P NMR (162 MHz, D₂O) δ −21.58 (dd, J=16.2 Hz, 14.6 Hz, 1P), −11.43 (d, J=16.2 Hz, 1P), −10.94 (d, J=16.2 Hz, 1P), −1.31 (s, 1P), −1.26 (s, 1P), −1.00 (s, 1P). ESI-HRMS calculated for C₅₅H₇₂N₂₅O₃₆P₆ 1844.3003 [M-H]⁻, found 1844.3033 [M-H]⁻.

Synthesis of ^{m7}GpppG_{Ina}pA_{2′OMe}pG

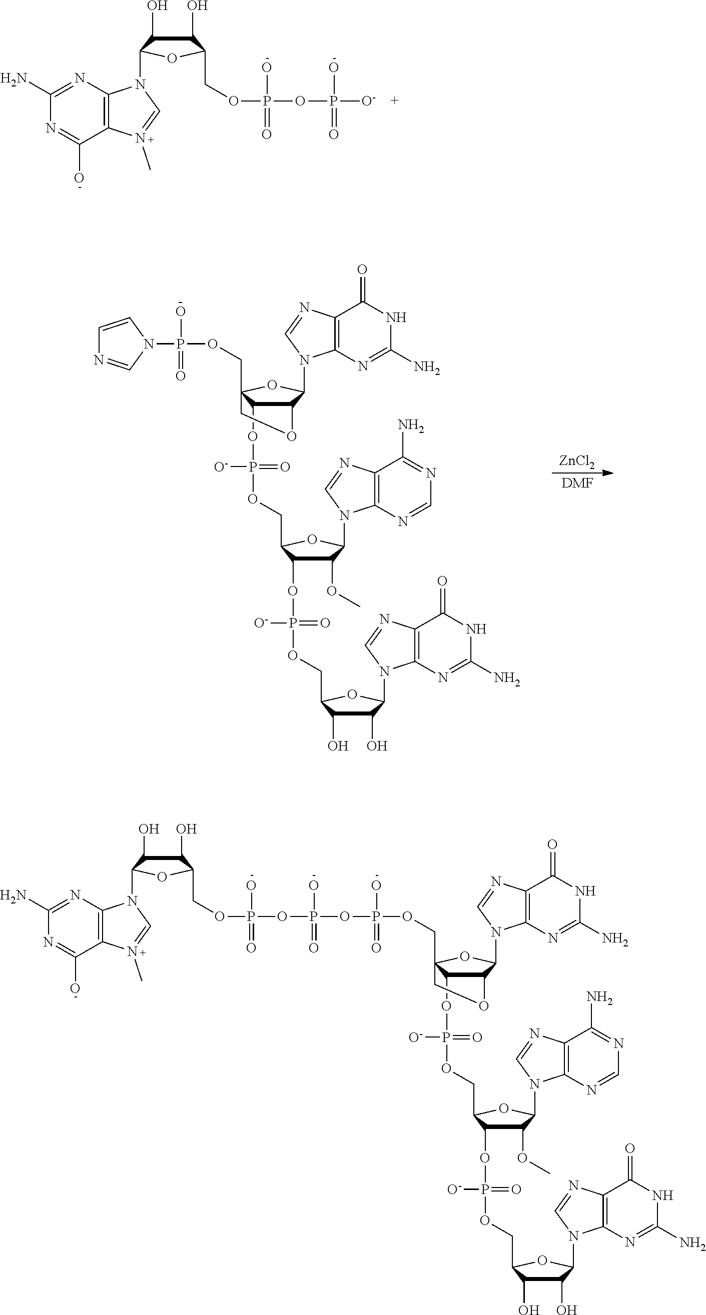

To a solution of pG_{Ina}pA_{2′OMe}pG (26 mg, 18 μmol) in DMF (2 mL), imidazole (12 mg, 175 mol), 2,2′-dithiodipyridine (23 mg, 105 μmol), TEA (15 μL, 105 μmol) and triphenylphosphine (28 mg, 105 μmol) was added and the mixture was stirred at room temperature for 5.5 hour. The phosphorimidazolide of pG_{Ina}pA_{2′OMe}pG was precipitated by addition of sodium perchlorate (31 mg, 253 μmol) in acetone (15 mL). The solution was centrifuged at 4° C. washed with chilled acetone 3 times and dried under reduced pressure to give pG_{Ina}pA_{2′OMe}pG-Im.

pG_{Ina}pA_{2′OMe}pG-Im was dissolved in DMF (0.5 mL), followed by addition of ^{m7}GDP·TEA salt (27 mg, 35 μmol) and ZnCl₂ (12 mg, 175 μmol). The mixture was stirred at room temperature for 19 hours, and the reaction was quenched by addition of 0.5 M EDTA aq. and 0.1 M TEAA. The tetranucleotide product was purified by RP-HPLC (Yamazen Flush ODS Premium M size, Buffer A: 0.1M TEAA, Buffer B: ACN, linear gradient 1-20% of Buffer B over 20 min.). The isolated product was concentrated by evaporator and precipitated by sodium perchlorate (22 mg) in acetone (10 mL). The solution was centrifuged at 4° C. washed with chilled acetone 3 times and dried under reduced pressure to obtain ^{m7}GpppG_{Ina}pA_{2′OMe}pG·Na salt (11 mg, 6.7 μmol, 38% yield).

¹H NMR (700 MHz, D₂O) δ 3.55 (s, 3H), 4.05 (s, 3H), 4.06-4.11 (m, 3H), 4.13-4.15 (m, 1H), 4.21-4.23 (m, 2H), 4.26-4.29 (m, 2H), 4.33-4.41 (m, 7H), 4.45 (d, J=6.3 Hz, 2H), 4.55 (dd, J=5.6 Hz, 4.9 Hz, 2H), 4.64 (d, J=4.9 Hz, s, 1H), 4.68 (s, 1H), 5.21 (s, 1H), 5.69 (d, J=5.6 Hz, 1H), 5.74 (d, J=2.8 Hz, 1H), 6.00 (s, 1H), 7.63 (s, 1H), 7.78 (s, 1H), 8.05 (s, 1H). ³¹P NMR (162 MHz, D₂O) δ −22.87 (dd, J=19.4 Hz, 17.8 Hz, 1P), −11.44 (d, J=16.2 Hz, 2P), −2.41 (s, 1P), −1.05 (s, 1P). ESI-HRMS calculated for C₄₃H₅₄N₂₀O₃₁P₅ 1501.1957 [M-H]⁻, found 1501.1970 [M-H]⁻.

Synthesis of ^{m7}GpppG_{2′OMe}pSA_{2′OMe}psG

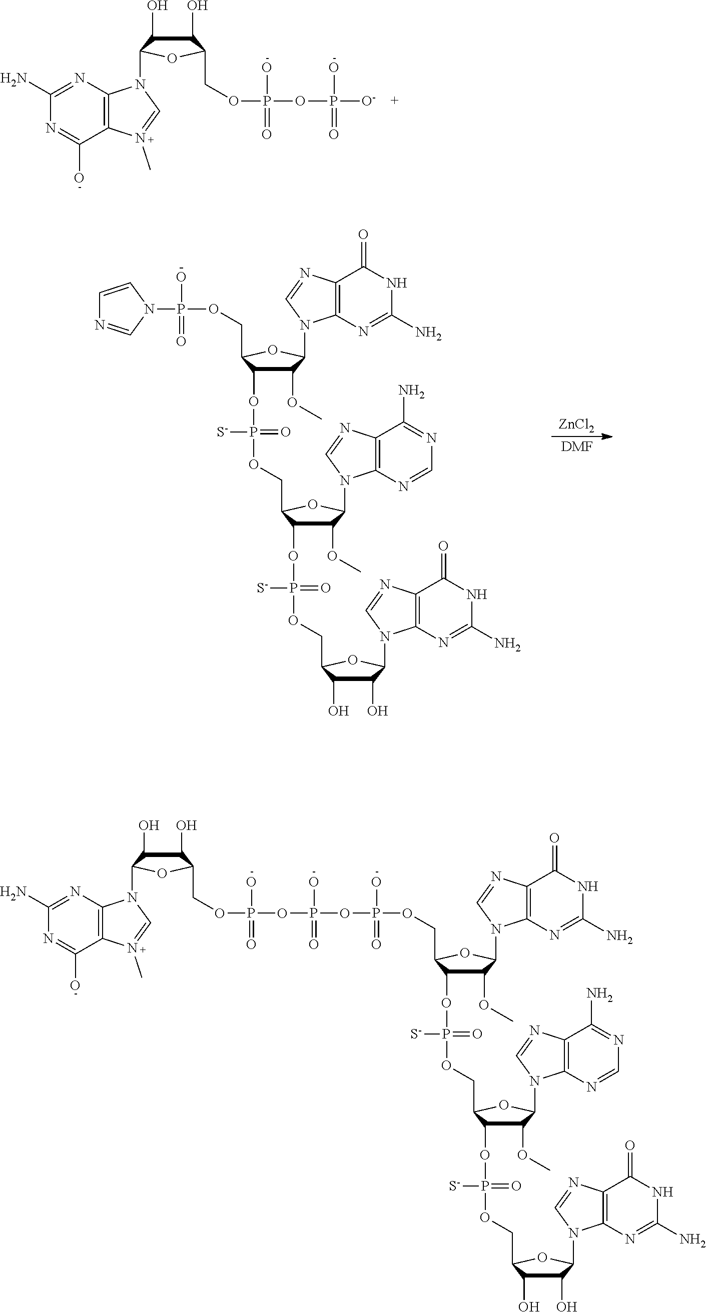

To a solution of pG_{2′OMe}psA_{2′OMe}psG (25 mg, 17 μmol) in DMF (0.5 mL), imidazole (12 mg, 169 μmol), 2,2′-dithiodipyridine (22 mg, 101 μmol), TEA (14 μL, 101 μmol) and triphenylphosphine (27 mg, 101 μmol) was added and the mixture was stirred at room temperature for 1 hour. The phosphorimidazolide of pG_{2′OMe}psA_{2′OMe}psG was precipitated by addition of sodium perchlorate (31 mg, 253 μmol) in acetone (15 mL). The solution was centrifuged at 4° C. washed with chilled acetone 3 times and dried under reduced pressure to give pG_{2′OMe}psA_{2′OMe}psG-Im.

pG_{2′OMe}psA_{2′OMe}psG-Im was dissolved in DMF (0.5 mL), followed by addition of ^{m7}GDP·TEA salt (26 mg, 34 μmol) and ZnCl₂ (12 mg, 85 μmol). The mixture was stirred at room temperature for 18 hours, and the reaction was quenched by addition of 0.5 M EDTA aq. and 0.1 M TEAA. The tetranucleotide product was purified by RP-HPLC (Yamazen Flush ODS Premium M size, Buffer A: 0.1M TEAA, Buffer B: ACN, linear gradient 1-20% of Buffer B over 20 min.). The isolated product was concentrated by evaporator and precipitated by sodium perchlorate (21 mg) in acetone (10 mL). The solution was centrifuged at 4° C. washed with chilled acetone 3 times and dried under reduced pressure to obtain ^{m7}GpppG_{2′OMe}psA_{2′OMe}psG·Na salt (22 mg, 13 μmol, 79% yield).

³¹P NMR (162 MHz, D₂O) δ −22.78 (d, J=19.4 Hz), −11.42 (d, J=17.8 Hz, 1P), −11.37 (d, J=17.8 Hz, 1P), 54.84 (s, 1P), 55.04 (s, 1P), 56.06 (s, 1P), 56.26 (s, 1P), 56.37 (s, 1P), 56.52 (s, 1P), 56.62 (s, 1P), 56.76 (s, 1P). ESI-HRMS calculated for C₄₃H₅₆N₂₀O₂₅P₅S₂1535.1657 [M-H]⁻, found 1535.1611 [M-H]⁻.

Synthesis of ^{m7}GpppG_{2′OMe}pA_{2′OMe}pA_{2′OMe}pG

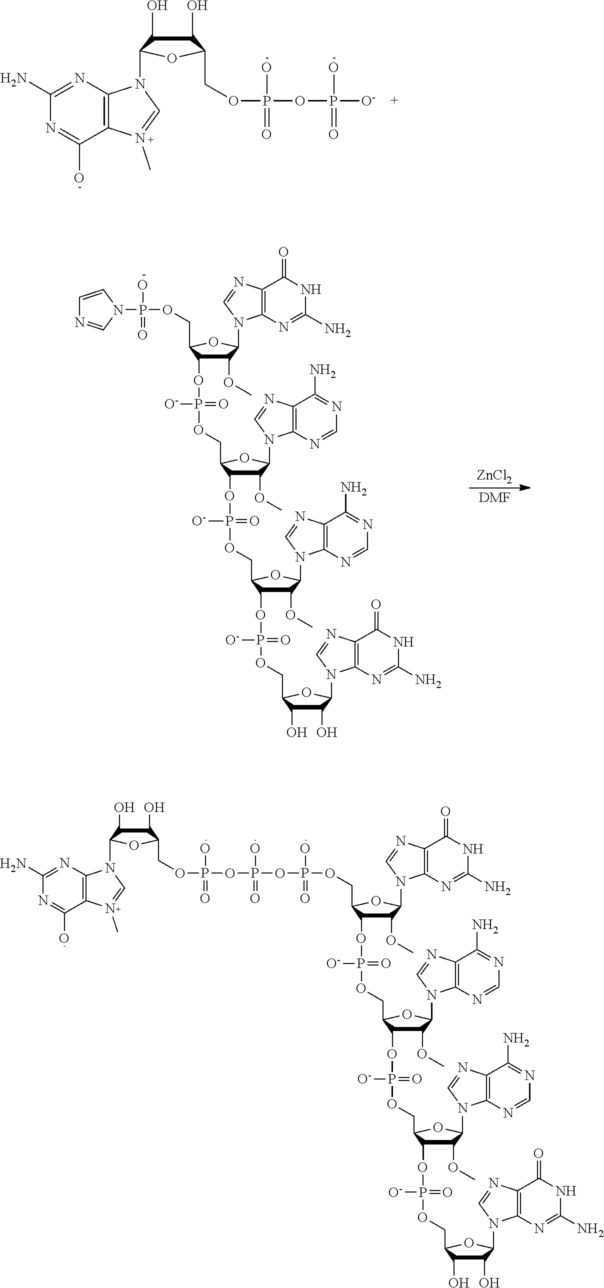

To a solution of pG_{2′OMe}pA_{2′OMe}pA_{2′OMe}pG (49 mg, 26 μmol) in DMF (4 mL), imidazole (18 mg, 256 μmol), 2,2′-dithiodipyridine (34 mg, 154 μmol), TEA (21 μL, 154 μmol) and triphenylphosphine (40 mg, 154 μmol) was added and the mixture was stirred at room temperature for 1 hour. The phosphorimidazolide of pG_{2′OMe}pA_{2′OMe}pA_{2′OMe}pG was precipitated by addition of sodium perchlorate (31 mg, 253 μmol) in acetone (20 mL). The solution was centrifuged at 4° C. washed with chilled acetone 3 times and dried under reduced pressure to give pG_{2′OMe}pA_{2′OMe}pA_{2′OMe}pG-Im.

pG_{2′OMe}pA_{2′OMe}pA_{2′OMe}pG-Im was dissolved in DMF (1 mL), followed by addition of ^{m7}GDP·TEA salt (39 mg, 51 μmol) and ZnCl₂ (18 mg, 256 μmol). The mixture was stirred at room temperature for 19 hours, and the reaction was quenched by addition of 0.5 M EDTA aq. and 0.1 M TEAA. The tetranucleotide product was purified by RP-HPLC (Yamazen Flush ODS Premium M size, Buffer A: 0.1M TEAA, Buffer B: ACN, linear gradient 1-20% of Buffer B over 20 min.). The isolated product was concentrated by evaporator and precipitated by sodium perchlorate (62 mg) in acetone (10 mL). The solution was centrifuged at 4° C. washed with chilled acetone 3 times and dried under reduced pressure to obtain ^{m7}GpppG_{2′OMe}pA_{2′OMe}pA_{2′OMe}pG·Na salt (28 mg, 14 μmol, 55% yield).

¹H NMR (700 MHz, D₂O) δ 3.44 (s, 3H), 3.47 (s, 3H), 3.49 (s, 3H), 3.94 (s, 3H), 4.05-4.07 (m, 1H), 4.09-4.19 (m, 8H), 4.20-4.21 (m, 1H), 4.22-4.27 (m, 4H), 4.31-4.35 (m, 3H), 4.37-4.41 (m, 5H), 4.59 (dd, J=5.6 Hz, 4.9 Hz, 2H), 5.53 (d, J=6.3 Hz, 1H), 5.70 (d, J=3.5 Hz, 1H), 5.72 (d, J=5.6 Hz, 1H), 5.90 (d, J=4.2 Hz, 1H), 5.93 (d, J=3.5 Hz, 1H), 7.83 (s, 1H), 7.85 (s, 1H), 7.91 (s, 1H), 8.01 (s, 1H), 8.09 (s, 1H), 8.16 (s, 1H). ³¹P NMR (162 MHz, D₂O) δ −22.93 (t, J=17.8 Hz, 1P), −11.44 (d, J=19.4 Hz, 1P), −11.40 (d, J=16.2 Hz, 1P), −1.15 (s, 1P), −0.91 (s, 1P), −0.82 (s, 1P). ESI-HRMS calculated for C₅₄H₇₀N₂₅O₃₇P₆ 1846.2796 [M-H]⁻, found 1846.2728 [M-H]⁻.

Synthesis of ^{m7}GpppU_{2′OMe}pA_{2′OMe}pA_{2′OMe}pG

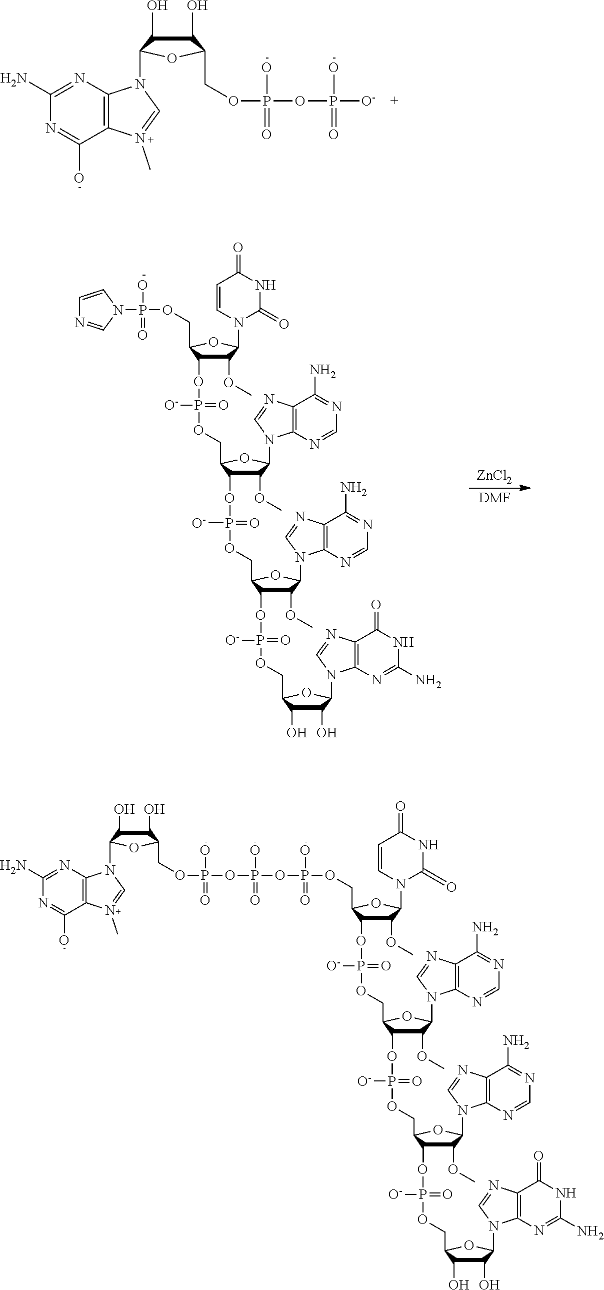

To a solution of pU_{2′OMe}pA_{2′OMe}pA_{2′OMe}pG (35 mg, 19 μmol) in DMF (1.5 mL), imidazole (13 mg, 187 μmol), 2,2′-dithiodipyridine (25 mg, 112 μmol), TEA (16 μL, 112 μmol) and triphenylphosphine (29 mg, 112 μmol) was added and the mixture was stirred at room temperature for 1 hour. The phosphorimidazolide of pU_{2′OMe}pA_{2′OMe}pA_{2′OMe}pG was precipitated by addition of sodium perchlorate (92 mg, 753 μmol) in acetone (15 mL). The solution was centrifuged at 4° C. washed with chilled acetone 3 times and dried under reduced pressure to give pU_{2′OMe}pA_{2′OMe}pA_{2′OMe}pG-Im.

pU_{2′OMe}pA_{2′OMe}pA_{2′OMe}pG-Im was dissolved in DMF (0.5 mL), followed by addition of ^{m7}GDP·TEA salt (28 mg, 37 μmol) and ZnCl₂ (13 mg, 93 μmol). The mixture was stirred at room temperature for 18 hours, and the reaction was quenched by addition of 0.5 M EDTA aq. and 0.1 M TEAA. The tetranucleotide product was purified by RP-HPLC (Yamazen Flush ODS Premium M size, Buffer A: 0.1M TEAA, Buffer B: ACN, linear gradient 1-20% of Buffer B over 20 min.). The isolated product was concentrated by evaporator and precipitated by sodium perchlorate (23 mg) in acetone (10 mL). The solution was centrifuged at 4° C. washed with chilled acetone 3 times and dried under reduced pressure to obtain ^{m7}GpppU_{2′OMe}pA_{2′OMe}pA_{2′OMe}pG·Na salt (21 mg, 11 μmol, 57% yield).

¹H NMR (700 MHz, D₂O) δ 3.34 (s, 3H), 3.46 (s, 3H), 3.48 (s, 3H), 3.98 (t, J=4.9 Hz, 1H), 3.23 (s, 3H), 4.10 (br s, 3H), 4.11-4.13 (m, 1H), 4.14-4.18 (m, 5H), 4.28-4.29 (m, 6H), 4.36 (br s, 1H), 4.38-4.45 (m, 6H), 4.56 (t, J=4.2 Hz, 1H), 4.62 (dd, J=5.6 Hz, 4.9 Hz, 1H), 5.71 (d, J=4.9 Hz, 1H), 5.73 (d, J=4.9 Hz, 1H), 5.85 (d, J=8.4 Hz, 1H), 5.91 (d, J=3.5 Hz, 1H), 5.95 (d, J=4.2 Hz, 1H), 5.96 (d, J=3.5 Hz, 1H), 7.77 (d, J=8.4 Hz, 1H), 7.83 (s, 1H), 7.89 (s, 1H), 8.03 (s, 1H), 8.17 (s, 2H), 8.28 (s, 1H). ³¹P NMR (162 MHz, D₂O) δ −22.91 (dd, J=19.4 Hz, 16.2 Hz, 1P), −11.57 (d, J=16.2 Hz, 1P), −11.38 (d, J=19.4 Hz, 1P), −1.10 (s, 2P), −0.87 (s, 1P). ESI-HRMS calculated for C₅₃H₆₉N₂₂O₃₈P₆ 1807.2574 [M-H]⁻, found 1807.2494 [M-H]⁻.

FIGS. 1A-1D show structures for exemplary tetranucleotide 5′-capping primers having Cap-2 structures used in the Examples. FIG. 1A shows the structure for ^{m7}GpppA_{2′OMe}pA_{2′OMe}pG. FIG. 1B shows the structure for ^{m7}G_{3′OMe}pppA_{2′OMe}pA_{2′OMe}pG. FIG. 1C shows the structure for ^{m7}GpppU_{2′OMe}pA_{2′OMe}pG. FIG. 1D shows the structure for ^{m7}GpppA_{2′OMe}pG_{2′OMe}pG. FIG. 1E shows the structure for ^{m7}GpppC_{2′OMe}pA_{2′OMe}pG. FIG. 1F shows the structure for ^{m7}GpppG_{2′OMe}pA_{2′OMe}pG. FIG. 1G shows the structure for ^{m7}GpppA_{2′OMe}pC_{2′OMe}pG. FIG. 1H shows the structure for ^{m7}GpppA_{2′OMe}pU_{2′OMe}pG. FIG. 1I shows the structure for ^{m7}GpppA_{2′OMe}pA_{2′OMe}pA. FIG. 1J shows the structure for ^{m7}GpppA_{2′OMe}pA_{2′OMe}pC. FIG. 1K shows the structure for ^{m7}GpppA_{2′OMe}pA_{2′OMe}pU. FIG. 1L shows the structure for ^{m7}GpppdTpA_{2′OMe}pG. FIG. 1M shows the structure for ^{m7}GpppI_{2′OMe}pA_{2′OMe}pG. FIG. 1N shows the structure for ^{m7}GpppA_{2′F}pA_{2′OMe}pG. FIG. 1O shows the structure for ^{m7}GpppA_{2′OMe}A_{2′OMe}pG. FIG. 1P shows the structure for ^{m7}GppprApA_{2′OMe}pG. FIG. 1Q shows the structure for ^{m7}Gppp^{m5}C_{2′OMe}pA_{2′OMe}pG. FIG. 1R shows the structure for ^{m7}Gppp^{m5}U_{2′OMe}pA_{2′OMe}pG. FIG. 1S shows the structure for ^{m7}G_{3′OMe}ppp^{m6}A_{2′OMe}pA_{2′OMe}pG. FIG. 1T shows the structure for ^{m7}G_{3′OMe}pppA_{2′OMe}pA_{2′OMe}pA_{2′OMe}pG. FIG. 1U shows the structure for ^{m7}GpppG_{Ina}pA_{2′OMe}pG. FIG. 1V shows the structure for ^{m7}GpppG_{2′OMe}psA_{2′OMe}psG. FIG. 1W shows the structure for ^{m7}GpppG_{2′OMe}pA_{2′OMe}pA_{2′OMe}pG. FIG. 1X shows the structure for ^{m7}GpppU_{2′OMe}pA_{2′OMe}pA_{2′OMe}pG.

### Example 2: In Vitro Transcription (IVT) with Exemplary 5′-Capping Primers

The exemplary capping primers described in Example 1 were evaluated for capping efficiency in an IVT reaction to generate mRNA using a TC template. Comparison was made to the IVT reaction using a DNA template having a cytidine nucleobase at position +1 and position +2 (“CC template”).

### (i) DNA Synthesis of TC Template for In Vitro Transcription

To prepare the template plasmid DNA for in vitro transcription (IVT), a plasmid containing the T7 promoter sequence, 5′-UTR sequence, KOZAK sequence, each ORF sequence, and 3′-UTR sequence in order was synthesized.

For the CC template, the −1 template nucleotide was 2′-deoxythymidine, the +1 template nucleotide was 2′-deoxycytidine, and the +2 template nucleotide was 2′-deoxycytidine.

For the TC template (first version, referred to as the “TC template”), the −1 template nucleotide was 2′-deoxythymidine, the +1 template nucleotide was 2′-deoxythymidine, and the +2 template nucleotide was 2′-deoxycytidine.

For the TC template (second version, referred to as the “TCφ2.5 template”), the −1 template nucleotide was 2′-deoxyadenosine, the +1 template nucleotide was 2′-deoxythymidine, and the +2 template nucleotide was 2′-deoxycytidine.

Sequence information for the DNA templates evaluated in this Example are identified in Table 6.

TABLE 6
Sense Strand Sequences for Exemplary DNA Templates

| | T7 promoter sequence | RNA-coding region |
|---|---|---|
| | (SEQ ID NO) | (SEQ ID NO) |
| CC Template | 1 | 2 |
| TC Template | 1 | 3 |
| TCφ2.5 template | 4 | 5 |

The plasmid (1 μg) was dissolved in nuclease-free water (210 μL), and Q5 Hot Start High-Fidelity 2× Master Mix (250 μL, NEB #M0494L), 10 μM sense primer (20 μL), and 10 μM antisense primer containing poly-T (20 μL) were added. After incubation at 95° C. for 1 minute, the mixture was subjected to 35 cycles of 95° C. for 30 seconds, 60° C. for 30 seconds, and 72° C. for 3 minutes, followed by a final incubation at 72° C. for 5 minutes to amplify the template DNA by PCR. After the reaction, isopropanol (500 μL) was added, and the mixture was left at −20° C. for over 1 hour. It was then centrifuged (4° C., 15000 rpm, 30 minutes), and the supernatant was discarded. This precipitate was further washed by 75% ethanol three times. The resulting precipitate was dissolved in nuclease-free water to obtain the template DNA containing the ORF of Firefly luciferase.

### (ii) In Vitro Transcription (IVT)

mRNA was prepared by IVT using the DNA templates described above. The IVT reaction was performed using the following conditions.

### Condition 1—Equimolar GTP

The following conditions combine primer and NTPs at a molar ratio of 1 to 1.

The IVT reaction was performed by mixing 50 g/mL template DNA, 5 mM Cap analog, 5 mM ATP (TriLink catalog #N-1510), 5 mM CTP (TriLink catalog #N-1511), 5 mM GTP (TriLink catalog #N-1512), 5 mM N1-methyl-W-uridine-5′-triphosphate (^{1m}ψTP), 40 mM Tris-HCl (pH 8), 10 mM DTT, 2 mM Spermidine, 0.002% Triton, 30 mM Mg(OAc)₂, 1000 U/μL RNase inhibitor (NEB, catalog #M0314L), 2 U/μL Yeast Inorganic pyrophosphatase (32 μL, NEB, catalog #M2403L), and T7 RNA Polymerase (Roche catalog #08140669103). The mixture was incubated at 37° C. for 3 hours. Following this, RNase-Free DNase I (6 U/μg template DNA, TaKaRa catalog #2270A) was added and the mixture was incubated at 37° C. for 30 minutes. Subsequently, 10× phosphatase buffer (NEB, catalog #B0289S) and 0.25 U/μL Antarctic phosphatase (NEB, catalog #M0289L) were added and incubated at 37° C. for 30 minutes. An 8 M LiCl solution (Sigma-Aldrich catalog #L7026) was added to the reaction mixture to a final concentration of 8/3 M, and the mixture was left at −20° C. for over 1 hour. After centrifugation (4° C., 15000 rpm, 30 minutes), the supernatant was discarded. The pellet was washed three times with 75% ethanol, followed by centrifugation (4° C., 15000 rpm, 5 minutes) and discarding the supernatant each time. The resulting pellet was dissolved in nuclease-free water. The mRNA yield was quantified by measuring absorbance at 260 nm.

### Condition 2—Low GTP

The following conditions combine capping primer, GTP, and NTPs at a molar ratio of 3 to 1 to 4.

The IVT reaction was performed by mixing 50 g/mL template DNA, 1.5 mM Cap analog, 2 mM ATP (TriLink catalog #N-1510), 2 mM CTP (TriLink catalog #N-1511), 0.5 mM GTP (TriLink catalog #N-1512), 2 mM N1-methyl-W-uridine-5′-triphosphate (^{1m}ψTP), 40 mM Tris-HCl (pH 8), 10 mM DTT, 2 mM Spermidine, 0.002% Triton, 30 mM Mg(OAc)₂, 1000 U/μL RNase inhibitor (NEB, catalog #M0314L), 2 U/μL Yeast Inorganic pyrophosphatase (32 μL, NEB, catalog #M2403L), and T7 RNA Polymerase (Roche catalog #08140669103). The mixture was incubated at 37° C. for 3 hours. Following this, RNase-Free DNase I (6 U/μg template DNA, TaKaRa catalog #2270A) was added and the mixture was incubated at 37° C. for 30 minutes. Subsequently, 10× phosphatase buffer (NEB, catalog #B0289S) and 0.25 U/μL Antarctic phosphatase (NEB, catalog #M0289L) were added and incubated at 37° C. for 30 minutes. An 8 M LiCl solution (Sigma-Aldrich catalog #L7026) was added to the reaction mixture to a final concentration of 8/3 M, and the mixture was left at −20° C. for over 1 hour. After centrifugation (4° C., 15000 rpm, 30 minutes), the supernatant was discarded. The pellet was washed three times with 75% ethanol, followed by centrifugation (4° C., 15000 rpm, 5 minutes) and discarding the supernatant each time. The resulting pellet was dissolved in nuclease-free water. The mRNA yield was quantified by measuring absorbance at 260 nm.

(iii) Analysis of the Capping Efficiency

The mRNA synthesized by IVT was cleaved by treatment of DNAZyme10-23. The mRNA (26 g) and DNAZyme10-23 (100 μmol) were dissolved in 50 mM Tris-HCl buffer (40 μL, pH 8) containing 50 mM MgCl₂ buffer and reacted at 37° C. for 1 hour. After the reaction, 3M NaOAc (4 L) and isopropanol (40 L) were added, and the mixture was left at −20° C. for 1 hour. Each sample was centrifuged (14,000 g, 4° C., 30 min) and the supernatant was removed. 80% EtOH (100 L) was added, followed by centrifugation (14,000 g, 4° C., 5 min), and the supernatant was removed again. After another wash with 80% EtOH, the resulting pellet was dissolved in TE buffer (20 μL, Nacalai Tesque catalog #06890-54). The cleaved 5′-region of mRNA was analyzed by 15% denaturing PAGE and the capping efficacy was measured by ImageJ software.

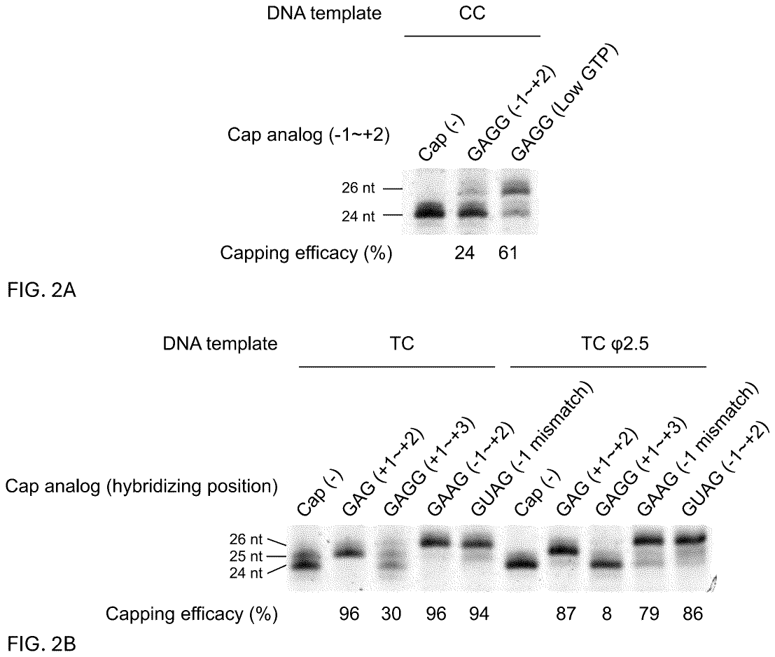

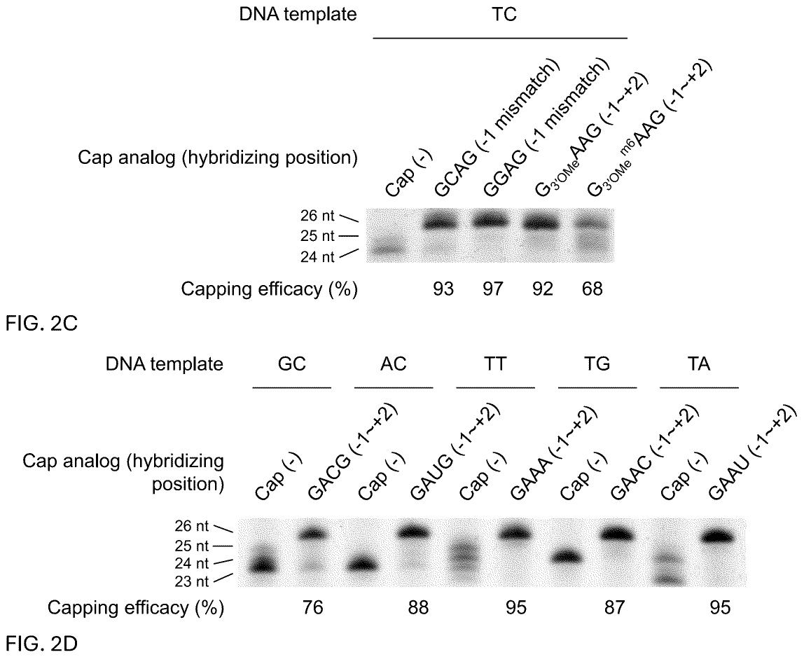

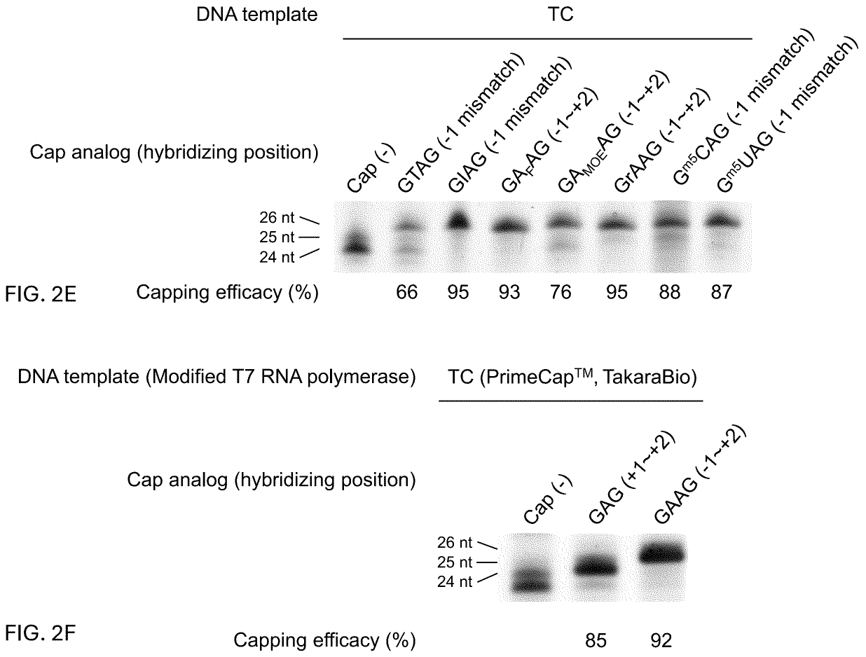

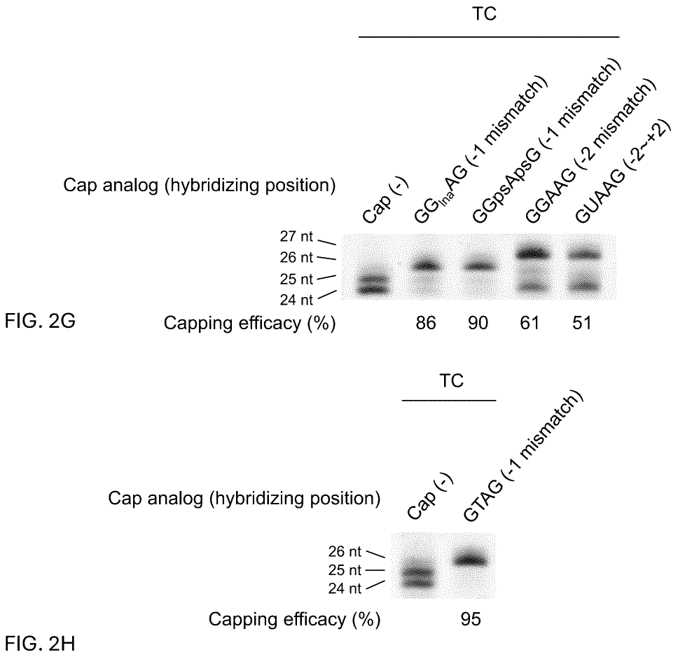

As shown in FIG. 2A, low capping efficiency (<30%) was observed for an IVT reaction combining a CC template, a capping primer having nucleobases complementary to positions −1 to +2 of the CC template, and equimolar GTP conditions (i.e., condition 1). The capping efficiency was increased under low GTP conditions (i.e., condition 2), e.g., due to decreased competition with de novo RNA synthesis from GTP. However, the yield of mRNA under low GTP conditions was substantially reduced as compared to yield under equimolar GTP conditions.

As shown in FIG. 2B, high capping efficiency (>90%) was observed for an IVT reaction combining a TC or TCφ2.5 template, a capping primer having nucleobases complementary to positions −1 to +2 of the template, and equimolar GTP conditions (i.e., condition 1). The capping efficiency achieved was comparable to a previously described Cap-1 trinucleotide capping primer having nucleobases complementary to positions +1 and +2 of the template (i.e., Cap-1-GAG) and substantially higher as compared to a previously described Cap-2 tetranucleotide capping primer having nucleobases complementary to positions +1 to +3 of the template (i.e., Cap-2-GAGG). Cap-1-AG and Cap-2-GAGG are further described in WO2017053297, which is herein incorporated by reference. The high capping efficiency was maintained whether the second nucleotide from the 5′-end of the capping primer was complementary or mismatched with respect to position −1 of the template (i.e., Cap-2-GAAG vs Cap-2-GUAG).

Table 7 summarizes the capping efficiency and the total yield of mRNA as a function of 5′-capping primer, DNA template, and reaction condition evaluated in the IVT reaction.

TABLE 7
Capping Efficiency and mRNA Yield for Exemplary
IVT Reactions (100 μL scale)

| Template | IVT | Cap primer | Capping primer | Capping | Yield |
|---|---|---|---|---|---|
| DNA | Condition | Name | sequence | (%) | (μg) |
| CC | 1 | - | - | - | 435.2 |
| | 1 | Cap-2-GAGG | ^{m7}GpppA_{2′OMe}pG_{2′OMe}pG | 24 | 423.0 |
| | 2 | Cap-2-GAGG | ^{m7}GpppA_{2′OMe}pG_{2′OMe}pG | 61 | 61.5 |
| TC | 1 | — | — | — | 281.7 |
| | 1 | Cap-1-GAG | ^{m7}GpppA_{2′OMe}pG | 96 | 361.7 |
| | 1 | Cap-2-GAGG | ^{m7}GpppA_{2′OMe}pG_{2′OMe}pG | 30 | 66.0 |
| | 1 | Cap-2-GAAG | ^{m7}GpppA_{2′OMe}pA_{2′OMe}pG | 96 | 410.1 |
| | 1 | Cap-2-GUAG | ^{m7}GpppU_{2′OMe}pA_{2′OMe}pG | 94 | 395.4 |
| TCφ2.5 | 1 | — | — | — | 362.7 |
| | 1 | Cap-1-GAG | ^{m7}GpppA_{2′OMe}pG | 87 | 464.4 |
| | 1 | Cap-2-GAGG | ^{m7}GpppA_{2′OMe}pG_{2′OMe}pG | 8 | 196.2 |
| | 1 | Cap-2-GAAG | ^{m7}GpppA_{2′OMe}pA_{2′OMe}pG | 79 | 331.4 |
| | 1 | Cap-2-GUAG | ^{m7}GpppU_{2′OMe}pA_{2′OMe}pG | 86 | 278.4 |

### Example 3: In Vitro Transcription (IVT) with Exemplary 5′-Capping Primers

The exemplary capping primers described in Example 1 were evaluated for capping efficiency in an IVT reaction to generate mRNA. IVT reactions were performed using DNA templates with specific nucleotides at positions +1 and +2, in combination with capping primers complementary to those positions.

### (i) DNA Synthesis for In Vitro Transcription

To prepare the template plasmid DNA for in vitro transcription (IVT), a plasmid containing the T7 promoter sequence, 5′-UTR sequence, KOZAK sequence, each ORF sequence, and 3′-UTR sequence in order was synthesized.

For the TC template, the −1 template nucleotide was 2′-deoxyribothymidine, the +1 template nucleotide was 2′-deoxyribothymidine, and the +2 template nucleotide was 2′-deoxyribocytidine.

For the GC template, the −1 template nucleotide was 2′-deoxyribothymidine, the +1 template nucleotide was 2′-deoxyriboguanosine, and the +2 template nucleotide was 2′-deoxyribocytidine.

For the AC template, the −1 template nucleotide was 2′-deoxyribothymidine, the +1 template nucleotide was 2′-deoxyriboadenosine, and the +2 template nucleotide was 2′-deoxyribocytidine.

For the TT template, the −1 template nucleotide was 2′-deoxyribothymidine, the +1 template nucleotide was 2′-deoxyribothymidine, and the +2 template nucleotide was 2′-deoxyribothymidine.

For the TG template, the −1 template nucleotide was 2′-deoxyribothymidine, the +1 template nucleotide was 2′-deoxyribothymidine, and the +2 template nucleotide was 2′-deoxyriboguanosine.

For the TA template, the −1 template nucleotide was 2′-deoxyribothymidine, the +1 template nucleotide was 2′-deoxyribothymidine, and the +2 template nucleotide was 2′-deoxyriboadenosine.

Sequence information for the DNA templates evaluated in this Example are identified in Table 8.

TABLE 8
Sense Strand Sequences for Exemplary DNA Templates

| | T7 promoter sequence | RNA-coding region |
|---|---|---|
| | (SEQ ID NO) | (SEQ ID NO) |
| TC Template | 1 | 3 |
| GC Template | 1 | 10 |
| AC Template | 1 | 11 |
| TT Template | 1 | 12 |
| TG Template | 1 | 13 |
| TA Template | 1 | 14 |

The plasmid (1 μg) was dissolved in nuclease-free water (210 μL), and Q5 Hot Start High-Fidelity 2× Master Mix (250 μL, NEB #M0494L), 10 μM sense primer (20 μL), and 10 μM antisense primer containing poly-T (20 μL) were added. After incubation at 95° C. for 1 minute, the mixture was subjected to 35 cycles of 95° C. for 30 seconds, 60° C. for 30 seconds, and 72° C. for 3 minutes, followed by a final incubation at 72° C. for 5 minutes to amplify the template DNA by PCR. After the reaction, isopropanol (500 μL) was added, and the mixture was left at −20° C. for over 1 hour. It was then centrifuged (4° C., 15000 rpm, 30 minutes), and the supernatant was discarded. This precipitate was further washed by 75% ethanol three times. The resulting precipitate was dissolved in nuclease-free water to obtain the template DNA containing the ORF of Firefly luciferase.

### (ii) In Vitro Transcription (IVT)

mRNA was prepared by IVT using the DNA templates described above. The IVT reaction was performed using condition 1 as described in Example 2, and condition 3 described below.

### Condition 3—Modified T7 RNA Polymerase

The following conditions combine primer and NTPs at a molar ratio of 10 to 4.

The IVT reaction was performed by mixing 50 g/mL template DNA, 4 mM Cap analog, 10 mM ATP (TriLink catalog #N-1510), 10 mM CTP (TriLink catalog #N-1511), 10 mM GTP (TriLink catalog #N-1512), 10 mM N1-methyl-W-uridine-5′-triphosphate (1mψTP), T7 RNA Polymerase Buffer (Takara Bio), 1000 U/μL RNase inhibitor (NEB, catalog #M0314L), 2 U/μL Yeast Inorganic pyrophosphatase (NEB, catalog #M2403L), and PrimeCap T7 RNA Polymerase (Takara Bio, catalog #2560A). The mixture was incubated at 37° C. for 3 hours. Following this, RNase-Free DNase I (6 U/μg template DNA, TaKaRa catalog #2270A) was added and the mixture was incubated at 37° C. for 30 minutes. Subsequently, 10× phosphatase buffer (NEB, catalog #B0289S) and 0.25 U/μL Antarctic phosphatase (NEB, catalog #M0289L) were added and incubated at 37° C. for 30 minutes. An 8 M LiCl solution (Sigma-Aldrich catalog #L7026) was added to the reaction mixture to a final concentration of 8/3 M, and the mixture was left at −20° C. for over 1 hour. After centrifugation (4° C., 15000 rpm, 30 minutes), the supernatant was discarded. The pellet was washed three times with 75% ethanol, followed by centrifugation (4° C., 15000 rpm, 5 minutes) and discarding the supernatant each time. The resulting pellet was dissolved in nuclease-free water. The mRNA yield was quantified by measuring absorbance at 260 nm.

(iii) Analysis of the Capping Efficiency

The mRNA synthesized by IVT was cleaved by treatment of DNAZyme10-23. The mRNA (26 g) and DNAZyme10-23 (100 μmol) were dissolved in 50 mM Tris-HCl (pH 8) containing 50 mM MgCl2 buffer and reacted at 37° C. for 1 hour. After the reaction, 3M NaOAc (NIPPON GENE, catalog #316-90081) and isopropanol were added, and the mixture was left at −20° C. for 1 hour. Each sample was centrifuged (14,000 g, 4° C., 30 min) and the supernatant was removed. 80% EtOH (100 L) was added, followed by centrifugation (14,000 g, 4° C., 5 min), and the supernatant was removed again. After another wash with 80% EtOH, the resulting pellet was dissolved in TE buffer (Nacalai Tesque, catalog #06890-54). The cleaved 5′-region of mRNA was analyzed by 15% denaturing PAGE and the capping efficacy was measured by ImageJ software.

As shown in FIG. 2C and Table 9, a high capping efficiency and mRNA yield were observed for an IVT reaction combining a TC template, a capping primer having nucleobases complementary to positions +1 to +2 of the template, and equimolar GTP conditions (i.e., condition 1). The high capping efficiency was maintained whether the second nucleotide from the 5′-end of the capping primer was mismatched with respect to position −1 of the template (i.e., Cap-2-GCAG and Cap-2-GGAG) or if the capping primer contained an anti-reverse cap analog (i.e., Cap2-G_{3′OMe}AAG). A reduction in mRNA yield was observed for a capping primer that contained an anti-reverse cap analog and m6A at the second nucleotide from the 5′-end of the capping primer (i.e., Cap-2-G_{3′OMe}^{m6}AAG).

As shown in FIG. 2D and Table 9, a high capping efficiency and mRNA yield were observed for an IVT reaction combining a GC, AC, TT, TG, or TA template, a capping primer having nucleobases complementary to positions −1 to +2 of the template, and equimolar GTP conditions (i.e., condition 1).

As shown in FIG. 2E and Table 9, a high capping efficiency and mRNA yield were observed for an IVT reaction combining a TC template, a capping primer having nucleobases complementary to positions +1 to +2 of the template, and equimolar GTP conditions (i.e., condition 1). The high capping efficiency and yield were maintained if the second nucleotide from the 5′-end of the capping primer was unnatural but complimentary with respect to position −1 of the template (i.e., Cap-2-GA_{F}AG, and Cap-2-GA_{MOE}AG), a modified nucleoside mismatched with respect to position −1 (i.e., Cap-1-GTAG, Cap-2-GIAG, Cap-2-G^{m5}CAG and Cap-2-G^{m5}UAG), or unmethylated at the 2′—OH group (i.e., Cap-1-GrAAG). As shown in FIG. 2F and Table 10, a high capping efficiency and mRNA yield was observed for an IVT reaction combining a TC template, a capping primer having nucleobases complementary to positions −1 to +2 of the template (i.e., Cap-2-GAAG), and a modified T7 RNA polymerase (i.e., condition 3).

TABLE 9
Capping Efficiency and mRNA Yield Exeplary IVT Reactions (100 μL scale)

| Template | IVT | | Capping primer | Capping | Yield |
|---|---|---|---|---|---|
| DNA | Condition | Cap primer Name | sequence | (%) | (μg) |
| TC | 1 | - | - | - | 281.7 |
| | 1 | Cap-2-GCAG | ^{m7}GpppC_{2′OMe}pA_{2′OMe}pG | 93 | 397.9 |
| | 1 | Cap-2-GGAG | ^{m7}GpppG_{2′OMe}pA_{2′OMe}pG | 97 | 425.7 |
| | 1 | Cap-1-GTAG | ^{m7}GpppdTpA_{2′OMe}pG | 66 | 288.5 |
| | 1 | Cap-2-GIAG | ^{m7}GpppI_{2′OMe}pA_{2′OMe}pG | 95 | 440.6 |
| | 1 | Cap-2-G^{m5}CAG | ^{m7}Gppp^{m5}C_{2′OMe}pA_{2′OMe}pG | 88 | 384.2 |
| | 1 | Cap-2-G^{m5}UAG | ^{m7}Gppp^{m5}U_{2′OMe}pA_{2′OMe}pG | 87 | 441.5 |
| | 1 | Cap-1-GrAAG | GppprApA_{2′OMe}pG | 95 | 410.5 |
| | 1 | Cap-2-GA_{F}AG | ^{m7}GpppA_{2′F}pA_{2′OMe}pG | 93 | 336.8 |
| | 1 | Cap-2-GA_{MOE}AG | ^{m7}GpppA_{2′OMe}DA_{2′OMe}pG | 76 | 397.8 |
| | 1 | Cap-2-G_{3′OMe}AAG | ^{m7}G_{3′OMe}pppG_{2′OMe}pA_{2′OMe}pG | 92 | 376.0 |
| | 1 | Cap-2-G_{3′OMe}^{m6}AAG | ^{m7}G_{3′OMe}ppp^{m6}A_{2′OMe}pA_{2′OMe}pG | 68 | 32.1 |
| GC | 1 | — | — | — | 228.8 |
| | 1 | Cap-2-GACG | ^{m7}GpppA_{2′OMe}pC_{2′OMe}pG | 76 | 130.8 |
| AC | 1 | — | — | — | 276.6 |
| | 1 | Cap-2-GAUG | ^{m7}GpppA_{2′OMe}pU_{2′OMe}pG | 88 | 422.7 |
| TT | 1 | — | — | — | 161.0 |
| | 1 | Cap-2-GAAA | ^{m7}GpppA_{2′OMe}pA_{2′OMe}pA | 95 | 366.7 |
| TG | 1 | — | — | — | 179.9 |
| | 1 | Cap-2-GAAC | ^{m7}GpppA_{2′OMe}pA_{2′OMe}pC | 87 | 355.6 |
| TA | 1 | — | — | — | 33.1 |
| | 1 | Cap-2-GAAU | ^{m7}GpppA_{2′OMe}pA_{2′OMe}pU | 95 | 293.9 |

TABLE 10
Capping Efficiency and mRNA Yield Exemplary
IVT Reactions (50 μL scale)

| | IVT | Cap | Capping | | |
|---|---|---|---|---|---|
| Template | Condi- | primer | primer | Capping | Yield |
| DNA | tion | Name | sequence | (%) | (μg) |
| TC | 3 | Cap-1- | ^{m7}Gpppp | 85 | 181.0 |
| | | GAG | A_{2′OMe}pG | | |
| | 3 | Cap-2- | ^{m7}GpppA_{2′OMe} | 92 | 313.6 |
| | | GAAG | pA_{2′OMe}pG | | |

### Example 4: In Vitro Transcription (IVT) with Exemplary 5′-Capping Primers

The exemplary capping primers described in Example 1 were evaluated for capping efficiency in an IVT reaction to generate mRNA. IVT reactions were performed using DNA templates with specific nucleotides at positions +1 and +2, in combination with capping primers complementary to those positions.

### i) DNA Synthesis for In Vitro Transcription

To prepare the template plasmid DNA for in vitro transcription (IVT), a plasmid containing the T7 promoter sequence, 5′-UTR sequence, KOZAK sequence, each ORF sequence, and 3′-UTR sequence in order was synthesized.

For the TC template, the −1 template nucleotide was 2′-deoxyribothymidine, the +1 template nucleotide was 2′-deoxyribothymidine, and the +2 template nucleotide was 2′-deoxyribocytidine.

TABLE 11
Sense Strand Sequences for Exemplary DNA Template

| | T7 promoter sequence | RNA-coding region |
|---|---|---|
| | (SEQ ID NO) | (SEQ ID NO) |
| TC Template | 1 | 3 |

The plasmid (1 μg) was dissolved in nuclease-free water (210 μL), and Q5 Hot Start High-Fidelity 2× Master Mix (250 μL, NEB #M0494L), 10 μM sense primer (20 μL), and 10 μM antisense primer containing poly-T (20 μL) were added. After incubation at 95° C. for 1 minute, the mixture was subjected to 35 cycles of 95° C. for 30 seconds, 60° C. for 30 seconds, and 72° C. for 3 minutes, followed by a final incubation at 72° C. for 5 minutes to amplify the template DNA by PCR. After the reaction, isopropanol (500 μL) was added, and the mixture was left at −20° C. for over 1 hour. It was then centrifuged (4° C., 15000 rpm, 30 minutes), and the supernatant was discarded. This precipitate was further washed by 75% ethanol three times. The resulting precipitate was dissolved in nuclease-free water to obtain the template DNA containing the ORF of Firefly luciferase.

### (ii) In Vitro Transcription (IVT)

mRNA was prepared by IVT using the DNA template described above. The IVT reaction was performed using condition 1 as described in Example 2.

(iii) Analysis of the Capping Efficiency

The mRNA synthesized by IVT was cleaved by treatment of DNAZyme10-23. The mRNA (26 g) and DNAZyme10-23 (100 μmol) were dissolved in 50 mM Tris-HCl (pH 8) containing 50 mM MgCl2 buffer and reacted at 37° C. for 1 hour. After the reaction, 3M NaOAc (NIPPON GENE, catalog #316-90081) and isopropanol were added, and the mixture was left at −20° C. for 1 hour. Each sample was centrifuged (14,000 g, 4° C., 30 min) and the supernatant was removed. 80% EtOH (100 μL) was added, followed by centrifugation (14,000 g, 4° C., 5 min), and the supernatant was removed again. After another wash with 80% EtOH, the resulting pellet was dissolved in TE buffer (Nacalai Tesque, catalog #06890-54). The cleaved 5′-region of mRNA was analyzed by 15% denaturing PAGE and the capping efficacy was measured by ImageJ software.

As shown in FIG. 2G and Table 12, a moderate to high capping efficiency and mRNA yield were observed for an IVT reaction combining a TC template, a capping primer having nucleobases complementary to positions +1 to +2 of the template, and equimolar GTP conditions (i.e., condition 1). The high capping efficiency and yield were maintained if the second nucleotide from the 5′-end of the capping primer was modified nucleoside mismatched with respect to position −1 ((i.e., Cap-2-GG_{Ina}AG), or the phosphodiester linkages were unnatural (i.e., Cap-2-GGpsApsG). A moderate capping efficiency and mRNA yield were observed for an IVT reaction combining a TC template, a pentanucleotide capping primer having nucleobases fully complementary to positions −2 to +2 of the template (i.e., Cap-3-GUAAG), or a mismatched nucleobase with respect to position −2 (i.e., Cap-3-GGAAG).

TABLE 12
Capping Efficiency and mRNA Yield Exemplary IVT Reactions (100 μL scale)

| | IVT | Cap primer | Capping | Capping | Yield |
|---|---|---|---|---|---|
| Template DNA | Condition | Name | primer sequence | (%) | (μg) |
| TC | 1 | Cap-2-GGₗₙₐAG | ^{m7}GppppGₗₙₐpA_{2′OMe}pG | 86 | 380.5 |
| | 1 | Cap-2-GGpsApsG | ^{m7}GpppG_{2′OMe}psA_{2′OMe}psG | 90 | 270.1 |
| | 1 | Cap-3-GGAAG | ^{m7}GpppG_{2′OMe}pA_{2′OMe}pA_{2′OMep}pG | 61 | 258.7 |
| | 1 | Cap-3-GUAAG | ^{m7}GpppU_{2′OMe}pA_{2′OMe}pA_{2′OMep}pG | 51 | 236.2 |

### Example 5: In Vitro Transcription (IVT) with Exemplary 5′-Capping Primers

The exemplary capping primers described in Example 1 were evaluated for capping efficiency in an IVT reaction to generate mRNA. IVT reactions were performed using DNA templates with specific nucleotides at positions +1 and +2, in combination with capping primers complementary to those positions.

### i) DNA Synthesis for In Vitro Transcription

To prepare the template plasmid DNA for in vitro transcription (IVT), a plasmid containing the T7 promoter sequence, 5′-UTR sequence, KOZAK sequence, each ORF sequence, and 3′-UTR sequence in order was synthesized.

For the TC template, the −1 template nucleotide was 2′-deoxyribothymidine, the +1 template nucleotide was 2′-deoxyribothymidine, and the +2 template nucleotide was 2′-deoxyribocytidine.

TABLE 13
Sense Strand Sequences for Exemplary DNA Template

| | T7 promoter sequence | RNA-coding region |
|---|---|---|
| | (SEQ ID NO) | (SEQ ID NO) |
| TC Template | 1 | 3 |

The plasmid (1 μg) was dissolved in nuclease-free water (210 μL), and Q5 Hot Start High-Fidelity 2× Master Mix (250 μL, NEB #M0494L), 10 μM sense primer (20 μL), and 10 μM antisense primer containing poly-T (20 μL) were added. After incubation at 95° C. for 1 minute, the mixture was subjected to 35 cycles of 95° C. for 30 seconds, 60° C. for 30 seconds, and 72° C. for 3 minutes, followed by a final incubation at 72° C. for 5 minutes to amplify the template DNA by PCR. After the reaction, isopropanol (500 μL) was added, and the mixture was left at −20° C. for over 1 hour. It was then centrifuged (4° C., 15000 rpm, 30 minutes), and the supernatant was discarded. This precipitate was further washed by 75% ethanol three times. The resulting precipitate was dissolved in nuclease-free water to obtain the template DNA containing the ORF of Firefly luciferase.

### (ii) In Vitro Transcription (IVT)

### Condition 4—High Capping Primer

The following conditions combine primer and NTPs at a molar ratio of 2 to 1.

The IVT reaction was performed by mixing 50 g/mL template DNA, 10 mM Cap analog, 5 mM ATP (TriLink catalog #N-1510), 5 mM CTP (TriLink catalog #N-1511), 5 mM GTP (TriLink catalog #N-1512), 5 mM N1-methyl-ψ-uridine-5′-triphosphate (^{1m}ψTP), 40 mM Tris-HCl (pH 7.5), 15 mM HCl, 10 mM DTT, 2 mM Spermidine, 0.002% Triton, 30 mM MgCl2, 1000 U/μL RNase inhibitor (NEB, catalog #M0314L), 2 U/μL Yeast Inorganic pyrophosphatase (32 μL, NEB, catalog #M2403L), and T7 RNA Polymerase (Roche catalog #08140669103). The mixture was incubated at 37° C. for 3 hours. Following this, RNase-Free DNase I (6 U/μg template DNA, TaKaRa catalog #2270A) was added and the mixture was incubated at 37° C. for 30 minutes. Subsequently, 10× phosphatase buffer (NEB, catalog #B0289S) and 0.25 U/μL Antarctic phosphatase (NEB, catalog #M0289L) were added and incubated at 37° C. for 30 minutes. An 8 M LiCl solution (Sigma-Aldrich catalog #L7026) was added to the reaction mixture to a final concentration of 8/3 M, and the mixture was left at −20° C. for over 1 hour. After centrifugation (4° C., 15000 rpm, 30 minutes), the supernatant was discarded. The pellet was washed three times with 75% ethanol, followed by centrifugation (4° C., 15000 rpm, 5 minutes) and discarding the supernatant each time. The resulting pellet was dissolved in nuclease-free water. The mRNA yield was quantified by measuring absorbance at 260 nm.

(iii) Analysis of the Capping Efficiency

The mRNA synthesized by IVT was cleaved by treatment of DNAZyme10-23. The mRNA (26 g) and DNAZyme10-23 (100 pmol) were dissolved in 50 mM Tris-HCl (pH 8) containing 50 mM MgCl2 buffer and reacted at 37° C. for 1 hour. After the reaction, 3M NaOAc (NIPPON GENE, catalog #316-90081) and isopropanol were added, and the mixture was left at −20° C. for 1 hour. Each sample was centrifuged (14,000 g, 4 C, 30 mi) and the supernatant was removed. 8000 EtOH (100 μL) was added, followed by centrifugation (14,000 g, 4 (C, 5 m %), and the supernatant was removed again. After another wash with 80% EtOH, the resulting pellet was dissolved in TE buffer (Nacalai Tesque, catalog #06890-54). The cleaved 5′-region of mRNA was analyzed by 15% denaturing PAGE and the capping efficacy was measured by ImageJ software.

As shown in FIG. 211 and Table 14, higher capping efficiency and mRNA yield were observed in the IVT reaction using a TC template and a high concentration (10 mM) of capping primer (i.e., Cap-1 GTAG, condition 4), compared to the condition using the standard capping primer concentration (i.e., 5 mM, condition 1).

TABLE 14
Capping Efficiency and mRNA Yield Exemplary
IVT Reactions (100 μL scale)

| | IVT | Cap | Capping | | |
|---|---|---|---|---|---|
| Template | Condi- | primer | primer | Capping | Yield |
| DNA | tion | Name | sequence | (%) | (μg) |
| TC | 4 | Cap-1- | ^{m7}GpppdT | 95 | 135.4 |
| | | GTAG | pA_{2′OMe}pG | | |

LISTING OF SEQUENCES OF THE DISCLOSURE

| | | SEQ |
|---|---|---|
| Name | Sequence | ID NO |
| T7 promoter | TAATACGACTCACTATA | 1 |
| CC DNA | GGGAAATAAGAGAGAAAAGAAGAGTAAGAAGAAATATAAGAGCCACCATGGAGGACGCCAAGAA | 2 |
| template - | CATCAAGAAGGGCCCCGCCCCCTTCTACCCCCTGGAGGACGGCACCGCCGGCGAGCAGCTGCAC | |
| sense | AAGGCCATGAAGCGGTACGCCCTGGTGCCCGGCACCATCGCCTTCACCGACGCCCACATCGAGG | |
| strand | TGGACATCACCTACGCCGAGTACTTCGAGATGAGCGTGCGGCTGGCCGAGGCCATGAAGCGGTA | |
| RNA-coding | CGGCCTGAACACCAACCACCGGATCGTGGTGTGCAGCGAGAACAGCCTGCAGTTCTTCATGCCC | |
| region | GTGCTGGGCGCCCTGTTCATCGGCGTGGCCGTGGCCCCCGCCAACGACATCTACAACGAGCGGG | |
| | AGCTGCTGAACAGCATGGGCATCAGCCAGCCCACCGTGGTGTTCGTGAGCAAGAAGGGCCTGCA | |
| | GAAGATCCTGAACGTGCAGAAGAAGCTGCCCATCATCCAGAAGATCATCATCATGGACAGCAAG | |
| | ACCGACTACCAGGGCTTCCAGAGCATGTACACCTTCGTGACCAGCCACCTGCCCCCCGGCTTCA | |
| | ACGAGTACGACTTCGTGCCCGAGAGCTTCGACCGGGACAAGACCATCGCCCTGATCATGAACAG | |
| | CAGCGGCAGCACCGGCCTGCCCAAGGGCGTGGCCCTGCCCCACCGGACCGCCTGCGTGCGGTTC | |
| | AGCCACGCCCGGGACCCCATCTTCGGCAACCAGATCATCCCCGACACCGCCATCCTGAGCGTGG | |
| | TGCCCTTCCACCACGGCTTCGGCATGTTCACCACCCTGGGCTACCTGATCTGCGGCTTCCGGGT | |
| | GGTGCTGATGTACCGGTTCGAGGAGGAGCTGTTCCTGCGGAGCCTGCAGGACTACAAGATCCAG | |
| | AGCGCCCTGCTGGTGCCCACCCTGTTCAGCTTCTTCGCCAAGAGCACCCTGATCGACAAGTACG | |
| | ACCTGAGCAACCTGCACGAGATCGCCAGCGGCGGCGCCCCCCTGAGCAAGGAGGTGGGCGAGGC | |
| | CGTGGCCAAGCGGTTCCACCTGCCCGGCATCCGGCAGGGCTACGGCCTGACCGAGACCACCAGC | |
| | GCCATCCTGATCACCCCCGAGGGCGACGACAAGCCCGGCGCCGTGGGCAAGGTGGTGCCCTTCT | |
| | TCGAGGCCAAGGTGGTGGACCTGGACACCGGCAAGACCCTGGGCGTGAACCAGCGGGGCGAGCT | |
| | GTGCGTGCGGGGCCCCATGATCATGAGCGGCTACGTGAACAACCCCGAGGCCACCAACGCCCTG | |
| | ATCGACAAGGACGGCTGGCTGCACAGCGGCGACATCGCCTACTGGGACGAGGACGAGCACTTCT | |
| | TCATCGTGGACCGGCTGAAGAGCCTGATCAAGTACAAGGGCTACCAGGTGGCCCCCGCCGAGCT | |
| | GGAGAGCATCCTGCTGCAGCACCCCAACATCTTCGACGCCGGCGTGGCCGGCCTGCCCGACGAC | |
| | GACGCCGGCGAGCTGCCCGCCGCCGTGGTGGTGCTGGAGCACGGCAAGACCATGACCGAGAAGG | |
| | AGATCGTGGACTACGTGGCCAGCCAGGTGACCACCGCCAAGAAGCTGCGGGGCGGCGTGGTGTT | |
| | CGTGGACGAGGTGCCCAAGGGCCTGACCGGCAAGCTGGACGCCCGGAAGATCCGGGAGATCCTG | |
| | ATCAAGGCCAAGAAGGGCGGCAAGATCGCCGTGTGATAAGCTGCCTTCTGCGGGGCTTGCCTTC | |
| | TGGCCATGCCCTTCTTCTCTCCCTTGCACCTGTACCTCTTGGTCTTTGAATAAAGCCTGAGTAG | |
| | GAAGGCGGAAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAAAA | |
| TC DNA | AGGGAAATAAGAGAGAAAAGAAGAGTAAGAAGAAATATAAGAGCCACCATGGAGGACGCCAAGA | 3 |
| template - | ACATCAAGAAGGGCCCCGCCCCCTTCTACCCCCTGGAGGACGGCACCGCCGGCGAGCAGCTGCA | |
| sense | CAAGGCCATGAAGCGGTACGCCCTGGTGCCCGGCACCATCGCCTTCACCGACGCCCACATCGAG | |
| strand | GTGGACATCACCTACGCCGAGTACTTCGAGATGAGCGTGCGGCTGGCCGAGGCCATGAAGCGGT | |
| RNA-coding | ACGGCCTGAACACCAACCACCGGATCGTGGTGTGCAGCGAGAACAGCCTGCAGTTCTTCATGCC | |
| region | CGTGCTGGGCGCCCTGTTCATCGGCGTGGCCGTGGCCCCCGCCAACGACATCTACAACGAGCGG | |
| | GAGCTGCTGAACAGCATGGGCATCAGCCAGCCCACCGTGGTGTTCGTGAGCAAGAAGGGCCTGC | |
| | AGAAGATCCTGAACGTGCAGAAGAAGCTGCCCATCATCCAGAAGATCATCATCATGGACAGCAA | |
| | GACCGACTACCAGGGCTTCCAGAGCATGTACACCTTCGTGACCAGCCACCTGCCCCCCGGCTTC | |
| | AACGAGTACGACTTCGTGCCCGAGAGCTTCGACCGGGACAAGACCATCGCCCTGATCATGAACA | |
| | GCAGCGGCAGCACCGGCCTGCCCAAGGGCGTGGCCCTGCCCCACCGGACCGCCTGCGTGCGGTT | |
| | CAGCCACGCCCGGGACCCCATCTTCGGCAACCAGATCATCCCCGACACCGCCATCCTGAGCGTG | |
| | GTGCCCTTCCACCACGGCTTCGGCATGTTCACCACCCTGGGCTACCTGATCTGCGGCTTCCGGG | |
| | TGGTGCTGATGTACCGGTTCGAGGAGGAGCTGTTCCTGCGGAGCCTGCAGGACTACAAGATCCA | |
| | GAGCGCCCTGCTGGTGCCCACCCTGTTCAGCTTCTTCGCCAAGAGCACCCTGATCGACAAGTAC | |
| | GACCTGAGCAACCTGCACGAGATCGCCAGCGGCGGCGCCCCCCTGAGCAAGGAGGTGGGCGAGG | |
| | CCGTGGCCAAGCGGTTCCACCTGCCCGGCATCCGGCAGGGCTACGGCCTGACCGAGACCACCAG | |
| | CGCCATCCTGATCACCCCCGAGGGCGACGACAAGCCCGGCGCCGTGGGCAAGGTGGTGCCCTTC | |
| | TTCGAGGCCAAGGTGGTGGACCTGGACACCGGCAAGACCCTGGGCGTGAACCAGCGGGGCGAGC | |
| | TGTGCGTGCGGGGCCCCATGATCATGAGCGGCTACGTGAACAACCCCGAGGCCACCAACGCCCT | |
| | GATCGACAAGGACGGCTGGCTGCACAGCGGCGACATCGCCTACTGGGACGAGGACGAGCACTTC | |
| | TTCATCGTGGACCGGCTGAAGAGCCTGATCAAGTACAAGGGCTACCAGGTGGCCCCCGCCGAGC | |
| | TGGAGAGCATCCTGCTGCAGCACCCCAACATCTTCGACGCCGGCGTGGCCGGCCTGCCCGACGA | |
| | CGACGCCGGCGAGCTGCCCGCCGCCGTGGTGGTGCTGGAGCACGGCAAGACCATGACCGAGAAG | |
| | GAGATCGTGGACTACGTGGCCAGCCAGGTGACCACCGCCAAGAAGCTGCGGGGCGGCGTGGTGT | |
| | TCGTGGACGAGGTGCCCAAGGGCCTGACCGGCAAGCTGGACGCCCGGAAGATCCGGGAGATCCT | |
| | GATCAAGGCCAAGAAGGGCGGCAAGATCGCCGTGTGATAAGCTGCCTTCTGCGGGGCTTGCCTT | |
| | CTGGCCATGCCCTTCTTCTCTCCCTTGCACCTGTACCTCTTGGTCTTTGAATAAAGCCTGAGTA | |
| | GGAAGGCGGAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAAAA | |
| | A | |
| TCφ2.5 DNA | TAATACGACTCACTATT | 4 |
| template - | | |
| sense | | |
| strand | | |
| T7 promoter | | |
| TCφ2.5 DNA | AGGGAAATAAGAGAGAAAAGAAGAGTAAGAAGAAATATAAGAGCCACCATGGAGGACGCCAAGA | 5 |
| template - | ACATCAAGAAGGGCCCCGCCCCCTTCTACCCCCTGGAGGACGGCACCGCCGGCGAGCAGCTGCA | |
| sense | CAAGGCCATGAAGCGGTACGCCCTGGTGCCCGGCACCATCGCCTTCACCGACGCCCACATCGAG | |
| strand | GTGGACATCACCTACGCCGAGTACTTCGAGATGAGCGTGCGGCTGGCCGAGGCCATGAAGCGGT | |
| RNA-coding | ACGGCCTGAACACCAACCACCGGATCGTGGTGTGCAGCGAGAACAGCCTGCAGTTCTTCATGCC | |
| region | CGTGCTGGGCGCCCTGTTCATCGGCGTGGCCGTGGCCCCCGCCAACGACATCTACAACGAGCGG | |
| | GAGCTGCTGAACAGCATGGGCATCAGCCAGCCCACCGTGGTGTTCGTGAGCAAGAAGGGCCTGC | |
| | AGAAGATCCTGAACGTGCAGAAGAAGCTGCCCATCATCCAGAAGATCATCATCATGGACAGCAA | |
| | GACCGACTACCAGGGCTTCCAGAGCATGTACACCTTCGTGACCAGCCACCTGCCCCCCGGCTTC | |
| | AACGAGTACGACTTCGTGCCCGAGAGCTTCGACCGGGACAAGACCATCGCCCTGATCATGAACA | |
| | GCAGCGGCAGCACCGGCCTGCCCAAGGGCGTGGCCCTGCCCCACCGGACCGCCTGCGTGCGGTT | |
| | CAGCCACGCCCGGGACCCCATCTTCGGCAACCAGATCATCCCCGACACCGCCATCCTGAGCGTG | |
| | GTGCCCTTCCACCACGGCTTCGGCATGTTCACCACCCTGGGCTACCTGATCTGCGGCTTCCGGG | |
| | TGGTGCTGATGTACCGGTTCGAGGAGGAGCTGTTCCTGCGGAGCCTGCAGGACTACAAGATCCA | |
| | GAGCGCCCTGCTGGTGCCCACCCTGTTCAGCTTCTTCGCCAAGAGCACCCTGATCGACAAGTAC | |
| | GACCTGAGCAACCTGCACGAGATCGCCAGCGGCGGCGCCCCCCTGAGCAAGGAGGTGGGCGAGG | |
| | CCGTGGCCAAGCGGTTCCACCTGCCCGGCATCCGGCAGGGCTACGGCCTGACCGAGACCACCAG | |
| | CGCCATCCTGATCACCCCCGAGGGCGACGACAAGCCCGGCGCCGTGGGCAAGGTGGTGCCCTTC | |
| | TTCGAGGCCAAGGTGGTGGACCTGGACACCGGCAAGACCCTGGGCGTGAACCAGCGGGGCGAGC | |
| | TGTGCGTGCGGGGCCCCATGATCATGAGCGGCTACGTGAACAACCCCGAGGCCACCAACGCCCT | |
| | GATCGACAAGGACGGCTGGCTGCACAGCGGCGACATCGCCTACTGGGACGAGGACGAGCACTTC | |
| | TTCATCGTGGACCGGCTGAAGAGCCTGATCAAGTACAAGGGCTACCAGGTGGCCCCCGCCGAGC | |
| | TGGAGAGCATCCTGCTGCAGCACCCCAACATCTTCGACGCCGGCGTGGCCGGCCTGCCCGACGA | |
| | CGACGCCGGCGAGCTGCCCGCCGCCGTGGTGGTGCTGGAGCACGGCAAGACCATGACCGAGAAG | |
| | GAGATCGTGGACTACGTGGCCAGCCAGGTGACCACCGCCAAGAAGCTGCGGGGCGGCGTGGTGT | |
| | TCGTGGACGAGGTGCCCAAGGGCCTGACCGGCAAGCTGGACGCCCGGAAGATCCGGGAGATCCT | |
| | GATCAAGGCCAAGAAGGGCGGCAAGATCGCCGTGTGATAAGCTGCCTTCTGCGGGGCTTGCCTT | |
| | CTGGCCATGCCCTTCTTCTCTCCCTTGCACCTGTACCTCTTGGTCTTTGAATAAAGCCTGAGTA | |
| | GGAAGGCGGAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAAAA | |
| | A | |
| DNA | X⁶X⁵X⁴X³X²X¹X⁻¹ATAGTGAGTCGTATTA | |
| template - | X is any nucleotide | |
| antisense | | |
| strand | | |
| positions | | |
| −1 to +2 | | |
| (bold) | | |
| Consensus | TAATACGACTCACTATAGGGAGA | 7 |
| sequence | | |
| for T7 RNAP | | |
| - sense | | |
| strand | | |
| positions | | |
| −17 to +6 | | |
| (Bold | | |
| indicates | | |
| position | | |
| +1) | | |
| Consensus | ATTTAGGTGACACTATAGAAGAA | 8 |
| sequence | | |
| for SP6 | | |
| RNAP - | | |
| sense | | |
| strand | | |
| positions | | |
| −17 to +6, | | |
| (Bold | | |
| indicates | | |
| position | | |
| +1) | | |
| Consensus | AATTAACCCTCACTAAAGGGAGA | 9 |
| sequence | | |
| for T3 RNAP | | |
| - sense | | |
| strand | | |
| positions | | |
| −17 to +6, | | |
| (Bold | | |
| indicates | | |
| position | | |
| +1) | | |
| GC DNA | CGGGAAATAAGAGAGAAAAGAAGAGTAAGAAGAAATATAAGAGCCACCATGGAGGACGCCAAGA | 10 |
| template - | ACATCAAGAAGGGCCCCGCCCCCTTCTACCCCCTGGAGGACGGCACCGCCGGCGAGCAGCTGCA | |
| sense | CAAGGCCATGAAGCGGTACGCCCTGGTGCCCGGCACCATCGCCTTCACCGACGCCCACATCGAG | |
| strand | GTGGACATCACCTACGCCGAGTACTTCGAGATGAGCGTGCGGCTGGCCGAGGCCATGAAGCGGT | |
| RNA-coding | ACGGCCTGAACACCAACCACCGGATCGTGGTGTGCAGCGAGAACAGCCTGCAGTTCTTCATGCC | |
| region | CGTGCTGGGCGCCCTGTTCATCGGCGTGGCCGTGGCCCCCGCCAACGACATCTACAACGAGCGG | |
| | GAGCTGCTGAACAGCATGGGCATCAGCCAGCCCACCGTGGTGTTCGTGAGCAAGAAGGGCCTGC | |
| | AGAAGATCCTGAACGTGCAGAAGAAGCTGCCCATCATCCAGAAGATCATCATCATGGACAGCAA | |
| | GACCGACTACCAGGGCTTCCAGAGCATGTACACCTTCGTGACCAGCCACCTGCCCCCCGGCTTC | |
| | AACGAGTACGACTTCGTGCCCGAGAGCTTCGACCGGGACAAGACCATCGCCCTGATCATGAACA | |
| | GCAGCGGCAGCACCGGCCTGCCCAAGGGCGTGGCCCTGCCCCACCGGACCGCCTGCGTGCGGTT | |
| | CAGCCACGCCCGGGACCCCATCTTCGGCAACCAGATCATCCCCGACACCGCCATCCTGAGCGTG | |
| | GTGCCCTTCCACCACGGCTTCGGCATGTTCACCACCCTGGGCTACCTGATCTGCGGCTTCCGGG | |
| | TGGTGCTGATGTACCGGTTCGAGGAGGAGCTGTTCCTGCGGAGCCTGCAGGACTACAAGATCCA | |
| | GAGCGCCCTGCTGGTGCCCACCCTGTTCAGCTTCTTCGCCAAGAGCACCCTGATCGACAAGTAC | |
| | GACCTGAGCAACCTGCACGAGATCGCCAGCGGCGGCGCCCCCCTGAGCAAGGAGGTGGGCGAGG | |
| | CCGTGGCCAAGCGGTTCCACCTGCCCGGCATCCGGCAGGGCTACGGCCTGACCGAGACCACCAG | |
| | CGCCATCCTGATCACCCCCGAGGGCGACGACAAGCCCGGCGCCGTGGGCAAGGTGGTGCCCTTC | |
| | TTCGAGGCCAAGGTGGTGGACCTGGACACCGGCAAGACCCTGGGCGTGAACCAGCGGGGCGAGC | |
| | TGTGCGTGCGGGGCCCCATGATCATGAGCGGCTACGTGAACAACCCCGAGGCCACCAACGCCCT | |
| | GATCGACAAGGACGGCTGGCTGCACAGCGGCGACATCGCCTACTGGGACGAGGACGAGCACTTC | |
| | TTCATCGTGGACCGGCTGAAGAGCCTGATCAAGTACAAGGGCTACCAGGTGGCCCCCGCCGAGC | |
| | TGGAGAGCATCCTGCTGCAGCACCCCAACATCTTCGACGCCGGCGTGGCCGGCCTGCCCGACGA | |
| | CGACGCCGGCGAGCTGCCCGCCGCCGTGGTGGTGCTGGAGCACGGCAAGACCATGACCGAGAAG | |
| | GAGATCGTGGACTACGTGGCCAGCCAGGTGACCACCGCCAAGAAGCTGCGGGGCGGCGTGGTGT | |
| | TCGTGGACGAGGTGCCCAAGGGCCTGACCGGCAAGCTGGACGCCCGGAAGATCCGGGAGATCCT | |
| | GATCAAGGCCAAGAAGGGCGGCAAGATCGCCGTGTGATAAGCTGCCTTCTGCGGGGCTTGCCTT | |
| | CTGGCCATGCCCTTCTTCTCTCCCTTGCACCTGTACCTCTTGGTCTTTGAATAAAGCCTGAGTA | |
| | GGAAGGCGGAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAAAA | |
| | A | |
| AC DNA | TGGGAAATAAGAGAGAAAAGAAGAGTAAGAAGAAATATAAGAGCCACCATGGAGGACGCCAAGA | 11 |
| template - | ACATCAAGAAGGGCCCCGCCCCCTTCTACCCCCTGGAGGACGGCACCGCCGGCGAGCAGCTGCA | |
| sense | CAAGGCCATGAAGCGGTACGCCCTGGTGCCCGGCACCATCGCCTTCACCGACGCCCACATCGAG | |
| strand | GTGGACATCACCTACGCCGAGTACTTCGAGATGAGCGTGCGGCTGGCCGAGGCCATGAAGCGGT | |
| RNA-coding | ACGGCCTGAACACCAACCACCGGATCGTGGTGTGCAGCGAGAACAGCCTGCAGTTCTTCATGCC | |
| region | CGTGCTGGGCGCCCTGTTCATCGGCGTGGCCGTGGCCCCCGCCAACGACATCTACAACGAGCGG | |
| | GAGCTGCTGAACAGCATGGGCATCAGCCAGCCCACCGTGGTGTTCGTGAGCAAGAAGGGCCTGC | |
| | AGAAGATCCTGAACGTGCAGAAGAAGCTGCCCATCATCCAGAAGATCATCATCATGGACAGCAA | |
| | GACCGACTACCAGGGCTTCCAGAGCATGTACACCTTCGTGACCAGCCACCTGCCCCCCGGCTTC | |
| | AACGAGTACGACTTCGTGCCCGAGAGCTTCGACCGGGACAAGACCATCGCCCTGATCATGAACA | |
| | GCAGCGGCAGCACCGGCCTGCCCAAGGGCGTGGCCCTGCCCCACCGGACCGCCTGCGTGCGGTT | |
| | CAGCCACGCCCGGGACCCCATCTTCGGCAACCAGATCATCCCCGACACCGCCATCCTGAGCGTG | |
| | GTGCCCTTCCACCACGGCTTCGGCATGTTCACCACCCTGGGCTACCTGATCTGCGGCTTCCGGG | |
| | TGGTGCTGATGTACCGGTTCGAGGAGGAGCTGTTCCTGCGGAGCCTGCAGGACTACAAGATCCA | |
| | GAGCGCCCTGCTGGTGCCCACCCTGTTCAGCTTCTTCGCCAAGAGCACCCTGATCGACAAGTAC | |
| | GACCTGAGCAACCTGCACGAGATCGCCAGCGGCGGCGCCCCCCTGAGCAAGGAGGTGGGCGAGG | |
| | CCGTGGCCAAGCGGTTCCACCTGCCCGGCATCCGGCAGGGCTACGGCCTGACCGAGACCACCAG | |
| | CGCCATCCTGATCACCCCCGAGGGCGACGACAAGCCCGGCGCCGTGGGCAAGGTGGTGCCCTTC | |
| | TTCGAGGCCAAGGTGGTGGACCTGGACACCGGCAAGACCCTGGGCGTGAACCAGCGGGGCGAGC | |
| | TGTGCGTGCGGGGCCCCATGATCATGAGCGGCTACGTGAACAACCCCGAGGCCACCAACGCCCT | |
| | GATCGACAAGGACGGCTGGCTGCACAGCGGCGACATCGCCTACTGGGACGAGGACGAGCACTTC | |
| | TTCATCGTGGACCGGCTGAAGAGCCTGATCAAGTACAAGGGCTACCAGGTGGCCCCCGCCGAGC | |
| | TGGAGAGCATCCTGCTGCAGCACCCCAACATCTTCGACGCCGGCGTGGCCGGCCTGCCCGACGA | |
| | CGACGCCGGCGAGCTGCCCGCCGCCGTGGTGGTGCTGGAGCACGGCAAGACCATGACCGAGAAG | |
| | GAGATCGTGGACTACGTGGCCAGCCAGGTGACCACCGCCAAGAAGCTGCGGGGCGGCGTGGTGT | |
| | TCGTGGACGAGGTGCCCAAGGGCCTGACCGGCAAGCTGGACGCCCGGAAGATCCGGGAGATCCT | |
| | GATCAAGGCCAAGAAGGGCGGCAAGATCGCCGTGTGATAAGCTGCCTTCTGCGGGGCTTGCCTT | |
| | CTGGCCATGCCCTTCTTCTCTCCCTTGCACCTGTACCTCTTGGTCTTTGAATAAAGCCTGAGTA | |
| | GGAAGGCGGAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAAAA | |
| | A | |
| TT DNA | AAGGAAATAAGAGAGAAAAGAAGAGTAAGAAGAAATATAAGAGCCACCATGGAGGACGCCAAGA | 12 |
| template - | ACATCAAGAAGGGCCCCGCCCCCTTCTACCCCCTGGAGGACGGCACCGCCGGCGAGCAGCTGCA | |
| sense | CAAGGCCATGAAGCGGTACGCCCTGGTGCCCGGCACCATCGCCTTCACCGACGCCCACATCGAG | |
| strand | GTGGACATCACCTACGCCGAGTACTTCGAGATGAGCGTGCGGCTGGCCGAGGCCATGAAGCGGT | |
| RNA-coding | ACGGCCTGAACACCAACCACCGGATCGTGGTGTGCAGCGAGAACAGCCTGCAGTTCTTCATGCC | |
| region | CGTGCTGGGCGCCCTGTTCATCGGCGTGGCCGTGGCCCCCGCCAACGACATCTACAACGAGCGG | |
| | GAGCTGCTGAACAGCATGGGCATCAGCCAGCCCACCGTGGTGTTCGTGAGCAAGAAGGGCCTGC | |
| | AGAAGATCCTGAACGTGCAGAAGAAGCTGCCCATCATCCAGAAGATCATCATCATGGACAGCAA | |
| | GACCGACTACCAGGGCTTCCAGAGCATGTACACCTTCGTGACCAGCCACCTGCCCCCCGGCTTC | |
| | AACGAGTACGACTTCGTGCCCGAGAGCTTCGACCGGGACAAGACCATCGCCCTGATCATGAACA | |
| | GCAGCGGCAGCACCGGCCTGCCCAAGGGCGTGGCCCTGCCCCACCGGACCGCCTGCGTGCGGTT | |
| | CAGCCACGCCCGGGACCCCATCTTCGGCAACCAGATCATCCCCGACACCGCCATCCTGAGCGTG | |
| | GTGCCCTTCCACCACGGCTTCGGCATGTTCACCACCCTGGGCTACCTGATCTGCGGCTTCCGGG | |
| | TGGTGCTGATGTACCGGTTCGAGGAGGAGCTGTTCCTGCGGAGCCTGCAGGACTACAAGATCCA | |
| | GAGCGCCCTGCTGGTGCCCACCCTGTTCAGCTTCTTCGCCAAGAGCACCCTGATCGACAAGTAC | |
| | GACCTGAGCAACCTGCACGAGATCGCCAGCGGCGGCGCCCCCCTGAGCAAGGAGGTGGGCGAGG | |
| | CCGTGGCCAAGCGGTTCCACCTGCCCGGCATCCGGCAGGGCTACGGCCTGACCGAGACCACCAG | |
| | CGCCATCCTGATCACCCCCGAGGGCGACGACAAGCCCGGCGCCGTGGGCAAGGTGGTGCCCTTC | |
| | TTCGAGGCCAAGGTGGTGGACCTGGACACCGGCAAGACCCTGGGCGTGAACCAGCGGGGCGAGC | |
| | TGTGCGTGCGGGGCCCCATGATCATGAGCGGCTACGTGAACAACCCCGAGGCCACCAACGCCCT | |
| | GATCGACAAGGACGGCTGGCTGCACAGCGGCGACATCGCCTACTGGGACGAGGACGAGCACTTC | |
| | TTCATCGTGGACCGGCTGAAGAGCCTGATCAAGTACAAGGGCTACCAGGTGGCCCCCGCCGAGC | |
| | TGGAGAGCATCCTGCTGCAGCACCCCAACATCTTCGACGCCGGCGTGGCCGGCCTGCCCGACGA | |
| | CGACGCCGGCGAGCTGCCCGCCGCCGTGGTGGTGCTGGAGCACGGCAAGACCATGACCGAGAAG | |
| | GAGATCGTGGACTACGTGGCCAGCCAGGTGACCACCGCCAAGAAGCTGCGGGGCGGCGTGGTGT | |
| | TCGTGGACGAGGTGCCCAAGGGCCTGACCGGCAAGCTGGACGCCCGGAAGATCCGGGAGATCCT | |
| | GATCAAGGCCAAGAAGGGCGGCAAGATCGCCGTGTGATAAGCTGCCTTCTGCGGGGCTTGCCTT | |
| | CTGGCCATGCCCTTCTTCTCTCCCTTGCACCTGTACCTCTTGGTCTTTGAATAAAGCCTGAGTA | |
| | GGAAGGCGGAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAAAA | |
| | A | |
| TG DNA | ACGGAAATAAGAGAGAAAAGAAGAGTAAGAAGAAATATAAGAGCCACCATGGAGGACGCCAAGA | 13 |
| template - | ACATCAAGAAGGGCCCCGCCCCCTTCTACCCCCTGGAGGACGGCACCGCCGGCGAGCAGCTGCA | |
| sense | CAAGGCCATGAAGCGGTACGCCCTGGTGCCCGGCACCATCGCCTTCACCGACGCCCACATCGAG | |
| strand | GTGGACATCACCTACGCCGAGTACTTCGAGATGAGCGTGCGGCTGGCCGAGGCCATGAAGCGGT | |
| RNA-coding | ACGGCCTGAACACCAACCACCGGATCGTGGTGTGCAGCGAGAACAGCCTGCAGTTCTTCATGCC | |
| region | CGTGCTGGGCGCCCTGTTCATCGGCGTGGCCGTGGCCCCCGCCAACGACATCTACAACGAGCGG | |
| | GAGCTGCTGAACAGCATGGGCATCAGCCAGCCCACCGTGGTGTTCGTGAGCAAGAAGGGCCTGC | |
| | AGAAGATCCTGAACGTGCAGAAGAAGCTGCCCATCATCCAGAAGATCATCATCATGGACAGCAA | |
| | GACCGACTACCAGGGCTTCCAGAGCATGTACACCTTCGTGACCAGCCACCTGCCCCCCGGCTTC | |
| | AACGAGTACGACTTCGTGCCCGAGAGCTTCGACCGGGACAAGACCATCGCCCTGATCATGAACA | |
| | GCAGCGGCAGCACCGGCCTGCCCAAGGGCGTGGCCCTGCCCCACCGGACCGCCTGCGTGCGGTT | |
| | CAGCCACGCCCGGGACCCCATCTTCGGCAACCAGATCATCCCCGACACCGCCATCCTGAGCGTG | |
| | GTGCCCTTCCACCACGGCTTCGGCATGTTCACCACCCTGGGCTACCTGATCTGCGGCTTCCGGG | |
| | TGGTGCTGATGTACCGGTTCGAGGAGGAGCTGTTCCTGCGGAGCCTGCAGGACTACAAGATCCA | |
| | GAGCGCCCTGCTGGTGCCCACCCTGTTCAGCTTCTTCGCCAAGAGCACCCTGATCGACAAGTAC | |
| | GACCTGAGCAACCTGCACGAGATCGCCAGCGGCGGCGCCCCCCTGAGCAAGGAGGTGGGCGAGG | |
| | CCGTGGCCAAGCGGTTCCACCTGCCCGGCATCCGGCAGGGCTACGGCCTGACCGAGACCACCAG | |
| | CGCCATCCTGATCACCCCCGAGGGCGACGACAAGCCCGGCGCCGTGGGCAAGGTGGTGCCCTTC | |
| | TTCGAGGCCAAGGTGGTGGACCTGGACACCGGCAAGACCCTGGGCGTGAACCAGCGGGGCGAGC | |
| | TGTGCGTGCGGGGCCCCATGATCATGAGCGGCTACGTGAACAACCCCGAGGCCACCAACGCCCT | |
| | GATCGACAAGGACGGCTGGCTGCACAGCGGCGACATCGCCTACTGGGACGAGGACGAGCACTTC | |
| | TTCATCGTGGACCGGCTGAAGAGCCTGATCAAGTACAAGGGCTACCAGGTGGCCCCCGCCGAGC | |
| | TGGAGAGCATCCTGCTGCAGCACCCCAACATCTTCGACGCCGGCGTGGCCGGCCTGCCCGACGA | |
| | CGACGCCGGCGAGCTGCCCGCCGCCGTGGTGGTGCTGGAGCACGGCAAGACCATGACCGAGAAG | |
| | GAGATCGTGGACTACGTGGCCAGCCAGGTGACCACCGCCAAGAAGCTGCGGGGCGGCGTGGTGT | |
| | TCGTGGACGAGGTGCCCAAGGGCCTGACCGGCAAGCTGGACGCCCGGAAGATCCGGGAGATCCT | |
| | GATCAAGGCCAAGAAGGGCGGCAAGATCGCCGTGTGATAAGCTGCCTTCTGCGGGGCTTGCCTT | |
| | CTGGCCATGCCCTTCTTCTCTCCCTTGCACCTGTACCTCTTGGTCTTTGAATAAAGCCTGAGTA | |
| | GGAAGGCGGAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAAAA | |
| | A | |
| TA DNA | ATGGAAATAAGAGAGAAAAGAAGAGTAAGAAGAAATATAAGAGCCACCATGGAGGACGCCAAGA | 14 |
| template - | ACATCAAGAAGGGCCCCGCCCCCTTCTACCCCCTGGAGGACGGCACCGCCGGCGAGCAGCTGCA | |
| sense | CAAGGCCATGAAGCGGTACGCCCTGGTGCCCGGCACCATCGCCTTCACCGACGCCCACATCGAG | |
| strand | GTGGACATCACCTACGCCGAGTACTTCGAGATGAGCGTGCGGCTGGCCGAGGCCATGAAGCGGT | |
| RNA-coding | ACGGCCTGAACACCAACCACCGGATCGTGGTGTGCAGCGAGAACAGCCTGCAGTTCTTCATGCC | |
| region | CGTGCTGGGCGCCCTGTTCATCGGCGTGGCCGTGGCCCCCGCCAACGACATCTACAACGAGCGG | |
| | GAGCTGCTGAACAGCATGGGCATCAGCCAGCCCACCGTGGTGTTCGTGAGCAAGAAGGGCCTGC | |
| | AGAAGATCCTGAACGTGCAGAAGAAGCTGCCCATCATCCAGAAGATCATCATCATGGACAGCAA | |
| | GACCGACTACCAGGGCTTCCAGAGCATGTACACCTTCGTGACCAGCCACCTGCCCCCCGGCTTC | |
| | AACGAGTACGACTTCGTGCCCGAGAGCTTCGACCGGGACAAGACCATCGCCCTGATCATGAACA | |
| | GCAGCGGCAGCACCGGCCTGCCCAAGGGCGTGGCCCTGCCCCACCGGACCGCCTGCGTGCGGTT | |
| | CAGCCACGCCCGGGACCCCATCTTCGGCAACCAGATCATCCCCGACACCGCCATCCTGAGCGTG | |
| | GTGCCCTTCCACCACGGCTTCGGCATGTTCACCACCCTGGGCTACCTGATCTGCGGCTTCCGGG | |
| | TGGTGCTGATGTACCGGTTCGAGGAGGAGCTGTTCCTGCGGAGCCTGCAGGACTACAAGATCCA | |
| | GAGCGCCCTGCTGGTGCCCACCCTGTTCAGCTTCTTCGCCAAGAGCACCCTGATCGACAAGTAC | |
| | GACCTGAGCAACCTGCACGAGATCGCCAGCGGCGGCGCCCCCCTGAGCAAGGAGGTGGGCGAGG | |
| | CCGTGGCCAAGCGGTTCCACCTGCCCGGCATCCGGCAGGGCTACGGCCTGACCGAGACCACCAG | |
| | CGCCATCCTGATCACCCCCGAGGGCGACGACAAGCCCGGCGCCGTGGGCAAGGTGGTGCCCTTC | |
| | TTCGAGGCCAAGGTGGTGGACCTGGACACCGGCAAGACCCTGGGCGTGAACCAGCGGGGCGAGC | |
| | TGTGCGTGCGGGGCCCCATGATCATGAGCGGCTACGTGAACAACCCCGAGGCCACCAACGCCCT | |
| | GATCGACAAGGACGGCTGGCTGCACAGCGGCGACATCGCCTACTGGGACGAGGACGAGCACTTC | |
| | TTCATCGTGGACCGGCTGAAGAGCCTGATCAAGTACAAGGGCTACCAGGTGGCCCCCGCCGAGC | |
| | TGGAGAGCATCCTGCTGCAGCACCCCAACATCTTCGACGCCGGCGTGGCCGGCCTGCCCGACGA | |
| | CGACGCCGGCGAGCTGCCCGCCGCCGTGGTGGTGCTGGAGCACGGCAAGACCATGACCGAGAAG | |
| | GAGATCGTGGACTACGTGGCCAGCCAGGTGACCACCGCCAAGAAGCTGCGGGGCGGCGTGGTGT | |
| | TCGTGGACGAGGTGCCCAAGGGCCTGACCGGCAAGCTGGACGCCCGGAAGATCCGGGAGATCCT | |
| | GATCAAGGCCAAGAAGGGCGGCAAGATCGCCGTGTGATAAGCTGCCTTCTGCGGGGCTTGCCTT | |
| | CTGGCCATGCCCTTCTTCTCTCCCTTGCACCTGTACCTCTTGGTCTTTGAATAAAGCCTGAGTA | |
| | GGAAGGCGGAAAAAAAAAAAAAAAAAAAAAAAAAAAAAAAAAAAAAAAAAAAAAAAAAAAAAAA | |
| | AAAAAAAAAAAAAAAAAAAAAAAAAAAAAAAAAAAAAAAAAAAAAAAAAAAAAAAAAAAAAAAA | |
| | A | |
| Exemplary | TAATACGACTCACTATAAGGGAA | 15 |
| T7 RNAP | | |
| sequence - | | |
| sense | | |
| strand | | |
| (antisense | | |
| strand, | | |
| TC start) | | |
| positions | | |
| −17 to +6, | | |

1. A method of producing 5′-capped RNA molecules in an in vitro transcription (IVT) reaction comprising the steps of:
(i) mixing a capping primer, or a stereoisomer, tautomer, or salt form thereof, with a polynucleotide template, nucleoside 5′-triphosphates (NTPs), and an RNA polymerase,
wherein the capping primer comprises a structure according to Formula II: 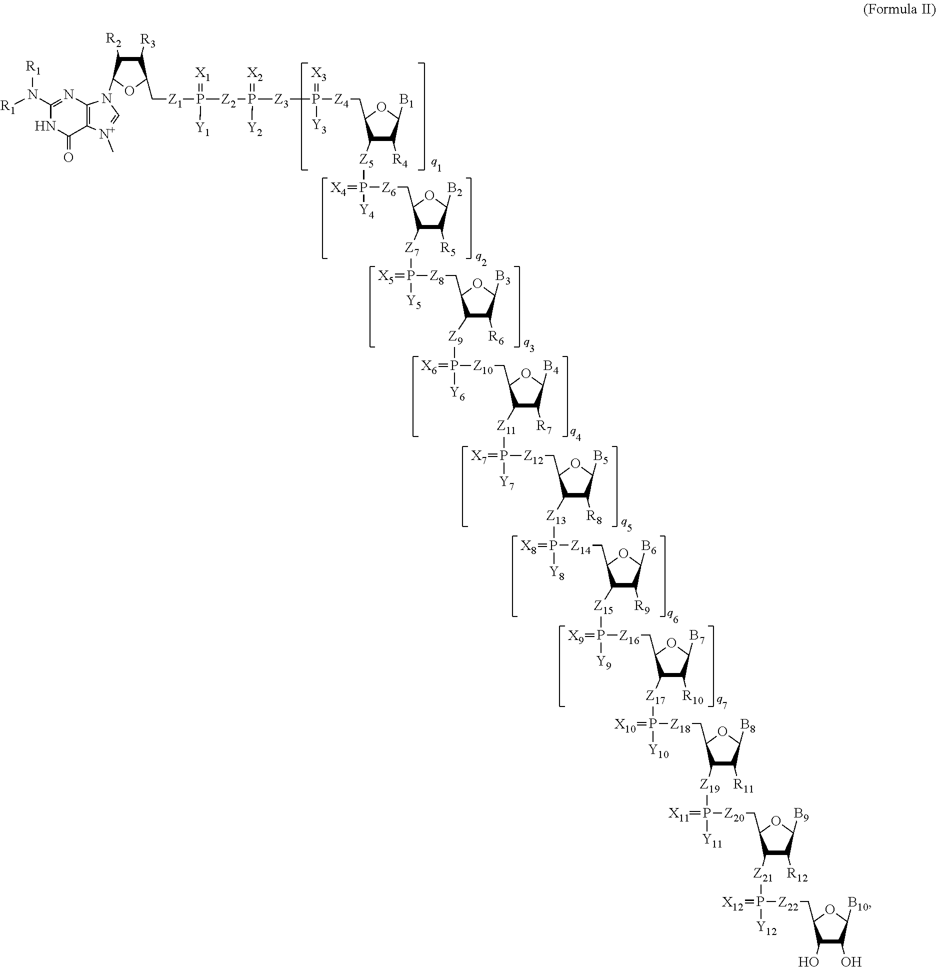
wherein:
q1 to q7 are 0;
B8 is a nucleobase selected from the group consisting of adenine, guanine, thymine, uracil, cytosine, hypoxanthine, and any derivative or analog of the foregoing;
B9 is a nucleobase selected from the group consisting of adenine, uracil, cytosine, and any derivative or analog of the foregoing;
B10 is a nucleobase selected from the group consisting of adenine, guanine, uracil, cytosine, and any derivative or analog of the foregoing;
R1 are each independently H, alkyl, acyl, benzyl, or a cleavable unit;
R2 is H, OH, alkyl, O-alkyl, O-acyl, O-benzyl, O-MOE, halogen, LNA, a linker, a cleavable unit, or a detectable marker;
R3 is H, OH, alkyl, O-alkyl, O-acyl, O-benzyl, O-MOE, halogen, a linker, a cleavable unit, or a detectable marker;
X1 to X2, and X10 to X12 are each independently O or S;
Y1 to Y2, and Y10 to Y12 are each independently O⁻, S⁻, BH₃⁻, aryl, alkyl, O-alkyl, or O-aryl; and
Z1 to Z3, and Z18 to Z22 are each independently O or S, NH, CH₂, C(halogen)₂ or CH(halogen);
wherein the polynucleotide template comprises a nucleobase at position −1 immediately adjacent to and downstream (3′) of a transcriptional start site, and a nucleobase at each of position +1, position +2 and position +3 immediately adjacent to and upstream (5′) of the transcriptional start site, and wherein
B8 is complementary or mismatched to a nucleobase at position −1 of the polynucleotide template;
B9 is complementary to a nucleobase at position +1 of the polynucleotide template;
B10 is complementary to a nucleobase at position +2 of the polynucleotide template; and B8,
B9, and B10 are not fully complementary to nucleobases at positions +1 to +3 of the polynucleotide template; and
(ii) incubating the mixture under transcription conditions, thereby resulting in synthesis of a 5′-capped RNA molecule.

2. The method of claim 1, wherein the capping primer consists of the structure according to Formula II.

3. The method of claim 1, wherein B8 is not N6-modified adenine.

4. The method of claim 1, wherein B8 is a nucleobase selected from the group consisting of adenine, uracil, thymine, cytosine, 5-methylcytosine, guanine, and hypoxanthine.

5. The method of claim 1, wherein B9 is selected from the group consisting of adenine, cytosine, 5-methylcytosine, thymine and uracil.

6. The method of claim 1, wherein B9 is selected from the group consisting of adenine, cytosine, and uracil.

7. The method of claim 1, wherein B9 is adenine or uracil.

8. The method of claim 1, wherein B10 is selected from the group consisting of adenine, guanine, cytosine, 5-methylcytosine, thymine and uracil.

9. The method of claim 1, wherein B10 is selected from the group consisting of adenine, guanine, cytosine, and uracil.

10. The method of claim 1, wherein R1 are each independently H or C1-C6 alkyl.

11. The method of claim 1, wherein R1 are each H.

12. The method of claim 1, wherein R2 is H, OH, O—(C1-C6 alkyl), O-MOE, fluoro, or LNA.

13. The method of claim 1, wherein R2 is OH.

14. The method of claim 1, wherein R3 is H, OH, O—(C1-C6 alkyl), O-MOE, or fluoro.

15. The method of claim 1, wherein R3 is OH or OMe.

16. The method of claim 1, wherein R11 and R12 are each independently H, OH, O—(C1-C6 alkyl), O-MOE, fluoro, or LNA.

17. The method of claim 1, wherein R11 is H, OH, OMe, O-MOE, fluoro, or LNA, and R12 is OH or OMe.

18. The method of claim 1, wherein R11 is OMe, and R12 is OMe.

19. The method of claim 1, wherein
X1 to X2, and X10 to X12 are each independently O;
Y1 to Y2, and Y10 to Y12 are each independently O⁻ or S⁻; and
Z1 to Z3, and Z18 to Z22 are each independently O.

20. The method of claim 1, wherein B8 is complementary to a nucleobase at position −1 of the polynucleotide template.

21. The method of claim 1, wherein B8 is mismatched to a nucleobase at position −1 of the polynucleotide template.

22. The method of claim 1, wherein the polynucleotide template is a DNA template.

23. The method of claim 1, wherein the RNA polymerase is a T7 RNA polymerase or a variant thereof.

24. The method of claim 1, wherein the NTPs comprise adenosine triphosphate (ATP) or a derivative or analog thereof, guanosine triphosphate (GTP) or a derivative or analog thereof, cytosine triphosphate (CTP) or a derivative or analog thereof, and uridine triphosphate (UTP) or a derivative or analog thereof, each at an approximately equimolar concentration, optionally wherein the derivative or analog of UTP is pseudouridine triphosphate (pseudo-UTP) or N1-methylpseudouridine triphosphate (N1-pseudo-UTP).

25. The method of claim 1, wherein the method further comprises a step of purifying the 5′-capped RNA molecules.

26. 5′-capped RNA molecules produced by the method of claim 1.

27. The 5′-capped RNA molecules of claim 26, comprising 5′-capped messenger RNAs (mRNAs).

28. A cell comprising the 5′-capped RNA molecules of claim 26.

29. A pharmaceutical composition comprising the 5′-capped RNA molecules of claim 26.

30. The pharmaceutical composition comprising the 5′-capped RNA molecules of claim 29, wherein the pharmaceutical composition is formulated as a lipid nanoparticle (LNP).
